SEQUENCE LISTING (462 sequences)

SEQ ID NO: 1 - protein, 12 aa, Homo sapiens
DLQGRDEQSEEK

SEQ ID NO: 2 - protein, 12 aa, Homo sapiens
GVTFNVTTVDTK

SEQ ID NO: 3 - protein, 16 aa, Homo sapiens
MNVDHEVNLLVEEIHR

SEQ ID NO: 4 - protein, 16 aa, Homo sapiens
TVEGAGSIAAATGFVK

SEQ ID NO: 5 - protein, 13 aa, Homo sapiens
IFQILYANEGESK

SEQ ID NO: 6 - protein, 9 aa, Homo sapiens
TLEAQLTPR

SEQ ID NO: 7 - protein, 23 aa, Homo sapiens
ALGISPFHEHAEVVFTANDSGPR

SEQ ID NO: 8 - protein, 9 aa, Homo sapiens
VYYDISTAK

SEQ ID NO: 9 - protein, 13 aa, Homo sapiens
AADDTWEPFASGK

SEQ ID NO: 10 - protein, 10 aa, Homo sapiens
LEGWLSLPVR

SEQ ID NO: 11 - protein, 37 aa, Homo sapiens
AYHPHCFTCVVCARPLEGTSFIVDQANRPHCVPDYHK

SEQ ID NO: 12 - protein, 19 aa, Homo sapiens
GGLFADIASHPWQAAIFAK

SEQ ID NO: 13 - protein, 12 aa, Homo sapiens
ALETMGLWVDCR

SEQ ID NO: 14 - protein, 15 aa, Homo sapiens
DSDWPFCSDEDWNYK

SEQ ID NO: 15 - protein, 10 aa, Homo sapiens
ILFYDSEQDK

SEQ ID NO: 16 - protein, 11 aa, Homo sapiens
ILMVGLDAAGK

SEQ ID NO: 17 - protein, 10 aa, Homo sapiens
GNFNYVEFTR

SEQ ID NO: 18 - protein, 13 aa, Homo sapiens
LSNDMMGSYAEMK

SEQ ID NO: 19 - protein, 16 aa, Homo sapiens
TTFANVVNVCGNQSIR

SEQ ID NO: 20 - protein, 9 aa, Homo sapiens
GYILVGQAK

SEQ ID NO: 21 - protein, 7 aa, Homo sapiens
VIEEEQR

SEQ ID NO: 22 - protein, 12 aa, Homo sapiens
NSNPALNDNLEK

SEQ ID NO: 23 - protein, 9 aa, Homo sapiens
ELLETVVNR

SEQ ID NO: 24 - protein, 13 aa, Homo sapiens
TAFQEALDAAGDK

SEQ ID NO: 25 - protein, 8 aa, Homo sapiens
EEVFIQQR

SEQ ID NO: 26 - protein, 13 aa, Homo sapiens
LGNQEPGGQTALK

SEQ ID NO: 27 - protein, 8 aa, Homo sapiens
ELPSFLGK

SEQ ID NO: 28 - protein, 10 aa, Homo sapiens
GLESTTLADK

SEQ ID NO: 29 - protein, 11 aa, Homo sapiens
VNPALAELNLR

SEQ ID NO: 30 - protein, 20 aa, Homo sapiens
CECGSCVCIQPGSYGDTCEK

SEQ ID NO: 31 - protein, 18 aa, Homo sapiens
LAALNPESNTAGLDIFAK

SEQ ID NO: 32 - protein, 13 aa, Homo sapiens
CANLFEALVGTLK

SEQ ID NO: 33 - protein, 9 aa, Homo sapiens
EYAPGETVK

SEQ ID NO: 34 - protein, 12 aa, Homo sapiens
TATLCPQSCEER

SEQ ID NO: 35 - protein, 24 aa, Homo sapiens
TPDFCAMSCPPSLVYNHCEHGCPR

SEQ ID NO: 36 - protein, 10 aa, Homo sapiens
GYLDDPTVPR

SEQ ID NO: 37 - protein, 14 aa, Homo sapiens
CLPSACEVVTGSPR

SEQ ID NO: 38 - protein, 13 aa, Homo sapiens
IYVDDGLISLQVK

SEQ ID NO: 39 - protein, 20 aa, Homo sapiens
QLGLPGPPDVPDHAAYHPFR

SEQ ID NO: 40 - protein, 12 aa, Homo sapiens
INVYYNEATGGK

SEQ ID NO: 41 - protein, 11 aa, Homo sapiens
AGFSWIEVTFK

SEQ ID NO: 42 - protein, 12 aa, Homo sapiens
IMNTFSVVPSPK

SEQ ID NO: 43 - protein, 21 aa, Homo sapiens
FDGALNVDLTEFQTNLVPYPR

SEQ ID NO: 44 - protein, 18 aa, Homo sapiens
TLDEFTIIQNLQPQYQFR

SEQ ID NO: 45 - protein, 20 aa, Homo sapiens
RLDYQEGPPGVEISCWSVEL

SEQ ID NO: 46 - protein, 22 aa, Homo sapiens
QGPVNLLSDPEQGVEVTGQYER

SEQ ID NO: 47 - protein, 27 aa, Homo sapiens
FWEVISDEHGIDPTGTYHGDSDLQLDR

SEQ ID NO: 48 - protein, 15 aa, Homo sapiens
ALTVPELTQQMFDAK

SEQ ID NO: 49 - protein, 9 aa, Homo sapiens
YESLTDPSK

SEQ ID NO: 50 - protein, 12 aa, Homo sapiens
ISVYYNEATGGK

SEQ ID NO: 51 - protein, 7 aa, Homo sapiens
FDLMYAK

SEQ ID NO: 52 - protein, 14 aa, Homo sapiens
LHFFMPGFAPLTSR

SEQ ID NO: 53 - protein, 9 aa, Homo sapiens
TAVCDIPPR

SEQ ID NO: 54 - protein, 7 aa, Homo sapiens
DFLQSLK

SEQ ID NO: 55 - protein, 8 aa, Homo sapiens
SPPGVCSR

SEQ ID NO: 56 - protein, 27 aa, Homo sapiens
HPPVVMNGAVADGILASYATGSSVEYR

SEQ ID NO: 57 - protein, 14 aa, Homo sapiens
NSSYFVEWIPNNVK

SEQ ID NO: 58 - protein, 8 aa, Homo sapiens
LCDNLSTK

SEQ ID NO: 59 - protein, 7 aa, Homo sapiens
LCMAALK

SEQ ID NO: 60 - protein, 21 aa, Homo sapiens
SYLSMVGSCCTSASPTVCFLK

SEQ ID NO: 61 - protein, 20 aa, Homo sapiens
SCESNSPFPVHPGTAECCTK

SEQ ID NO: 62 - protein, 15 aa, Homo sapiens
GPALLVLGPDAIQLR

SEQ ID NO: 63 - protein, 21 aa, Homo sapiens
KGEVLPLPEANFPSFPLPHHK

SEQ ID NO: 64 - protein, 7 aa, Homo sapiens
YTFELSR

SEQ ID NO: 65 - protein, 23 aa, Homo sapiens
HQPQEFPTYVEPTNDEICEAFRK

SEQ ID NO: 66 - protein, 15 aa, Homo sapiens
GEVQAMLGQSTEELR

SEQ ID NO: 67 - protein, 12 aa, Homo sapiens
YKEENDDFASFR

SEQ ID NO: 68 - protein, 13 aa, Homo sapiens
EDFTSLSLVLYSR

SEQ ID NO: 69 - protein, 18 aa, Homo sapiens
SLGECCDVEDSTTCFNAK

SEQ ID NO: 70 - protein, 11 aa, Homo sapiens
YWNDCEPPDSR

SEQ ID NO: 71 - protein, 9 aa, Homo sapiens
VDGALCMEK

SEQ ID NO: 72 - protein, 20 aa, Homo sapiens
GEVLPLPEANFPSFPLPHHK

SEQ ID NO: 73 - protein, 8 aa, Homo sapiens
ELSSFIDK

SEQ ID NO: 74 - protein, 16 aa, Homo sapiens
DSPVLIDFFEDTERYR

SEQ ID NO: 75 - protein, 19 aa, Homo sapiens
VIDFNCTTSSVSSALANTK

SEQ ID NO: 76 - protein, 8 aa, Homo sapiens
IADAHLDR

SEQ ID NO: 77 - protein, 8 aa, Homo sapiens
FEDCCQEK

SEQ ID NO: 78 - protein, 7 aa, Homo sapiens
EFSHLGK

SEQ ID NO: 79 - protein, 13 aa, Homo sapiens
LSVEIWDWDLTSR

SEQ ID NO: 80 - protein, 12 aa, Homo sapiens
CCESASEDCMAK

SEQ ID NO: 81 - protein, 7 aa, Homo sapiens
VLEPTLK

SEQ ID NO: 82 - protein, 18 aa, Homo sapiens
GQELCADYSENTFTEYKK

SEQ ID NO: 83 - protein, 8 aa, Homo sapiens
ELPEHTVK

SEQ ID NO: 84 - protein, 15 aa, Homo sapiens
KFPSGTFEQVSQLVK

SEQ ID NO: 85 - protein, 11 aa, Homo sapiens
HLSLLTTLSNR

SEQ ID NO: 86 - protein, 8 aa, Homo sapiens
VLDLSCNR

SEQ ID NO: 87 - protein, 17 aa, Homo sapiens
GQELCADYSENTFTEYK

SEQ ID NO: 88 - protein, 12 aa, Homo sapiens
CSLNPEWNETFR

SEQ ID NO: 89 - protein, 15 aa, Homo sapiens
THVADFAPEVAWVTR

SEQ ID NO: 90 - protein, 13 aa, Homo sapiens
DGWQWFWSPSTFR

SEQ ID NO: 91 - protein, 12 aa, Homo sapiens
LGCDWVATIFSR

SEQ ID NO: 92 - protein, 20 aa, Homo sapiens
FKLDDDLEHQGGHVLDHGHK

SEQ ID NO: 93 - protein, 11 aa, Homo sapiens
LFEAEEQDLFK

SEQ ID NO: 94 - protein, 15 aa, Homo sapiens
HGSALDVLLSMGFPR

SEQ ID NO: 95 - protein, 15 aa, Homo sapiens
AYWDIMISNHQNSNR

SEQ ID NO: 96 - protein, 11 aa, Homo sapiens
EALQGVGDMGR

SEQ ID NO: 97 - protein, 15 aa, Homo sapiens
VWALLYAGGPSGVAR

SEQ ID NO: 98 - protein, 10 aa, Homo sapiens
VHAYIISSLK

SEQ ID NO: 99 - protein, 9 aa, Homo sapiens
LPFPIIDDR

SEQ ID NO: 100 - protein, 8 aa, Homo sapiens
EMPNVFGK

SEQ ID NO: 101 - protein, 29 aa, Homo sapiens
GNNILIVAHASSLEACTCQLQGLSPQNSK

SEQ ID NO: 102 - protein, 7 aa, Homo sapiens
INVIVLR

SEQ ID NO: 103 - protein, 9 aa, Homo sapiens
SHALQLNNR

SEQ ID NO: 104 - protein, 14 aa, Homo sapiens
TFYEPGEEITYSCK

SEQ ID NO: 105 - protein, 12 aa, Homo sapiens
ELVNNLGEIYQK

SEQ ID NO: 106 - protein, 14 aa, Homo sapiens
VTNYLDWIRDNMRP

SEQ ID NO: 107 - protein, 8 aa, Homo sapiens
QLELYLPK

SEQ ID NO: 108 - protein, 8 aa, Homo sapiens
NLDENYCR

SEQ ID NO: 109 - protein, 7 aa, Homo sapiens
FSVVYAK

SEQ ID NO: 110 - protein, 22 aa, Homo sapiens
YWNDCEPPDSRRPSEIVIGQCK

SEQ ID NO: 111 - protein, 11 aa, Homo sapiens
NVIGLQMGTNR

SEQ ID NO: 112 - protein, 16 aa, Homo sapiens
NLIPMDPNGLSDPYVK

SEQ ID NO: 113 - protein, 8 aa, Homo sapiens
QINDYVAK

SEQ ID NO: 114 - protein, 8 aa, Homo sapiens
NVVFVIDK

SEQ ID NO: 115 - protein, 12 aa, Homo sapiens
LKDEEISAAAIK

SEQ ID NO: 116 - protein, 17 aa, Homo sapiens
DGLLDDEEFALANHLIK

SEQ ID NO: 117 - protein, 14 aa, Homo sapiens
STLNPQWNESFTFK

SEQ ID NO: 118 - protein, 9 aa, Homo sapiens
RWELCDIPR

SEQ ID NO: 119 - protein, 13 aa, Homo sapiens
GHFFVEDQIYCEK

SEQ ID NO: 120 - protein, 7 aa, Homo sapiens
YEFLNGR

SEQ ID NO: 121 - protein, 10 aa, Homo sapiens
LALDNGGLAR

SEQ ID NO: 122 - protein, 22 aa, Homo sapiens
TSESGELHGLTTEEEFVEGIYK

SEQ ID NO: 123 - protein, 18 aa, Homo sapiens
GHYTEGAELVDSVLDVVR

SEQ ID NO: 124 - protein, 21 aa, Homo sapiens
PLSIEADDNGCFPLDGHVLCR

SEQ ID NO: 125 - protein, 25 aa, Homo sapiens
LTTPTYGDLNHLVSATMSGVTTCLR

SEQ ID NO: 126 - protein, 27 aa, Homo sapiens
FWEVISDEHGIDPTGTYHGDSDLQLER

SEQ ID NO: 127 - protein, 16 aa, Homo sapiens
LQDRGPDVLTATVSGK

SEQ ID NO: 128 - protein, 9 aa, Homo sapiens
DGYLFQLLR

SEQ ID NO: 129 - protein, 8 aa, Homo sapiens
DYFIATCK

SEQ ID NO: 130 - protein, 8 aa, Homo sapiens
NGDLDEVK

SEQ ID NO: 131 - protein, 7 aa, Homo sapiens
LSVDYGK

SEQ ID NO: 132 - protein, 20 aa, Homo sapiens
TIGGGDDSFNTFFSETGAGK

SEQ ID NO: 133 - protein, 21 aa, Homo sapiens
EVITNSITQPTLHSFDAAQSR

SEQ ID NO: 134 - protein, 15 aa, Homo sapiens
IPGIFELGISSQSDR

SEQ ID NO: 135 - protein, 12 aa, Homo sapiens
HELQANCYEEVK

SEQ ID NO: 136 - protein, 14 aa, Homo sapiens
YAGSQVASTSEVLK

SEQ ID NO: 137 - protein, 9 aa, Homo sapiens
SVNDLYIQK

SEQ ID NO: 138 - protein, 8 aa, Homo sapiens
ITPELLTR

SEQ ID NO: 139 - protein, 8 aa, Homo sapiens
NIILEEGK

SEQ ID NO: 140 - protein, 11 aa, Homo sapiens
FNTSDVSAIEK

SEQ ID NO: 141 - protein, 9 aa, Homo sapiens
VDVYGYVVK

SEQ ID NO: 142 - protein, 20 aa, Homo sapiens
NSTIVFPLPIDMLQGIIGAK

SEQ ID NO: 143 - protein, 13 aa, Homo sapiens
SQHLDNFSNQIGK

SEQ ID NO: 144 - protein, 11 aa, Homo sapiens
NGNMAGISDQR

SEQ ID NO: 145 - protein, 11 aa, Homo sapiens
LVDEYSLNAGK

SEQ ID NO: 146 - protein, 14 aa, Homo sapiens
VPTANVSVVDLTCR

SEQ ID NO: 147 - protein, 9 aa, Homo sapiens
SGVSLAALK

SEQ ID NO: 148 - protein, 11 aa, Homo sapiens
GAALITAVGVR

SEQ ID NO: 149 - protein, 24 aa, Homo sapiens
EKPQSTISGLLGPTLYAEVGDIIK

SEQ ID NO: 150 - protein, 7 aa, Homo sapiens
QIGSVYR

SEQ ID NO: 151 - protein, 13 aa, Homo sapiens
PHEHGPPPPPDER

SEQ ID NO: 152 - protein, 35 aa, Homo sapiens
DHSHGPPLPQGPPPLLPMSCSSCQHATFGTNGAQR

SEQ ID NO: 153 - protein, 16 aa, Homo sapiens
VYTAQNPSAQALGLGK

SEQ ID NO: 154 - protein, 31 aa, Homo sapiens
IADAHLDRVENTTVYYLVLDVQESDCSVLSR

SEQ ID NO: 155 - protein, 13 aa, Homo sapiens
SATLASIDAELQK

SEQ ID NO: 156 - protein, 10 aa, Homo sapiens
RDGYLFQLLR

SEQ ID NO: 157 - protein, 7 aa, Homo sapiens
ALDLINK

SEQ ID NO: 158 - protein, 16 aa, Homo sapiens
ELGCGGPQQPDPAAGR

SEQ ID NO: 159 - protein, 9 aa, Homo sapiens
HPNVFGFCR

SEQ ID NO: 160 - protein, 13 aa, Homo sapiens
CQCLQTLQGIHLK

SEQ ID NO: 161 - protein, 11 aa, Homo sapiens
LLTMMPTEEER

SEQ ID NO: 162 - protein, 18 aa, Homo sapiens
IGWPNAPILIQDFETLPR

SEQ ID NO: 163 - protein, 9 aa, Homo sapiens
NAFVTGIAR

SEQ ID NO: 164 - protein, 18 aa, Homo sapiens
GSLDYRPLTTADPIDEHR

SEQ ID NO: 165 - protein, 12 aa, Homo sapiens
YSNSEVVTGSGR

SEQ ID NO: 166 - protein, 15 aa, Homo sapiens
RPDSLQHVLLPVLDR

SEQ ID NO: 167 - protein, 10 aa, Homo sapiens
LSINTHPSQK

SEQ ID NO: 168 - protein, 12 aa, Homo sapiens
SGSDEVQVGQQR

SEQ ID NO: 169 - protein, 16 aa, Homo sapiens
VHQYFNVELIQPGAVK

SEQ ID NO: 170 - protein, 9 aa, Homo sapiens
TNLLQVCER

SEQ ID NO: 171 - protein, 10 aa, Homo sapiens
SVQLNGLTLK

SEQ ID NO: 172 - protein, 8 aa, Homo sapiens
FYYIYNEK

SEQ ID NO: 173 - protein, 7 aa, Homo sapiens
IWDVVEK

SEQ ID NO: 174 - protein, 12 aa, Homo sapiens
TIYTPGSTVLYR

SEQ ID NO: 175 - protein, 12 aa, Homo sapiens
ELNGTYAIAGGR

SEQ ID NO: 176 - protein, 13 aa, Homo sapiens
STAALEEDAQILK

SEQ ID NO: 177 - protein, 8 aa, Homo sapiens
VVLVAVDK

SEQ ID NO: 178 - protein, 34 aa, Homo sapiens
LGQHVPTLHPTSEELTIAGMTFTTFDLGGHEQAR

SEQ ID NO: 179 - protein, 9 aa, Homo sapiens
NTLIIYLDK

SEQ ID NO: 180 - protein, 16 aa, Homo sapiens
SNLDEDIIAEENIVSR

SEQ ID NO: 181 - protein, 9 aa, Homo sapiens
LIDIGNSCR

SEQ ID NO: 182 - protein, 15 aa, Homo sapiens
TVMVNIENPEGIPVK

SEQ ID NO: 183 - protein, 10 aa, Homo sapiens
HQQTVTIPPK

SEQ ID NO: 184 - protein, 8 aa, Homo sapiens
YLSNAYAR

SEQ ID NO: 185 - protein, 19 aa, Homo sapiens
SVMAPFTMTIDAHTNGNGK

SEQ ID NO: 186 - protein, 11 aa, Homo sapiens
GLSAEPGWQAK

SEQ ID NO: 187 - protein, 20 aa, Homo sapiens
LVFQQFDLEPSEGCFYDYVK

SEQ ID NO: 188 - protein, 18 aa, Homo sapiens
IACVLPVLMDGIQSHPQK

SEQ ID NO: 189 - protein, 11 aa, Homo sapiens
TLNICEVGTIR

SEQ ID NO: 190 - protein, 9 aa, Homo sapiens
QWNVNWDIR

SEQ ID NO: 191 - protein, 9 aa, Homo sapiens
FISLGEACK

SEQ ID NO: 192 - protein, 11 aa, Homo sapiens
CCSGAIIVLTK

SEQ ID NO: 193 - protein, 21 aa, Homo sapiens
VIISAPSADAPMFVMGVNHEK

SEQ ID NO: 194 - protein, 11 aa, Homo sapiens
DEITFVSGAPR

SEQ ID NO: 195 - protein, 8 aa, Homo sapiens
EDLAALEK

SEQ ID NO: 196 - protein, 23 aa, Homo sapiens
SEALAVDGAGKPGAEEAQDPEGK

SEQ ID NO: 197 - protein, 12 aa, Homo sapiens
CIVEVISDTLSK

SEQ ID NO: 198 - protein, 19 aa, Homo sapiens
GHYTEGAELVDSVLDVVRK

SEQ ID NO: 199 - protein, 9 aa, Homo sapiens
EIIDPVLDR

SEQ ID NO: 200 - protein, 26 aa, Homo sapiens
HSCQAECSSELYTEASGYISSLEYPR

SEQ ID NO: 201 - protein, 15 aa, Homo sapiens
EEEEEMAVVPQGLFR

SEQ ID NO: 202 - protein, 15 aa, Homo sapiens
AILVDLEPGTMDSVR

SEQ ID NO: 203 - protein, 15 aa, Homo sapiens
AVFVDLEPTVIDEIR

SEQ ID NO: 204 - protein, 11 aa, Homo sapiens
MREWFSETFQK

SEQ ID NO: 205 - protein, 14 aa, Homo sapiens
LPVANPQACENWLR

SEQ ID NO: 206 - protein, 12 aa, Homo sapiens
GCTDNLTLTVAR

SEQ ID NO: 207 - protein, 16 aa, Homo sapiens
SAMPFTASPASSTTAR

SEQ ID NO: 208 - protein, 19 aa, Homo sapiens
MNLASEPQEVLHIGSAHNR

SEQ ID NO: 209 - protein, 16 aa, Homo sapiens
HSQSMIEDAQLPLEQK

SEQ ID NO: 210 - protein, 8 aa, Homo sapiens
FFQYDTWK

SEQ ID NO: 211 - protein, 17 aa, Homo sapiens
QSTSFLVLQEILESEEK

SEQ ID NO: 212 - protein, 10 aa, Homo sapiens
DFEQPLAISR

SEQ ID NO: 213 - protein, 9 aa, Homo sapiens
LDELRDEGK

SEQ ID NO: 214 - protein, 13 aa, Homo sapiens
LQLFGQDFSHAAR

SEQ ID NO: 215 - protein, 12 aa, Homo sapiens
KYWNDCEPPDSR

SEQ ID NO: 216 - protein, 8 aa, Homo sapiens
ASVDGWFK

SEQ ID NO: 217 - protein, 12 aa, Homo sapiens
MTSILEAVPQVK

SEQ ID NO: 218 - protein, 28 aa, Homo sapiens
VITNQYNNPAGLYSSENISNFNNALESK

SEQ ID NO: 219 - protein, 22 aa, Homo sapiens
PQVQLHVQSQTQPVSLANTQPR

SEQ ID NO: 220 - protein, 12 aa, Homo sapiens
TINEVENQILTR

SEQ ID NO: 221 - protein, 8 aa, Homo sapiens
ISCTIANR

SEQ ID NO: 222 - protein, 8 aa, Homo sapiens
LAQNIDVK

SEQ ID NO: 223 - protein, 18 aa, Homo sapiens
VNPTVFFDIAVDGEPLGR

SEQ ID NO: 224 - protein, 10 aa, Homo sapiens
MLEENTNILK

SEQ ID NO: 225 - protein, 10 aa, Homo sapiens
IHWESASLLR

SEQ ID NO: 226 - protein, 22 aa, Homo sapiens
HSGFEDELSEVLENQSSQAELK

SEQ ID NO: 227 - protein, 11 aa, Homo sapiens
NILTSNNIDVK

SEQ ID NO: 228 - protein, 11 aa, Homo sapiens
VCSQYAAYGEK

SEQ ID NO: 229 - protein, 13 aa, Homo sapiens
SSLSVPYVIVPLK

SEQ ID NO: 230 - protein, 9 aa, Homo sapiens
SALVLQYLR

SEQ ID NO: 231 - protein, 11 aa, Homo sapiens
VWSPLVTEEGK

SEQ ID NO: 232 - protein, 10 aa, Homo sapiens
EIYMTFLSSK

SEQ ID NO: 233 - protein, 10 aa, Homo sapiens
LQDQIFNLMK

SEQ ID NO: 234 - protein, 14 aa, Homo sapiens
AGDFLEANYMNLQR

SEQ ID NO: 235 - protein, 16 aa, Homo sapiens
YWPLYGEDPITFAPFK

SEQ ID NO: 236 - protein, 11 aa, Homo sapiens
TAEEICESSSK

SEQ ID NO: 237 - protein, 14 aa, Homo sapiens
LVAYYTLIGASGQR

SEQ ID NO: 238 - protein, 21 aa, Homo sapiens
YIPYTLYSVFNPSDHDLVLIR

SEQ ID NO: 239 - protein, 10 aa, Homo sapiens
NTMILEICTR

SEQ ID NO: 240 - protein, 12 aa, Homo sapiens
ENEGFTVTAEGK

SEQ ID NO: 241 - protein, 21 aa, Homo sapiens
LHEWTKPENLDFIEVNVSLPR

SEQ ID NO: 242 - protein, 19 aa, Homo sapiens
DAPDHQELNLDVSLQLPSR

SEQ ID NO: 243 - protein, 9 aa, Homo sapiens
IVEVEQDNK

SEQ ID NO: 244 - protein, 10 aa, Homo sapiens
DFDFVPPVVR

SEQ ID NO: 245 - protein, 24 aa, Homo sapiens
KLEGDSTDLSDQIAELQAQIAELK

SEQ ID NO: 246 - protein, 12 aa, Homo sapiens
TLMNLGGLAVAR

SEQ ID NO: 247 - protein, 19 aa, Homo sapiens
NMDPLNDNIATLLHQSSDK

SEQ ID NO: 248 - protein, 7 aa, Homo sapiens
WIADGQR

SEQ ID NO: 249 - protein, 7 aa, Homo sapiens
NMACVQR

SEQ ID NO: 250 - protein, 9 aa, Homo sapiens
PYCQPCFLK

SEQ ID NO: 251 - protein, 20 aa, Homo sapiens
NLSLSGHVGFDSLPDQLVNK

SEQ ID NO: 252 - protein, 21 aa, Homo sapiens
LGQSVTTIPTVGFNVETVTYK

SEQ ID NO: 253 - protein, 16 aa, Homo sapiens
ANLQIDQINTDLNLER

SEQ ID NO: 254 - protein, 9 aa, Homo sapiens
GYTQQLAFR

SEQ ID NO: 255 - protein, 14 aa, Homo sapiens
AWGESTPLANKPGK

SEQ ID NO: 256 - protein, 9 aa, Homo sapiens
EDAANNYAR

SEQ ID NO: 257 - protein, 9 aa, Homo sapiens
EAVEEPSSK

SEQ ID NO: 258 - protein, 12 aa, Homo sapiens
EVDEQMLNVQNK

SEQ ID NO: 259 - protein, 8 aa, Homo sapiens
PVNPVEQR

SEQ ID NO: 260 - protein, 31 aa, Homo sapiens
AEGNNQAPGEEEEEEEEATNTHPPASLPSQK

SEQ ID NO: 261 - protein, 21 aa, Homo sapiens
RAFVHWYVGEGMEEGEFSEAR

SEQ ID NO: 262 - protein, 12 aa, Homo sapiens
EVDQQLLSVQTR

SEQ ID NO: 263 - protein, 12 aa, Homo sapiens
GVCEETSGAYEK

SEQ ID NO: 264 - protein, 10 aa, Homo sapiens
DVNAAIAAIK

SEQ ID NO: 265 - protein, 8 aa, Homo sapiens
NIGEFCGK

SEQ ID NO: 266 - protein, 11 aa, Homo sapiens
SEETLDEGPPK

SEQ ID NO: 267 - protein, 10 aa, Homo sapiens
TEISLVLTSK

SEQ ID NO: 268 - protein, 9 aa, Homo sapiens
VLDSEGQLR

SEQ ID NO: 269 - protein, 10 aa, Homo sapiens
VSYLQLSFWK

SEQ ID NO: 270 - protein, 9 aa, Homo sapiens
QVSDDLTER

SEQ ID NO: 271 - protein, 7 aa, Homo sapiens
MDMTFSK

SEQ ID NO: 272 - protein, 9 aa, Homo sapiens
SEIDLVQIK

SEQ ID NO: 273 - protein, 9 aa, Homo sapiens
NLCQELEAK

SEQ ID NO: 274 - protein, 13 aa, Homo sapiens
SEHPGLSIGDTAK

SEQ ID NO: 275 - protein, 11 aa, Homo sapiens
YVDILPYDYNR

SEQ ID NO: 276 - protein, 11 aa, Homo sapiens
TTVGVDGSLYK

SEQ ID NO: 277 - protein, 17 aa, Homo sapiens
VPTANVSVVDLTCRLEK

SEQ ID NO: 278 - protein, 8 aa, Homo sapiens
DLNLMDIK

SEQ ID NO: 279 - protein, 15 aa, Homo sapiens
TMQFEPSTMVYDACR

SEQ ID NO: 280 - protein, 22 aa, Homo sapiens
FGFCPMAAHEEICTTNEGVMYR

SEQ ID NO: 281 - protein, 16 aa, Homo sapiens
AAFYEEQINYYDTYIK

SEQ ID NO: 282 - protein, 21 aa, Homo sapiens
WGDAGAEYVVESTGVFTTMEK

SEQ ID NO: 283 - protein, 8 aa, Homo sapiens
TPENFPCK

SEQ ID NO: 284 - protein, 10 aa, Homo sapiens
VAASIGNAQK

SEQ ID NO: 285 - protein, 11 aa, Homo sapiens
ADIGCTPGSGK

SEQ ID NO: 286 - protein, 13 aa, Homo sapiens
VYAYYNLEESCTR

SEQ ID NO: 287 - protein, 23 aa, Homo sapiens
GTHSLTESGASCLPWNSMILIGK

SEQ ID NO: 288 - protein, 17 aa, Homo sapiens
GEVQAMLGQSTEELRVR

SEQ ID NO: 289 - protein, 20 aa, Homo sapiens
LVQYRGEVQAMLGQSTEELR

SEQ ID NO: 290 - protein, 23 aa, Homo sapiens
WVQTLSEQVQEELLSSQVTQELR

SEQ ID NO: 291 - protein, 27 aa, Homo sapiens
YASICQQNGIVPIVEPEILPDGDHDLK

SEQ ID NO: 292 - protein, 9 aa, Homo sapiens
TGLQEVEVK

SEQ ID NO: 293 - protein, 16 aa, Homo sapiens
VELLHNPAFCSLATTK

SEQ ID NO: 294 - protein, 10 aa, Homo sapiens
SSVLENFVGR

SEQ ID NO: 295 - protein, 7 aa, Homo sapiens
FAFNLYR

SEQ ID NO: 296 - protein, 24 aa, Homo sapiens
NQINALTSFVDASMVYGSEEPLAR

SEQ ID NO: 297 - protein, 10 aa, Homo sapiens
ELFDPIISDR

SEQ ID NO: 298 - protein, 8 aa, Homo sapiens
QVALWDTK

SEQ ID NO: 299 - protein, 14 aa, Homo sapiens
DSTYSLSSTLTLSK

SEQ ID NO: 300 - protein, 10 aa, Homo sapiens
QQTEWQSGQR

SEQ ID NO: 301 - protein, 18 aa, Homo sapiens
LIRDVWGIEGPIDAAFTR

SEQ ID NO: 302 - protein, 26 aa, Homo sapiens
LPPLRKGEVLPLPEANFPSFPLPHHK

SEQ ID NO: 303 - protein, 10 aa, Homo sapiens
GLGDVDQLVK

SEQ ID NO: 304 - protein, 9 aa, Homo sapiens
LTPYADEFK

SEQ ID NO: 305 - protein, 27 aa, Homo sapiens
VSNQTLSLFFTVLQDVPVRDLKPAIVK

SEQ ID NO: 306 - protein, 10 aa, Homo sapiens
TFYSCTTEGR

SEQ ID NO: 307 - protein, 21 aa, Homo sapiens
VLRENADSLQASLRPHADELK

SEQ ID NO: 308 - protein, 17 aa, Homo sapiens
YGFCEAADQFHVLDEVR

SEQ ID NO: 309 - protein, 10 aa, Homo sapiens
NSGLTEEEAK

SEQ ID NO: 310 - protein, 8 aa, Homo sapiens
AELQEGAR

SEQ ID NO: 311 - protein, 7 aa, Homo sapiens
DRLDEVK

SEQ ID NO: 312 - protein, 15 aa, Homo sapiens
FVSDYETHGAGFSIR

SEQ ID NO: 313 - protein, 13 aa, Homo sapiens
MLAAQGVDPGLAR

SEQ ID NO: 314 - protein, 9 aa, Homo sapiens
SVGFHLPSR

SEQ ID NO: 315 - protein, 11 aa, Homo sapiens
LMIEMDGTENK

SEQ ID NO: 316 - protein, 10 aa, Homo sapiens
LKDDEVAQLK

SEQ ID NO: 317 - protein, 11 aa, Homo sapiens
AEDTAVYYCAR

SEQ ID NO: 318 - protein, 19 aa, Homo sapiens
YPSLSIHGIEGAFDEPGTK

SEQ ID NO: 319 - protein, 15 aa, Homo sapiens
FTVDRPFLFLIYEHR

SEQ ID NO: 320 - protein, 21 aa, Homo sapiens
EINLAPDSSSVVVSGLMVATK

SEQ ID NO: 321 - protein, 28 aa, Homo sapiens
EEPPRPEFLEQPLLGELTVTGVTPDSLR

SEQ ID NO: 322 - protein, 15 aa, Homo sapiens
IFYNQQNHYDGSTGK

SEQ ID NO: 323 - protein, 49 aa, Homo sapiens
PLHTLMHGQGVCMELAEIEAIQESLQPSDKDEGDHPNNSFSPCSAHDRR

SEQ ID NO: 324 - protein, 10 aa, Homo sapiens
FNAPFDVGIK

SEQ ID NO: 325 - protein, 18 aa, Homo sapiens
GGETAQSADPQWEQLNNK

SEQ ID NO: 326 - protein, 15 aa, Homo sapiens
PGQSPQLLIYLGSNR

SEQ ID NO: 327 - protein, 8 aa, Homo sapiens
AQMDLSGR

SEQ ID NO: 328 - protein, 11 aa, Homo sapiens
CSNEEVAAMIR

SEQ ID NO: 329 - protein, 14 aa, Homo sapiens
CIHPCIITEENMNK

SEQ ID NO: 330 - protein, 11 aa, Homo sapiens
ATAVMPDGQFK

SEQ ID NO: 331 - protein, 8 aa, Homo sapiens
EVLLEVQK

SEQ ID NO: 332 - protein, 8 aa, Homo sapiens
LFLEPTRK

SEQ ID NO: 333 - protein, 14 aa, Homo sapiens
DLYQLNPNAEWVIK

SEQ ID NO: 334 - protein, 13 aa, Homo sapiens
EDSLEAGLPLQVR

SEQ ID NO: 335 - protein, 9 aa, Homo sapiens
CTEGFNVDK

SEQ ID NO: 336 - protein, 10 aa, Homo sapiens
IIGIDINSEK

SEQ ID NO: 337 - protein, 19 aa, Homo sapiens
IRFHDFLGDSWGILFSHPR

SEQ ID NO: 338 - protein, 10 aa, Homo sapiens
GTLSGWILSK

SEQ ID NO: 339 - protein, 22 aa, Homo sapiens
GAGSLELYLDCIQVDSVHNLPR

SEQ ID NO: 340 - protein, 18 aa, Homo sapiens
SLAELGGHLDQQVEEFRR

SEQ ID NO: 341 - protein, 7 aa, Homo sapiens
PVAIGGK

SEQ ID NO: 342 - protein, 10 aa, Homo sapiens
DYYVSTAVCR

SEQ ID NO: 343 - protein, 8 aa, Homo sapiens
ELQAAQAR

SEQ ID NO: 344 - protein, 15 aa, Homo sapiens
HLLGPDYTETLYSPR

SEQ ID NO: 345 - protein, 15 aa, Homo sapiens
EVPFADLSNMEIGMK

SEQ ID NO: 346 - protein, 21 aa, Homo sapiens
GLEWIGYIYYSGSTNYNPSLK

SEQ ID NO: 347 - protein, 13 aa, Homo sapiens
VTWDSSFCAVNPR

SEQ ID NO: 348 - protein, 9 aa, Homo sapiens
LEEQAQQIR

SEQ ID NO: 349 - protein, 11 aa, Homo sapiens
LLPHANEVSQK

SEQ ID NO: 350 - protein, 12 aa, Homo sapiens
RELDESLQVAER

SEQ ID NO: 351 - protein, 9 aa, Homo sapiens
LAVYQAGAR

SEQ ID NO: 352 - protein, 12 aa, Homo sapiens
RALDFAVGEYNK

SEQ ID NO: 353 - protein, 7 aa, Homo sapiens
EAAQFAR

SEQ ID NO: 354 - protein, 8 aa, Homo sapiens
NPANPVQR

SEQ ID NO: 355 - protein, 17 aa, Homo sapiens
FLATTPNSLLVSWQPPR

SEQ ID NO: 356 - protein, 7 aa, Homo sapiens
NFGYTLR

SEQ ID NO: 357 - protein, 8 aa, Homo sapiens
SEEEVAAR

SEQ ID NO: 358 - protein, 17 aa, Homo sapiens
ISDLGLAVHVPEGQTIK

SEQ ID NO: 359 - protein, 9 aa, Homo sapiens
LEPYADQLR

SEQ ID NO: 360 - protein, 8 aa, Homo sapiens
FLENEDRR

SEQ ID NO: 361 - protein, 9 aa, Homo sapiens
ETWTANVGK

SEQ ID NO: 362 - protein, 19 aa, Homo sapiens
WQQGNIFSCSVMHEALHNR

SEQ ID NO: 363 - protein, 8 aa, Homo sapiens
YLDSTFTK

SEQ ID NO: 364 - protein, 8 aa, Homo sapiens
AVSNEIVR

SEQ ID NO: 365 - protein, 10 aa, Homo sapiens
GAPPLPPIPR

SEQ ID NO: 366 - protein, 9 aa, Homo sapiens
NLLDHLEEK

SEQ ID NO: 367 - protein, 15 aa, Homo sapiens
PNMIISVNGDVITIK

SEQ ID NO: 368 - protein, 9 aa, Homo sapiens
WIISSLTPK

SEQ ID NO: 369 - protein, 10 aa, Homo sapiens
GNPEQTPVLK

SEQ ID NO: 370 - protein, 11 aa, Homo sapiens
ENQFDPPIVAR

SEQ ID NO: 371 - protein, 13 aa, Homo sapiens
PGLDHTEASFSPR

SEQ ID NO: 372 - protein, 31 aa, Homo sapiens
LESEETMVLEAHDAQGDVPVTVTVHDFPGKK

SEQ ID NO: 373 - protein, 11 aa, Homo sapiens
FDGGVEAIATR

SEQ ID NO: 374 - protein, 18 aa, Homo sapiens
ILGDPEALRDLLNNHILK

SEQ ID NO: 375 - protein, 8 aa, Homo sapiens
LAAEFASK

SEQ ID NO: 376 - protein, 48 aa, Homo sapiens
EYTYEWSISEDSGPTHDDPPCLTHIYYSHENLIEDFNSGLIGPLLICK

SEQ ID NO: 377 - protein, 11 aa, Homo sapiens
HEDTLTLFPMR

SEQ ID NO: 378 - protein, 11 aa, Homo sapiens
NTLYLQMSSLR

SEQ ID NO: 379 - protein, 13 aa, Homo sapiens
LGEVNTYAGDLQK

SEQ ID NO: 380 - protein, 9 aa, Homo sapiens
VTLEERLDK

SEQ ID NO: 381 - protein, 12 aa, Homo sapiens
CCEDGMRENPMR

SEQ ID NO: 382 - protein, 12 aa, Homo sapiens
SLAPYAQDTQEK

SEQ ID NO: 383 - protein, 9 aa, Homo sapiens
IDQNVEELK

SEQ ID NO: 384 - protein, 25 aa, Homo sapiens
LPEGSVSLIILLTDGDPTVGETNPR

SEQ ID NO: 385 - protein, 12 aa, Homo sapiens
GEWTCIAYSQLR

SEQ ID NO: 386 - protein, 19 aa, Homo sapiens
LHTEAQIQEEGTVVELTGR

SEQ ID NO: 387 - protein, 30 aa, Homo sapiens
ADNFLLENTLPAQSTFTLAISAYALSLGDK

SEQ ID NO: 388 - protein, 11 aa, Homo sapiens
LLGELLQDNAK

SEQ ID NO: 389 - protein, 9 aa, Homo sapiens
GLEVTITAR

SEQ ID NO: 390 - protein, 13 aa, Homo sapiens
GTFTLHVPQDTER

SEQ ID NO: 391 - protein, 12 aa, Homo sapiens
VVEQMCITQYER

SEQ ID NO: 392 - protein, 14 aa, Homo sapiens
SRLDTLAQEVALLK

SEQ ID NO: 393 - protein, 10 aa, Homo sapiens
DGAGDVAFIR

SEQ ID NO: 394 - protein, 10 aa, Homo sapiens
LIESAEELIR

SEQ ID NO: 395 - protein, 18 aa, Homo sapiens
NPDGSEAPWCFTLRPGMR

SEQ ID NO: 396 - protein, 21 aa, Homo sapiens
EVEELEQLTQQLMQDMEHPQR

SEQ ID NO: 397 - protein, 20 aa, Homo sapiens
SWFEPLVEDMQRQWAGLVEK

SEQ ID NO: 398 - protein, 7 aa, Homo sapiens
PISINYR

SEQ ID NO: 399 - protein, 9 aa, Homo sapiens
ALTDMPQMR

SEQ ID NO: 400 - protein, 9 aa, Homo sapiens
EIPSLDQEK

SEQ ID NO: 401 - protein, 7 aa, Homo sapiens
LHELQEK

SEQ ID NO: 402 - protein, 12 aa, Homo sapiens
FFSASCVPGADK

SEQ ID NO: 403 - protein, 13 aa, Homo sapiens
WYVVGLAGNAILR

SEQ ID NO: 404 - protein, 16 aa, Homo sapiens
HGGTIPIVPTAEFQDR

SEQ ID NO: 405 - protein, 11 aa, Homo sapiens
SWWGDYWEPFR

SEQ ID NO: 406 - protein, 21 aa, Homo sapiens
DFSAEYEEDGKYEGLQEWEGK

SEQ ID NO: 407 - protein, 9 aa, Homo sapiens
LGFTDLFSK

SEQ ID NO: 408 - protein, 10 aa, Homo sapiens
TLLSNLEEAK

SEQ ID NO: 409 - protein, 7 aa, Homo sapiens
IGDTWSK

SEQ ID NO: 410 - protein, 9 aa, Homo sapiens
LPDSPALAK

SEQ ID NO: 411 - protein, 7 aa, Homo sapiens
MEEMGSR

SEQ ID NO: 412 - protein, 14 aa, Homo sapiens
NYLGGFALSVAHGR

SEQ ID NO: 413 - protein, 20 aa, Homo sapiens
HADGSFSDEMNTILDNLAAR

SEQ ID NO: 414 - protein, 22 aa, Homo sapiens
LVQYRGEVQAMLGQSTEELRVR

SEQ ID NO: 415 - protein, 22 aa, Homo sapiens
PGVVYEGQLISIQQYGHQEVTR

SEQ ID NO: 416 - protein, 11 aa, Homo sapiens
ALDGAFTEENR

SEQ ID NO: 417 - protein, 13 aa, Homo sapiens
AATGECTATVGKR

SEQ ID NO: 418 - protein, 16 aa, Homo sapiens
TVLDLAVVLFETATLR

SEQ ID NO: 419 - protein, 31 aa, Homo sapiens
VTIMWTPPESAVTGYRVDVIPVNLPGEHGQR

SEQ ID NO: 420 - protein, 11 aa, Homo sapiens
ASSLESGVPSR

SEQ ID NO: 421 - protein, 11 aa, Homo sapiens
RVEPYGENFNK

SEQ ID NO: 422 - protein, 38 aa, Homo sapiens
ALCGFTEAAAQAAYLVGVSDPNSQAGQQGLVEPTQFAR

SEQ ID NO: 423 - protein, 17 aa, Homo sapiens
LLRDPADASEAHESSSR

SEQ ID NO: 424 - protein, 26 aa, Homo sapiens
FYDRSDIDAVYVTTNGIIATSEPPAK

SEQ ID NO: 425 - protein, 9 aa, Homo sapiens
QTVAVGVIK

SEQ ID NO: 426 - protein, 10 aa, Homo sapiens
MYYSAVDPTK

SEQ ID NO: 427 - protein, 9 aa, Homo sapiens
QTMVDSSCR

SEQ ID NO: 428 - protein, 15 aa, Homo sapiens
ALTVPELTQQVFDAK

SEQ ID NO: 429 - protein, 16 aa, Homo sapiens
GIISALLVPPETEEAK

SEQ ID NO: 430 - protein, 8 aa, Homo sapiens
QLTPYAQR

SEQ ID NO: 431 - protein, 11 aa, Homo sapiens
DLQFVEVTDVK

SEQ ID NO: 432 - protein, 9 aa, Homo sapiens
ISASAEELR

SEQ ID NO: 433 - protein, 15 aa, Homo sapiens
VPGTSTSATLTGLTR

SEQ ID NO: 434 - protein, 12 aa, Homo sapiens
EHALLAYTLGVK

SEQ ID NO: 435 - protein, 16 aa, Homo sapiens
EYLGAICSCTCFGGQR

SEQ ID NO: 436 - protein, 11 aa, Homo sapiens
MTDQVNYQAMK

SEQ ID NO: 437 - protein, 9 aa, Homo sapiens
TGFQAVTGK

SEQ ID NO: 438 - protein, 9 aa, Homo sapiens
DYSVTANSK

SEQ ID NO: 439 - protein, 15 aa, Homo sapiens
VDVIPVNLPGEHGQR

SEQ ID NO: 440 - protein, 10 aa, Homo sapiens
EPAVLELEGK

SEQ ID NO: 441 - protein, 13 aa, Homo sapiens
CLAENAGDVAFVK

SEQ ID NO: 442 - protein, 11 aa, Homo sapiens
LDPHLVLDQLR

SEQ ID NO: 443 - protein, 14 aa, Homo sapiens
YSFCTDHTVLVQTR

SEQ ID NO: 444 - protein, 9 aa, Homo sapiens
FLILLGSPK

SEQ ID NO: 445 - protein, 22 aa, Homo sapiens
TEIDKPSQMQVTDVQDNSISVK

SEQ ID NO: 446 - protein, 15 aa, Homo sapiens
LCMGSGLNLCEPNNK

SEQ ID NO: 447 - protein, 14 aa, Homo sapiens
SELTQQLNALFQDK

SEQ ID NO: 448 - protein, 7 aa, Homo sapiens
IFTVNHK

SEQ ID NO: 449 - protein, 10 aa, Homo sapiens
NQADCIPFFR

SEQ ID NO: 450 - protein, 15 aa, Homo sapiens
VLVHTSVSPSPDNLR

SEQ ID NO: 451 - protein, 8 aa, Homo sapiens
VFAVSHGR

SEQ ID NO: 452 - protein, 9 aa, Homo sapiens
VWSDYVGGR

SEQ ID NO: 453 - protein, 12 aa, Homo sapiens
VYSGILNQSEIK

SEQ ID NO: 454 - protein, 19 aa, Homo sapiens
IPEISIQDMTAQVTSPSGK

SEQ ID NO: 455 - protein, 13 aa, Homo sapiens
GFSVVADTPELQR

SEQ ID NO: 456 - protein, 8 aa, Homo sapiens
EAVEHLQK

SEQ ID NO: 457 - protein, 7 aa, Homo sapiens
ECLQTCR

SEQ ID NO: 458 - protein, 15 aa, Homo sapiens
FLEQELETITIPDLR

SEQ ID NO: 459 - protein, 17 aa, Homo sapiens
FSPATHPSEGLEENYCR

SEQ ID NO: 460 - protein, 7 aa, Homo sapiens
GLEWLGR

SEQ ID NO: 461 - protein, 11 aa, Artificial Sequence
  Description of Artificial Sequence Synthetic peptide
YLAGVRITCFL

SEQ ID NO: 462 - protein, 14 aa, Artificial Sequence
  Description of Artificial Sequence Synthetic peptide
TCFLYLVRGITCLA

(19) United States
(12) Patent Application Publication
(10) Pub. No.: US 2022/0260559 A1
(43) Pub. Date: Aug. 18, 2022

(54) BIOMARKERS FOR DIAGNOSING ALZHEIMER'S DISEASE

(71) Applicant: Seer, Inc., Redwood City, CA (US)

(72) Inventors: John BLUME, Bellingham, WA (US); Ryan BENZ, Huntington Beach, CA (US); Asim SIDDIQUI, San Francisco, CA (US); Philip MA, San Jose, CA (US)

(21) Appl. No.: 17/518,016

(22) Filed: Nov. 3, 2021

Related U.S. Application Data
(60) Provisional application No. 63/109,806, filed on Nov. 4, 2020.
(60) Provisional application No. 63/149,047, filed on Feb. 12, 2021.

(51) Int. Cl. G01N 33/543; G01N 33/551; G01N 33/68

(52) U.S. Cl. CPC G01N 33/54326; G01N 33/6848; G01N 33/551; G01N 33/54346

(57) ABSTRACT

Disclosed herein are compositions, systems, and methods for identifying neurological diseases from biological sample analysis. A biological sample from a subject may be contacted to a particle to form a biomolecule corona, which may contain a subset of biomolecules from the biological sample and which can have utility for diagnosing a neurological disease state. Further disclosed herein are machine learning algorithms and trained classifiers for distinguishing neurological disease states based on biological data.

### CROSS-REFERENCE

The present application claims the benefit of U.S. Provisional Application No. 63/109,806, filed Nov. 4, 2020; and U.S. Provisional Application No. 63/149,047, filed Feb. 12, 2021, each of which is incorporated herein by reference in their entirety.

### SEQUENCE LISTING

The instant application contains a Sequence Listing which has been submitted electronically in ASCII format and is hereby incorporated by reference in its entirety. Said ASCII copy, created on Jan. 27, 2022, is named 53344-729_201_SL.txt and is 85,016 bytes in size.

### BACKGROUND

Few methods exist for accurate neurodegenerative diagnosis. Primary screening for neurodegeneration is typically based on cognitive assessment (e.g., Mini-Mental State Examinations and Memory Impairment Screens), and therefore typically identifies cognitive decline without providing insight into underlying causes, pathologies, and risk factors. While medical imaging (e.g., Magnetic Resonance Imaging) and tissue analysis can, in certain cases, distinguish neurological conditions, these methods may struggle with early phase detection and tracking disease progression, and may be prohibitively invasive and cost intensive for routine use.

### SUMMARY

Responsive to the need for faster and less intensive methods for neurological disease diagnosis, aspects of the present disclosure provide compositions, systems, and methods for identifying pluralities of neurological disease biomarkers from biological samples. As individual biomarker analysis has proven to typically be ineffective for identifying neurological disease states, aspects of the present disclosure provide methods which can identify tens, hundreds, thousands, or tens of thousands of biomolecules from biological samples, as well as patterns of biomolecule abundances and biomolecule-particle binding. Further disclosed herein are computer-implemented systems for identifying biological state information, for example neurological disease information, from biological data.

In some aspects, the present disclosure describes a method, comprising: obtaining a data set comprising protein or peptide information from biomolecule coronas that correspond to physiochemically distinct particles incubated with a biofluid sample from a subject; and using a classifier to identify the biofluid sample being indicative of a biological state comprising healthy state, a neurocognitive disorder, or a neurodegenerative disease, in the subject, based on the data set.

In some embodiments, the neurocognitive disorder comprises a mild cognitive impairment (MCI). In some embodiments, the neurodegenerative disease comprises Alzheimer's disease (AD).

In some embodiments, the protein information comprises expression information for a protein provided in a table or figure included herein. In some embodiments, the peptide information comprises expression information for a protein provided in any table or figure included herein.

In some embodiments, obtaining a data set comprises contacting the biofluid sample with the physiochemically distinct particles to form the biomolecule coronas. In some embodiments, obtaining a data set comprises detecting proteins of the biomolecule coronas by mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof. In some embodiments, obtaining a data set comprises detecting the proteins of the biomolecule coronas by mass spectrometry. In some embodiments, obtaining a data set comprises measuring a readout indicative of the presence, absence or amount of proteins of the biomolecule coronas.

In some embodiments, the physiochemically distinct particles comprise lipid particles, metal particles, silica particles, or polymer particles. In some embodiments, the physiochemically distinct particles comprise polystyrene particles, magnetizable particles, dextran particles, silica particles, dimethylamine particles, carboxylate particles, amino particles, benzoic acid particles, or agglutinin particles.

In some embodiments, the method further comprises administering a neurocognitive disorder treatment or a neurodegenerative disease treatment to the subject based on the biological state.

In some embodiments, the biofluid comprises a blood sample, a serum sample, or a plasma sample. In some embodiments, the biofluid comprises a blood sample that has had red blood cells removed. In some embodiments, the biofluid is plasma.

In some aspects, the present disclosure describes a method of evaluating a status of a biological state, comprising: measuring biomarkers in a biofluid sample from a subject suspected of having the neurocognitive disorder or the neurodegenerative disease to obtain biomarker measurements, wherein the biomarkers comprise one or more biomarkers selected from a table or figure included herein.

In some embodiments, the biological state comprises healthy state, a neurocognitive disorder, or a neurodegenerative disease. In some embodiments, the neurocognitive disorder comprises a mild cognitive impairment (MCI). In some embodiments, the neurodegenerative disease comprises Alzheimer's disease (AD).

In some embodiments, measuring the biomarkers comprises using a detection reagent that binds to a protein and yields a detectable signal.

In some embodiments, measuring the biomarkers comprises measuring a readout indicative of the presence, absence or amounts of the one or more biomarkers. In some embodiments, measuring the biomarkers comprises performing mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof. In some embodiments, measuring the biomarkers comprises performing mass spectrometry. In some embodiments, measuring the biomarkers comprises performing an immunoassay. In some embodiments, measuring the biomarkers comprises contacting the biofluid sample with a plurality of physiochemically distinct nanoparticles.

In some embodiments, the method further comprises applying a classifier to the biomarker measurements. In some embodiments, the classifier distinguishes any of the healthy state, the neurocognitive disorder, or the neurodegenerative disease, from each other.

In some embodiments, the method further comprises identifying the subject as having the neurocognitive disorder or the neurodegenerative disease based on the biomarker measurements.

In some embodiments, the method further comprises administering a neurocognitive disorder treatment or a neurodegenerative disease treatment to the subject.

In some embodiments, the biofluid comprises blood, plasma, or serum.

In some embodiments, the subject is human.

In some aspects, the present disclosure describes a method, comprising: assaying a biological sample from a subject to identify biomolecules; using a trained classifier to identify that the sample or the subject is positive or negative for Alzheimer's disease (AD) based on the biomolecules identified in (a), wherein the trained classifier is trained using data from training samples comprising known healthy samples and known Alzheimer's disease (AD) samples, and wherein the training samples were assayed using a plurality of particles having physicochemically distinct properties to yield the data.

In some aspects, the present disclosure describes a method, comprising: (a) assaying a biological sample from a subject to identify biomolecules; (b) using a trained classifier to identify that the sample or the subject is positive or negative for mild cognitive impairment (MCI) based on the biomolecules identified in (a), wherein the trained classifier is trained using data from training samples comprising known healthy samples and known mild cognitive impairment (MCI) samples, and wherein the training samples were assayed using a plurality of particles having physicochemically distinct properties to yield the data.

In some embodiments, the biomolecules comprise proteins.

In some embodiments, the proteins are selected from proteins included in a table or figure disclosed herein.

In some embodiments, the data comprises proteomic data identifying a presence or an absence of proteins in the training samples.

In some embodiments, the method further comprises obtaining a biological sample from a subject. In some embodiments, the biological sample is a complex biological sample. In some embodiments, the complex biological sample is a plasma sample or a serum sample.

In some embodiments, the plurality of particles having physicochemically distinct properties comprise two or more particles described herein.

In some embodiments, the assaying comprises performing mass spectrometry or ELISA, and wherein the biomolecules comprise protein. In some embodiments, the assaying comprises targeted mass spectrometry.

In some embodiments, the trained classifier is a trained algorithm.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a computer system that is programmed or otherwise configured to implement methods provided herein, in accordance with some embodiments.

FIG. 2 provides a workflow for collecting biomolecules from a biological sample onto particles, in accordance with some embodiments.

FIG. 3 provides a workflow for a particle-based assay for analyzing biomolecules from a biological sample, in accordance with some embodiments.

FIG. 4 provides a workflow for assaying biomolecules from a biological sample with magnetic particles, in accordance with some embodiments.

FIG. 5A summarizes the date, site, and class for 200 samples collected for Alzheimer's disease (AD) and mild cognitive impairment (MCI) analysis.

FIG. 5B outlines the numbers of samples collected for each diagnosis class (MCI, AD, healthy) across collection sites.

FIG. 6 summarizes age and gender distributions across healthy, AD, and MCI study groups.

FIG. 7A provides female and male gender counts for an AD and MCI diagnostic study.

FIG. 7B summarizes Fisher test for proportionality comparisons for AD, MCI, and control (healthy) subjects.

FIG. 8 provides the numbers of control, MCI, and AD samples per plate, as well as the identities of the particle panels used to interrogate the samples.

FIG. 9 provides the dates of mass spectrometry runs for particle panel-interrogated AD, MCI, and healthy samples.

FIG. 10 provides peptide yields for each of 10 particle types, with panel A providing results for SP-003 particles, panel B providing results for SP-006 particles, panel C providing results for SP-007 particles, panel D providing results for SP-008 particles, panel E providing results for SP-333 particles, panel F providing results for SP-339 particles, panel G providing results for SP-347 particles, panel H providing results for SP-353 particles, panel I providing results for SP-373 particles, and panel J providing results for SP-389 particles.

FIG. 11A provides a layout of an assay plate for biomolecule corona analysis.

FIG. 11B outlines an example of an assay which can utilize the assay plate of FIG. 11A.

FIG. 12 provides peptide and protein counts for the indicated process controls outlined in FIGS. 11A-11B.

FIG. 13 provides the median numbers of protein groups detected on each of 10 particle types following incubation with control, MCI, and AD samples.

FIG. 14A summarizes the percentage of samples in which identified data features were observed across 200 total AD, MCI, and healthy subject samples.

FIG. 14B summarizes the percentage of samples in which protein groups were observed across 200 total AD, MCI, and healthy subject samples.

FIG. 15 summarizes coefficient of variation values for proteins observed in 200 total AD, MCI, and healthy subject samples on 10 particle types.

FIG. 16 provides the number of unique peptides identified from each of 200 total AD, MCI, and healthy subject samples on each of the 10 particle types.

FIG. 17A summarizes the percentage of samples out of 200 total AD, MCI, and healthy subject samples in which individual data features were observed with a 10 particle panel.

FIG. 17B summarizes the percentage of samples out of 200 total AD, MCI, and healthy subject samples in which individual peptides were observed with a 10 particle panel.

FIG. 18 summarizes coefficient of variation values for mass spectrometric intensities of peptides observed in 200 AD, MCI, and healthy subject samples on each of 10 particle types.

FIG. 19A provides a volcano plot comparison of features observed with a 10 particle panel in AD and MCI samples. FIG. 19B provides a volcano plot comparison of features observed with a 10 particle panel in control and diseased samples. FIG. 19C provides a volcano plot comparison of features observed with a 10 particle panel in control and AD samples. FIG. 19D provides a volcano plot comparison of features observed with a 10 particle panel in control and MCI samples. FIGS. 19E-F provide the volcano plots of FIGS. 19C-D, respectively, with features associated with OpenTarget AD scores of 0.7 or greater circled and labeled.

FIG. 20 summarizes OpenTarget (OT) AD scores for AD (panel A), MCI (panel B), and disease (panel C) relevant protein groups identified in 100 AD and MCI samples.

FIG. 21 provides classification models for distinguishing samples from AD and healthy subjects. FIG. 21 panel A provides classification models for the particle SP-003; FIG. 21 panel B provides classification models for the particle SP-006; FIG. 21 panel C provides classification models for the particle SP-007; FIG. 21 panel D provides classification models for the particle SP-339; and FIG. 21 panel E provides classification models for the particle SP-373.

FIG. 22 provides classification models for distinguishing samples from MCI and healthy subjects. FIG. 22 panel A provides classification models for the particle SP-003; FIG. 22 panel B provides classification models for the particle SP-006; FIG. 22 panel C provides classification models for the particle SP-007; FIG. 22 panel D provides classification models for the particle SP-339; and FIG. 22 panel E provides classification models for the particle SP-373.

FIG. 23 provides classification models for distinguishing samples from MCI and AD subjects. FIG. 23 panel A provides classification models for the particle SP-003; FIG. 23 panel B provides classification models for the particle SP-006; FIG. 23 panel C provides classification models for the particle SP-007; FIG. 23 panel D provides classification models for the particle SP-229; and FIG. 23 panel E provides classification models for the particle SP-373.

FIG. 24 provides the overlap of top peptide features in AD versus control, MCI versus control, and AD versus MCI classifiers. FIG. 24 panel A provides peptide features for the control versus AD classifier. FIG. 24 panel B provides peptide features for the control versus MCI classifier. FIG. 24 panel C provides peptide features for the MCI versus AD classifier. In each panel, the columns are ordered, from left to right, by peptide features for SP-003 particles, SP-006 particles, SP-007 particles, SP-008 particles, SP-333 particles, SP-339 particles, SP-347 particles, SP-353 particles, SP-373 particles, and SP-389 particles.

FIG. 25 details the 20 top features of the MCI versus AD model peptide features outlined in FIG. 24.

FIG. 26 summarizes 2,085 protein groups detected in at least 25% of 200 total AD, MCI, and healthy subject samples, with the y-axis providing estimated human plasma concentrations in units of ng/ml.

FIG. 27A provides total protein group counts for each of 200 total AD, MCI, and healthy subject samples with a 10 particle panel.

FIG. 27B provides total protein group counts for each of 200 total AD, MCI, and healthy subject samples with a 10 particle panel.

FIG. 28 summarizes coefficients of variation for intensities of protein groups identified from 200 total AD, MCI, and healthy subject samples on a 10 particle panel.

FIG. 29 provides an empirical power curve for biomolecule corona data generated from 200 total AD, MCI, and healthy subject samples with a 10 particle panel.

FIG. 30 provides an ROC plot for an AD versus control classification model utilizing data from 10 particle types.

FIG. 31 summarizes features from a Random Forest classifier for distinguishing healthy and AD samples based on biomolecule corona data generated with 10 particle types on 50 AD samples and 100 healthy subject samples.

FIG. 32 provides an ROC plot for an MCI versus control classification model utilizing data from 10 particle types.

FIG. 33 summarizes features from a Random Forest classifier for distinguishing healthy and MCI samples based on biomolecule corona data generated with 10 particle types on 50 MCI samples and 100 healthy subject samples.

FIG. 34 provides an ROC plot for an MCI versus AD classification model utilizing data from 10 particle types.

FIG. 35 summarizes features from a Random Forest classifier for distinguishing MCI and AD samples based on biomolecule corona data generated with 10 particle types on 50 MCI samples and 50 AD subject samples.

FIG. 36 illustrates a workflow utilizing assay instrumentation and materials and a computer-implemented system for biological state analysis. FIG. 36 discloses SEQ ID NOS 461 and 462, respectively, in order of appearance.

FIGS. 37A-37B shows microscope images of citrate coated superparamagnetic iron oxide nanoparticles. The particles had a mean size of 150 nm (as determined by dynamic light scattering), and zeta potentials of around −30 mV.

FIGS. 38A-38B shows microscope images of the silica coated SPIONs. The particles had a mean size of 250-280 nm (as determined by dynamic light scattering), and zeta potentials of around −40 mV.

FIGS. 39A-39B shows microscope images of amine coated SPIONs. The particles had a mean size of 280 nm (as determined by dynamic light scattering), and zeta potentials of around +30 mV.

FIGS. 40A-40B shows microscope images of PDMAPMA coated SPIONs. The particles had a mean size of 400 nm (as determined by dynamic light scattering), and zeta potentials of around +30 mV.

FIGS. 41A-41B shows microscope images of carboxylate, polyacrylic acid (PAA) SPIONs. The particles had a mean size of 380 nm (as determined by dynamic light scattering), and zeta potentials of around −38 mV.

FIGS. 42A-42B shows microscope images of polystyrene carboxyl functionalized particles. The particles had a mean size of 229 nm±15 nm (as determined by transmission electron microscope imaging), and zeta potentials of about −36 to −40 mV.

FIGS. 43A-43B shows microscope images of amine functionalized silica-coated SPIONs. The particles had a mean size of 280 nm (as determined by dynamic light scattering), and zeta potentials of around +30 mV.

FIG. 44 shows a microscope image of glucose-6-phosphate functionalized SPIONs. The particles had a mean size of 175 nm±10 nm (as determined by dynamic light scattering), and zeta potentials of around −30 to −36 mV.

FIG. 45 outlines properties of a particle panel with SP-003, SP-006, SP-007, SP-373, and SP-125 particles.

FIG. 46 outlines properties of particles of two particle panels.

FIG. 47 compares physicochemical properties of two particle panels.

FIGS. 48A-48C shows studies where the number of protein groups unique to AD or MCI, or common to both were identified.

### DETAILED DESCRIPTION

From a molecular perspective, neurological disease progression is often difficult to assess, as neurodegeneration is typically associated with multiple underlying and often independent causes. For example, presently recognized mild cognitive impairment (MCI) and Alzheimer's disease (AD) risk factors and indicators may include vascular damage, hypertension, atherosclerosis, infection (including numerous forms of herpes simplex infections), personality changes, cognitive decline, or metabolic abnormalities, with some researchers even positing Alzheimer's disease as “Type 3” diabetes. As many neurological disease risk factors and indicators overlap with those of non-neurological conditions (e.g., liver disease and cirrhosis), identifying and distinguishing neurological diseases is often infeasible with standard pathological and biomarker analysis methods. Further complicating neurological disease analysis, neurological diseases may manifest negligible changes outside of affected tissues, rendering many forms of non-intensive (e.g., blood-based) neurological disease analysis poorly prognostic. Accordingly, options for neurological disease diagnoses absent expensive imaging and intensive nerve biopsy analyses have remained limited.

Responsive to the need for rapid, accurate, and minimally intensive neurological disease diagnostics, the present disclosure provides a range of compositions, systems, and methods for assessing neurological diseases from patient samples. In some cases, the compositions, systems, and methods may be configured to utilize blood or components thereof (e.g., whole blood, plasma, serum) to determine the presence of a neurological disease, such as Alzheimer's disease. The methods, systems, and compositions of the present disclosure may identify a plurality of biomolecules from sample and may furthermore determine relative or absolute abundances of at least a subset of the biomolecules. This may be compared to other blood biomarker tests, some of which may be used identify only a single biomolecule (e.g., a particular protein) from blood samples.

A method of the present disclosure may comprise contacting a biological sample (e.g., plasma) with a particle under conditions suitable for biomolecule collection (e.g., non-covalent adsorption of a protein) on the particle. The collection of biomolecules on the surface of the particle may be referred to as a ‘biomolecule corona’. The biomolecule corona that forms on a particle may comprise a complex mixture of biomolecules from the biological sample. A biomolecule corona may include nucleic acids, small molecules, proteins, lipids, polysaccharides, or any combination thereof. The biomolecule corona may compress the abundance ratios of biomolecules from a sample, thereby enabling analysis of dilute, and in many cases difficult to analyze, biomolecules.

A method of the present disclosure may comprise fractionating a biological sample with a particle. In some cases, the method comprises contacting the biological sample with the particle to form thereon a biomolecule corona which comprises biomolecules from the biological sample. The method may comprise separating the biomolecule corona from the biological sample, for example by immobilizing (e.g., magnetically trapping) the particle within a volume and removing unbound components of the biological sample from the volume (e.g., through a series of wash steps). The method may also comprise analyzing a biomolecule of the biomolecule corona. The analyzing may identify the biomolecule, determine an abundance of the biomolecule, identify a state (e.g., post-transcriptional processing of RNA or a post-translational modification of a protein) or form (e.g., a conformation) of the biomolecule, or identify a biomolecule-biomolecule interaction (e.g., a protein-protein interaction reflected, for example, by the formation of a multi-protein complex). As a biomolecule corona may comprise a compressed dynamic range relative to a sample, the analyzing may identify biomolecules over a broader dynamic range (in terms of biological sample concentrations of the biomolecules) than if the analyzing were performed directly on the biological sample (e.g., without particle-based fractionation of the biological sample).

In some cases, the method comprises contacting the biological sample with a plurality of particles. As biomolecule corona composition may depend on a number of factors, including biological sample composition, biological sample conditions (e.g., pH and salinity), particle concentration, and particle physicochemical properties (e.g., surface charge, hydrophilicity, density, roughness), contacting a sample with a plurality of particles may generate a plurality of biomolecule coronas which reflect different characteristics of the sample. For example, a biomolecule corona of a first particle may be sensitive to sample lipid levels, while a biomolecule of a second particle may be sensitive to nanomolar-scale changes in cytokine concentrations. Furthermore, two biomolecule coronas may comprise different subsets of biomolecules from a sample. Accordingly, the method may not only identify a plurality of biomolecules from a biological sample, but may also generate additional information by identifying one or more relationships between biomolecule corona composition, particle type, and sample conditions.

Aspects of the present disclosure provide compositions, systems, and methods for collecting biomolecules on particles, as well as particle panels of multiple distinct particle types, which may enrich proteins from a sample onto distinct biomolecule coronas formed on the surface of the distinct particle types. The particle panels disclosed herein can be used in methods of corona analysis to detect tens, hundreds, thousands, or tens of thousands of proteins across a wide dynamic range in the span of hours. In some cases, the composition, system, or method may utilize one particle. In some cases, the composition, system, or method may utilize at least two particles. In some cases, the composition, system, or method may utilize at least three particles. In some cases, the composition, system, or method may utilize at least four particles. In some cases, the composition, system, or method may utilize at least five particles. In some cases, the composition, system, or method may utilize at least six particles. In some cases, the composition, system, or method may utilize at least eight particles. In some cases, the composition, system, or method may utilize at least ten particles. In some cases, the composition, system, or method may utilize at least twelve particles. In some cases, the composition, system, or method may utilize at least fifteen particles. In some cases, the composition, system, or method may contact a sample with a particle under at least two conditions (e.g., at least two temperatures), and may compare the biomolecule corona formed under each of the at least two conditions. In some cases, the method may comprise identifying an abundance ratio of a biomolecule on two or more particles. In some cases, the method may comprise identifying an abundance ratio of a plurality of biomolecules on a particle. In some cases, the method may comprise identifying an abundance ratio of a first biomolecule on a first particle and a second biomolecule on a second particle.

In some cases, the a method of the present disclosure may be used to identify a biological state, such as a neurological disease state. In some cases, the method may distinguish a healthy biological state from a diseased biological state, or may identify a stage of a biological state, for example early stage Alzheimer's disease from biomolecule corona data of a biological sample. In some cases, the method may identify a subject or a biological sample as healthy. In some cases, a healthy state may exclude a disease state. For example, a healthy state may exclude having a neurological disorder. In some cases, a disease state may exclude being healthy.

### Particle Properties and Types

Particle types consistent with the methods disclosed herein can be made from various materials. For example, particle materials of the present disclosure may include metals, polymers, magnetic materials, and lipids. Magnetic particles may be iron oxide particles. Examples of metals include any one of gold, silver, copper, nickel, cobalt, palladium, platinum, iridium, osmium, rhodium, ruthenium, rhenium, vanadium, chromium, manganese, niobium, molybdenum, tungsten, tantalum, iron, cadmium, any other material described in U.S. Pat. No. 7,749,299, or any combination thereof. In some cases, a particle may be a superparamagnetic iron oxide nanoparticle (SPION). A magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material). For example, a particle core may comprise superparamagnetic γ-ferric iron oxide. In some cases, a particle may comprise a distinct core (e.g., the innermost portion of the particle), shell (e.g., the outermost layer of the particle), and shell or shells (e.g., portions of the particle disposed between the core and the shell). In some cases, a core may comprise a metal, an oxide, a nitride, a ceramic, a carbon material, a silicon material, a polymer, or any combination thereof. In some cases, a shell may comprise a polymer, a saccharide, a lipid, a peptide, a self-assembled monolayer, a sol-gel, a hydrogel, a glass, or any combination thereof. In some cases, a shell may comprise polystyrene, N-(3-(Dimethylamino)propyl)methacrylamide (DMAPMA), or a combination thereof. In some cases, a shell material may comprise a small molecule functionalization. In some cases, a shell material may comprise a biomolecular functionalization (e.g., a peptide or saccharide functional appendage). In some cases, a particle may comprise a uniform composition. In some cases, a core or a shell may comprise a plurality of materials comprising a degree of phase separation. For example, a shell may comprise two phase separated polymers. In some cases, a particle core and shell may comprise different densities. In some cases, a shell material may comprise a thickness of at least 2 nm, at least 4 nm, at least 5 nm, at least 8 nm, at least 10 nm, at least 15 nm, at least 20 nm, at least 25 nm, at least 30 nm, or at least 35 nm. In some cases, a shell material may comprise a thickness of at most 35 nm, at most 30 nm, at most 25 nm, at most 20 nm, at most 15 nm, at most 10 nm, at most 8 nm, at most 5 nm, at most 4 nm, or at most 2 nm.

In some cases, a particle may comprise a polymer. In some cases, the polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. In some cases, the polymer may comprise a polyethylene, a polycarbonate, a polyanhydride, a polyhydroxyacid, a polypropylfumerate, a polycaprolactone, a polyamide, a polyacetal, a polyether, a polyester, a poly(orthoester), a polycyanoacrylate, a polyvinyl alcohol, a polyurethane, a polyphosphazene, a polyacrylate, a polymethacrylate, a polycyanoacrylate, a polyurea, a polystyrene, a polyamine, a polyalkylene glycol (e.g., polyethylene glycol (PEG)), a polyester (e.g., poly(lactide-co-glycolide) (PLGA) or a polylactic acid), a copolymer of two or more polymers (e.g., a copolymer of a polyalkylene glycol (e.g., PEG) and a polyester (e.g., PLGA)), or any combination thereof. In some cases, the polymer may be a lipid-terminated polyalkylene glycol and a polyester, or any other material disclosed in U.S. Pat. No. 9,549,901.

In some cases, a particle may comprise a lipid. In some cases, a lipid-containing particle may comprise a lipid coupled to its surface (e.g., covalently attached to a surface amine of the particle or non-covalently bound by a particle-bound lipid binding protein). In some cases, a lipid-containing particle may comprise a lipid within a monolayer or bilayer comprising the lipid. In some cases, the lipid monolayer or bilayer may comprise non-lipidic biomolecules, including sterols, proteins (e.g., clathrins), and saccharides. In some cases, a plurality of lipids associated with a particle may be fully or partially polymerized. In some cases, a particle may comprise a liposome. Examples of lipids that can be used to form the particles of the present disclosure include cationic, anionic, and neutrally charged lipids. In some cases, particles can be made of any one of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidylethanolamine (DSPE), palmitoyloleoyl-phosphatidylethanolamine (POPE) palmitoyloleoylphosphatidylcholine (POPC), egg phosphatidylcholine (EPC), di stearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, cholesterol, any other material listed in U.S. Pat. No. 9,445,994 (which is incorporated herein by reference in its entirety), or any combination thereof.

Examples of particles of the present disclosure are provided in TABLE 1.

TABLE 1
Example particles of the present disclosure

| Batch No. | Type | Particle ID | Description |
|---|---|---|---|
| S-001-001 | HX-13 | SP-001 | Carboxylate (Citrate) superparamagnetic |
| | | | iron oxide NPs (SPION) |
| S-002-001 | HX-19 | SP-002 | Phenol-formaldehyde coated SPION |
| S-003-001 | HX-20 | SP-003 | Silica-coated superparamagnetic iron |
| | | | oxide NPs (SPION) |
| S-004-001 | HX-31 | SP-004 | Polystyrene coated SPION |
| S-005-001 | HX-38 | SP-005 | Carboxylated Poly(styrene-co- |
| | | | methacrylic acid), P(St- |
| | | | co-MAA) coated SPION |
| S-006-001 | HX-42 | SP-006 | N-(3-Trimethoxysilylpropyl) |
| | | | diethylenetriamine coated SPION |
| S-007-001 | HX-56 | SP-007 | poly(N-(3-(dimethylamino) |
| | | | propyl) methacrylamide) |
| | | | (PDMAPMA)-coated SPION |
| S-008-001 | HX-57 | SP-008 | 1,2,4,5-Benzenetetracarboxylic |
| | | | acid coated SPION |
| S-009-001 | HX-58 | SP-009 | poly(vinylbenzyltrimethyl- |
| | | | ammonium chloride) |
| | | | (PVBTMAC) coated SPION |
| S-010-001 | HX-59 | SP-010 | Carboxylate, PAA coated SPION |
| S-011-001 | HX-86 | SP-011 | poly(oligo(ethylene glycol) methyl |
| | | | ether methacrylate) |
| | | | (POEGMA)-coated SPION |
| P-033-001 | P33 | SP-333 | Carboxylate microparticle, |
| | | | surfactant free |
| P-039-003 | P39 | SP-339 | Polystyrene carboxyl functionalized |
| P-041-001 | P41 | SP-341 | Carboxylic acid |
| P-047-001 | P47 | SP-365 | Silica |
| P-048-001 | P48 | SP-348 | Carboxylic acid, 150 nm |
| P-053-001 | P53 | SP-353 | Amino surface microparticle, |
| | | | 0.4-0.6 μm |
| P-056-001 | P56 | SP-356 | Silica amino functionalized |
| | | | microparticle, 0.1-0.39 μm |
| P-063-001 | P63 | SP-363 | Jeffamine surface, 0.1-0.39 μm |
| P-064-001 | P64 | SP-364 | Polystyrene microparticle, 2.0-2.9 μm |
| P-065-001 | P65 | SP-365 | Silica |
| P-069-001 | P69 | SP-369 | Carboxylated Original coating, 50 nm |
| P-073-001 | P73 | SP-373 | Dextran based coating, 0.13 μm |
| P-074-001 | P74 | SP-374 | Silica Silanol coated with lower acidity |
| — | S-118 | SP-118 | 1,6-hexanediamine functionalized |
| | | | SPION |
| — | S-125 | SP-125 | Amine functionalized silica-coated |
| | | | SPION |
| — | S-128 | SP-128 | Mixed amide, carboxylate |
| | | | functionalized, silica-coated SPION |
| — | S-199 | SP-199 | Epichlorohydrin crosslinked Dextran- |
| | | | coated SPION |
| — | S-229 | SP-229 | N¹-(3-(trimethoxysilyl)propyl) |
| | | | hexane-1,6-diamine functionalized, |
| | | | silica-coated SPION |

A particle of the present disclosure may be synthesized, or a particle of the present disclosure may be purchased from a commercial vendor. For example, some particles of the present disclosure may be purchased from commercial vendors including Sigma-Aldrich, Life Technologies, Fisher Biosciences, nanoComposix, Nanopartz, Spherotech, and other commercial vendors. In some cases, a particle of the present disclosure may be purchased from a commercial vendor and further modified, coated, or functionalized.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo(ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity. An example of a particle type of the present disclosure may be a mixed amide, carboxylate functionalized, silica-coated SPION having a mean size of about 280 nm and a zeta potential of about 50 mV. An example of a particle type of the present disclosure may be an epichlorohydrin crosslinked Dextran-coated SPION having a mean size of about 275+/−30 nm and a zeta potential of about 15 to 20 mV. An example of a particle type of the present disclosure may be a N¹-(3-(trimethoxysilyl)propyl)hexane-1,6-diamine functionalized, silica-coated SPION having a mean size of about 280 nm and a zeta potential of about 40 mV.

Particles of the present disclosure can be made and used in methods of forming protein coronas after incubation in a biofluid at a wide range of sizes. In some cases, a particle of the present disclosure may be a nanoparticle. In some cases, a nanoparticle of the present disclosure may be from about 10 nm to about 1000 nm in diameter. In some cases, a nanoparticle may be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm in diameter. In some cases, a nanoparticle may be less than 1000 nm in diameter. In some cases, a particle may comprise a diameter of about 30 nm to about 800 nm. In some cases, a particle comprises a diameter of about 60 nm to about 600 nm. In some cases, a particle comprises a diameter of about 60 nm to about 500 nm. In some cases, a particle comprises a diameter of about 60 nm to about 400 nm. In some cases, a particle comprises a diameter of about 60 nm to about 300 nm. In some cases, a particle comprises a diameter of about 60 nm to about 200 nm. In some cases, a particle comprises a diameter of about 60 nm to about 150 nm. In some cases, a particle comprises a diameter of about 80 nm to about 500 nm. In some cases, a particle comprises a diameter of about 80 nm to about 400 nm. In some cases, a particle comprises a diameter of about 80 nm to about 300 nm. In some cases, a particle comprises a diameter of about 80 nm to about 200 nm. In some cases, a particle comprises a diameter of about 80 nm to about 150 nm. In some cases, a particle comprises a diameter of about 100 nm to about 500 nm. In some cases, a particle comprises a diameter of about 100 nm to about 400 nm. In some cases, a particle comprises a diameter of about 100 nm to about 300 nm. In some cases, a particle comprises a diameter of about 100 nm to about 200 nm. In some cases, a particle comprises a diameter of about 100 nm to about 150 nm. In some cases, a particle comprises a diameter of about 120 nm to about 600 nm. In some cases, a particle comprises a diameter of about 120 nm to about 500 nm. In some cases, a particle comprises a diameter of about 120 nm to about 400 nm. In some cases, a particle comprises a diameter of about 120 nm to about 350 nm. In some cases, a particle comprises a diameter of about 120 nm to about 300 nm. In some cases, a particle comprises a diameter of about 120 nm to about 200 nm. In some cases, a particle comprises a diameter of about 150 nm to about 600 nm. In some cases, a particle comprises a diameter of about 150 nm to about 500 nm. In some cases, a particle comprises a diameter of about 150 nm to about 400 nm. In some cases, a particle comprises a diameter of about 150 nm to about 300 nm. In some cases, a particle comprises a diameter of about 200 nm to about 400 nm. In some cases, a particle comprises a diameter of about 200 nm to about 600 nm. In some cases, a particle comprises a diameter of at least about 100 nm. In some cases, a particle comprises a diameter of at most 500 nm.

In some cases, a particle of the present disclosure may be a microparticle. A microparticle may be a particle that is from about 1 μm to about 1000 μm in diameter. For example, the microparticles disclosed here can be at least 1 μm, at least 10 μm, at least 100 μm, at least 200 μm, at least 300 μm, at least 400 μm, at least 500 μm, at least 600 μm, at least 700 μm, at least 800 μm, at least 900 μm, from 10 μm to 50 μm, from 50 μm to 100 μm, from 100 μm to 150 μm, from 150 μm to 200 μm, from 200 μm to 250 μm, from 250 μm to 300 μm, from 300 μm to 350 μm, from 350 μm to 400 μm, from 400 μm to 450 μm, from 450 μm to 500 μm, from 500 μm to 550 μm, from 550 μm to 600 μm, from 600 μm to 650 μm, from 650 μm to 700 μm, from 700 μm to 750 μm, from 750 μm to 800 μm, from 800 μm to 850 μm, from 850 μm to 900 μm, from 100 μm to 300 μm, from 150 μm to 350 μm, from 200 μm to 400 μm, from 250 μm to 450 μm, from 300 μm to 500 μm, from 350 μm to 550 μm, from 400 μm to 600 μm, from 450 μm to 650 μm, from 500 μm to 700 μm, from 550 μm to 750 μm, from 600 μm to 800 μm, from 650 μm to 850 μm, from 700 μm to 900 μm, or from 10 μm to 900 μm in diameter. In some cases, a microparticle may be less than 1000 μm in diameter. In some cases, a microparticle may comprise a diameter of about 1 μm to about 2 μm. In some cases, a microparticle may comprise a diameter of about 1 μm to about 1.5 μm.

A substrate (such as a particle) may comprise a degree of shape or size uniformity or non-uniformity. A physical measure of such heterogeneity may be polydispersity, which tracks size uniformity of a substrate, and may be defined as the square of the ratio of the standard deviation and the mean of substrate size (e.g., particle diameter). Alternatively, polydispersity may be a ratio of (1) weight average molecular weight to (2) number average molecular weight for a substrate (e.g., for a collection of particles), and therefore serves as a measure of mass variance for the substrate. A substrate may comprise a low polydispersity value, indicating a high degree of size uniformity. For example, a substrate (e.g., a collection of a substrate comprising a plurality of copies of the substrate) may comprise a polydispersity index of at most 1.6, at most 1.4, at most 1.2, at most 1, at most 0.8, at most 0.6, at most 0.5, at most 0.4, at most 0.3, at most 0.25, at most 0.2, at most 0.15, at most 0.1, at most 0.05, at most 0.03, or at most 0.02. Alternatively, a substrate may comprise a high polydispersity index, indicating a degree of size and/or mass variation. For example, a substrate (e.g., a collection of a substrate comprising a plurality of copies of the substrate) may comprise a polydispersity index of at least 0.3, at least 0.4, at least 0.5, at least 0.6, at least 0.8, at least 1, at least 1.2, at least 1.4, at least 1.6, at least 1.8, at least 2, at least 2.2, at least 2.5, or at least 3.

A particle may be substantially spherical. A particle may comprise an oblong geometry. A particle may comprise a surface feature, such as a well, a trench, or a substantially flat region.

A particle may be provided at a range of concentrations. A particle may be provided at a concentration of at least 10 pM. A particle may be provided at a concentration of at least 100 pM. A particle may be provided at a concentration of at least 1 nM. A particle may be provided at a concentration of at least 10 nM. A particle may be provided at a concentration of at most 100 nM. A particle may be provided at a concentration of at most 10 nM. A particle may be provided at a concentration of at most 1 nM. A particle may be provided at a concentration of at most 100 pM. A particle may be provided at a concentration of at most 10 pM. A particle may be provided at a concentration of at most 1 pM. A particle may be provided at a concentration between 100 fM and 100 nM. A particle may be provided at a concentration between 100 fM and 10 pM. A particle may be provided at a concentration between 1 pM and 100 pM. A particle may be provided at a concentration between 10 pM and 1 nM. A particle may be provided at a concentration between 100 pM and 10 nM. A particle may be provided at a concentration between 1 nM and 100 nM. A particle may be provided at a concentration of at least 10 ng/ml. A particle may be provided at a concentration of at least 100 ng/ml. A particle may be provided at a concentration of at least 1 μg/ml. A particle may be provided at a concentration of at least 10 μg/ml. A particle may be provided at a concentration of at least 100 μg/ml. A particle may be provided at a concentration of at least 1 mg/ml. A particle may be provided at a concentration of at least mg/ml. A particle may be provided at a concentration of at least 10 mg/ml. A particle may be provided at a concentration of at most 10 mg/ml. A particle may be provided at a concentration of at most 1/ml. A particle may be provided at a concentration of at most 100 μg/ml. A particle may be provided at a concentration of at most 10 μg/ml. A particle may be provided at a concentration of at most 1 μg/ml. A particle may be provided at a concentration of at most 100 ng/ml. A particle may be provided at a concentration of at most 10 ng/ml.

A particle may be contacted to a biological sample at a range of volume ratios. A solution comprising a particle may be combined with a biological sample, at a volume ratio of greater than about 100:1, about 100:1, about 80:1, about 60:1, about 50:1, about 40:1, about 30:1, about 25:1, about 20:1, about 15:1, about 12:1, about 10:1, about 8:1, about 6:1, about 5:1, about 4:1, about 3:1, about 5:2, about 2:1, about 3:2, about 1:1, about 2:3, about 1:2, about 2:5, about 1:3, about 1:4, about 1:5, about 1:6, about 1:8, about 1:10, about 1:12, about 1:15, about 1:20, about 1:25, about 1:30, about 1:40, about 1:50, about 1:60, about 1:80, about 1:100, or less than about 1:100.

In some cases, the ratio between surface area and mass can be a determinant of a particle's properties. In some cases, the number and types of biomolecules that a particle adsorbs from a solution varies with the particle's surface area to mass ratio. In some cases, a particle can have a surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. In some cases, small particles (e.g., with diameters of 50 nm or less) can have significantly higher surface area to mass ratios, stemming in part from the higher order dependence on diameter by mass than by surface area. In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg.

In some cases, a plurality of particles (e.g., of a particle panel) used with the methods described herein may have a range of surface area to mass ratios. In some cases, the range of surface area to mass ratios for a plurality of particles is less than 100 cm²/mg, 80 cm²/mg, 60 cm²/mg, 40 cm²/mg, 20 cm²/mg, 10 cm²/mg, 5 cm²/mg, or 2 cm²/mg. In some cases, the surface area to mass ratios for a plurality of particles varies by no more than 40%, 30%, 20%, 10%, 5%, 3%, 2%, or 1% between the particles in the plurality. In some cases, the plurality of particles may comprise at least 2, 3, 4, 5, 6, 7, 8, 9, 10, 12, 15, 20, or more different types of particles.

In some cases, a plurality of particles (e.g., in a particle panel) may comprise a range of surface area to mass ratios. In some cases, the range of surface area to mass ratios for a plurality of particles is greater than 100 cm²/mg, 150 cm²/mg, 200 cm²/mg, 250 cm²/mg, 300 cm²/mg, 400 cm²/mg, 500 cm²/mg, 800 cm²/mg, 1000 cm²/mg, 1200 cm²/mg, 1500 cm²/mg, 2000 cm²/mg, 3000 cm²/mg, 5000 cm²/mg, 6000 cm²/mg, 7500 cm²/mg, 10000 cm²/mg, or more. In some cases, the surface area to mass ratios for a plurality of particles (e.g., within a panel) can vary by more than 100%, 200%, 300%, 400%, 500%, 1000%, 10000% or more. In some cases, the plurality of particles with a wide range of surface area to mass ratios may comprise at least 2, 3, 4, 5, 6, 7, 8, 9, 10, 12, 15, 20, or more different types of particles.

A particle may comprise a wide range of physical properties. A physical property of a particle may comprise composition, size, surface charge, hydrophobicity, hydrophilicity, surface functionalization, surface topography, surface curvature, porosity, core material, shell material, shape, or any combination thereof.

A surface functionalization may comprise a polymerizable functional group, a positively or negatively charged functional group, a zwitterionic functional group, an acidic or basic functional group, a polar functional group, or any combination thereof. In some cases, a surface functionalization comprises a polar functional group, an acidic functional group, a basic functional group, a charged functional group, a polymerizable functional group, or any combination thereof. In some cases, a surface functionalization may comprise an aminopropyl functionalization, an amine functionalization, an amide functionalization, a boronic acid functionalization, a carboxylic acid functionalization, a methyl functionalization, an N-succinimidyl ester functionalization, a PEG functionalization, a streptavidin functionalization, a methyl ether functionalization, a triethoxylpropylaminosilane functionalization, a thiol functionalization, a PCP functionalization, a citrate functionalization, a lipoic acid functionalization, a BPEI functionalization, carboxyl functionalization, a hydroxyl functionalization, or any combination thereof. In some cases, a surface functionalization may comprise carboxyl groups, hydroxyl groups, thiol groups, cyano groups, nitro groups, ammonium groups, alkyl groups, imidazolium groups, sulfonium groups, pyridinium groups, pyrrolidinium groups, phosphonium groups, aminopropyl groups, amine groups, amide groups, boronic acid groups, N-succinimidyl ester groups, PEG groups, streptavidin, methyl ether groups, triethoxylpropylaminosilane groups, PCP groups, citrate groups, lipoic acid groups, BPEI groups, or any combination thereof. In some cases, a surface functionalization may be present at various ranges of densities on a particle. In some cases, a surface functionalization comprises an average density of at least about 1 functional group per 20 nm² on a surface of a particle. In some cases, a surface functionalization may comprise an average density of at least about 1 functional group per 30 nm² on a surface of a particle. In some cases, a surface functionalization may comprise an average density of at least about 1 functional group per 40 nm² on a surface of a particle. In some cases, a surface functionalization may comprise an average density of at least about 1 functional group per 50 nm² on a surface of a particle. In some cases, a surface functionalization may comprise an average density of at least about 1 functional group per 60 nm² on a surface of a particle. In some cases, a surface functionalization may comprise an average density of at least about 1 functional group per 80 nm² on a surface of a particle. In some cases, a surface functionalization may comprise an average density of at most about 1 functional group per 80 nm² on a surface of a particle. In some cases, a surface functionalization may comprise an average density of at most about 1 functional group per 60 nm² on a surface of a particle. In some cases, a surface functionalization may comprise an average density of at most about 1 functional group per 50 nm² on a surface of a particle. In some cases, a surface functionalization may comprise an average density of at most about 1 functional group per 40 nm² on a surface of a particle. In some cases, a surface functionalization may comprise an average density of at most about 1 functional group per 30 nm² on a surface of a particle. In some cases, a surface functionalization may comprise an average density of at most about 1 functional group per 20 nm² on a surface of a particle. In some cases, a surface functionalization may comprise an average density about 1 functional group per 20 nm² to at most about 1 functional group per 60 nm² on a surface of a particle.

In some cases, a particle may be selected from the group consisting of: micelles, liposomes, iron oxide particles, silver particles, gold particles, palladium particles, quantum dots, platinum particles, titanium particles, silica particles, metal or inorganic oxide particles, synthetic polymer particles, copolymer particles, terpolymer particles, polymeric particles with metal cores, polymeric particles with metal oxide cores, polystyrene sulfonate particles, polyethylene oxide particles, polyoxyethylene glycol particles, polyethylene imine particles, polylactic acid particles, polycaprolactone particles, polyglycolic acid particles, poly(lactide-co-glycolide polymer particles, cellulose ether polymer particles, polyvinylpyrrolidone particles, polyvinyl acetate particles, polyvinylpyrrolidone-vinyl acetate copolymer particles, polyvinyl alcohol particles, acrylate particles, polyacrylic acid particles, crotonic acid copolymer particles, polyethlene phosphonate particles, polyalkylene particles, carboxy vinyl polymer particles, sodium alginate particles, carrageenan particles, xanthan gum particles, gum acacia particles, Arabic gum particles, guar gum particles, pullulan particles, agar particles, chitin particles, chitosan particles, pectin particles, karaya tum particles, locust bean gum particles, maltodextrin particles, amylose particles, corn starch particles, potato starch particles, rice starch particles, tapioca starch particles, pea starch particles, sweet potato starch particles, barley starch particles, wheat starch particles, hydroxypropylated high amylose starch particles, dextrin particles, levan particles, elsinan particles, gluten particles, collagen particles, whey protein isolate particles, casein particles, milk protein particles, soy protein particles, keratin particles, polyethylene particles, polycarbonate particles, polyanhydride particles, polyhydroxyacid particles, polypropylfumerate particles, polycaprolactone particles, polyamine particles, polyacetal particles, polyether particles, polyester particles, poly(orthoester) particles, polycyanoacrylate particles, polyurethane particles, polyphosphazene particles, polyacrylate particles, polymethacrylate particles, polycyanoacrylate particles, polyurea particles, polyamine particles, polystyrene particles, poly(lysine) particles, chitosan particles, dextran particles, poly(acrylamide) particles, derivatized poly(acrylamide) particles, gelatin particles, starch particles, chitosan particles, dextran particles, gelatin particles, starch particles, poly-β-amino-ester particles, poly(amido amine) particles, poly lactic-co-glycolic acid particles, polyanhydride particles, bioreducible polymer particles, 2-(3-aminopropylamino)ethanol particles, and any combination thereof.

In some cases, particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, an amide functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some cases, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group.

A small molecule functionalization may comprise a polymerizable functional group. Non-limiting examples of the polymerizable functional group include a vinyl group and a (meth)acrylic group. In some cases, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. A zwitterionic particle surface may be zwitterionic over at least 1, at least 2, at least 3, at least 4, at least 5, at least 6 or more pH units. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle. In some cases, a charge of a particle may be determined by measuring the zeta potential of the particle.

### Particle Panels

The present disclosure provides compositions and methods of use thereof for assaying a sample for proteins. Compositions described herein may include particle panels comprising one or more than one distinct particle types. Particle panels described herein can vary in the number of particle types and the diversity of particle types in a single panel. For example, particles in a panel may vary based on size, polydispersity, shape and morphology, surface charge, surface chemistry and functionalization, and base material. Panels may be incubated with a sample to be analyzed for protein composition. Proteins in the sample may adsorb to the surface of the different particle types in the particle panel to form a protein corona. The types of proteins which adsorb to a certain particle type in the particle panel may depend on the composition, size, and surface charge of the particle type. Thus, each particle type in a panel may have different protein coronas due to adsorbing a different set of proteins, different concentrations of a particular protein, or a combination thereof. Each particle type in a panel may have mutually exclusive protein coronas or may have overlapping protein coronas. Overlapping protein coronas can overlap in protein identity, in protein concentration, or both.

The present disclosure also provides methods for selecting a particle types for inclusion in a panel depending on the sample type. Particle types included in a panel may be a combination of particles that are optimized for removal of highly abundant proteins. Particle types also consistent for inclusion in a panel are those selected for adsorbing particular proteins of interest. In some cases, the particles may be nanoparticles. In some cases, the particles may be microparticles. In some cases, the particles may be a combination of nanoparticles and microparticles.

A particle panel including any number of distinct particle types disclosed herein, may enrich and identify a single protein or protein group. In some cases, the single protein or protein group may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the particle panel including any number of distinct particle types disclosed herein, may enrich and identify a single protein or protein group by binding different domains, sequences, or epitopes of the single protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel of the present disclosure can also comprise two or more particles selected from the group consisting of silica coated SPION, an N-(3-Trimethoxysilylpropyl) diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel of the present disclosure may also comprise two or more particles selected from the group consisting of a surfactant free carboxylate microparticle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino)propyl]methacrylamide-co-[2-(methacryloyloxy)ethyl]dimethyl-(3-sulfopropyl)ammonium hydroxide, P(DMAPMA-co-SBMA) coated particle. A particle panel of the present disclosure may comprise silica-coated particles, N-(3-Trimethoxysilylpropyl)diethylenetriamine coated particles, poly(N-(3-(dimethylamino)propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel of the present disclosure may comprise a silica functionalized particle, an amine functionalized particle, a silicon alkoxide functionalized particle, a carboxylate functionalized particle, and a benzyl or phenyl functionalized particle. A particle panel of the present disclosure may comprise a silica functionalized particle, an amine functionalized particle, a silicon alkoxide functionalized particle, a polystyrene functionalized particle, and a saccharide functionalized particle. A particle panel of the present disclosure may comprise a silica functionalized particle, an N-(3-Trimethoxysilylpropyl)diethylenetriamine functionalized particle, a PDMAPMA functionalized particle, a dextran functionalized particle, and a polystyrene carboxyl functionalized particle. A particle panel of the present disclosure may comprise 5 particles including a silica functionalized particle, an amine functionalized particle, a silicon alkoxide functionalized particle.

A particle panel of the present disclosure may comprise a silica particle, an amine functionalized particle, and a polyethylene glycol-functionalized particle. The particle panel may further comprise a carboxylate functionalized particle, such as a carboxylate functionalized styrene particle. The particle panel may further comprise a saccharide-coated particle. In some cases, the saccharide-coated particle is a dextran-coated particle. The particle panel may further comprise a sulfuryl functionalized particle. The sulfuryl functionalized particle may comprise a positively charged surface functionalization such as an amine, and thereby may be zwitterionic. The particle panel may further comprise a particle with a boronated or boronic acid functionalized surface. The particle panel may further comprise a particle with an oleic acid functionalized surface. The particle panel may comprise at least one microparticle.

The present disclosure includes compositions (e.g., particle panels) and methods that comprise two or more particles differing in at least one physicochemical property. A composition or method of the present disclosure may comprise 3 to 6 particles differing in at least one physicochemical property. A composition or method of the present disclosure may comprise 4 to 8 particles differing in at least one physicochemical property. A composition or method of the present disclosure may comprise 4 to 10 particles differing in at least one physicochemical property. A composition or method of the present disclosure may comprise 5 to 12 particles differing in at least one physicochemical property. A composition or method of the present disclosure may comprise 6 to 14 particles differing in at least one physicochemical property. A composition or method of the present disclosure may comprise 8 to 15 particles differing in at least one physicochemical property. A composition or method of the present disclosure may comprise 10 to 20 particles differing in at least one physicochemical property. A composition or method of the present disclosure may comprise at least 2 distinct particle types, at least 3 distinct particle types, at least 4 distinct particle types, at least 5 distinct particle types, at least 6 distinct particle types, at least 7 distinct particle types, at least 8 distinct particle types, at least 9 distinct particle types, at least 10 distinct particle types, at least 11 distinct particle types, at least 12 distinct particle types, at least 13 distinct particle types, at least 14 distinct particle types, at least 15 distinct particle types, at least 20 distinct particle types, at least 25 particle types, or at least 30 distinct particle types.

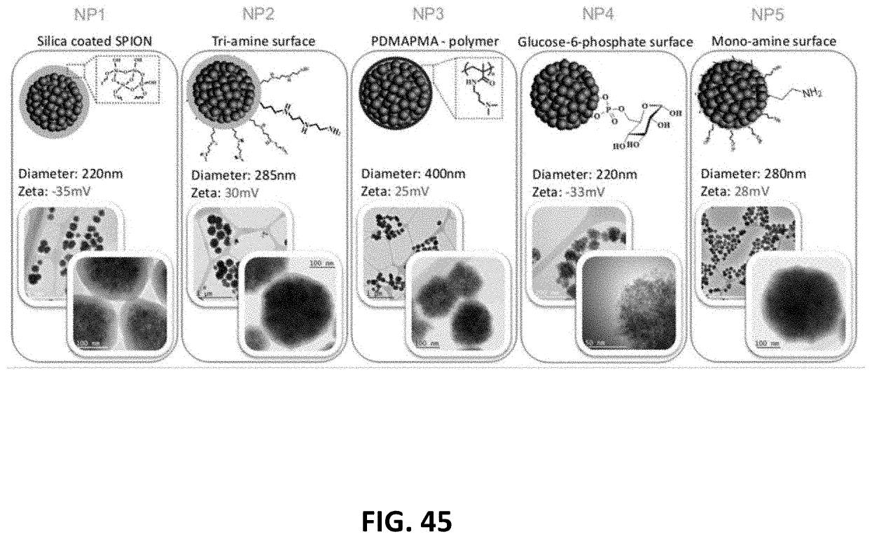

An example of a particle panel of the present disclosure is provided in FIG. 45, which provides physicochemical properties for the 5 particles SP-003, SP-006, SP-007, SP-373, and SP-125 particles. The particles in this panel range in size from 220 nm to 400 nm, and span zeta potentials of −35 mV to +30 mV.

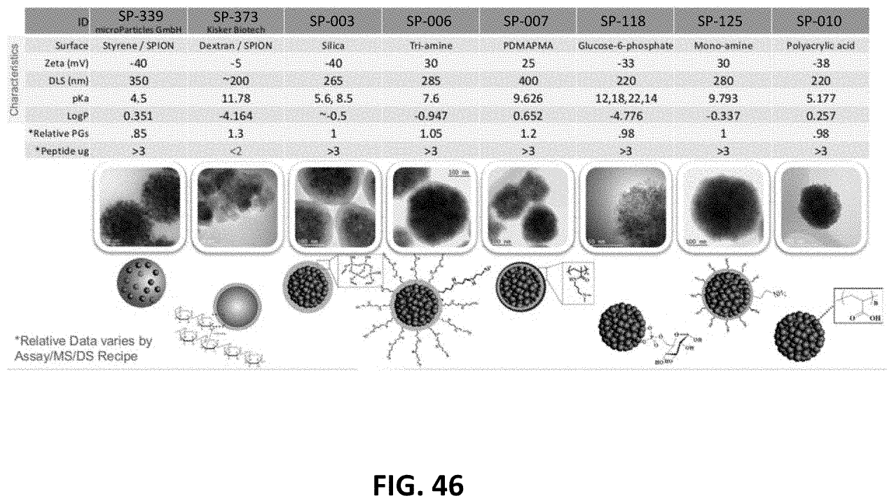

A further example of particle panels is provided in FIG. 46. This figure provides two particle panels, along with a number of physicochemical characteristics. Particle Panel A includes SP-039, SP-373, SP-003, SP-006, and SP-007 particles (summarized in TABLE 3, below), and spans sizes of about 200 nm to about 400 nm, zeta potentials of about −40 mV to about 30 mV, pKa values of about 4.5 to about 11.78, Log P (log of partition coefficient) values of about −4.2 to about 0.7, relative PGs (ratio of the number of detected protein groups relative to the number of protein groups detected by SP-003) values of about 0.8 to about 1.3, and peptide mass (collected from the particles before mass spectrometry) values of about less than about 2 μg to greater than about 3 μg. Particle Panel B comprises SP-003, SP-006, SP-007, SP-118, and SP-125 particles, and spans sizes of about 220 nm to about 400 nm, zeta potentials of about −40 mV to about 30 mV. Log P values of about −5 to about 0.7, relative PGs values of about 0.98 to about 1.2, and peptide mass of greater than about 3 μg.

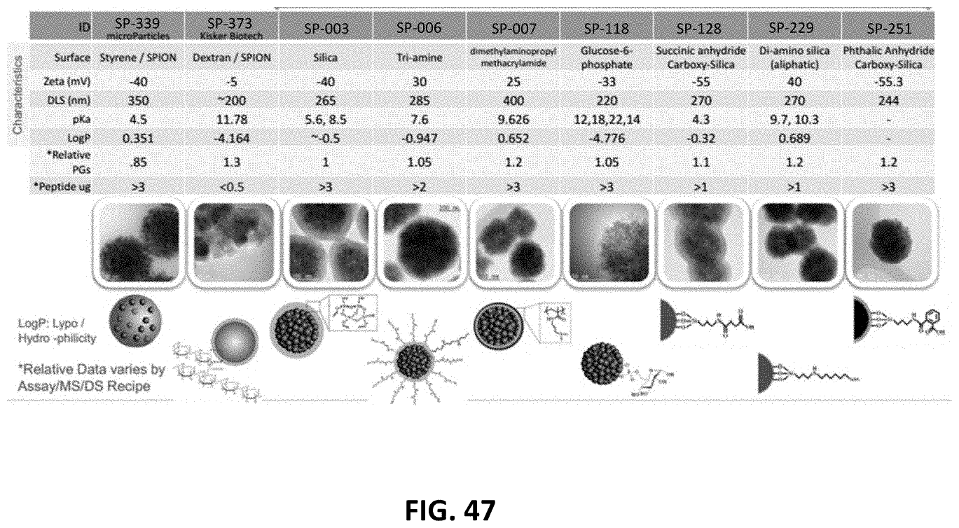

FIG. 47 compares physicochemical properties of two particle panels. Particle Panel A comprises SP-339, SP-373, SP-003, SP-006, and SP-007 particles (summarized in TABLE 3, below), and spans sizes of 200 nm to 400 nm, zeta potentials of −40 mV to 30 mV, pKa values of 4.5 to 11.78, log P values of about −4.2 to about 0.65, relative PGs of about 0.85 to 1.3, and peptide mass of less than about 0.5 μg to greater than about 3 μg. Particle Panel C comprises 2, 3, 4, 5, 6, or 7 particles from the group SP-003, SP-006, SP-007, SP-118, SP-128, SP-229, and SP-251. For example, the particle panel summarized in TABLE 2 comprises SP-003, SP-007, SP-118, SP-128, and SP-229. The 7 particles which may be utilized for Particle Panel C span sizes of 220 nm to 400 nm, zeta potentials of −55.3 mV to 40 mV, pKa values of 4.6 to 12, log P of about −5 to about 0.7, relative PGs of about 1 to about 1.2, and peptide mass greater than 1 or greater than 3 μg.

In some cases, a particle panel may comprise a particle listed in TABLE 2, below. A particle panel may comprise at least two particles listed in TABLE 2. In some cases, a particle panel may comprise at least three particles listed in TABLE 2. In some cases, a particle panel may comprise at least four particles listed in TABLE 2. In some cases, a particle panel may comprise the particles listed in TABLE 2.

TABLE 2
Example of a particle panel of the present disclosure

| Particle | |
|---|---|
| Name | Description |
| SP-003 | Silica-Coated SPION |
| SP-007 | Poly(dimethylaminopropylmethacrylamide)-coated SPION |
| SP-118 | Glucose-6-phosphate functionalized SPION |
| SP-128 | Mixed amide, carboxylate functionalized, silica-coated SPION |
| SP-229 | N¹-(3-(trimethoxysilyl)propyl)hexane-1,6- |
| | diamine functionalized, silica-coated SPION |

In some cases, a particle panel may comprise a particle listed in TABLE 3, below. In some cases, a particle panel may comprise at least two particles listed in TABLE 3. In some cases, a particle panel may comprise at least three particles listed in TABLE 3. In some cases, a particle panel may comprise at least four particles listed in TABLE 3. In some cases, a particle panel may comprise the particles listed in TABLE 3.

TABLE 3
Example of a particle panel of the present disclosure

| Particle | |
|---|---|
| Name | Description |
| SP-003 | Silica-Coated SPION |
| SP-006 | N-(3-Trimethoxysilylpropyl)diethylenetriamine-coated SPION |
| SP-007 | Poly(dimethylaminopropylmethacrylamide)-coated SPION |
| SP-339 | Carboxyl functionalized polystyrene-coated SPION |
| SP-373 | Dextran-coated SPION |

In some cases, a particle panel may comprise a particle listed in TABLE 4, below. In some cases, a particle panel may comprise at least two particles listed in TABLE 4. In some cases, a particle panel may comprise at least three particles listed in TABLE 4. In some cases, a particle panel may comprise at least four particles listed in TABLE 4. In some cases, a particle panel may comprise the particles listed in TABLE 4.

TABLE 4
Example of a particle panel of the present disclosure

| Particle | |
|---|---|
| ID | Description |
| SP-339 | Polystyrene particles, Paramagnetic, Carboxyl- |
| | functionalized (PS-MAG-COOH) |
| SP-373 | Magnetizable Nanoparticles and magnetizable |
| | microparticles, Dextran based//plain/25 mg/ml |
| SP-003 | Superparamagnetic, silica coated |
| SP-006 | Silica coated, amine |
| SP-007 | PDMAPMA coated (Dimethylamine) |

In some cases, a particle panel may comprise a particle listed in TABLE 5, below. In some cases, a particle panel may comprise at least two particles listed in TABLE 5. In some cases, a particle panel may comprise at least three particles listed in TABLE 5. In some cases, a particle panel may comprise at least four particles listed in TABLE 5. In some cases, a particle panel may comprise the particles listed in TABLE 5.

TABLE 5
Example of a particle panel of the present disclosure

| Particle | |
|---|---|
| ID | Description |
| SP-333 | Carboxylate |
| SP-347 | Silica |
| SP-353 | Amino |
| SP-389 | Wheat Germ Agglutinin |
| SP-008 | 1,2,4,5-Benzenetetracarboxylic acid coated SPION |

In some cases, a particle panel of the present disclosure may comprise at least one, at least two, at least 3, at least 4, or at least 5 particles, each particle selected from the group consisting of a superparamagnetic iron oxide particle (SPION) comprising a silica surface, a SPION comprising an N-(3-Trimethoxysilylpropyl)diethylenetriamine surface, a SPION comprising a Poly(dimethyl aminopropyl methacrylamide) (Dimethylamine) surface, a SPION comprising a carboxyl functionalized polystyrene surface, and a SPION comprising a dextran coating. In some cases, a particle panel of the present disclosure may comprise a SPION comprising a poly(N-(3-(dimethylamino)propyl) methacrylamide) (PDMAPMA) surface. In some cases, a particle panel of the present disclosure may comprise a SPION comprising a poly(oligo(ethylene glycol) methyl ether methacrylate) (POEGMA) surface. In some cases, a particle panel of the present disclosure may comprise a SPION comprising an N-(3-Trimethoxysilylpropyl)diethylenetriamine surface. In some cases, a particle panel of the present disclosure may comprise a SPION comprising a Poly(dimethyl aminopropyl methacrylamide) (Dimethylamine) surface. In some cases, a particle panel of the present disclosure may comprise a SPION comprising a dextran surface. In some cases, a particle panel of the present disclosure may comprise a SPION comprising a surface with a mixed chemistry based on amine-epoxy chemistry. In some cases, a particle panel of the present disclosure may comprise a SPION comprising a Polyzwitterion coated (Poly(N-[3-(Dimethylamino)propyl]methacrylamide-co-[2-(methacryloyloxy)ethyl]dimethyl-(3-sulfopropyl)ammonium hydroxide, P(DMAPMA-co-SBMA)) surface. In some cases, a particle panel of the present disclosure may comprise a SPION comprising styrene surface comprising an oleic acid functionalization. In some cases, a particle panel of the present disclosure may comprise a SPION comprising a boronated styrene surface. In some cases, a particle panel of the present disclosure may comprise a SPION comprising a carboxylated styrene surface. In some cases, a particle panel of the present disclosure may comprise a SPION comprising a carboxylated styrene surface. In some cases, a particle panel of the present disclosure may comprise a SPION comprising a strongly acidic silica surface. A particle panel of the present disclosure may comprise at least one particle, at least 2 particles, at least 3 particles, or at least 4 particles, or at least 5 particles, each selected from the group consisting of a silica-coated SPION, a poly(dimethylaminopropylmethacrylamide)-coated SPION, an N-(3-Trimethoxysilylpropyl)diethylenetriamine-coated SPION, a 1,6-hexanediamine-coated SPION, and an N1-(3-(trimethoxysilyl)propyl)hexane-1,6-diamine functionalized, silica-coated SPION. A particle panel of the present disclosure may comprise a silica-coated SPION, a poly(dimethylaminopropylmethacrylamide)-coated SPION, an N-(3-Trimethoxysilylpropyl)diethylenetriamine-coated SPION, a 1,6-hexanediamine-coated SPION, and an N′-(3-(trimethoxysilyl)propyl)hexane-1,6-diamine functionalized, silica-coated SPION.

In some cases, particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

### Protein Groups

The particle panels disclosed herein can be used to identify a number of proteins, peptides, or protein groups using a method disclosed herein. Feature intensities, as disclosed herein, may refer to the intensity of a discrete spike (“feature”) seen on a plot of mass to charge ratio versus intensity from a mass spectrometry run of a sample. These features can correspond to variably ionized fragments of peptides and/or proteins. Feature intensities can be sorted into protein groups. Protein groups refer to two or more proteins that are identified by a shared peptide sequence. Alternatively, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). Alternatively, if the peptide sequence is unique to a single protein (Protein 1), a protein group could be the “ZZX” protein group having one member (Protein 1). Each protein group can be supported by more than one peptide sequence. Protein detected or identified according to the instant disclosure can refer to a distinct protein detected in the sample (e.g., distinct relative other proteins detected using mass spectrometry). Thus, analysis of proteins present in distinct coronas corresponding to the distinct particle types in a particle panel, yields a high number of feature intensities. This number decreases as feature intensities are processed into distinct peptides, further decreases as distinct peptides are processed into distinct proteins, and further decreases as peptides are grouped into protein groups (two or more proteins that share a distinct peptide sequence),

### Biomolecule Coronas

Aspects of the present disclosure provide compositions, systems, and methods for collecting biomolecules on nanoparticles and microparticles (as well as other types of sensor elements such as polymer matrices, filters, rods, and extended surfaces). In some cases, a particle may adsorb a plurality of biomolecules upon contact with a biological sample, thereby forming a biomolecule corona on the surfaces of the particles. In some cases, the biomolecule corona may comprise proteins, lipids, nucleic acids, metabolites, saccharides, small molecules (e.g., sterols), and other biological species present in a sample. In some cases, a biomolecule corona comprising proteins may also be referred to as a ‘protein corona’, and may refer to all constituents adsorbed to a particle (e.g., proteins, lipids, nucleic acids, and other biomolecules), or may refer only to proteins adsorbed to the particle.

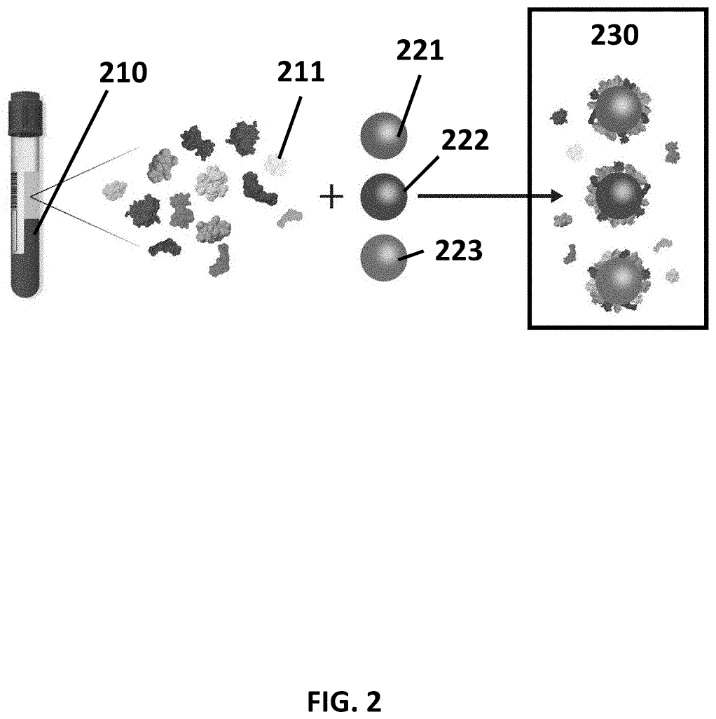

FIG. 2 provides a schematic overview of biomolecule formation, wherein a plurality of particles 221, 222, & 223 are contacted with a biological sample 210 comprising biomolecules molecules 211, and wherein each particle adsorbs a plurality of biomolecules from the biological sample to its surface 230. The different particles may be distinct particle types (depicted in the center of the figure, with the top, middle, and bottom spheres representing the three distinct particle types), such that each particle differs from the other particles by at least one physicochemical property. This difference in physicochemical properties can lead to the formation of different protein corona compositions on the particle surfaces.

The composition of the biomolecule corona may depend on a property of the particle. In many cases, the composition of the biomolecule corona is strongly dependent on the surface of the particle. Characteristics such as particle surface material (e.g., ceramic, polymer, metal, metal oxide, graphite, silicon dioxide, etc.), surface texture (rough, smooth, grooved, etc.), surface functionalization (e.g., carboxylate functionalized, amine functionalized, small molecule (e.g., saccharide) functionalized, etc.), shape, curvature, and size can each independently serve as determinants for biomolecule corona composition. In addition to surface features, the particle core composition, particle density, and particle surface area to mass ratio may each influence biomolecule corona composition. For example, two particles comprising the same surfaces and different cores may form different biomolecule coronas upon contact with the same sample.

Biomolecule corona formation may also be influenced by sample composition. For example, a first sample condition (e.g., low salinity) might favor the solubility of a particular analyte (e.g., an isoform of Bone Morphogenic Protein 1 (BMP1)), and thereby disfavor its binding in a biomolecule corona, while a second sample condition (e.g., high salinity) may diminish the solubility of the analyte, thereby driving its incorporation into a biomolecule corona.

Biomolecule corona composition may also depend on molecular level interactions between the biomolecules, themselves. An energetically favorable interaction between two biomolecules may promote their co-incorporation into a biomolecule corona. For example, if a first protein adsorbed to a particle comprises an affinity for a second protein in solution, the first protein may bind to a portion of the second protein, thereby driving its binding to the particle or to other proteins of the biomolecule corona of the particle. Analogously, a first biomolecule disposed within a biomolecule corona may comprise an energetically unfavorable interaction with a second biomolecule in a biological sample, thereby disfavoring its incorporation into a biomolecule corona. In part owing to these inter-biomolecule dependencies, biomolecule coronas provide sensitive platforms for directly and indirectly sensing biomolecules from a biological sample.

### Protein Analysis Methods

Biomolecules collected on a particle may be subjected to further analysis. A method may comprise collecting a biomolecule corona or a subset of biomolecules from a biomolecule corona. The collected biomolecule corona or the collected subset of biomolecules from the biomolecule corona may be subjected to further particle-based analysis (e.g., particle adsorption). The collected biomolecule corona or the collected subset of biomolecules from the biomolecule corona may be purified or fractionated (e.g., by a chromatographic method). The collected biomolecule corona or the collected subset of biomolecules from the biomolecule corona may be analyzed (e.g., by mass spectrometry). Furthermore, as biomolecule corona composition is dependent on solution-phase and particle-bound biomolecules as well as sample conditions (e.g., pH, osmolarity, lipid concentration), biomolecule corona composition can provide a sensitive measure of biomolecules which are not bound to a particle and of sample conditions.

The particles and methods of use thereof disclosed herein can bind a large number of unique biomolecules (e.g., proteins) in a biological sample (e.g., a biofluid). For example, a particle or particle panel disclosed herein can be incubated with a biological sample to form a protein corona comprising at least 5 protein groups, at least 10 protein groups, at least 15 protein groups, at least 20 protein groups, at least 25 protein groups, at least 50 protein groups, at least 80 protein groups, at least 100 protein groups, least 150 protein groups, at least 180 protein groups, at least 200 protein groups, at least 250 protein groups, at least 300 protein groups, at least 350 protein groups, at least 400 protein groups, at least 450 protein groups, at least 500 protein groups, at least 600 protein groups, at least 700 protein groups, at least 800 protein groups, at least 900 protein groups, at least 1000 protein groups, at least 1100 protein groups, at least 1200 protein groups, at least 1300 protein groups, at least 1400 protein groups, at least 1500 protein groups, at least 1600 protein groups, at least 1800 protein groups, at least 2000 protein groups, at least 2500, at least 5000 protein groups, at least 10000 protein groups, at least 15000 protein groups, at least 20000 protein groups, at least 25000 protein groups, at least 30000 protein groups, at least 35000 protein groups, at least 45000 protein groups, at least 50000 protein groups, at least 60000 protein groups, at least 70000 protein groups, at least 80000 protein groups, at least 90000 protein groups, or at least 100000 protein groups. A particle or particle panel disclosed herein can be incubated with a biological sample to form a protein corona comprising at most 5 protein groups, at most 10 protein groups, at most 20 protein groups, at most 30 protein groups, at most 40 protein groups, at most 50 protein groups, at most 60 protein groups, at most 80 protein groups, at most 100 protein groups, at most 150 protein groups, at most 200 protein groups, at most 250 protein groups, at most 300 protein groups, at most 400 protein groups, at most 500 protein groups, at most 600 protein groups, at most 800 protein groups, at most 1000 protein groups, at most 1200 protein groups, at most 1500 protein groups, at most 1800 protein groups, at most 2000 protein groups, at most 2500 protein groups, at most 3000 protein groups, at most 4000 protein groups, at most 5000 protein groups, at most 7500 protein groups, at most 10000 protein groups, at most 15000 protein groups, at most 20000 protein groups, at most 25000 protein groups, at most 50000 protein groups, at most 75000 protein groups, or at most 100000 protein groups. A particle disclosed herein can be incubated with a biological sample to form a protein corona comprising from 5 to 2500 protein groups. A particle or particle panel disclosed herein can be incubated with a biological sample to form a protein corona comprising from 5 to 50 protein groups. A particle disclosed herein can be incubated with a biological sample to form a protein corona comprising from 10 to 100 protein groups. A particle or particle panel disclosed herein can be incubated with a biological sample to form a protein corona comprising from 20 to 100 protein groups. A particle disclosed herein can be incubated with a biological sample to form a protein corona comprising from 20 to 400 protein groups. A particle or particle panel disclosed herein can be incubated with a biological sample to form a protein corona comprising from 50 to 500 protein groups. A particle disclosed herein can be incubated with a biological sample to form a protein corona comprising from 100 to 800 protein groups. A particle or particle panel disclosed herein can be incubated with a biological sample to form a protein corona comprising from 200 to 1000 protein groups. A particle or particle panel disclosed herein can be incubated with a biological sample to form a protein corona comprising from 300 to 1200 protein groups. A particle or particle panel disclosed herein can be incubated with a biological sample to form a protein corona comprising from 400 to 1500 protein groups. A particle or particle panel disclosed herein can be incubated with a biological sample to form a protein corona comprising from 500 to 2000 protein groups. A particle or particle panel disclosed herein can be incubated with a biological sample to form a protein corona comprising from 800 to 2500 protein groups. A particle or particle panel disclosed herein can be incubated with a biological sample to form a protein corona comprising from 1000 to 3000 protein groups. A particle or particle panel disclosed herein can be incubated with a biological sample to form a protein corona comprising from 1000 to 5000 protein groups. A particle or particle panel disclosed herein can be incubated with a biological sample to form a protein corona comprising from 2000 to 10000 protein groups. A particle or particle panel disclosed herein can be incubated with a biological sample to form a protein corona comprising from 5000 to 25000 protein groups. In some cases, several different types of particles can be used, separately or in combination, to identify large numbers of proteins in a particular biological sample. In other words, particles can be multiplexed in order to bind and identify large numbers of proteins in a biological sample. Protein corona analysis may compress the dynamic range of the analysis compared to a protein analysis of the original sample.

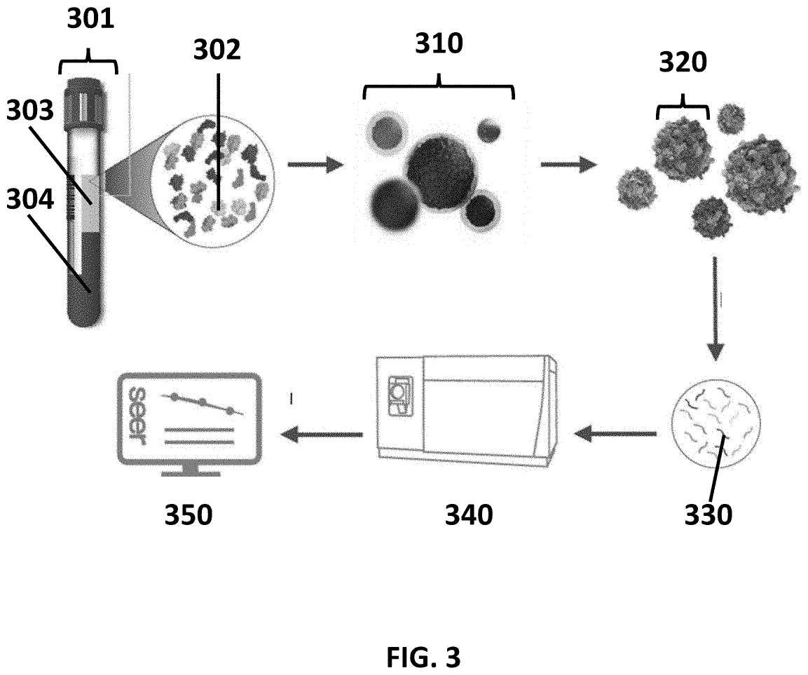

FIG. 3 provides an example of a particle-based biomolecule corona (e.g., protein corona) assay of the present disclosure. A biological sample (e.g., human plasma) 301 comprising a plurality of biomolecules 302 may be contacted to a plurality of particles 310. The sample may be treated, diluted, or split into a plurality of fractions 303 and 304 prior to analysis. For example, a whole blood sample may be fractionated into plasma and erythrocyte portions. Upon contact with the particles, a subset or the entirety of the plurality of biomolecules may adsorb to the particles, thereby forming biomolecule coronas 320 bound to the surfaces of the particles. Unbound biomolecules may be separated from the biomolecule coronas (e.g., through wash steps). The biomolecule coronas, or subsets thereof, may be collected from the particles. Alternatively, biomolecules of the biomolecule coronas may be fragmented or chemically treated while bound to the particles. In some assays, biomolecules (e.g., proteins) are fragmented (e.g., digested) while disposed in the biomolecule coronas to yield biomolecule (e.g., peptide) fragments 330. Biomolecules (or their chemically treated or fragmented derivatives) may be analyzed 340, for example by mass spectrometry, to yield data 350 representative of biomolecules 302 from the biological sample 301. The data may be analyzed to identify a biological state of the biological sample.

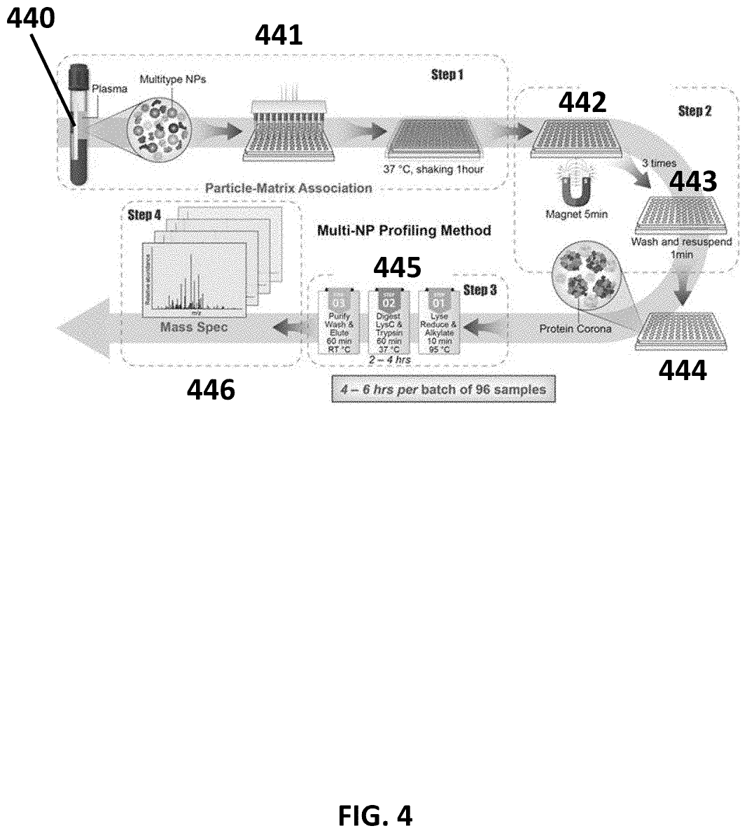

FIG. 4 illustrates an example of a biomolecule corona (e.g., protein corona) analysis workflow of the present disclosure which includes: particle incubation with a biological sample 440 (e.g., plasma) under conditions suitable for adsorption of biomolecules from the biological sample to the particles to form biomolecule coronas; partitioning 441 of the particle-plasma sample mixture into a plurality of partitions (e.g., wells on a multi-well plate); particle collection 442 (e.g., with a magnet); a wash step or plurality of wash steps 443 to remove analytes not adsorbed to the particles; 444 resuspension of the particles and the biomolecules adsorbed thereto; biomolecule corona digestion or chemical treatment 445 (e.g., protein reduction and digestion); and analysis of the biomolecule coronas or of biomolecules derived therefrom 446 (e.g., by liquid chromatography-mass spectrometry (LC-MS) analysis). While this example provides parallel analyses across multiple wells of a multi-well plate, a method may comprise a single sample volume or a plurality of sample volumes, for example 2 volumes, 3 volumes, 4 volumes, 5 volumes, 6 volumes, 7 volumes, 8 volumes, 9 volumes, 10 volumes, 11 volumes, 12 volumes, 15 volumes, 18 volumes, 20 volumes, 22 volumes, 24 volumes, 25 volumes, 28 volumes, 30 volumes, 36 volumes, 40 volumes, 48 volumes, 50 volumes, 60 volumes, 70 volumes, 80 volumes, 90 volumes, 96 volumes, 128 volumes, 150 volumes, 192 volumes, 200 volumes, 250 volumes, 256 volumes, 300 volumes, 384 volumes, 400 volumes, 500 volumes, 512 volumes, 600 volumes, or more. For example, the method may be performed on a 96, 192, or 384 well plate. Furthermore, while this example provides contacting a sample with particles prior to partitioning, a method may alternatively comprise partitioning a sample (e.g., into separate wells of a well plate) prior to contacting with particles. Each sample volume may be separately mixed with particles prior to, concurrent with, or subsequent to addition into a partition. In particular cases, the particles are present in a partition (for example in dry form or in solution) prior to addition of the sample into the partition. In some cases, sample may be added to partitions comprising particles. For example, a well plate may be provided with particles, buffer, and reagents in dry form, such that a method of use may comprise adding solution to the wells to resuspend the particles and dissolve the buffer and reagents, and then adding sample to the wells.

An assay utilizing a plurality of particles may distinguish which particle a specific biomolecule, biomolecule fragment (e.g., peptide generated by digesting a biomolecule corona protein), or signal corresponding to a biomolecule (e.g., one of ten mass spectrometric signals associated with a specific peptide fragment of a biomolecule corona protein). As biomolecule corona composition is dependent on sample conditions (e.g., salinity, temperature, pH), biomolecular composition, and particle physicochemical properties, two particles may develop different biomolecule coronas upon contacting a sample. Accordingly, the type or types of particles on which a particular biomolecule is observed comprise biological state information which may be utilized for analysis. A method may identify the type of particle on which a biomolecule, biomolecule fragment, or signal corresponding to a biomolecule is observed. A method may identify a ratio of abundances of a biomolecule or biomolecule fragment on a plurality of particles. A method may identify a ratio of signal intensities associated with a biomolecule identified on a plurality of particles.

Annotating biomolecules, biomolecule fragments, and signals by particle type can increase the amount of information derived from an assay. While many methods generate lists of biomolecules associated with samples, the present disclosure provides methods which differentiate the binding affinity of individual biomolecules across multiple particle types. As demonstrated in examples 1 and 2, differences in biomolecule abundance across two particles can comprise greater diagnostic utility than simple identification of a biomolecule within a sample. For example, 17 of the top 20 features in the trained Alzheimer's disease (AD) Random Forest classifier presented in example 2 are associated with proteins with OpenTarget Alzheimer's disease scores of less than 0.04, indicating that their sample-level abundances likely contain negligible diagnostic utility for Alzheimer's disease detection, but that their particle-specific detection can generate accurate Alzheimer's disease diagnoses.

A method (e.g., computer-implemented analysis with a trained classifier) of the present disclosure can comprise identifying a particle on which a biomolecule, biomolecule fragment, or signal was derived. A method of the present disclosure can comprise identifying an abundance ratio of a biomolecule or a biomolecule fragment across at least 2 particle types. A method of the present disclosure can comprise identifying an intensity ratio of a signal associated with a biomolecule or a biomolecule fragment across at least 2 particle types. A method of the present disclosure can comprise identifying an abundance ratio of a biomolecule or a biomolecule fragment across at least 3 particle types. A method of the present disclosure can comprise identifying an intensity ratio of a signal associated with a biomolecule or a biomolecule fragment across at least 3 particle types. A method of the present disclosure can comprise identifying an abundance ratio of a biomolecule or a biomolecule fragment across at least 4 particle types. A method of the present disclosure can comprise identifying an intensity ratio of a signal associated with a biomolecule or a biomolecule fragment across at least 4 particle types. A method of the present disclosure can comprise identifying an abundance ratio of a biomolecule or a biomolecule fragment across at least 5 particle types. A method of the present disclosure can comprise identifying an intensity ratio of a signal associated with a biomolecule or a biomolecule fragment across at least 5 particle types. A method of the present disclosure can comprise identifying an abundance ratio of a biomolecule or a biomolecule fragment across at least 6 particle types. A method of the present disclosure can comprise identifying an intensity ratio of a signal associated with a biomolecule or a biomolecule fragment across at least 6 particle types. A method of the present disclosure can comprise identifying an abundance ratio of a biomolecule or a biomolecule fragment across at least 8 particle types. A method of the present disclosure can comprise identifying an intensity ratio of a signal associated with a biomolecule or a biomolecule fragment across at least 8 particle types. A method of the present disclosure can comprise identifying an abundance ratio of a biomolecule or a biomolecule fragment across at least 10 particle types. A method of the present disclosure can comprise identifying an intensity ratio of a signal associated with a biomolecule or a biomolecule fragment across at least 10 particle types.

A method of the present disclosure may also identify an abundance or signal intensity ratio associated with different biomolecules or biomolecule fragments. For example, rather than exclusively utilizing an individual biomolecule abundance as an input, a trained classifier of the present disclosure may utilize an abundance ratio of a first biomolecule observed on a first particle and a second biomolecule observed on a second particle. As many biomolecules, and in particular many blood biomolecules, are ubiquitous across healthy and neurodegenerative disease samples (for example albumin, globulins, iron storage proteins), changes in their abundances may not be diagnostic for neurodegenerative disease states or progressions. However, a change in a ratio of two biomolecules, such as the iron storage proteins ferritin and transferrin can comprise information relevant for neurodegenerative disease and biological state diagnosis. Furthermore, as biomolecule particle adsorption can comprise a dependence on sample composition, an abundance or signal intensity ratio of two biomolecules on two particles can reflect biological state-relevant changes in a sample. Accordingly, a method of the present disclosure may identify an abundance ratio of a first biomolecule on a first particle and a second biomolecule on a second particle. A method of the present disclosure may also identify an intensity ratio of a first signal associated with a first biomolecule on a first particle and a second signal associated with a second biomolecule on a second particle.

Protein corona analysis may comprise an automated component. For example, an automated instrument may contact a sample with a particle or particle panel, identify proteins on the particle or particle panel (e.g., digest the proteins on the particle or particle panel and perform mass spectrometric analysis), and generate data for identifying a specific biomolecule or a biological state of a sample. The automated instrument may divide a sample into a plurality of volumes, and perform analysis on each volume or a subset of the plurality. The automated instrument may analyze multiple separate samples, for example by disposing multiple samples within multiple wells in a well plate, and performing parallel analysis on each sample or a subset of samples within the well plate.

The particle panels disclosed herein can be used to identify a number of proteins, peptides, protein groups, or protein classes using a protein analysis workflow described herein (e.g., a protein corona analysis workflow). Protein corona analysis may comprise contacting a sample to distinct particle types (e.g., a particle panel), forming biomolecule corona on the distinct particle types, and identifying the biomolecules in the biomolecule corona (e.g., by mass spectrometry). Feature intensities, as disclosed herein, refers to the intensity of a discrete spike (“feature”) seen on a plot of mass to charge ratio versus intensity from a mass spectrometry run of a sample. These features can correspond to variably ionized fragments of peptides and/or proteins. Using the data analysis methods described herein, feature intensities can be sorted into protein groups. Protein groups refer to two or more proteins that are identified by a shared peptide sequence. Alternatively, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2) which share the identifiable XYZ motif. Alternatively, if the peptide sequence is unique to a single protein (Protein 1), a protein group could be the “ZZX” protein group having one member (Protein 1). A protein group can be supported by more than one peptide sequence. Protein detected or identified according to the instant disclosure can refer to a distinct protein detected in the sample (e.g., distinct relative other proteins detected using mass spectrometry). Thus, analysis of proteins present in distinct coronas corresponding to the distinct particle types in a particle panel yields a high number of feature intensities. In some cases, multiple features are associated with a single peptide, such that processing feature intensities yields a lower number of peptides. As an illustrative example, during data processing, 6000 feature intensities (e.g., mass spectrometric signals) may be assigned to 1200 peptides, yielding an average of one peptide per 5 feature intensities. Furthermore, in some cases, multiple peptides may be associated with individual proteins or protein groups, such that processing peptides yields a lower number of proteins or protein groups. As another illustrative example, 1200 peptides may be assigned to 300 protein groups, yielding an average of one protein group per 4 peptides. In some cases, a single feature intensity may identify a peptide. In some cases, a single peptide may identify a protein group. In some cases, a single feature intensity may be divided between multiple peptides. For example, tandem mass spectrometric analysis (MS/MS) of a feature intensity may identify that two separate peptides contribute to the feature intensity.

The methods disclosed herein include isolating one or more particle types from a sample or from more than one sample (e.g., a biological sample or a serially interrogated sample). The particle types can be isolated or separated from the sample using a magnet. Moreover, multiple samples that are spatially isolated can be processed in parallel. Thus, the methods disclosed herein provide for isolating or separating a particle type from unbound protein in a sample. A particle type may be separated using methods including but not limited to magnetic separation, centrifugation, filtration, or gravitational separation. Particle panels may be incubated with a plurality of spatially isolated samples, wherein each spatially isolated sample is in a well in a well plate (e.g., a 96-well plate, a 192-well plate, or a 384-well plate). After incubation, the particle types in each of the wells of the well plate can be separated from unbound protein present in the spatially isolated samples by placing the entire plate on a magnet. This pulls down the superparamagnetic particles in the particle panel. The supernatant in each sample can be removed to remove the unbound protein. These steps (incubate, pull down) can be repeated to effectively wash the particles, thus removing residual background unbound protein that may be present in a sample. This is one example, but one of skill in the art could envision numerous other scenarios in which superparamagnetic particles are rapidly isolated from one or more than one spatially isolated samples at the same time.

In some cases, the methods and compositions of the present disclosure may provide identification and measurement of particular proteins in the biological samples by processing of the proteomic data via digestion of coronas formed on the surface of particles. Examples of proteins that can be identified and measured include highly abundant proteins, proteins of medium abundance, and low-abundance proteins. A low abundance protein may be present in a sample at concentrations at or below about 10 ng/mL. A high abundance protein may be present in a sample at concentrations at or above about 10 μg/mL. A protein of moderate abundance may be present in a sample at concentrations between about 10 ng/mL and about 10 μg/mL. Examples of proteins that are highly abundant proteins include albumin, IgG, and the top 14 proteins in abundance that contribute 95% of the analyte mass in plasma. Additionally, any proteins that may be purified using a conventional depletion column may be directly detected in a sample using the particle panels disclosed herein. Examples of proteins may be any protein listed in published databases such as Keshishian et al. (Mol Cell Proteomics. 2015 September; 14(9):2375-93. doi: 10.1074/mcp.M114.046813. Epub 2015 Feb. 27.), Farr et al. (J Proteome Res. 2014 Jan. 3; 13(1):60-75. doi: 10.1021/pr4010037. Epub 2013 Dec. 6.), or Pernemalm et al. (Expert Rev Proteomics. 2014 August; 11(4):431-48. doi: 10.1586/14789450.2014.901157. Epub 2014 Mar. 24.).

The proteomic data of the biological sample can be identified, measured, and quantified using a number of different analytical techniques. For example, proteomic data can be generated using SDS-PAGE or any gel-based separation technique. Peptides and proteins can also be identified, measured, and quantified using an immunoassay, such as ELISA. Alternatively, proteomic data can be identified, measured, and quantified using mass spectrometry, high performance liquid chromatography, LC-MS/MS, Edman Degradation, immunoaffinity techniques, methods disclosed in EP3548652, WO2019083856, WO2019133892, each of which is incorporated herein by reference in its entirety, and other protein separation techniques.

An assay may comprise protein collection of particles, protein digestion, and mass spectrometric analysis (e.g., MS, LC-MS, LC-MS/MS). The digestion may comprise chemical digestion, such as by cyanogen bromide or 2-Nitro-5-thiocyanatobenzoic acid (NTCB). The digestion may comprise enzymatic digestion, such as by trypsin or pepsin. The digestion may comprise enzymatic digestion by a plurality of proteases. The digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. The digestion may cleave peptides at random positions. The digestion may cleave peptides at a specific position (e.g., at methionines) or sequence (e.g., glutamate-histidine-glutamate). The digestion may enable similar proteins to be distinguished. For example, an assay may resolve 8 distinct proteins as a single protein group with a first digestion method, and as 8 separate proteins with distinct signals with a second digestion method. The digestion may generate an average peptide fragment length of 8 to 15 amino acids. The digestion may generate an average peptide fragment length of 12 to 18 amino acids. The digestion may generate an average peptide fragment length of 15 to 25 amino acids. The digestion may generate an average peptide fragment length of 20 to 30 amino acids. The digestion may generate an average peptide fragment length of 30 to 50 amino acids.

An assay may rapidly generate and analyze proteomic data. Beginning with an input biological sample (e.g., a buccal or nasal smear, plasma, or tissue), an assay of the present disclosure may generate and analyze proteomic data in less than 7 hours. Beginning with an input biological sample, an assay of the present disclosure may generate and analyze proteomic data in 5-7 hours. Beginning with an input biological sample, an assay of the present disclosure may generate and analyze proteomic data in less than 5 hours. Beginning with an input biological sample, an assay of the present disclosure may generate and analyze proteomic data in 3-5 hours. Beginning with an input biological sample, an assay of the present disclosure may generate and analyze proteomic data in 2-4 hours. Beginning with an input biological sample, an assay of the present disclosure may generate and analyze proteomic data in 2-3 hours. Beginning with an input biological sample, an assay of the present disclosure may generate and analyze proteomic data in less than 3 hours. Beginning with an input biological sample, an assay of the present disclosure may generate and analyze proteomic data in less than 2 hours. The analyzing may comprise identifying a protein group. The analyzing may comprise identifying a protein class. The analyzing may comprise quantifying an abundance of a biomolecule, a peptide, a protein, protein group, or a protein class. The analyzing may comprise identifying a ratio of abundances of two biomolecules, peptides, proteins, protein groups, or protein classes. The analyzing may comprise identifying a biological state.

### Dynamic Range

The biomolecule corona analysis methods described herein may comprise assaying biomolecules in a sample of the present disclosure across a wide dynamic range. The dynamic range of biomolecules assayed in a sample may be a range of measured signals of biomolecule abundances as measured by an assay method (e.g., mass spectrometry, chromatography, gel electrophoresis, spectroscopy, or immunoassays) for the biomolecules contained within a sample. For example, an assay capable of detecting proteins across a wide dynamic range may be capable of detecting proteins of very low abundance to proteins of very high abundance. The dynamic range of an assay may be directly related to the slope of assay signal intensity as a function of biomolecule abundance. For example, an assay with a low dynamic range may have a low (but positive) slope of the assay signal intensity as a function of biomolecule abundance, e.g., the ratio of the signal detected for a high abundance biomolecule to the ratio of the signal detected for a low abundance biomolecule may be lower for an assay with a low dynamic range than an assay with a high dynamic range. In specific cases, dynamic range may refer to the dynamic range of proteins within a sample or assaying method.

The particle panels disclosed herein can be used to identify the number of distinct proteins disclosed herein, and/or any of the specific proteins disclosed herein, over a wide dynamic range. As used herein, a dynamic range may denote a log₁₀ value of a ratio of the highest and lowest abundance species of a specified type. Enriching or assaying species over a dynamic range may refer to the abundances of those species in the sample from which they were assayed or derived. For example, the particle panels disclosed herein comprising distinct particle types, can enrich for proteins in a sample over the entire dynamic range at which proteins are present in a sample (e.g., a plasma sample). In some cases, a particle panel including any number of distinct particle types disclosed herein, enriches and identifies proteins over a dynamic range of about 2 to about 12. In some cases, a particle panel including any number of distinct particle types disclosed herein, enriches and identifies proteins over a dynamic range of about 3 to about 12. In some cases, a particle panel including any number of distinct particle types disclosed herein, enriches and identifies proteins over a dynamic range of about 4 to about 12. In some cases, a particle panel including any number of distinct particle types disclosed herein, enriches and identifies proteins over a dynamic range of a about 5 to about 12. In some cases, a particle panel including any number of distinct particle types disclosed herein, enriches and identifies proteins over a dynamic range of about 6 to about 12. In some cases, a particle panel including any number of distinct particle types disclosed herein, enriches and identifies proteins over a dynamic range of about 7 to about 12. In some cases, a particle panel including any number of distinct particle types disclosed herein, enriches and identifies proteins over a dynamic range of about 8 to about 12. In some cases, a particle panel including any number of distinct particle types disclosed herein, enriches and identifies proteins over a dynamic range of about 9 to about 12. In some cases, a particle panel including any number of distinct particle types disclosed herein, enriches and identifies proteins over a dynamic range of about 10 to about 12. In some cases, a particle panel including any number of distinct particle types disclosed herein, enriches and identifies proteins over a dynamic range of about 11 to about 12. In some cases, a particle panel including any number of distinct particle types disclosed herein, enriches and identifies proteins over a dynamic range of about 12. In some cases, a particle panel including any number of distinct particle types disclosed herein, enriches and identifies proteins over a dynamic range of from about 2 to about 6. In some cases, a particle panel including any number of distinct particle types disclosed herein, enriches and identifies proteins over a dynamic range of from about 3 to about 8. In some cases, a particle panel including any number of distinct particle types disclosed herein, enriches and identifies proteins over a dynamic range of from about 4 to 8. In some cases, a particle panel including any number of distinct particle types disclosed herein, enriches and identifies proteins over a dynamic range of from about 5 to about 10. In some cases, a particle panel including any number of distinct particle types disclosed herein, enriches and identifies proteins over a dynamic range of from about 6 to about 10. In some cases, a particle panel including any number of distinct particle types disclosed herein, enriches and identifies proteins over a dynamic range of from about 6 to about 12.

The biomolecule corona analysis methods described herein may compress the dynamic range of an assay. The dynamic range of an assay may be compressed relative to another assay if the slope of the assay signal intensity as a function of biomolecule abundance is lower than that of the other assay. For example, a plasma sample assayed using protein corona analysis with mass spectrometry may have a compressed dynamic range compared to a plasma sample assayed using mass spectrometry alone, directly on the sample or compared to provided abundance values for plasma proteins in databases (e.g., the database provided in Keshishian et al., Mol. Cell Proteomics 14, 2375-2393 (2015), also referred to herein as the “Carr database”). The compressed dynamic range may enable the detection of more low abundance biomolecules in a biological sample using biomolecule corona analysis with mass spectrometry than using mass spectrometry alone.

The dynamic range of a proteomic analysis assay may be the ratio of the signal produced by highest abundance proteins (e.g., the highest 10% of proteins by abundance) to the signal produced by the lowest abundance proteins (e.g., the lowest 10% of proteins by abundance). Compressing the dynamic range of a proteomic analysis may comprise decreasing the ratio of the signal produced by the highest abundance proteins to the signal produced by the lowest abundance proteins for a first proteomic analysis assay relative to that of a second proteomic analysis assay. The protein corona analysis assays disclosed herein may compress the dynamic range relative to the dynamic range of a total protein analysis method (e.g., mass spectrometry, gel electrophoresis, or liquid chromatography).

Provided herein are several methods for compressing the dynamic range of a biomolecular analysis assay to facilitate the detection of low abundance biomolecules relative to high abundance biomolecules. For example, a particle type of the present disclosure can be used to serially interrogate a sample. Upon incubation of the particle type in the sample, a biomolecule corona comprising forms on the surface of the particle type. If biomolecules are directly detected in the sample without the use of the particle types, for example by direct mass spectrometric analysis of the sample, the dynamic range may span a wider range of concentrations, or more orders of magnitude, than if the biomolecules are directed on the surface of the particle type. Thus, using the particle types disclosed herein may be used to compress the dynamic range of biomolecules in a sample. Without being limited by theory, this effect may be observed due to more capture of higher affinity, lower abundance biomolecules in the biomolecule corona of the particle type and less capture of lower affinity, higher abundance biomolecules in the biomolecule corona of the particle type.

A dynamic range of a proteomic analysis assay may be illustrated by the slope of a plot of a protein signal measured by the proteomic analysis assay as a function of total abundance of the protein in the sample. Compressing the dynamic range may comprise decreasing the slope of the plot of a protein signal measured by a proteomic analysis assay as a function of total abundance of the protein in the sample relative to the slope of the plot of a protein signal measured by a second proteomic analysis assay as a function of total abundance of the protein in the sample. The protein corona analysis assays disclosed herein may compress the dynamic range relative to the dynamic range of a total protein analysis method (e.g., mass spectrometry, gel electrophoresis, or liquid chromatography).

### Kits

Provided herein are kits comprising compositions of the present disclosure that may be used to perform the methods of the present disclosure. A kit may comprise one or more particle types to interrogate a sample to identify a biological state of a sample. In some cases, a kit may comprise a particle type provided in TABLES 1-5. A kit may comprise a reagent for functionalizing a particle (e.g., a reagent for tethering a small molecule functionalization to a particle surface). The kit may be pre-packaged in discrete aliquots. In some cases, the kit can comprise a plurality of different particle types that can be used to interrogate a sample. The plurality of particle types can be pre-packaged where each particle type of the plurality is packaged separately. Alternately, the plurality of particle types can be packaged together to contain combination of particle types in a single package. A particle may be provided in dried (e.g., lyophilized) form, or may be provided in a suspension or solution. The particles may be provided in a well plate. For example, a kit may contain an 8 well plate, an 8-384 well plate with particles provided (e.g., sealed) within the wells. For example, a well plate may comprise at least 8, at least 16, at least 24, at least 32, at least 40, at least 48, at least 56, at least 64, at least 72, at least 80, at least 88, at least 96, at least 104, at least 112, at least 120, at least 128, at least 136, at least 144, at least 152, at least 160, at least 168, at least 176, at least 184, at least 192, at least 200, at least 208, at least 216, at least 224, at least 232, at least 240, at least 248, at least 256, at least 264, at least 272, at least 280, at least 288, at least 296, at least 304, at least 312, at least 320, at least 328, at least 336, at least 344, at least 352, at least 360, at least 368, at least 376, at least 384, at least 392, at least 400 wells comprising particles. Two wells in such a well plate may contain different particles or different concentrations of particles. Two wells may comprise different buffers or chemical conditions. For example, a well plate may be provided with different particles in each row of wells and different buffers in each column of rows. A well may be sealed by a removable covering. For example, a kit may comprise a well plate comprising a plastic slip covering a plurality of wells. A well may be sealed by a pierceable covering. For example, a well may be covered by a septum that a needle can pierce to facilitate sample movement into and out of the well.

### Samples

The present disclosure provides a range of samples that can be assayed using the particles and the methods provided herein. A sample may be a biological sample (e.g., a sample derived from a living organism). A sample may comprise a cell or be cell-free. A sample may comprise a biofluid, such as blood, serum, plasma, urine, or cerebrospinal fluid (CSF). Samples of the present disclosure include biological samples from a subject. A method may include analyzing a sample from a single subject, or analyzing samples from multiple subjects. The subject may be a human or a non-human animal. The biological samples can contain a plurality of proteins or proteomic data, which may be analyzed after adsorption of proteins to the surface of the various sensor element (e.g., particle) types in a panel and subsequent digestion of protein coronas. Proteomic data can comprise nucleic acids, peptides, or proteins. A biofluid may be a fluidized solid, for example a tissue homogenate, or a fluid extracted from a biological sample. A biological sample may be, for example, a tissue sample or a fine needle aspiration (FNA) sample. A biological sample may be a cell culture sample. For example, a biofluid may be a fluidized cell culture extract.

A wide range of samples are compatible for use within the methods and compositions of the present disclosure. The biological sample may comprise plasma, serum, urine, cerebrospinal fluid, synovial fluid, tears, saliva, whole blood, a blood component (e.g., plasma or white blood cells), milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. The biological sample may comprise blood or a blood component. The biological sample may comprise multiple biological samples (e.g., pooled plasma from multiple subjects, or multiple tissue samples from a single subject). The biological sample may comprise a single type of biofluid or biomaterial from a single source. A biological sample may comprise a nerve biopsy.

Various methods of the present disclosure utilize blood or blood components (e.g., red blood cells, buffy coats, plasma). Contrasting many tissue biopsies, which can be damaging and cost intensive, blood collection is often relatively facile and benign, and is therefore suitable for routine and low-risk patient monitoring. Furthermore, as human blood is estimated to contain over 5000 types of protein groups whose abundances and forms (e.g., post-translationally modifications and variant types) can be responsive to, the blood proteome offers a biological state changes are often evidenced by subtle changes in blood protein composition. A method of the present disclosure may use whole blood (e.g., untreated blood drawn from a subject). A method of the present disclosure may also use a treated or partitioned blood sample. In some cases, a sample comprises plasma, buffy coat, white blood cells, platelets, hematocrit, red blood cells, serum, blood clots or any combination thereof. In some cases, plasma, buffy coat, white blood cells, platelets, hematocrit, red blood cells, serum, blood clots or any combination thereof are extracted from a blood sample for use in a method disclosed herein.

In some cases, a method utilizes serum. As used herein, “serum” may denote the liquid fraction remaining after a blood sample clots. As a blood sample left at room temperature will typically clot within 15-60 minutes, serum may be prepared by incubating a blood sample at or above room temperature, for example at 25° C. or at 37° C., respectively. After at least about 10 minutes, at least about 15 minutes, at least about 20 minutes, at least about 30 minutes, at least about 40 minutes, at least about 50 minutes, or at least about 60 minutes, the blood clots may be separated from solution through centrifugation. While serum is often prepared non-hemolyzed (e.g., wherein blood cells remain intact through clotting and removal), some methods of the present disclosure may utilize serum derived from hemolyzed blood samples.

In some cases, a method utilizes plasma. As used herein, “plasma” may denote a fraction collected from blood pretreated with an anticoagulant and separated from blood cells and platelets. Contrasting with serum, plasma typically contains an array of clotting factors, such as fibrinogen, prothrombin, and proaccelerin. As the concentrations and forms of these species can reflect certain health conditions, plasma analysis can provide greater diagnostic insight than serum analysis for some biological states. Plasma samples can be prepared treating blood with an anticoagulant, and then centrifuging the treated blood. The anticoagulant may comprise citrate, ethylenediaminetetraaceticacid (EDTA), potassium oxalate, hirudin, argatroban, ximelagatran, heparin, fondaparinux, or any combination thereof.

Centrifugation parameters affect the proteins which remain in solution, and therefore may be modified depending on the biomolecules of interest for detection from plasma or serum. Centrifugation may be performed for at least 2 minutes, at least 4 minutes, at least 6 minutes, at least 8 minutes, at least 10 minutes, at least 12 minutes, at least 15 minutes, at least 20 minutes, or at least 30 minutes. Centrifugation may be performed for at most 30 minutes, at most 20 minutes, at most 15 minutes, at most 10 minutes, at most 8 minutes, at most 6 minutes, at most 4 minutes, or at most 2 minutes. Centrifugation may impart at least 100 gravitational force equivalents (g), at least 200 g, at least 300 g, at least 400 g, at least 500 g, at least 600 g, at least 800 g, at least 1000 g, at least 1200 g, at least 1500 g, at least 1800 g, at least 2000 g, at least 2500 g, at least 3000 g, at least 4000 g, at least 5000 g, at least 6000 g, at least 8000 g, or at least 10000 g. The centrifugation may impart at most 100 g, at most 200 g, at most 300 g, at most 400 g, at most 500 g, at most 600 g, at most 800 g, at most 1000 g, at most 1200 g, at most 1500 g, at most 1800 g, at most 2000 g, at most 2500 g, at most 3000 g, at most 4000 g, at most 5000 g, at most 6000 g, at most 8000 g, or at most 10000 g.

The biological sample may be diluted or pre-treated. The biological sample may undergo depletion (e.g., albumin removal from serum or plasma) prior to or following contact with a particle or plurality of particles. The biological sample may also undergo physical (e.g., homogenization or sonication) or chemical treatment prior to or following contact with a particle or plurality of particles. The biological sample may be diluted prior to or following contact with a particle or plurality of particles. The dilution medium may comprise buffer or salts, or be purified water (e.g., distilled water). Different partitions of a biological sample may undergo different degrees of dilution. A biological sample or a portion thereof may undergo a 1.1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1000-fold dilution. For example, a plasma sample may be subjected to a 5-fold dilution with buffer prior to analysis.

The compositions and methods of the present disclosure can be used to measure, detect, and identify specific proteins from biological samples. Examples of proteins that can be identified and measured include highly abundant proteins, proteins of medium abundance, and low-abundance proteins. For example, a composition or method may identify at least 1, at least 2, at least 3, at least 4, at least 5, at least 6, at least 7, at least 8, at least 10, at least 12, at least 15, at least 18, at least 20, at least 25, at least 30, at least 35, at least 40, or at least 50 human plasma proteins from the group consisting of albumin, immunoglobulin G (IgG), lysozyme, carcino embryonic antigen (CEA), receptor tyrosine-protein kinase erbB-2 (HER-2/neu), bladder tumor antigen, thyroglobulin, alpha-fetoprotein, prostate specific antigen (PSA), mucin 16 (CA125), carbohydrate antigen 19-9 (CA19.9), carcinoma antigen 15-3 (CA15.3), leptin, prolactin, osteopontin, insulin-like growth factor 2 (IGF-II), 4F2 cell-surface antigen heavy chain (CD98), fascin, sPigR, 14-3-3 eta, troponin I, B-type natriuretic peptide, breast cancer type 1 susceptibility protein (BRCA1), c-Myc proto-oncogene protein (c-Myc), interleukin-6 (IL-6), fibrinogen, epidermal growth factor receptor (EGFR), gastrin, PH, granulocyte colony-stimulating factor (G CSF), desmin, enolase 1 (NSE), folice-stimulating hormone (FSH), vascular endothelial growth factor (VEGF), P21, Proliferating cell nuclear antigen (PCNA), calcitonin, pathogenesis-related proteins (PR), luteinizing hormone (LH), somatostatin S100, insulin. alpha-prolactin, adrenocorticotropic hormone (ACTH), B-cell lymphoma 2 (Bcl 2), estrogen receptor alpha (ER alpha), antigen k (Ki-67), tumor protein (p53), cathepsin D, beta catenin, von Willebrand factor (VWF), CD15, k-ras, caspase 3, ENTH domain-containing protein (EPN), CD10, FAS, breast cancer type 2 susceptibility protein (BRCA2), CD30L, CD30, CGA, CRP, prothrombin, CD44, APEX, transferrin, GM-CSF, E-cadherin, interleukin-2 (IL-2), Bax, IFN-gamma, beta-2-MG, tumor necrosis factor alpha (TNF alpha), cluster of differentiation 340, trypsin, cyclin D1, MG B, XBP-1, HG-1, YKL-40, S-gamma, ceruloplasmin, NESP-55, netrin-1, geminin, GADD45A, CDK-6, CCL21, breast cancer metastasis suppressor 1 (BrMS1), 17betaHDI, platelet-derived growth factor receptor A (PDGRFA), P300/CBP-associated factor (Pcaf), chemokine ligand 5 (CCLS), matrix metalloproteinase-3 (MMP3), claudin-4, and claudin-3

### Neurodegenerative Disease Detection

The compositions and methods disclosed herein can be used to identify various biological states of samples and subjects from which samples are derived. As an example, biological state can refer to an elevated or low level of a particular biomolecule or set of biomolecules, such as elevated blood glucose or misfolded alpha synuclein. Biological state may also refer to a particular pathology, such as Alzheimer's disease, or a stage of the pathology, such as early, middle, or late stage dementia. In other examples, a biological state can refer to identification of a disease, such as cancer. The particles and methods of us thereof can be used to distinguish between two biological states. The two biological states may be related diseases states (e.g., mild cognitive impairment and Alzheimer's disease). The two biological states may be different phases of a disease, such as pre-Alzheimer's and mild Alzheimer's. The two biological states may be distinguished with a high degree of accuracy (e.g., the percentage of accurately identified biological states among a population of samples). For example, the compositions and methods of the present disclosure may distinguish two biological states with at least 60% accuracy, at least 70% accuracy, at least 75% accuracy at least 80% accuracy, at least 85% accuracy, at least 90% accuracy, at least 95% accuracy, at least 98% accuracy, or at least 99% accuracy. The two biological states may be distinguished with a high degree of specificity (e.g., the rate at which negative results are correctly identified among a population of samples). For example, the compositions and methods of the present disclosure may distinguish two biological states with at least 60% specificity, at least 70% specificity, at least 75% specificity at least 80% specificity, at least 85% specificity, at least 90% specificity, at least 95% specificity, at least 98% specificity, or at least 99% specificity.

The methods, compositions, and systems of the present disclosure may detect a neurological disease state. Neurological disorders or neurological diseases are used interchangeably and refer to diseases associated with neurological tissues, such as the brain, the spinal chord, and the nerves that connect them. Neurological diseases include, but are not limited to, brain tumors, epilepsy, Parkinson's disease, Alzheimer's disease, ALS, arteriovenous malformation, cerebrovascular disease, brain aneurysms, epilepsy, multiple sclerosis, Peripheral Neuropathy, Post-Herpetic Neuralgia, stroke, frontotemporal dementia, demyelinating disease (including but are not limited to, multiple sclerosis, Devic's disease (i.e. neuromyelitis optica), central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, and anti-MAG peripheral neuropathy) and the like. Neurological disorders also include immune-mediated neurological disorders (IMNDs), which include diseases with at least one component of the immune system reacts against host proteins present in the central or peripheral nervous system and contributes to disease pathology. IMNDs may include, but are not limited to, demyelinating disease, paraneoplastic neurological syndromes, immune-mediated encephalomyelitis, immune-mediated autonomic neuropathy, myasthenia gravis, autoantibody-associated encephalopathy, and acute disseminated encephalomyelitis.

Methods, systems, and/or apparatuses of the present disclosure may be able to accurately distinguish between patients with or without Alzheimer's disease. These may also be able to detect patients who are pre-symptomatic and may develop Alzheimer's disease several years after the screening. This provides advantages of being able to treat a disease at a very early stage, even before development of the disease.

The methods, compositions, and systems of the present disclosure can detect a pre-disease stage of a disease or disorder. A pre-disease stage is a stage at which the patient has not developed any signs or symptoms of the disease. A pre-neurological disease stage would be a stage in which a person has not developed one or more symptom of the neurological disease. The ability to diagnose a disease before one or more sign or symptom of the disease is present allows for close monitoring of the subject and the ability to treat the disease at a very early stage, increasing the prospect of being able to halt progression or reduce the severity of the disease.

The methods, compositions, and systems of the present disclosure may detect the early stages of a disease or disorder. Early stages of the disease can refer to when the first signs or symptoms of a disease may manifest within a subject. The early stage of a disease may be a stage at which there are no outward signs or symptoms. For example, in Alzheimer's disease an early stage may be a pre-Alzheimer's stage in which no symptoms are detected yet the patient will develop Alzheimer's months or years later.

Identifying a disease in either pre-disease development or in the early states may often lead to a higher likelihood for a positive outcome for the patient. For example, diagnosing dementia at an early stage (stage 0 or stage 1) can enable early stage interventions, which may slow or even halt its progression, and increase the quality of life and life expectancy of the patient.

In some cases, the methods, compositions, and systems of the present disclosure are able to detect intermediate stages of the disease. Intermediate states of the disease describe stages of the disease that have passed the first signs and symptoms and the patient is experiencing one or more symptom of the disease. Further, the methods, compositions, and systems of the present disclosure may be able to detect late or advanced stages of the disease. Late or advanced stages of the disease may also be called “severe” or “advanced” and usually indicates that the subject is suffering from multiple symptoms and effects of the disease.

The methods of the present disclosure can include processing the biomolecule corona data of a sample against a collection of biomolecule corona datasets representative of a plurality of diseases and/or a plurality of disease states to determine if the sample indicates a disease and/or disease state. For example, samples can be collected from a population of subjects over time. Once the subjects develop a disease or disorder, the present disclosure allows for the ability to characterize and detect the changes in biomolecule fingerprints over time in the subject by computationally analyzing the biomolecule fingerprint of the sample from the same subject before they have developed a disease to the biomolecule fingerprint of the subject after they have developed the disease. Samples can also be taken from cohorts of patients who all develop the same disease, allowing for analysis and characterization of the biomolecule fingerprints that are associated with the different stages of the disease for these patients (e.g. from pre-disease to disease states).

In some cases, the methods, compositions, and systems of the present disclosure are able to distinguish not only between different types of diseases, but also between the different stages of the disease (e.g. early stages of disease). This can comprise distinguishing healthy subjects from pre-disease state subjects. The pre-disease state may be, for example, a neurodegenerative disease, dementia.

### Computer Control Systems

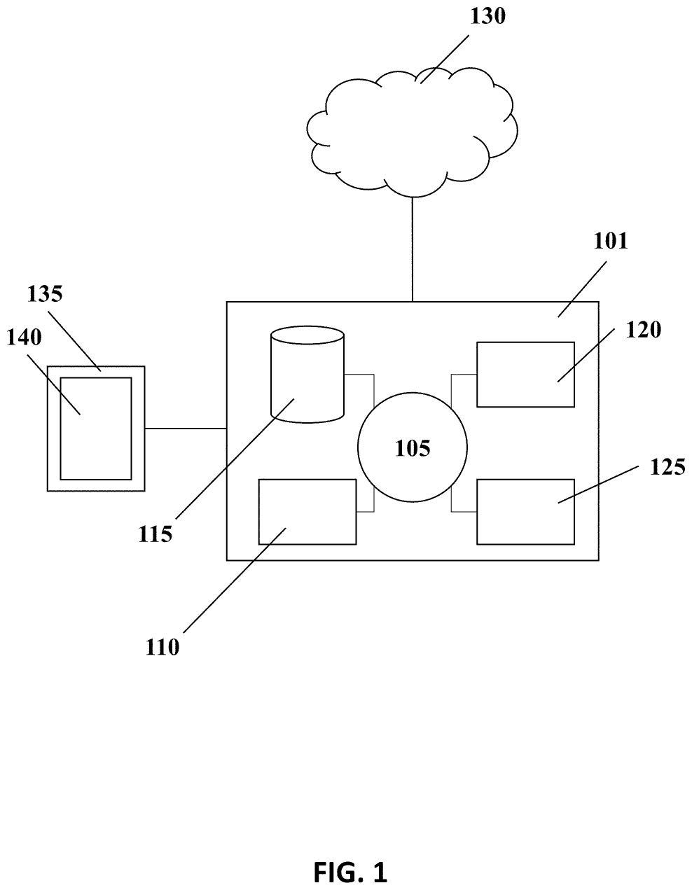

The present disclosure provides computer control systems that are programmed to implement methods of the disclosure. FIG. 1 shows a computer system that is programmed or otherwise configured to implement methods provided herein. The computer system 101 can regulate various aspects of the assays disclosed herein, which are capable of being automated (e.g., movement of any of the reagents disclosed herein on a substrate). The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example a readout of the proteins identified using the methods disclosed herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 105.

Determination, analysis or statistical classification can be performed using methods, including, but not limited to, for example, a supervised and unsupervised data analysis and clustering approaches such as hierarchical cluster analysis (HCA), principal component analysis (PCA), Partial least squares Discriminant Analysis (PLSDA), machine learning (e.g., Random Forest), logistic regression, decision trees, support vector machine (SVM), k-nearest neighbors, naive Bayes, linear regression, polynomial regression, SVM for regression, K-means clustering, and hidden Markov models, among others. The computer system can perform various aspects of analyzing the protein sets or protein corona of the present disclosure, such as, for example, comparing/analyzing the biomolecule corona of several samples to determine with statistical significance what patterns are common between the individual biomolecule coronas to determine a protein set that is associated with the biological state. The computer system can be used to develop classifiers to detect and discriminate different protein sets or protein corona (e.g., characteristic of the composition of a protein corona). Data collected from the presently disclosed sensor array can be used to train a machine learning algorithm, specifically an algorithm that receives array measurements from a patient and outputs specific biomolecule corona compositions from each patient. Before training the algorithm, raw data from the array can be first denoised to reduce variability in individual variables.

Machine learning can be generalized as the ability of a learning machine to perform accurately on new, unseen examples/tasks after having experienced a learning data set. Machine learning may include the following concepts and methods. Supervised learning concepts may include AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher's linear discriminant, Linear regression, Logistic regression, Multinomial logistic regression, Naive Bayes classifier, Perceptron, Support vector machines; Quadratic classifiers; k-nearest neighbor; Boosting; Decision trees, such as C4.5, Random forests, ID3, CART, SLIQ SPRINT; Bayesian networks, such as Naive Bayes; and Hidden Markov models. Unsupervised learning concepts may include; Expectation-maximization algorithm; Vector Quantization; Generative topographic map; Information bottleneck method; Artificial neural network, such as Self-organizing map; Association rule learning, such as, Apriori algorithm, Eclat algorithm, and FPgrowth algorithm; Hierarchical clustering, such as Singlelinkage clustering and Conceptual clustering; Cluster analysis, such as, K-means algorithm, Fuzzy clustering, DBSCAN, and OPTICS algorithm; and Outlier Detection, such as Local Outlier Factor. Semi-supervised learning concepts may include; Generative models; Low-density separation; Graph-based methods; and Co-training. Reinforcement learning concepts may include; Temporal difference learning; Q-learning; Learning Automata; and SARSA. Deep learning concepts may include; Deep belief networks; Deep Boltzmann machines; Deep Convolutional neural networks; Deep Recurrent neural networks; and Hierarchical temporal memory. A computer system may be adapted to implement a method described herein. The system includes a central computer server that is programmed to implement the methods described herein. The server includes a central processing unit (CPU, also “processor”) which can be a single core processor, a multi core processor, or plurality of processors for parallel processing. The server also includes memory (e.g., random access memory, read-only memory, flash memory); electronic storage unit (e.g. hard disk); communications interface (e.g., network adaptor) for communicating with one or more other systems; and peripheral devices which may include cache, other memory, data storage, and/or electronic display adaptors. The memory, storage unit, interface, and peripheral devices are in communication with the processor through a communications bus (solid lines), such as a motherboard. The storage unit can be a data storage unit for storing data. The server is operatively coupled to a computer network (“network”) with the aid of the communications interface. The network can be the Internet, an intranet and/or an extranet, an intranet and/or extranet that is in communication with the Internet, a telecommunication or data network. The network in some cases, with the aid of the server, can implement a peer-to-peer network, which may enable devices coupled to the server to behave as a client or a server.

The storage unit can store files, such as subject reports, and/or communications with the data about individuals, or any aspect of data associated with the present disclosure.

The computer server can communicate with one or more remote computer systems through the network. The one or more remote computer systems may be, for example, personal computers, laptops, tablets, telephones, Smart phones, or personal digital assistants.

In some applications the computer system includes a single server. In other situations, the system includes multiple servers in communication with one another through an intranet, extranet and/or the internet.

The server can be adapted to store measurement data or a database as provided herein, patient information from the subject, such as, for example, medical history, family history, demographic data and/or other clinical or personal information of potential relevance to a particular application. Such information can be stored on the storage unit or the server and such data can be transmitted through a network.

Methods as described herein can be implemented by way of machine (or computer processor) executable code (or software) stored on an electronic storage location of the server, such as, for example, on the memory, or electronic storage unit. During use, the code can be executed by the processor. In some cases, the code can be retrieved from the storage unit and stored on the memory for ready access by the processor. In some situations, the electronic storage unit can be precluded, and machine-executable instructions are stored on memory. Alternatively, the code can be executed on a second computer system.

Aspects of the systems and methods provided herein, such as the server, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” can refer to any medium that participates in providing instructions to a processor for execution.

The computer systems described herein may comprise computer-executable code for performing any of the algorithms or algorithms-based methods described herein. In some applications the algorithms described herein will make use of a memory unit that is comprised of at least one database.

Data relating to the present disclosure can be transmitted over a network or connections for reception and/or review by a receiver. The receiver can be but is not limited to the subject to whom the report pertains; or to a caregiver thereof, e.g., a health care provider, manager, other health care professional, or other caretaker; a person or entity that performed and/or ordered the analysis. The receiver can also be a local or remote system for storing such reports (e.g. servers or other systems of a “cloud computing” architecture). In one embodiment, a computer-readable medium includes a medium suitable for transmission of a result of an analysis of a biological sample using the methods described herein.

Aspects of the systems and methods provided herein can be embodied in programming. Various aspects of the technology may be thought of as “products” or “articles of manufacture” typically in the form of machine (or processor) executable code and/or associated data that is carried on or embodied in a type of machine readable medium. Machine executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide nontransitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

### Computer-Implemented Systems

Further disclosed herein are computer-implemented systems for identifying biological state information from biomolecule corona data. The computer-implemented system may comprise a communication interface configured to receive data, such as biomolecule corona data. The communication interface may receive data over a communication network, such as a cloud-based network or a computer server-based network, or a storage device such as a flash drive memory device or a compact disc. The computer-implemented system may comprise a computer in communication with the communication interface. The computer may comprise one or more processors, as well as computer readable medium comprising machine-executable code which may be executed by the one or more processors, and which may be configured to implement a method. The method may process biomolecule corona data, for example by filtering or baseline correcting a portion of the data. The method may identify a biomolecule (e.g., a protein, a protein group, a saccharide, a nucleic acid, or a metabolite). The method may identify an abundance of a biomolecule or an intensity of a signal (e.g., by performing a Gaussian or Lorentzian fit to a peak in the data). The method may identify a ratio of two or more biomolecule abundances or two or more signal intensities. The method may comprise a machine learning algorithm or a trained algorithm for biological state analysis. The method may identify a biological state based at least in part on the biomolecule corona data.

The computer may comprise one or more processors, as well as computer readable medium which may be executed by the one or more processors to communicate with an instrument through the communication interface, and operate or provide parameters (e.g., temperatures, incubation times, number of wash cycles) the instrument to perform biomolecule corona analysis (e.g., perform biological sample-particle incubation, wash, digestion, and solid-phase extraction). For example, upon input of a sample and reagents into an automated instrument for biomolecule corona analysis, the computer may prompt a user for information regarding the sample or intended assay, and then execute a biomolecule corona analysis method based on the information by the user, such as sample type, intended depth of sample coverage (e.g., in some cases, the length of particle-biological sample incubation times may affect the number of protein groups identified in an assay).

The computer may comprise one or more processors, as well as computer readable medium which may be executed by the one or more processors to communicate with an instrument configured to analyze a sample which has been subjected to biomolecule corona analysis through the communication interface, and to operate or provide parameters to the instrument, as well as computer readable medium which may be executed by the one or more processors to operate an instrument configured to perform biomolecule corona analysis. For example, the computer may provide parameters to a mass spectrometer for analysis of a protease digested biomolecule corona.

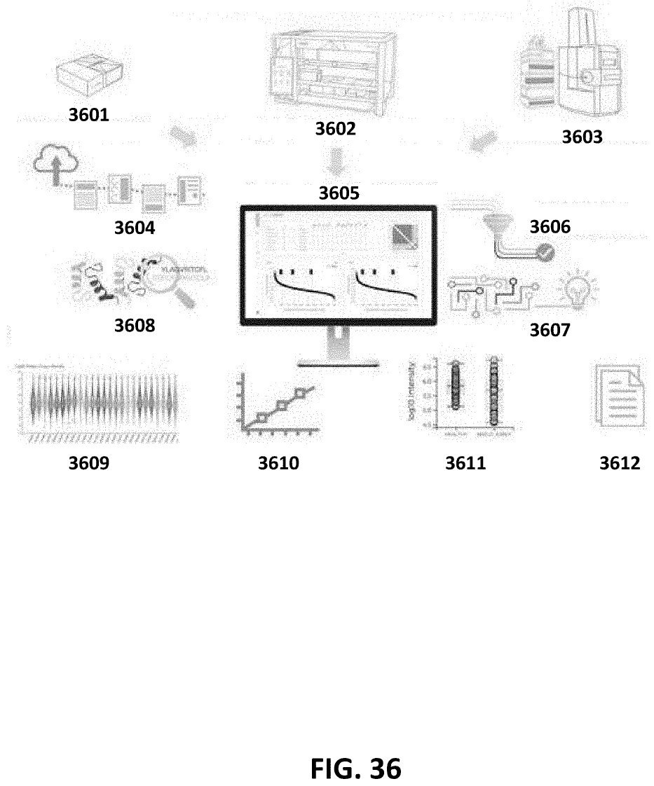

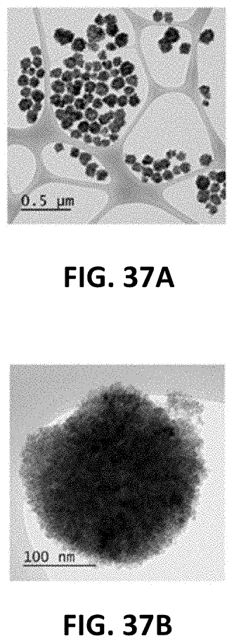

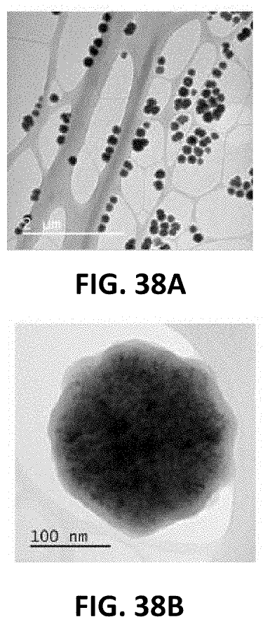

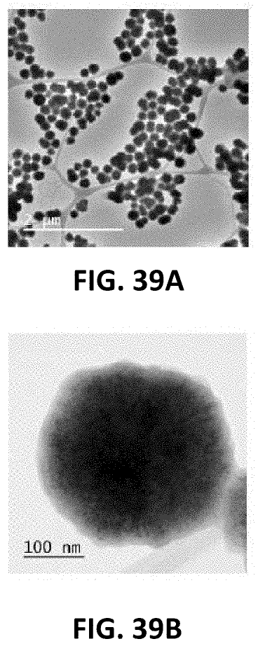

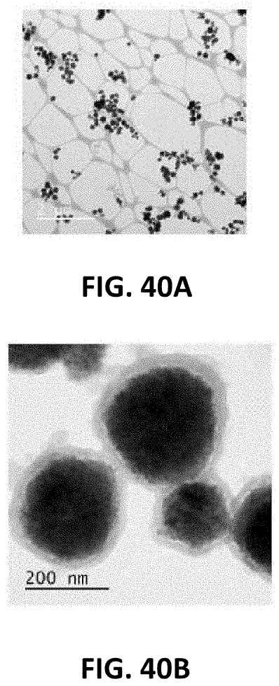

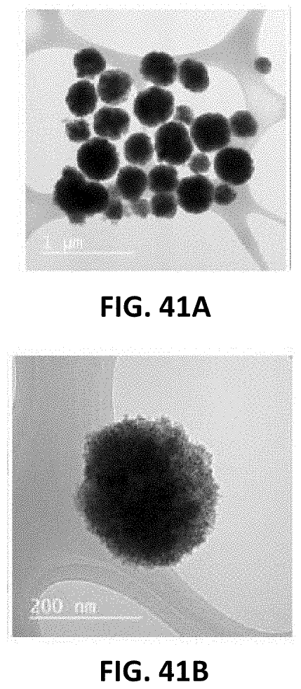

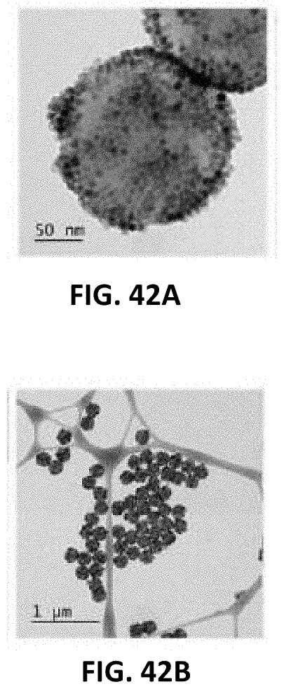

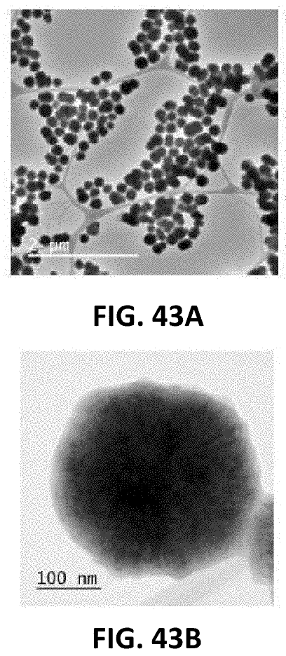

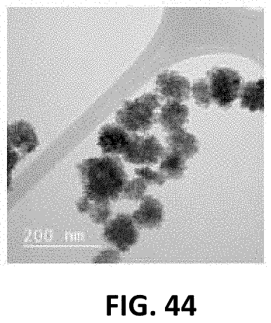

FIG. 36 illustrates a workflow utilizing assay instrumentation and materials and a computer-implemented system of the present disclosure. An assay kit 3601 comprising reagents for biomolecule corona analysis, an instrument configured to perform automated biomolecule corona analysis 3602, and an analytical instrument 3603 for identifying biomolecules from a biomolecule corona analysis method (e.g., a mass spectrometer) may be used to generate biomolecule corona data. A computer 3604 may communicate with the instrument configured to perform automated biomolecule corona analysis 3602, the analytical instrument 3603, or both instruments. The computer 3604 may provide parameters to or operate one or both instruments. The computer may receive data from the instrument configured to perform automated biomolecule corona analysis 3602, the analytical instrument 3603, or both. The computer 3604 may be in communication with a server 3605 (e.g., a cloud-based server), and may be configured to upload data to the server 3605. The computer 3604 may comprise one or more processors, as well as computer readable medium comprising machine-executable code which may be executed by the one or more processors, and which may be configured to implement a method 3606 for analyzing data from the instrument configured to perform automated biomolecule corona analysis 3602, the analytical instrument 3603, or both. The machine-executable code may also be configured to identify and annotate 3607 biomolecules from the biomolecule corona data. The computer may be configured to display 3608 the analyzed data or unanalyzed data, display metrics generated from analysis of the data 3609, display performance metrics 3610 (e.g., performance metrics derived from analysis of the data or received from the instrument configured to perform automated biomolecule corona analysis 3602, the analytical instrument 3603, or both. The machine-executable code may be configured to identify abundance ratios 3611 of species identified or annotated 3607. The machine-executable code may generate results files 3612, which may be transmitted through to the server 3605, to another device in communication with the computer 3604, such as a flash drive memory device.

### Classifiers for Neurodegenerative Disease Analysis

The method of determining a set of proteins associated with the disease or disorder and/or disease state include the analysis of the corona of the at least two samples. This determination, analysis or statistical classification can be performed using methods, including, but not limited to, for example, supervised and unsupervised data analysis, machine learning, deep learning, and clustering approaches including hierarchical cluster analysis (HCA), principal component analysis (PCA), Partial least squares Discriminant Analysis (PLS-DA), random forest, logistic regression, decision trees, support vector machine (SVM), k-nearest neighbors, naive bayes, linear regression, polynomial regression, SVM for regression, K-means clustering, and hidden Markov models, among others. In other words, the proteins in the corona of each sample can be compared/analyzed with each other to determine with statistical significance what patterns are common between the individual corona to determine a set of proteins that is associated with the disease or disorder or disease state.

Generally, machine learning algorithms are used to construct models that accurately assign class labels to datasets or features within datasets based on a set of input features. In some case it may be advantageous to employ machine learning and/or deep learning approaches for the methods described herein. For example, machine learning can be used to associate the protein corona with various disease states (e.g. no disease, precursor to a disease, having early or late stage of the disease, etc.). For example, in some cases, one or more machine learning algorithms are employed in connection with a method of the invention to analyze data detected and obtained by the protein corona and sets of proteins derived therefrom. For example, a machine learning algorithm may be trained to distinguish subjects with Alzheimer's disease from healthy subjects.

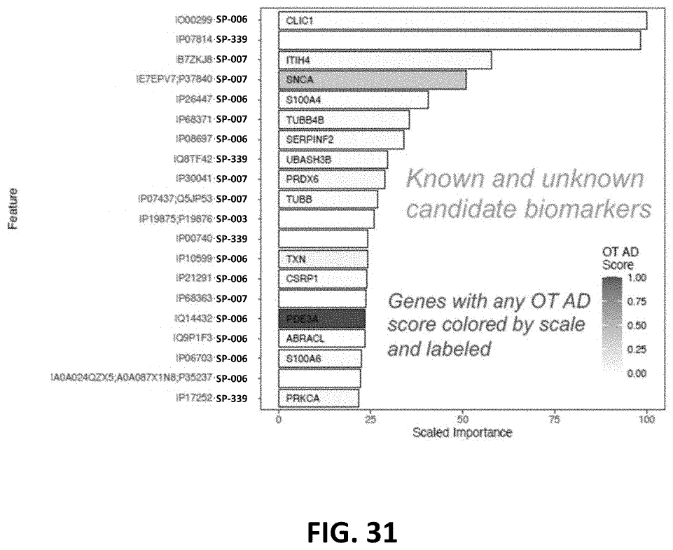

A method or system (e.g., a computer-implemented system) may utilize biomolecule corona data for classifier training and as an input on which a trained classifier may perform analysis. The biomolecule corona data may comprise raw data (data acquired directly from an instrument such as a mass spectrometer, or data which has been subjected to basic pre-processing and filtering steps, such as baseline flattening), processed data (e.g., a list of mass spectrometry peaks identified above a baseline signal-to-noise threshold, a ratio of two mass spectrometry peak intensities), annotated data (e.g., a list of peptides identified from mass spectrometric data), or any combination thereof. As the present disclosure provides methods for identifying biomolecules spanning broad dynamic ranges, biomolecule corona data used for training or biological sample analysis may span about 2 to about 12 orders of magnitude in terms of biomolecule concentration in the biological sample, about 4 to about 12 orders of magnitude in terms of biomolecule concentration in the biological sample, about 5 to about 12 orders of magnitude in terms of biomolecule concentration in the biological sample, about 6 to about 12 orders of magnitude in terms of biomolecule concentration in the biological sample, about 7 to about 12 orders of magnitude in terms of biomolecule concentration in the biological sample, about 8 to about 12 orders of magnitude in terms of biomolecule concentration in the biological sample, about 4 to about 10 orders of magnitude in terms of biomolecule concentration in the biological sample, about 5 to about 10 orders of magnitude in terms of biomolecule concentration in the biological sample, about 6 to about 10 orders of magnitude in terms of biomolecule concentration in the biological sample, about 7 to about 10 orders of magnitude in terms of biomolecule concentration in the biological sample, about 8 to about 10 orders of magnitude in terms of biomolecule concentration in the biological sample, about 2 to about 8 orders of magnitude in terms of biomolecule concentration in the biological sample, about 4 to about 8 orders of magnitude in terms of biomolecule concentration in the biological sample, about 6 to about 8 orders of magnitude in terms of biomolecule concentration in the biological sample, about 2 to about 6 orders of magnitude in terms of biomolecule concentration in the biological sample, about 4 to about 6 orders of magnitude in terms of biomolecule concentration in the biological sample, about 2 to about 4 orders of magnitude in terms of biomolecule concentration in the biological sample, or about 2 to about 3 orders of magnitude in terms of biomolecule concentration in the biological sample. For example, the top 20 particle-specific protein biomarkers from the Random Forest model summarized in FIG. 31 includes inter-alpha-trypsin inhibitor heavy chain family member 4 (ITIH4), which is typically present in plasma at around 100 μg/mL, and bifunctional glutamate/proline—tRNA ligase, which is typically present in plasma at around 20 pg/mL, or at about 7 orders of magnitude lower abundance than ITIH4.

Aspects of the present disclosure increase the amount of information derived from biological sample analysis. Some biological states are not distinguishable solely through biomolecule identification. For example, identifying concentrations for the thirty most abundant proteins in a plasma sample is often insufficient for distinguishing subjects afflicted with Alzheimer's disease from healthy subjects. The present disclosure provides a range of approaches for increasing the dimensionality of biological sample data, and for using the data to identify biological states. In some cases, biomolecule corona data may comprise a ratio of two or more biomolecule abundances or signal intensities. For example, a datapoint may be a ratio of three mass spectrometric peak intensities, and which may comprise greater diagnostic utility than the intensities of all three mass spectrometric peak intensities taken individually.

In some cases, biomolecule corona data comprises particle-level annotations which identify the type of particle a biomolecule was identified on, and further may optionally comprise an abundance of or a signal intensity associated with the biomolecule. For example, in some cases, alpha-2-antiplasmin plasma levels may be weakly diagnostic for Alzheimer's disease, but alpha-2-antiplasmin abundance in biomolecule coronas of a (PDMAPMA)-coated SPION contacted to plasma may vary with a high degree of statistical significance between healthy and Alzheimer's disease samples. In some cases, biomolecule corona data comprises particle-level annotations which identify the type of particle a peptide was identified on. In some cases, a plurality of peptides from a single protein are identified on a single particle. In some cases, biomolecule corona data comprises an abundance ratio of two peptides associated with a single protein on two different particles. In some cases, biomolecule corona data comprises sample condition annotations which identify a condition under which the biomolecule was observed. For example, a datapoint may comprise an abundance of a peptide identified from a biological sample, a particle type on which the peptide was identified, and the osmolarity and pH of the sample.

The present disclosure also identifies a number of proteins which can be diagnostic for neurological diseases. In some cases, a trained classifier utilizes a protein, a peptide fragment of a protein, or a signal associated with a protein in any one of TABLES 7-12. In some cases, a trained classifier utilizes at least two proteins (or associated peptides or signals) from any one of TABLES 7-12. In some cases, a trained classifier utilizes at least three proteins (or associated peptides or signals) from any one of TABLES 7-12. In some cases, a trained classifier utilizes at least four proteins (or associated peptides or signals) from any one of TABLES 7-12. In some cases, a trained classifier utilizes at least five proteins (or associated peptides or signals) from any one of TABLES 7-12. In some cases, a trained classifier utilizes about 2 to about 10, about 4 to about 10, about 5 to about 15, about 5 to about 20, about 8 to about 20, about 10 to about 25, or about 15 to about 30 proteins (or associated peptides or signals) from any one of TABLES 7-12. In some cases, a protein (or associated peptide or signal) is annotated with a particle type or condition used for its detection.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” “less than or equal to,” or “at most” precedes the first numerical value in a series of two or more numerical values, the term “no more than,” “less than” or “less than or equal to,” or “at most” applies to each of the numerical values in that series of numerical values. For example, less than or equal to 3, 2, or 1 is equivalent to less than or equal to 3, less than or equal to 2, or less than or equal to 1.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

### EXAMPLES

The following examples are illustrative and non-limiting to the scope of the compositions, devices, systems, kits, and methods described herein.

### Example 1

### Particle-Based Plasma Protein Profiling of Alzheimer's and Mild Cognitive Impairment Subjects

This example covers plasma biomarker identification for Alzheimer's disease (AD) and mild cognitive impairment (MCI). While Alzheimer's disease and mild cognitive impairment can affect homeostasis, expression, and morphology of nervous tissues, profiling these tissues is often intensive, expensive, and can impart permanent damage. The identification of clinically useful biomarkers for Alzheimer's disease and mild cognitive impairment from blood has thus been a long-standing goal. This example covers a particle-based assay for deep plasma proteomic profiling and candidate protein biomarker analysis for Alzheimer's disease and mild cognitive impairment. 200 subject plasma samples, comprising 50 Alzheimer's disease, 50 mild cognitive impairment, and 100 Controls were profiled with two separate 5-particle panels, summarized in TABLE 6 below. Using the 10-particle panel and 85 μL of plasma per nanoparticle, proteins were quantified by data-independent acquisition (DIA) liquid-chromatography mass-spectrometry (LC-MS) over about 6 weeks. Normalized peptide intensities were used in ten rounds of 10-fold cross-validation to develop random forest models for class discrimination.

TABLE 6
Particles used in Alzheimer's disease and mild
cognitive impairment study

| Particle | | Panel |
|---|---|---|
| ID | Description | ID |
| SP-339 | Polystyrene particles, Paramagnetic, Carboxyl- | Panel |
| | functionalized (PS-MAG-COOH) | A |
| SP-373 | Magnetizable Nanoparticles and magnetizable | Panel |
| | microparticles, Dextran based//plain/25 mg/ml | A |
| SP-003 | Superparamagnetic, silica coated | Panel |
| | | A |
| SP-006 | Silica coated, amine | Panel |
| | | A |
| SP-007 | PDMAPMA coated (Dimethylamine) | Panel |
| | | A |
| SP-333 | Carboxylate | Panel |
| | | D |
| SP-347 | Silica | Panel |
| | | D |
| SP-353 | Amino | Panel |
| | | D |
| SP-389 | Wheat Germ Agglutinin | Panel |
| | | D |
| SP-008 | 1,2,4,5-Benzenetetracarboxylic | Panel |
| | acid coated SPION | D |

The data from all 200 subjects (comprising approximately 2,000 nanoparticle corona preparations and MS data acquisition runs) were collected over a period of approximately one month using the 10 particle panel outlined in TABLE 6 for sample processing. A total of 2,617 proteins were detected by the 10 particle panel, with 2,232 proteins present in at least 25% of the samples. Forty proteins with the highest possible Alzheimer's OpenTargets scores were part of this list, including Amyloid beta, ApoE and Clusterin. Median protein counts per nanoparticle ranged from 747 to 1,209. A total of 26,264 peptides were detected, with 16,323 peptides present in at least 25% of the samples. Median peptide counts per nanoparticle ranged from 5,273 to 8,785.

### Inclusion Criteria and Sample Classification

Inclusion criteria for participation in the study included a Mini-Mental State Examination (see Folstein et al. “Mini-mental state”. A practical method for grading the cognitive state of patients for the clinician. J Psychiatr Res. 1975 November; 12(3):189-98.) score of between 14 and 28, age of at least 50, a magnetic-resonance imaging (MM) or computerized tomography (CT) scan within the past two years excluding other pathologies, and a Hachinski score of less than 4. General exclusion criteria included evidence of multi-infarct dementia, drug intoxication, thyroid disease, pernicious anemia, tertiary syphilis, chronic infections of the nervous system, normal pressure hydrocephalus, Huntington's disease, Creutzfeldt-Jakob disease and brain tumors, polypharmacy, or Korsakoffs syndrome as a cause of dementia.

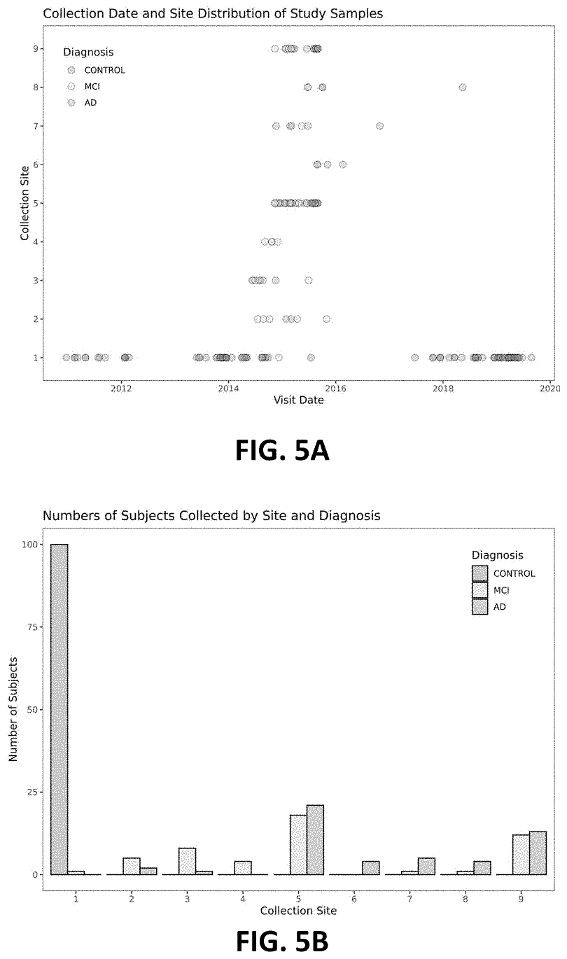

FIG. 5A summarizes the date, site, and class for the 200 collected samples. Several important features in the sample collection design are revealed in this plot. First, all of the Control group samples come from one collection site (Site 1) and were collected in three distinct periods between 2011 and 2020. Second, all of the AD and MCI subject samples (except one) were collected across the remaining 8 sites, primarily during late 2014 and 2015. Third, site 5 and site 9 supplied most of the AD and MCI samples. Based on the subject notation, it is likely that different collection protocols were used for the Control, AD and MCI samples. FIG. 5B outlines the numbers of samples collected for each diagnosis class across the collection sites, with sites 1, 5, and 9 providing the vast majority of 200 samples.

Sample annotations, provided after blinded sample processing, were evaluated in order to understand the study design and any potential issues with respect to between-sample or between-group comparisons. Probable Alzheimer's disease classifications were ascribed to subjects meeting NINCDS-ARDA criteria (McKhann G, Drachman D, Folstein M, Katzman R, Price D, Stadlan E M (1984). “Clinical diagnosis of Alzheimer's disease: report of the NINCDS-ADRDA Work Group under the auspices of Department of Health and Human Services Task Force on Alzheimer's Disease”. Neurology. 34 (7): 939-44.), including Mini-Mental State Examination scores of between 14 and 26, and exhibiting progressive deterioration of specific cognitive functions, impaired activities of daily living and altered patterns of behavior. Probable mild cognitive impairment classifications were ascribed to subjects determined to be memory compliant, not demented, and with preserved cognitive function; with abnormal memory function below education adjusted cutoff on Logical Memory II subscale from the Wechsler Memory Scale—Revised; and with Mini-Mental State Examination scores of between 22 and 28.

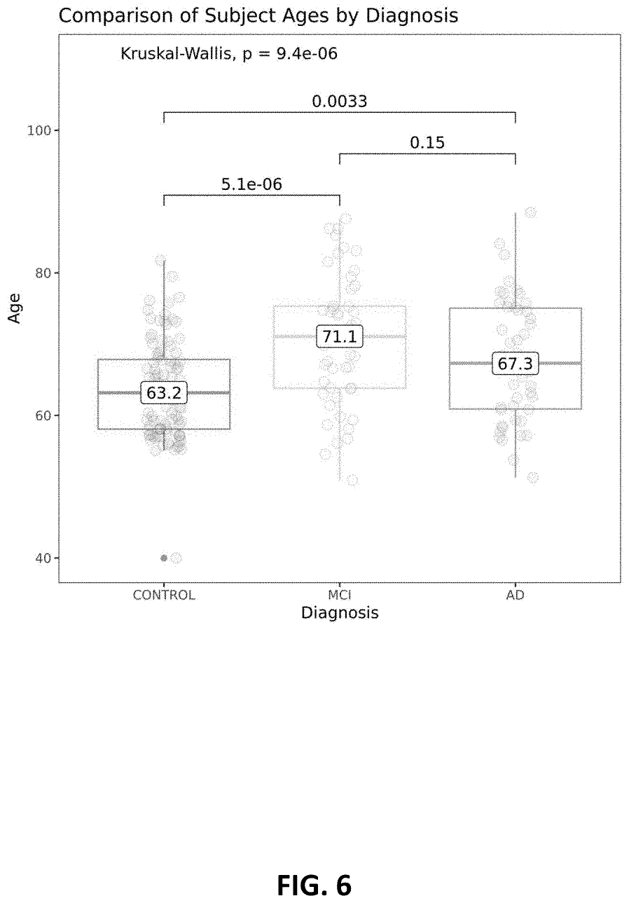

FIG. 6 summarizes the age and gender distributions between the diagnosis groups. In this figure, the ages of the subjects collected for each group are plotted along with a non-parametric test, Kruskal-Wallis, which analyzes whether or not the age distributions come from the same original distribution. As is shown in FIG. 6 there is a significant difference in the Control-vs-MCI and Control-vs-AD comparisons, but no significant difference in the MCI-v-AD comparisons. Although the differences for the Control comparisons may be formally statistically significant, the actual magnitude of the difference may not be clinically meaningful, given a difference in the medians of 7.9 years and 4.1 years for the MCI and AD comparisons, respectively.

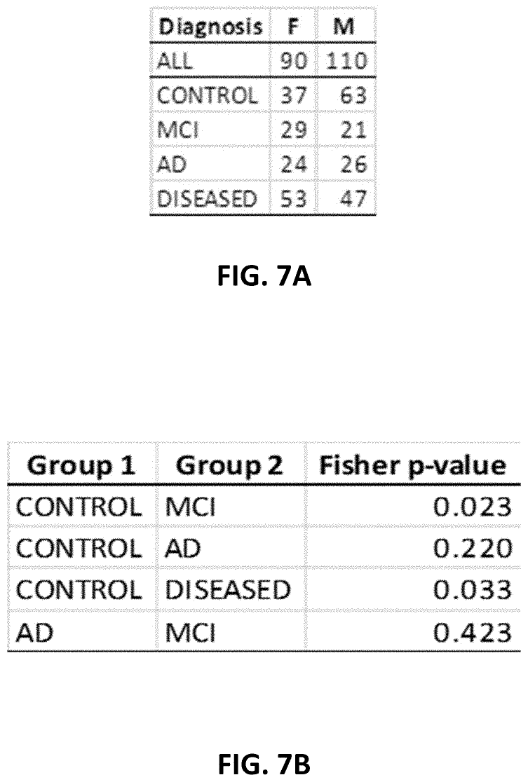

The reported gender status for each subject was also used to ascertain significant differences between the comparative groups. In FIG. 7A, the female and male gender counts are shown for the entire study cohort as well as the groupings intended for comparative analysis. Simple inspection suggests that the MCI group may have a significantly different gender proportion as compared to the CONTROL group, as well as the combined AD and MCI subjects in the DISEASED group.

Using a Fisher test for proportionality comparisons, that observation is confirmed with the gender proportions for the CONTROL-v-MCI as well as the CONTROL-v-DISEASED having significant different proportions (FIG. 7B). As these samples are likely based on convenience collection protocols, and not specific intent-to-test enrollment, the age imbalance is likely due to the small numbers of samples involved and not an underlying difference in diagnosis by gender, although this is not certain. Given that the subjects were not enrolled on an intent-to-test basis, by which these parameters would reflect the true test population, age and gender should not be used in this study as parameters in the development of diagnosis classification models.

### Particle-Based Proteomic Analysis

Protocols for processing the samples are generally described in Blume et al. Nature Communications. 2020; 11(1):3662. Briefly, the 10 particles were separately provided in dry form, and reconstituted with deionized water to final total particle concentrations of 2.5-15 mg/ml. The 200 plasma samples were subjected to 5-fold buffer dilutions, mixed with the particle solutions, and then sealed and incubated at 37° C. for 1 hour with shaking at 300 rpm to promote biomolecule corona formation. After incubation, the plate was placed on top of a magnetic collection device for 5 minutes to draw down the particles. While still magnetically immobilized, the particles were subjected to a series of wash steps with 150 mM KCl and 0.05% CHAPS in a pH 7.4 Tris EDTA buffer to remove non-biomolecule corona bound biomolecules. Next, Lyse buffer was added to each sample and heated at 95° C. for 10 min with agitation at 1000 rpm. Trypsin was added to the samples for protein digestion. After 3 hours at 37° C. and 500 rpm shaking, the trypsin digestion was stopped by lowering sample pH. The particles were magnetically removed from the digested samples. The digested samples were then twice eluted from the filter cartridge and combined. The peptides were analyzed with data-dependent liquid chromatography-tandem mass spectrometry (LC-MS/MS).

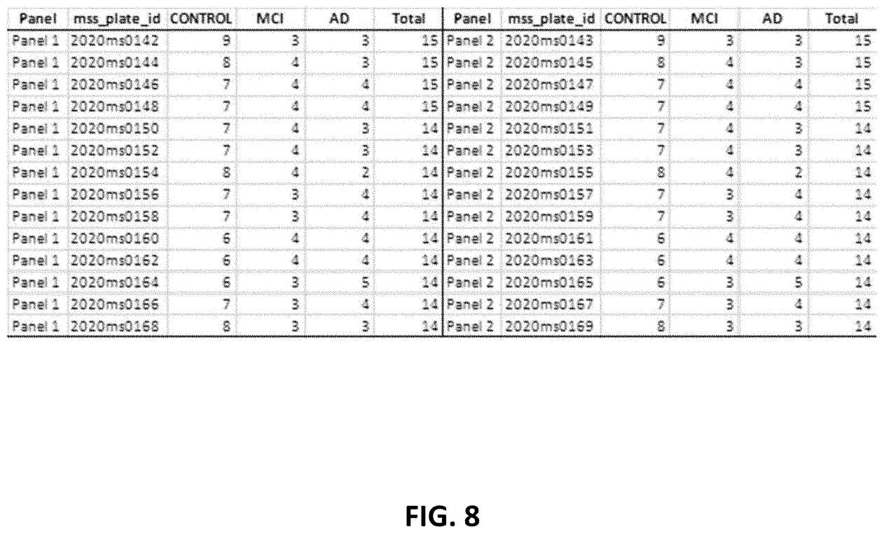

The experiments performed for this example used a 16 sample-per plate configuration, and interrogated each sample interrogated with 5 particles. Each sample was interrogated with one of two 5-particle panels, each of which is summarized in TABLE 6. The number of control, MCI, and AD samples per plate, as well as the identities of the particle panels used for interrogation, are provided in FIG. 8. Mass spectrometry data were collected using data-independent acquisition (DIA) on Seer's Sciex 6600+ platform.

Plasma samples for the 200 subjects were processed without prior knowledge of their diagnostic status using a randomization schema. The intent was to distribute the subject samples from the three classes across the sample preparation plates to avoid any systematic processing bias. The 200 samples in this study were randomized by class across sufficient plates (n=14). One automated biomolecule corona sample preparation instrument and one mass spectrometer were able to process and collect data from all 200 samples in about 6 weeks.

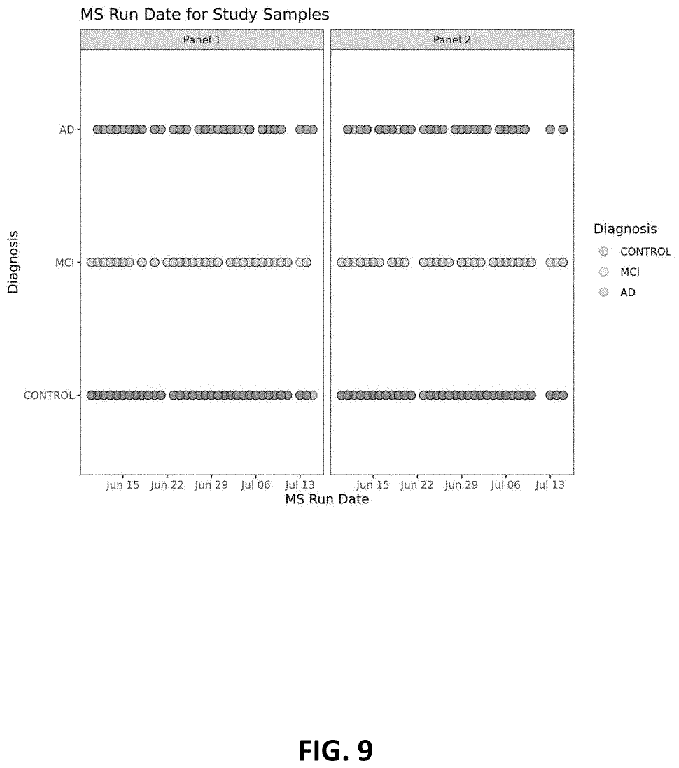

FIG. 9 provides the dates of mass spectrometry runs for the particle panel-interrogated samples. As would be expected, based on the plate layouts tabulated above, there is a relatively even time distribution for the processing of the samples, likely avoiding any bias in the particle sample preparations by class (i.e., control, MCI, and AD).

Sample preparation with the particle panels yielded digested peptides in solution which are quantified using ThermoFisher peptide quant kits prior to drying and subsequent resuspension before mass spectrometric analysis. At least in part due to differing physicochemical properties of the particles, peptide yields varied across the 10 particle types (both in terms of total peptide yield and peptide types). Nonetheless, the yields for each particle were fairly consistent across samples. Since constant sample volumes were used for each assay, differences in peptide yield across samples was taken as diagnostic of differences in plasma protein concentrations.

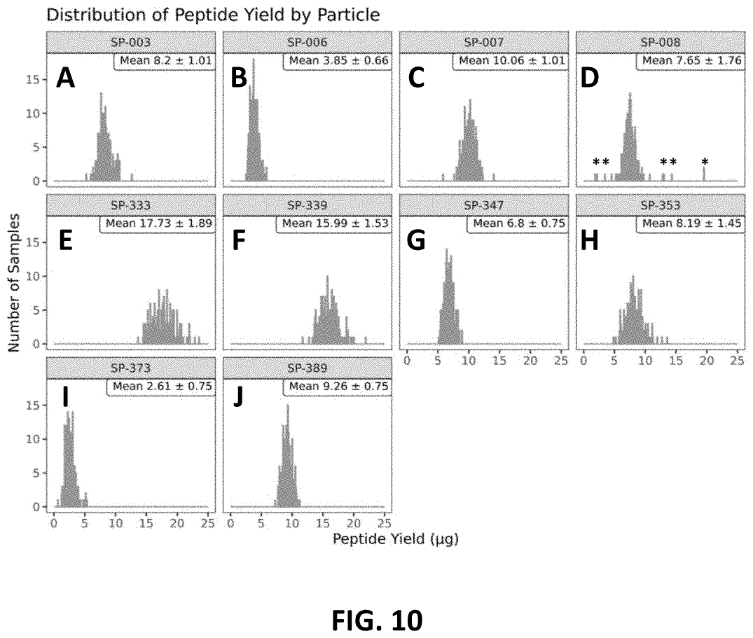

As is shown in FIG. 10, the yield of proteins for each particle is relatively consistent and roughly normally distributed. In this figure, panel A provides results for SP-003 particles, panel B provides results for SP-006 particles, panel C provides results for SP-007 particles, panel D provides results for SP-008 particles, panel E provides results for SP-333 particles, panel F provides results for SP-339 particles, panel G provides results for SP-347 particles, panel H provides results for SP-353 particles, panel I provides results for SP-373 particles, and panel J provides results for SP-389 particles. Although a few outlying samples are apparent (for example, the asterisked values (*) in the plot for SP-008 particles), no samples were rejected as outliers given the possibility that the differences could reflect true biological variation in particle corona formation and not merely artifacts of differential particle-based processing.

### Process Control Description

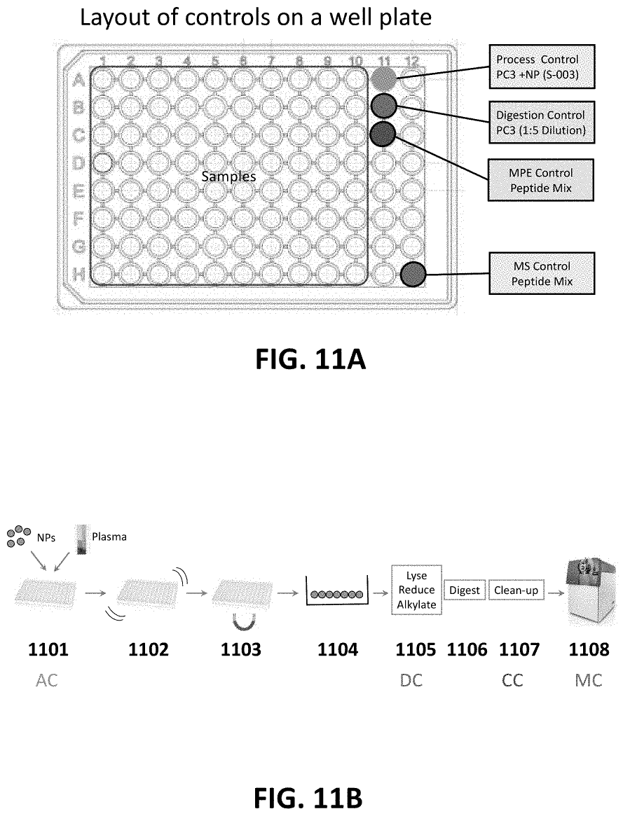

Each processing plate included control samples for various stages of the assay. These included an overall process control which went through the full assay with one nanoparticle as well as a digestion control, an MPE control for the filtration device, and a mass spectrometry control which comprised pre-prepared peptides for mass spectrometric data acquisition evaluation. The layout of the assay plate used in this example and the context of the controls are shown in FIG. 11A. An outline of the assay used in this example is shown in FIG. 11B, which follows an addition step 1101, in which samples and particles were combined in wells on the sample plate; an incubation step 1102, in which the samples were maintained under conditions suitable for biomolecule corona formation on the particles; a wash step 1103, in which the particles (with adsorbed biomolecule coronas) were magnetically immobilized within wells and the unbound content was removed through solvent washes, thereby yielding biomolecules coronas on the particles 1104; a digestion preparation step 1105, in which the biomolecule coronas were contacted with lyse buffer, reducing agents, and alkylating agents (for breaking disulfide bonds and alkylating thiols); a digestion step 1106 comprising protease digestion of biomolecule corona-bound biomolecules; a clean-up step 1107 including solid-phase extraction of the resulting fragmented biomolecule corona peptides; and mass spectrometric analysis 1108. The input of the controls is indicated at the bottom of FIG. 11B, with the process control (AC), digestion control (DC), MPE control (CC), and mass spectrometry control (MC) indicated below the various stages.

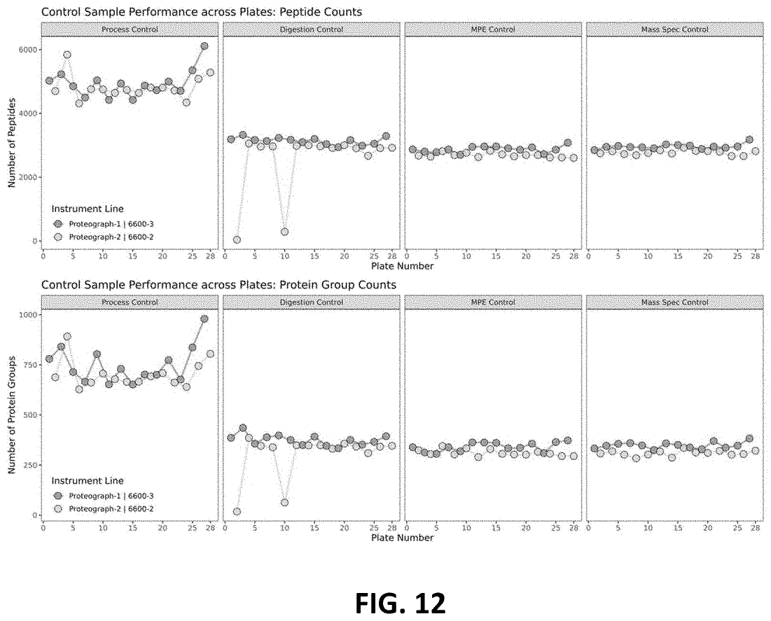

FIG. 12 provides peptide and protein counts for the process controls outlined in FIG. 11 for each of the sample plates as they were sequentially processed with particle panels and submitted for mass spectrometric evaluation. The samples were processed on separate pairs of instruments, indicated as “Proteograph-1” and “Proteograph-2,” each comprising automated particle assay control units and mass spectrometers. This processing strategy was employed to streamline the logistics of processing as well as reduce sources of variation.

### Protein Analysis

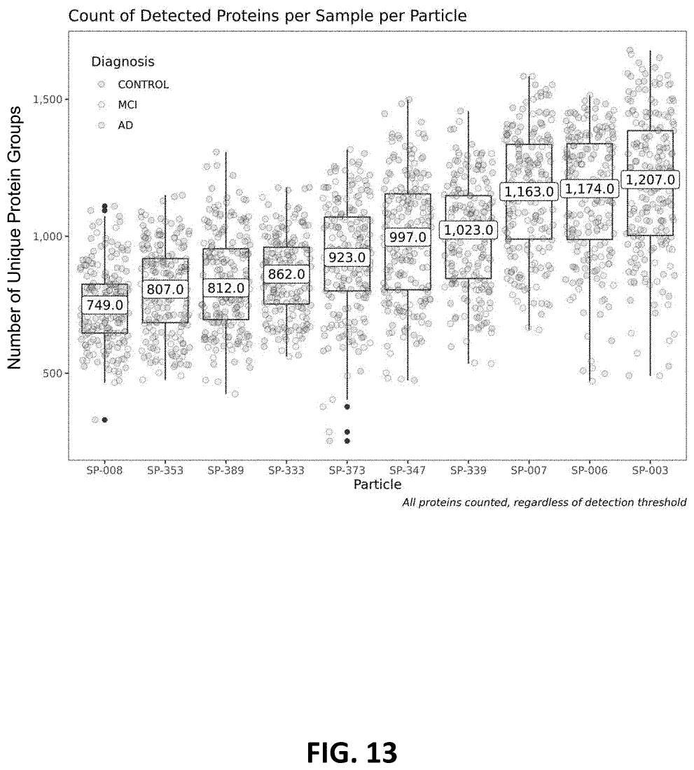

FIG. 13 provides the median numbers of protein groups detected on each particle type, with control, MCI, and AD samples shown in different colors. Given that each of the 10 particles has unique physicochemical properties, it was expected that the numbers of protein groups identified on each particle could vary. The median number of protein groups detected on each of the 10 particles ranges from 749 for SP-008 to 1,207 for SP-003. There appears to be little correlation between sample type (control, MCI, AD) and total protein count.

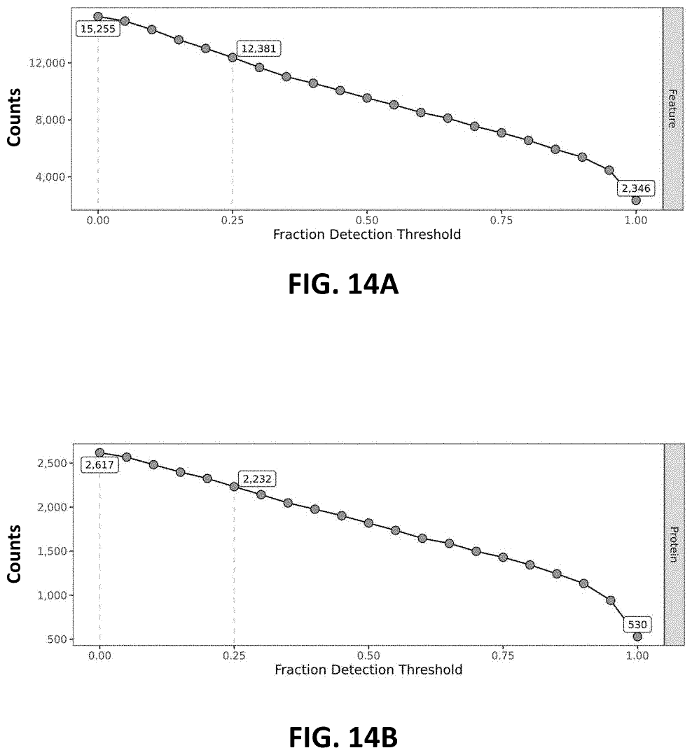

To control for measurement stochasticity and inter-sample variations not reflective of biological state, the results were filtered to exclude protein groups not observed in at least 25% of samples within the study. FIG. 14A summarizes the percentage of samples in which identified features (e.g., specific particle-protein intersections) were observed across the 200 samples. FIG. 4 summarizes the percentage of samples in which protein groups were observed across the 200 samples. 2,232 protein groups and 12,381 unique particle-protein intersections were detected in at least 25% of the study samples.

### Precision Analysis

As reproducible measurement is often a key requirement in proteomics profiling and biomarker studies, a reasonably robust and relatively simple normalization strategy was implemented. First, the protein log intensity data were median normalized using reference proteins defined as those present in all samples in the study for each given particle type. Then a scaling factor for each sample for each given particle was calculated so that the medians of the reference proteins (or peptides) for each sample were adjusted to the mean of the medians across all samples.

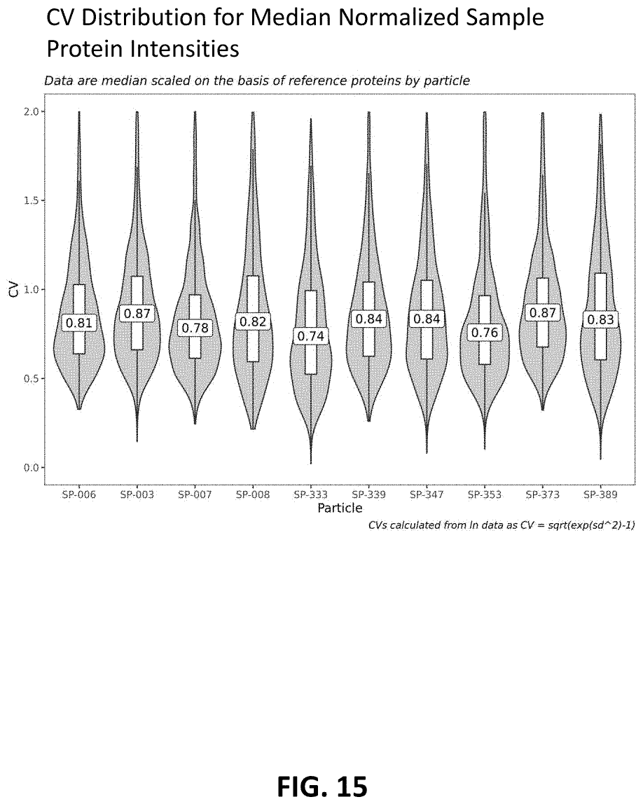

FIG. 15 summarizes the resulting coefficient of variation values for proteins observed in all 200 samples on each particle type. These values include both the biological variation across the samples within the study as well as the technical variation of the particle-based assay and subsequent mass spectrometric data collection.

### Overlap of Proteins to Annotated Alzheimer's Disease Targets

Coverage of high-value, annotated list Alzheimer's disease candidate biomarkers were evaluated against the full list of 2,617 protein groups detected across the study's 200 samples. 673 unique protein entries were selected from OpenTargets (https://www.opentargets.org) gene and protein annotations with Alzheimer's scores equal to 1. These entries include proteins from all tissues, not limited to blood, and represents a superset of potential targets from which a subset might be accessible in plasma. 40 high-value Alzheimer's targets were identified by overlapping the proteins detected in this study with the 673 protein entries from OpenTargets. Those proteins, and the fraction of the 200 samples in which those proteins were detected (column titled “Detected”), are shown in TABLE 7 below.

TABLE 7
Alzheimer's disease targets identified with particle panels

| Gene | Entry | Detected | Name |
|---|---|---|---|
| ADAM10 | 014672 | 0.93 | Disintegrin and metalloproteinase |
| | | | domain-containing protein 10 |
| APOCI | K7ERI9 | 1 | Apolipoprotein C-I |
| APOCI | P02654 | 1 | Apolipoprotein C-I |
| APOE | P02649 | 1 | Apolipoprotein E |
| APP | P05067 | 1 | Amyloid beta A4 protein |
| BCHE | P06276 | 0.465 | Cholinesterase |
| CAPN1 | P07384 | 1 | Calpain-1 catalytic subunit |
| CAPN2 | P17655 | 0.995 | Calpain-2 catalytic subunit |
| CAPNS1 | A0A0C4DGQ5 | 1 | Calcium-activated neutral |
| | | | proteinase small subunit |
| CAPNS1 | P04632 | 1 | Calpain small subunit |
| CAST | A0A0A0MR45 | 0.09 | Calpain inhibitor |
| CAST | A0A0C4DGB5 | 0.09 | Calpain inhibitor |
| CAST | A0A0C4DGD1 | 0.09 | Calpain inhibitor |
| CAST | B7Z574 | 0.09 | Calpain inhibitor |
| CAST | E7EQ12 | 0.09 | Calpain inhibitor |
| CAST | E7EQA0 | 0.09 | Calpain inhibitor |
| CAST | E7ES10 | 0.09 | Calpain inhibitor |
| CAST | E7EVY3 | 0.09 | Calpain inhibitor |
| CAST | E9PCH5 | 0.09 | Calpain inhibitor |
| CAST | E9PDE4 | 0.09 | Calpain inhibitor |
| CAST | H0Y7F0 | 0.09 | Calpain inhibitor |
| CAST | H0Y9H6 | 0.09 | Calpain inhibitor |
| CAST | H0YD33 | 0.09 | Calpain inhibitor |
| CAST | P20810 | 0.09 | Calpastatin |
| CLU | P10909 | 1 | Clusterin |
| CR1 | E9PDY4 | 0.43 | Complement receptor type 1 |
| CR1 | E9PQN4 | 0.43 | Complement receptor type 1 |
| CR1 | P17927 | 0.43 | Complement receptor type 1 |
| CR1 | Q5SR44 | 0.43 | Complement receptor type 1 |
| LMNA | P02545 | 0.995 | Prelamin-A/C |
| LMNA | Q5TCI8 | 0.995 | Prelamin-A/C |
| LMNB1 | P20700 | 0.985 | Lamin-B1 |
| MMP1 | P03956 | 0.68 | Interstitial collagenase |
| NECTIN2 | K7EKE8 | 0.33 | Nectin-2 |
| NECTIN2 | Q92692 | 0.33 | Nectin-2 |
| PDE3A | Q14432 | 0.77 | cGMP-inhibited 3′,5′- |
| | | | cyclic phosphodiesterase A |
| PRDX1 | Q06830 | 1 | Peroxiredoxin-1 |
| PRDX2 | P32119 | 1 | Peroxiredoxin-2 |
| PTGS1 | A0A087X296 | 0.96 | Cyclooxygenase-1 |
| PTGS1 | P23219 | 0.96 | Prostaglandin G/H synthase 1 |

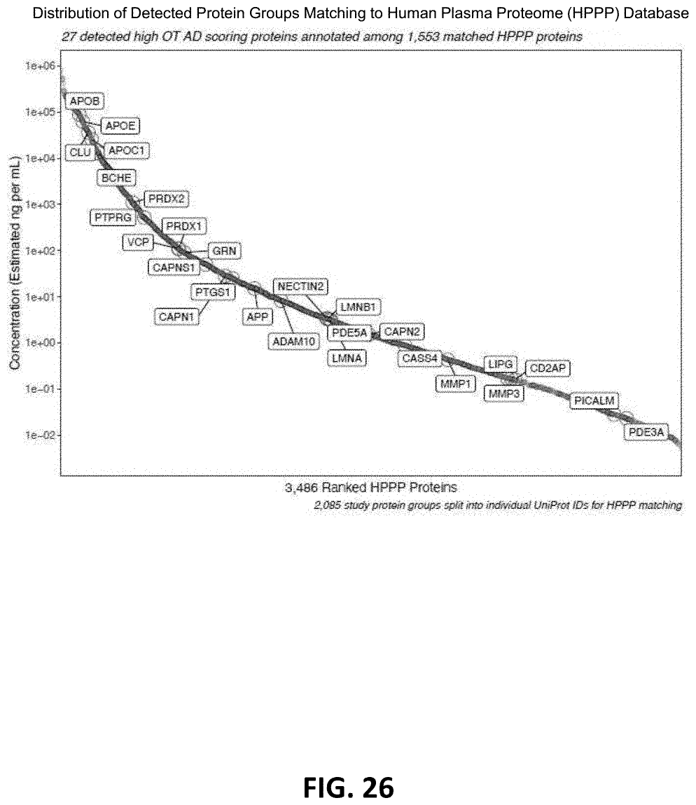

The particle assay profiled deep into the plasma dynamic range. Particle range compression enabled quantification of proteins spanning more than 8 orders of magnitude in concentration in the plasma samples. FIG. 26 summarizes the 2,085 protein groups detected in at least 25% of the 200 samples, with the y-axis providing estimated human plasma concentrations in units of ng/ml. The proteins were matched to the Human Plasma Proteome database of 3,486 proteins. Detected, overlapping proteins are marked on the ranked plot below to show the depth of plasma profiling using the systems and methods disclosed herein. 27 proteins with high OpenTargets Alzheimer's Disease association scores (Score ≥0.7) were captured as part of this overlap, and are summarized in TABLE 7 above.

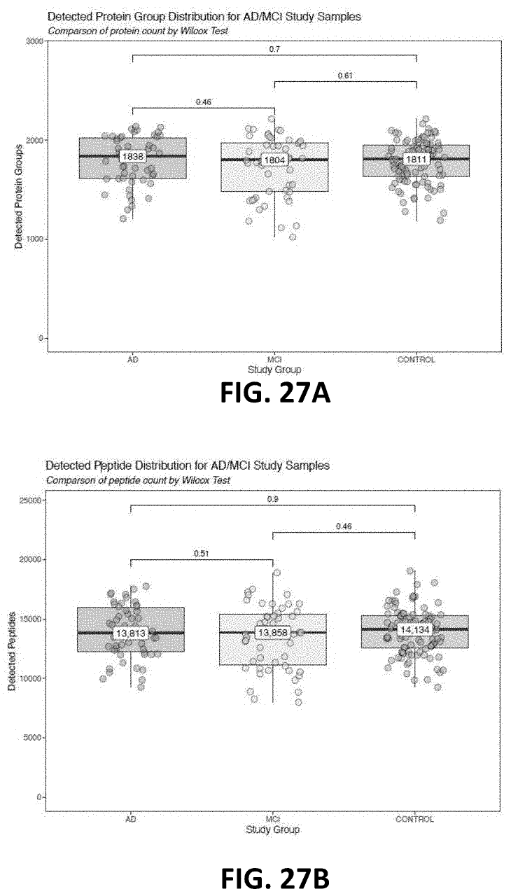

FIG. 27A provides total protein group counts, while FIG. 27B provides total peptide counts for each of the 200 samples used in the study. Each datapoint corresponds to the aggregate number of protein groups or peptides detected across the 10 particle types. Wilcoxson test scores are summarized in each plot, and highlight that each study group yielded similar peptide and protein group counts.

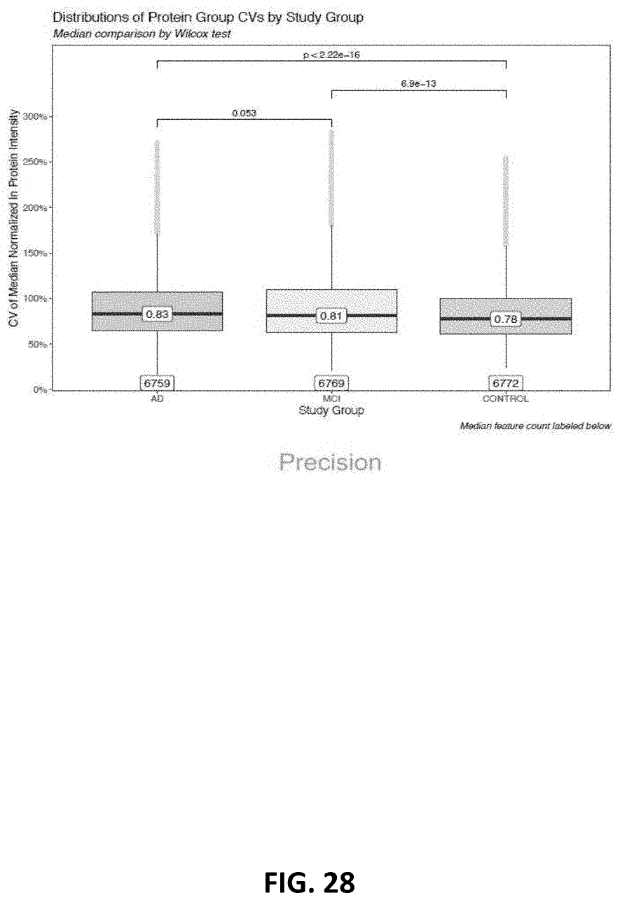

FIG. 28 summarizes coefficients of variation for protein group intensities of the AD, MCI, and control group samples (left to right). The median coefficients of variation were 0.83 for AD protein groups, 0.81 for MCI protein groups, and 0.78 for control protein groups. Wilcox test comparisons between the three groups are indicated by the bars and values at the top of the chart.

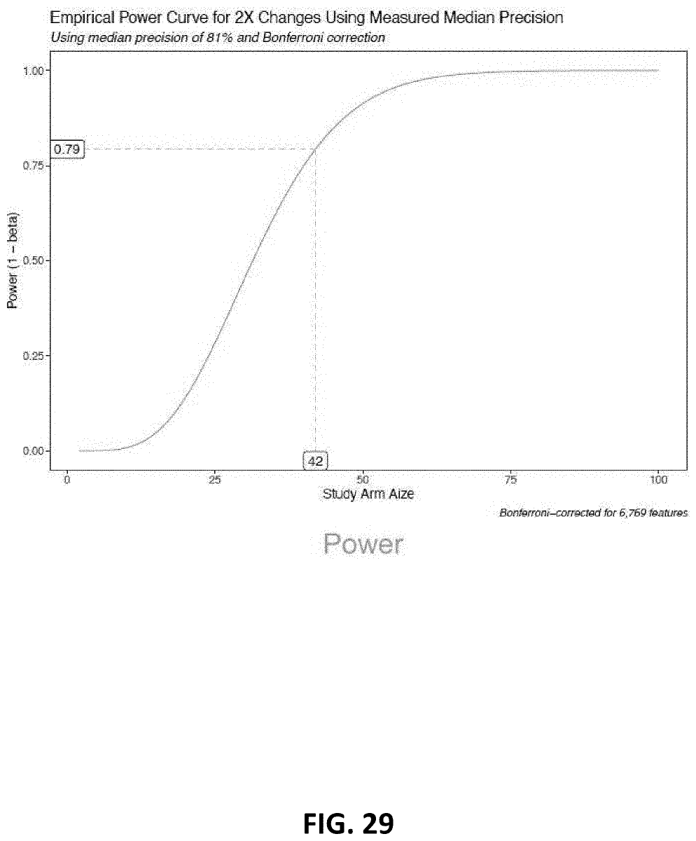

FIG. 29 provides an empirical power curve for 2-fold changes using measured median precision of 81% and Bonferroni correction. As can be seen in the figure, the assays were capable of resolving 2-fold changes with as few as n=42 subjects per study group.

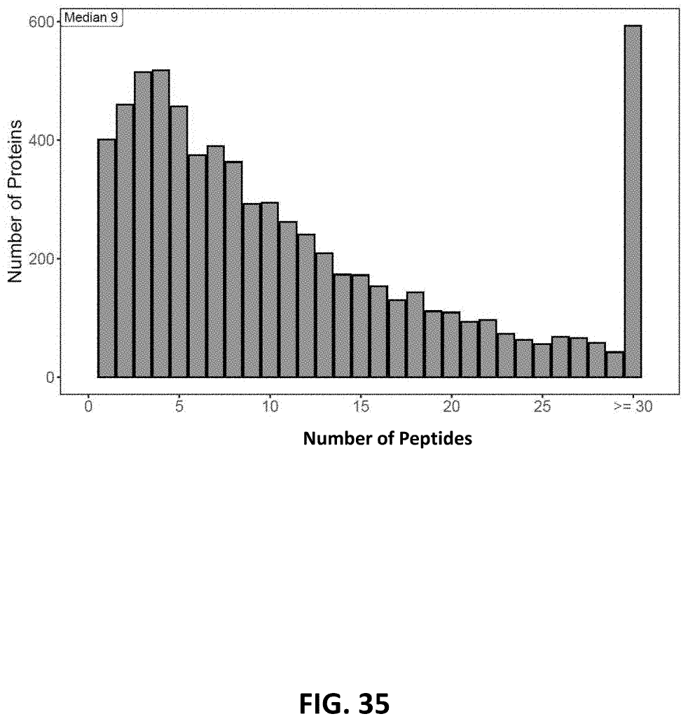

Multiple peptide identifications per protein group generated rich datasets for proteomics and multifold validation for protein group assignments. FIG. 35 summarizes the number of peptides detected for each identified protein group. 26,264 peptides were detected in total with a median 9 peptides per protein across the AD study, and less than about 20% of identified protein groups corresponding to fewer than 5 detected peptides.

### Peptide Analysis

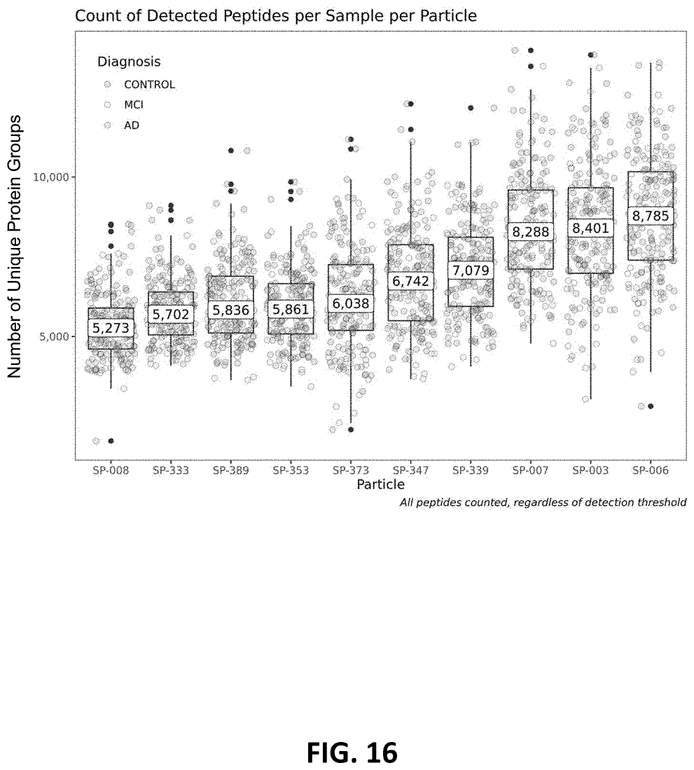

FIG. 16 provides the number of unique peptides identified from each sample on each of the 10 particle types. The median peptide number is provided for each particle type, with 1^{st} and 3^{rd} quartiles presented by the box plots. The median per-sample peptide counts spanned from 5,273 for SP-008 particles to 8,785 for SP-006 particles. As with the per-sample protein counts presented in FIG. 13, the number of peptides identified per sample correlated with particle type, but not with sample type.

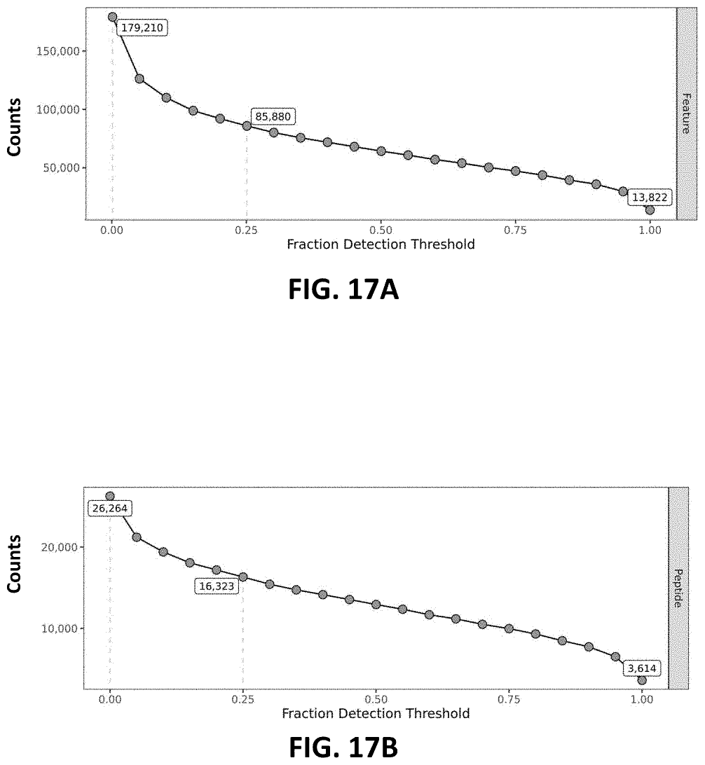

FIG. 17A summarizes the fraction of samples in which individual features (e.g., specific particle-peptide intersections) were observed. FIG. 17B summarizes the percentage of samples in which individual peptides were observed. 16,323 of 26,264 total peptides were detected in at least 25% of the study samples, while 85,880 particle-peptide intersections of 179,210 particle-peptide intersections were detected in at least 25% of the study samples, showing that particle-level variations capture additional complexities beyond those observed at the peptide level.

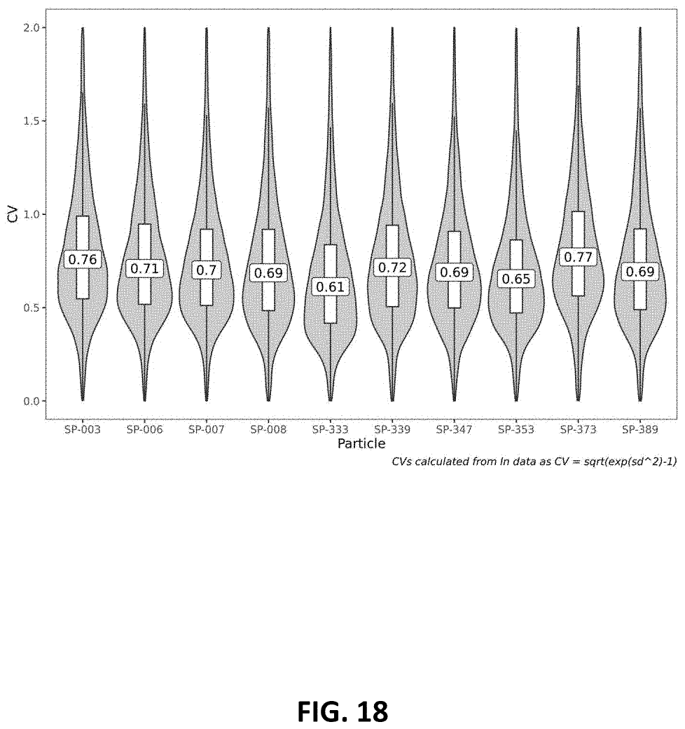

FIG. 18 summarizes coefficient of variation values for mass spectrometric intensities of peptides observed in all 200 samples on each particle type. Peptide-level precision analysis was performed as described above for proteins. Briefly, common peptides present in all study samples for a given particle were used to calculate scaling values to adjust the medians of those values for each sample to a common mean across the samples. These precision values for median normalized peptide intensities reflect the total variance across the study, including both biological variance from the subjects as well as pre-analytical and analytical noise.

### Univariate Comparisons and Biomarker Identification

As a first analysis for the potential to discriminate between sample types (i.e., control, MCI, AD) using the peptide data, an initial univariate analysis was performed. Using the peptide data, median normalized as described above, and filtered to include only those peptides which were present in at least 50% of at least one of the classes, a Wilcox test, non-parametric analysis was performed on a feature-by-feature basis. As above, a feature in this context is a particle-peptide intersection, meaning that more than one particle may provide unique intensity values for the same identified peptide sequence.

Four sample group comparisons were performed: CONTROL v AD, CONTROL v MCI, AD v MCI, and CONTROL v DISEASED, where DISEASED is defined as the combination of the 50 AD and 50 MCI samples. Multiple testing correction (Benjamini-Hochberg 5% FDR) was performed using all of the features from the ten nanoparticles.

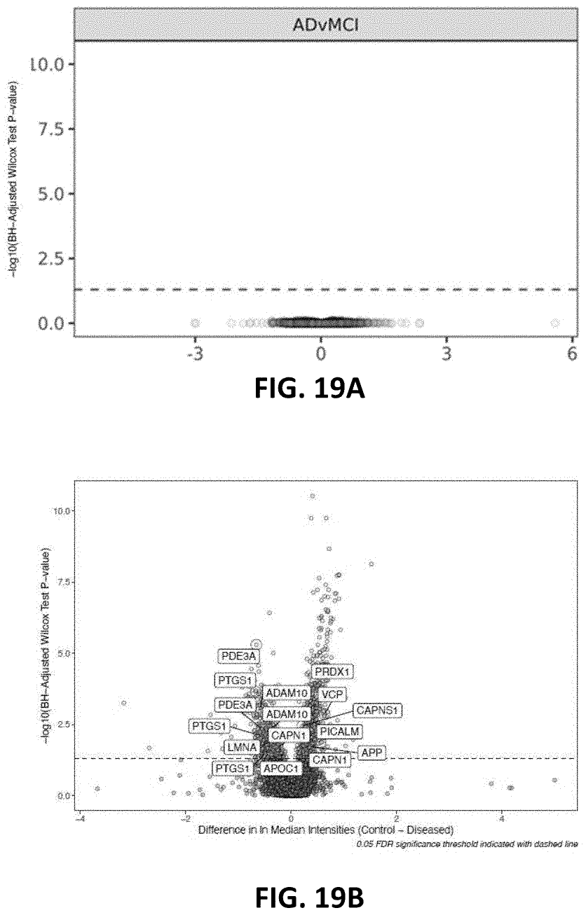

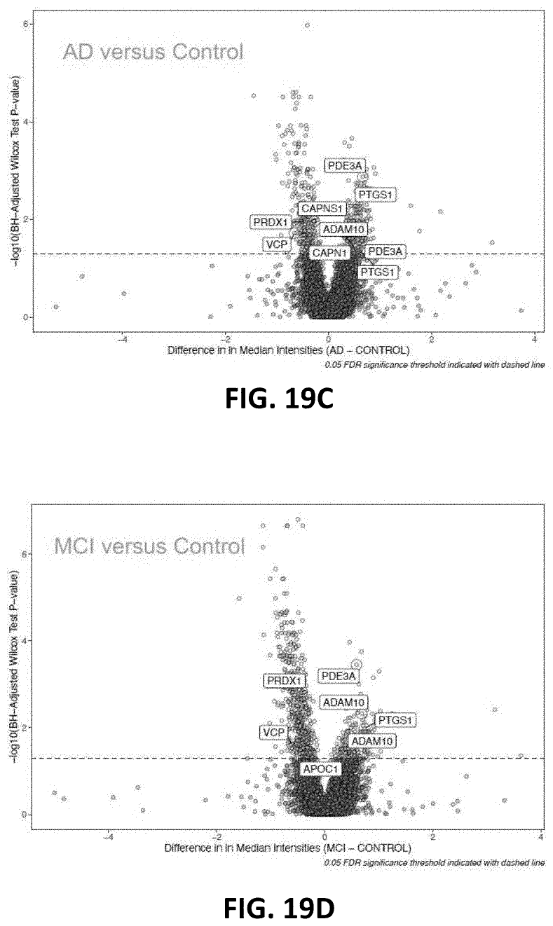

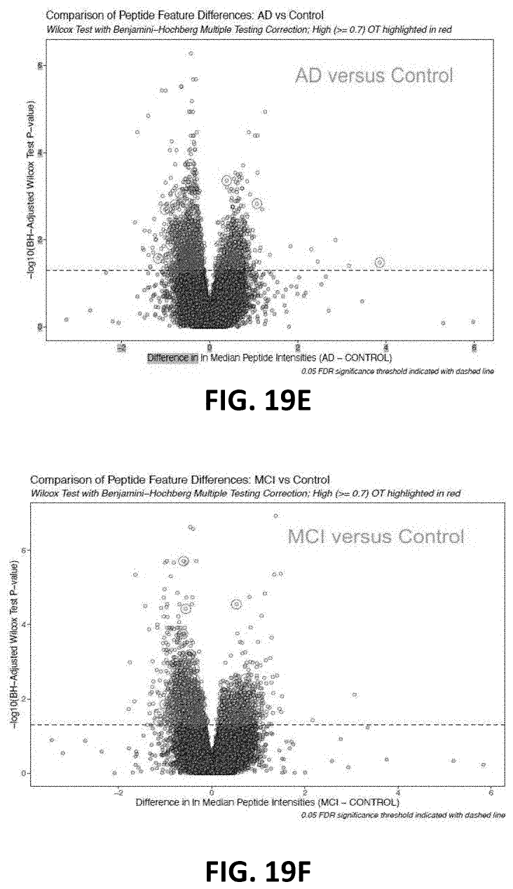

FIGS. 19A-19D provide volcano plots of the peptide features for each of four comparisons. FIG. 19A provides a volcano plot comparison of AD and MCI samples. FIG. 19B provides a volcano plot comparison of control and diseased samples. FIG. 19C provides a volcano plot comparison of control and AD samples. FIG. 19D provides a volcano plot comparison of control and MCI samples. FIGS. 19E-19F provide the volcano plots of FIGS. 19C-D, respectively, with features associated with OpenTarget AD scores of 0.7 or greater circled and labeled. The panels show differences in the median intensities for observed peptide features, plotted as natural log transformations of the original reported mass spectrometric intensities. The red lines in the plots show a false discovery rate. Log transformed protein intensity data were median normalized and filtered to those protein groups present in >25% of the samples. Univariate comparison was done by a Wilcox Test with Benjamini-Hochberg multiple-testing correction. Significant protein changes were observed for control versus AD and control versus MCI comparisons. As a first analysis, the combined diseased samples, AD and MCI, were compared to the control samples (FIG. 19B). Many significant protein differences existed between the disease and controls including a significant number of proteins with high OpenTarget AD scores.

The peptide feature data summarized in FIG. 19 were mapped to human proteins. Proteins were counted regardless of the number of peptides that achieved individual significance. In other words, each counted protein corresponded to at least one statistically significant peptide feature, with some proteins corresponding to multiple statistically significant peptide features.

A total of 825 different protein groups were derived from the AD and MCI models. Of these protein groups, 151 were unique to AD, 222 were unique to MCI, and 452 were common to both sets. Given both the biological overlap in diagnosis of AD and MCI that might exist in these samples as well as the potential sample collection stratification factors highlighted above, this degree of overlap as well as the overall number of protein groups that overlap may not be unexpected. Nonetheless, the large numbers of protein groups unique to AD and MCI show that the particle panel interrogation of the present example is capable of distinguishing AD and MCI.

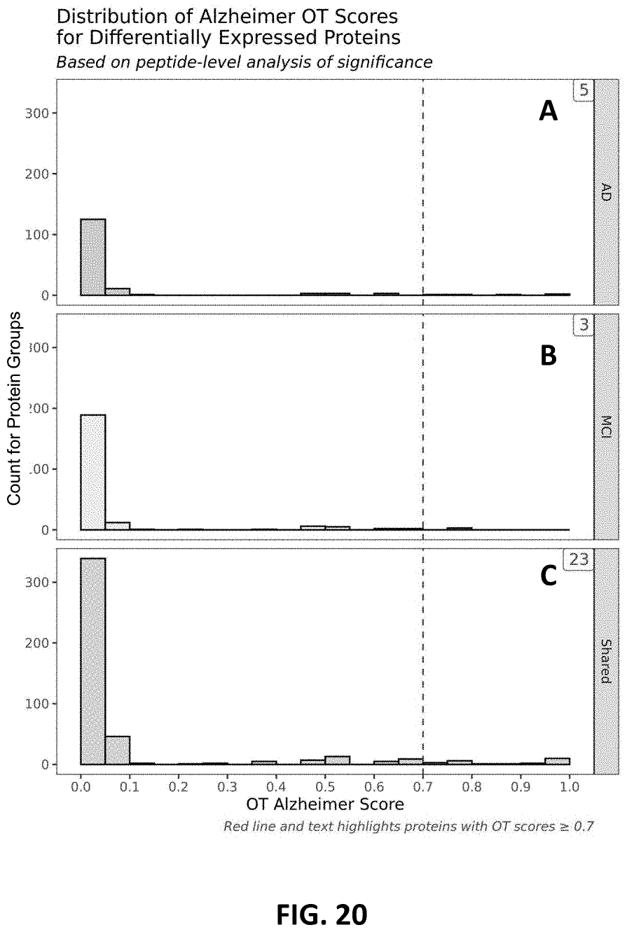

Given the overlap between the AD and MCI peptides outlined above, the identified protein groups were analyzed against previous annotations for Alzheimer's utility as annotated in the OpenTargets database. FIG. 20 summarizes the OpenTarget (OT) AD scores for the AD (panel A), MCI (panel B), and disease (panel C) relevant protein groups identified in the present example. All protein groups are plotted in the distribution. Scores of zero were provided to protein groups which did not AD-related OpenTarget scores. Using an OT score of 0.7 as a threshold for significant importance (a heuristic based on the distribution of all OT scores), the numbers of significantly different protein groups that achieve this threshold in annotated in the plot. TABLE 8 below shows the identity of the 31 protein groups which pass that threshold. Although OpenTarget score (OpenTarget ≥0.7) indicates that each of these protein groups may be relevant to AD several of these protein groups were also identified as differentially expressed across AD, MCI, and control samples, including ApoE, Amyloid beta A4, and Clusterin.

TABLE 8
Disease associated protein groups with AD
OpenTarget Scores of at least 0.7

| | | Max | |
|---|---|---|---|
| | | Open- | |
| | | Target | |
| Group | Protein | AD Score | Name |
| AD | A0A0C4DGQ5; | 1 | Calpain small subunit 1 |
| | P04632 | | |
| AD | P32119 | 1 | Peroxiredoxin-2 |
| Shared | A0A087X296; | 1 | Prostaglandin G/H |
| | P23219 | | synthase 1 |
| Shared | K7ERI9; | 1 | Apolipoprotein C-I |
| | P02654 | | |
| Shared | O14672 | 1 | Disintegrin and |
| | | | metalloproteinase |
| | | | domain-containing |
| | | | protein 10 |
| Shared | P02545; | 1 | Prelamin-A/C |
| | Q5TCI8 | | |
| Shared | P02649 | 1 | Apolipoprotein E |
| Shared | P05067 | 1 | Amyloid beta A4 protein |
| Shared | P07384 | 1 | Calpain-1 catalytic subunit |
| Shared | P10909 | 1 | Clusterin |
| Shared | Q06830 | 1 | Peroxiredoxin-1 |
| Shared | Q14432 | 1 | cGMP-inhibited 3′,5′-cyclic |
| | | | phosphodiesterase A |
| Shared | P55056 | 0.91897291 | Apolipoprotein C-IV |
| Shared | Q92619 | 0.89200963 | Minor histocompatibility |
| | | | protein HA-1 |
| AD | B4DDF4; | 0.88089852 | Calponin |
| | B4DUT8; | | |
| | Q99439 | | |
| AD | B4DDF4; | 0.88089852 | Calponin-2 |
| | B4DUT8; | | |
| | Q99439 | | |
| Shared | Q9NQ75 | 0.81854 | Cas scaffolding protein |
| | | | family member 4 |
| Shared | P04003 | 0.78716331 | C4b-binding protein |
| | | | alpha chain |
| Shared | Q15942 | 0.77111102 | Zyxin |
| AD | A0A087WT12; | 0.76871641 | Glutathione peroxidase |
| | A0A087X2I2; | | |
| | P36969 | | |
| AD | A0A087WT12; | 0.76871641 | Phospholipid |
| | A0A087X2I2; | | hydroperoxide |
| | P36969 | | glutathione |
| | | | peroxidase, |
| | | | mitochondrial |
| Shared | A0A087WUV8; | 0.76393924 | Basigin |
| | A0A087X2B5; | | |
| | P35613 | | |
| Shared | A0A0U1RRM4; | 0.76383493 | Polypyrimidine |
| | A6NLN1; | | tract-binding |
| | P26599 | | protein 1 |
| Shared | P08246 | 0.76319852 | Neutrolphil elastase |
| MCI | K7ERG9; | 0.75997352 | Complement factor D |
| | P00746 | | |
| MCI | Q14011 | 0.758953484 | Cold-inducible |
| | | | RNA-binding protein |
| Shared | P20851 | 0.755898526 | C4b-binding protein |
| | | | beta chain |
| MCI | Q86YW5 | 0.755294444 | Trem-like transcript |
| | | | 1 protein |
| Shared | CHROMO- | 0.7251811 | Chromogranin-A |
| | GRANIN-A | | |
| AD | P16885 | 0.712616278 | 1-phosphatidylinositol |
| | | | 4,5-biphosphate |
| | | | phosphodiesterase |
| | | | gamma-2 |
| Shared | P36542 | 0.711323503 | ATP synthase |
| | | | subunit gamma, |
| | | | mitochondrial |
| Shared | P30273 | 0.7019875 | High affinity |
| | | | immunoglobulin epsilon |
| | | | receptor subunit gamma |

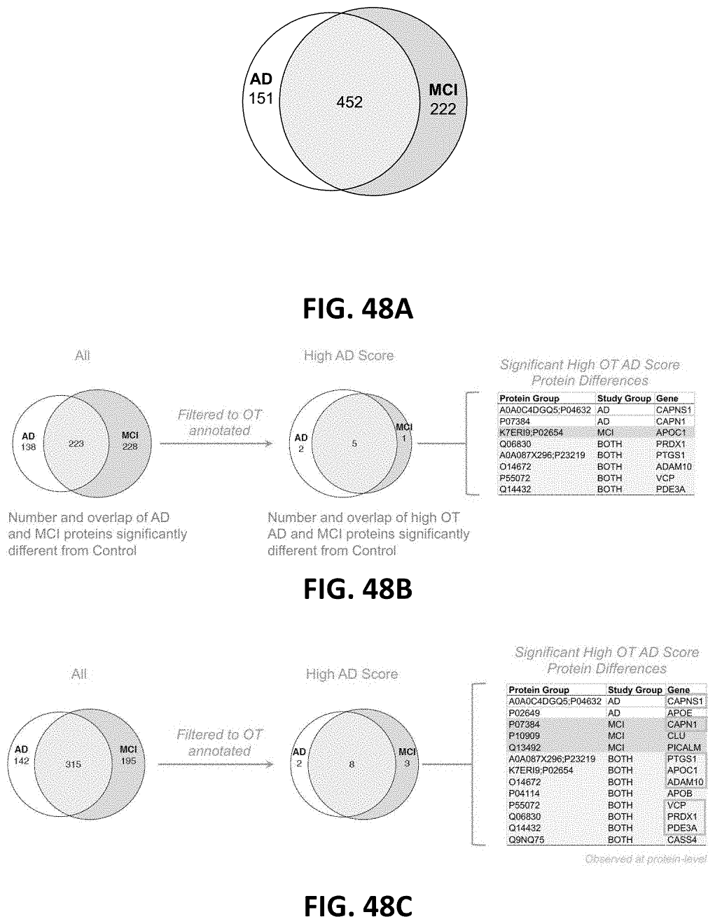

FIG. 48B and FIG. 48C each show a different set of studies where the number of protein groups unique to AD or MCI, or common to both were identified. The identified protein groups were filtered for annotated OT proteins having high AD score.

The studies described in this example provide particle profiling data, as well as the analyses of these data with respect to classification between for AD and MCI diagnostic groups as compared to age- and gender-matched controls. The particle panel platforms detected 2,232 protein groups (present in at least 25% of the 200 samples) and 16,323 unique peptides (also present in at least 25% of the samples). Univariate analysis of the pair-wise comparisons of the study classes using the peptide-level data revealed a significant number of protein groups with significantly different measured intensities. After multiple testing correction, 603, 674, and 930 protein groups were significantly different in the Control versus AD, Control versus MCI, and Control versus Diseased comparisons, respectively, with an overlap of 452 protein groups between the AD and MCI lists. The possibility of statistically significant subject sample blocking factors (i.e., age, gender, site, and time of collection) were reviewed, but the magnitude of the observed effects does not appear to be meaningful. However, there were no protein groups that achieved significant difference after multiple testing correction in the univariate MCI versus AD comparison.

### Example 2

### Trained Classifier for Biomarker and Disease Identification

This example demonstrates the potential for developing models based on biomolecule corona and mass spectrometric analysis, and outlines Random Forest (RF)-based models which use multiple rounds of cross-validation and are accurate models for biological state prediction. Peptide features (e.g., a specific peptide observed on a particle type) from Example 1 were used as the unit of data for training and development of a classifier to distinguish Alzheimer's disease (AD), mild cognitive impairment (MCI), and healthy (control) samples. As a feature is defined as a unique particle-peptide pair, the same peptide from a protein could be present on different particles and count as distinct inputs. Accordingly, the number of features is in significant excess to the number of peptides for any sample.

To prepare the data for classifier training, the data were median normalized using reference peptides as outlined in Example 1. The data were then filtered, such that only peptide features (i.e., nanoparticle-peptide pairs) that were present in at least 25% of the 200 samples were used for the classification analyses. After filtering, missing values were imputed by replacement with the lowest measured value for any feature from the given sample-particle combination. Although the replacement value for different peptide features from a sample-particle combination will be replaced by a common value, given the non-parametric nature of Random Forest classification models, monotonic replacement is unlikely to affect model performance.

After the data were prepared, ten rounds of 10-fold cross validation were performed for each class comparison, using a sparse tuning grid consisting of three different value for tree-node evaluation, namely

$$\frac{\sqrt{\text{number of features}}}{2} , \sqrt{\text{number of features}} , and 2 * \sqrt{\text{number of features}} .$$

While this does not represent an exhaustive tuning of the classification modeling process, it is useful for overall appraisal for model potential.

It is also worth pointing out that the large number of peptide features generated with the panel of 10 particles prevented all of the data from being used at one time for evaluation. There are many feature selection and reduction strategies that can be employed to reduce the dimensionality of classification problems (e.g., PCA transformations), but once again, Random Forests are relatively robust for correlating data for an initial approximation.

### Control Versus Alzheimer's Disease

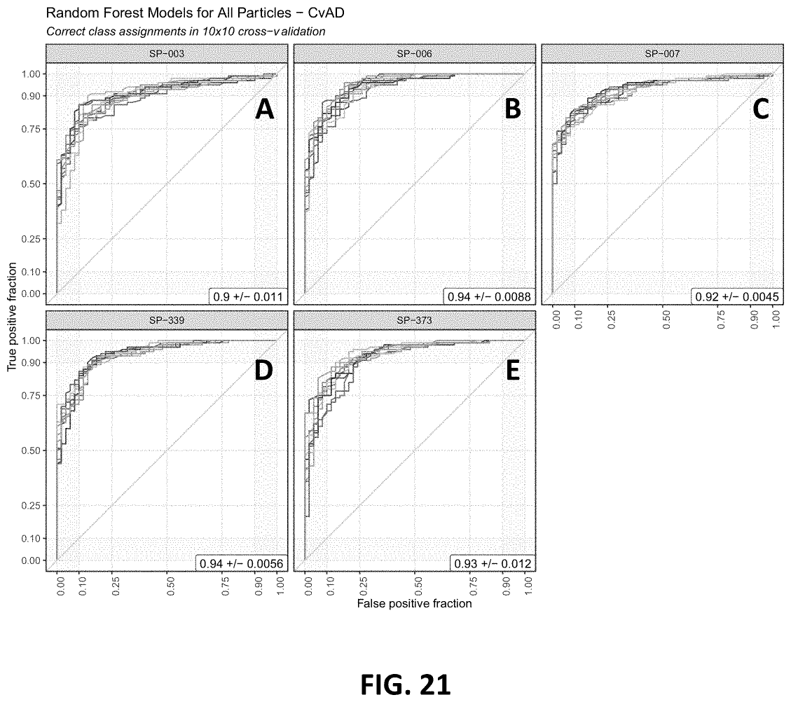

In the control versus AD classification model, the 100 control samples and 50 AD samples were used for training and analysis. As is shown in FIG. 21, the individual data from each of the 5 nanoparticles used in the study were able to generate robust, cross-validated classification models that ranged from area under curve (AUC) of about 0.90 to AUC of about 0.94 for the receiver operating characteristic (ROC) plots. Values at the bottom right of each plot shows the mean and the standard deviation of the AUC values for each particle. Given the errors in the models across the folds, it is not likely that the AUC differences between the particles are significant. As each of the folds and rounds develops its own RF model using the local, optimized features, the reported error is likely slightly underestimated. While particle SP-008 had an AUC curve which appears to be slightly different in shape (but not necessarily in AUC value, this may be related to the fact that SP-008 provided lowest number of median peptides identified across the samples, see FIG. 16).

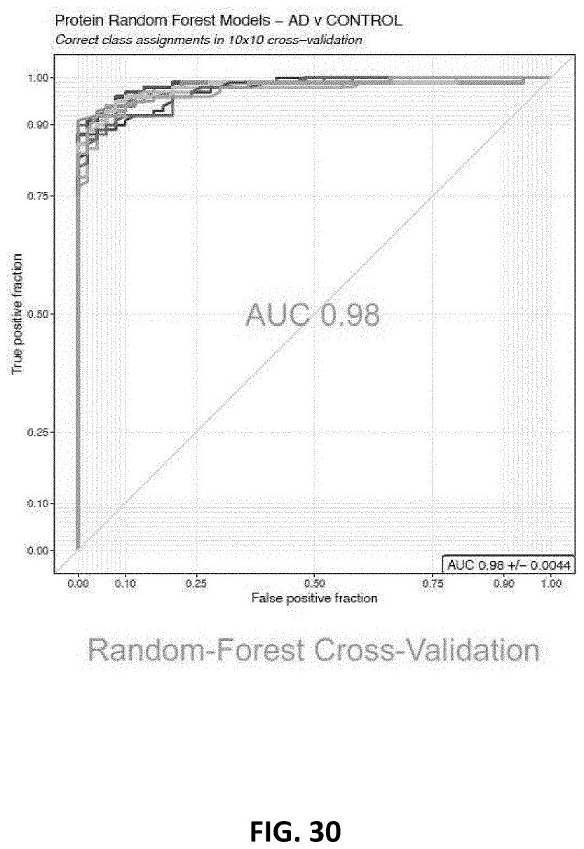

FIG. 30 provides an ROC plot for an AD versus control classification model utilizing data from 5 particles. The classifier utilized a combination of pre-existing and unknown biomarkers. As shown in the figure (with an AUC of 0.98), the ten rounds of 10-fold cross-validation used to generate the model yielded a high-performance classifier.

The 20 top features from the 5 particle Random Forest AD versus control sample classifier are provided in FIG. 31, with scaled importance provided on the x-axis and OpenTarget AD score indicated by red shading. As can be seen from the plot, only one feature was associated with a high OpenTarget AD score, while 18 of the features had OpenTarget AD scores of less than 0.05. The features are summarized in TABLE 9 below.

TABLE 9
Top features for AD versus control Random Forest classifier

| | Protein | | Open- | |
|---|---|---|---|---|
| Classifier | Group | | Target | |
| Importance | Uniprot ID | Protein Group | AD Score | Particle |
| 100 | O00299 | Chloride intracellular | 0.004 | SP-006 |
| | | channel protein 1 | | |
| 98.265 | P07814 | Bifunctional | 0 | SP-339 |
| | | glutamate/ | | |
| | | proline--tRNA ligase | | |
| 57.614 | B7ZKJ8 | ITIH-14 protein | 0.03121 | SP-007 |
| 50.978 | E7EPV7; | Alpha-synuclein | 0.39335 | SP-007 |
| | P37840 | | | |
| 40.638 | P26447 | Protein S100-A4 | 0.00357 | SP-006 |
| 35.449 | P68371 | Tubulin beta-4B | 0.02676 | SP-007 |
| | | chain | | |
| 33.963 | P08697 | Alpha-2-antiplasmin | 0.025 | SP-006 |
| 29.678 | Q8TF42 | Ubiquitin-associated | 0.01044 | SP-339 |
| | | and SH3 domain- | | |
| | | containing protein B | | |
| 28.946 | P30041 | Peroxiredoxin-6 | 0.03988 | SP-007 |
| 26.867 | P07437; | Tubulin beta chain | 0.02580 | SP-007 |
| | Q5JP53 | | | |
| 25.907 | P19875; | C-X-C motif | 0 | SP-004 |
| | P19876 | chemokine 3 | | |
| 24.267 | P00740 | Coagulation factor | 0 | SP-339 |
| | | IX | | |
| 24.116 | P10599 | Thioredoxin | 0.09903 | SP-006 |
| 23.915 | P21291 | Cysteine and glycine- | 0.01221 | SP-006 |
| | | rich protein 1 | | |
| 23.785 | P68363 | Tubulin alpha-1B | 0 | SP-007 |
| | | chain | | |
| 23.536 | Q14432 | cGMP-inhibited | 1 | SP-006 |
| | | 3′,5′-cyclic | | |
| | | phosphodiesterase A | | |
| 23.483 | Q9P1F3 | Costars family | 0.00767 | SP-006 |
| | | protein ABRACL | | |
| 22.476 | P06703 | Protein S100-A6 | 0.0299 | SP-006 |
| 22.261 | A0A024QZX5; | Serpin B6 | 0 | SP-006 |
| | A0A087X1N8; | | | |
| | P35237 | | | |
| 21.764 | P17252 | Protein kinase | 0.04900 | SP-339 |
| | | C alpha type | | |

### Control Versus Mild Cognitive Impairment

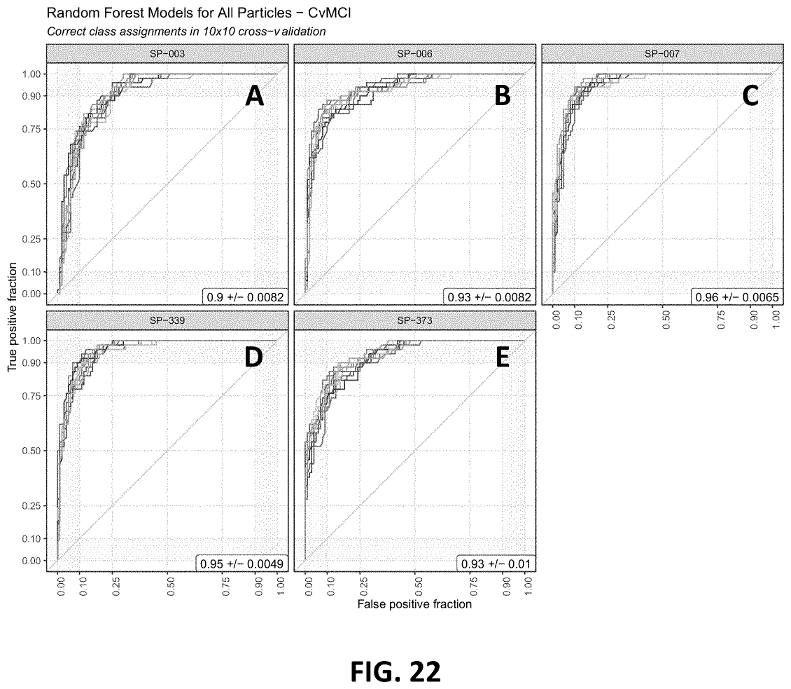

The control versus mild cognitive impairment classification models were trained using data from the 100 control samples and 50 MCI samples from Example 1. As outlined in FIG. 22, performances of classifiers corresponding to individual particles ranged from AUC of about 0.90 to AUC of about 0.96.

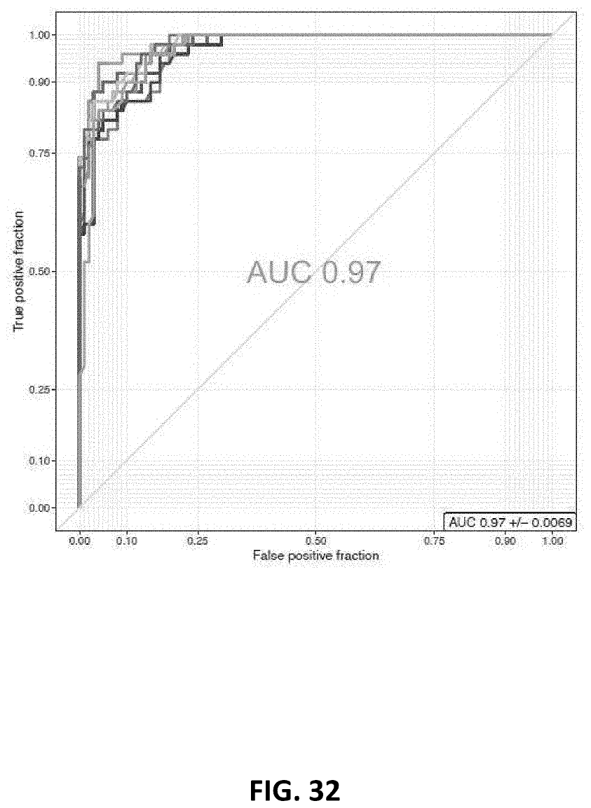

FIG. 32 provides an ROC plot for an MCI versus control classification model utilizing data from all 5 particles. As exhibited by the AUC of 0.97, data from all ten particles yielded a high accuracy MCI versus control classification model.

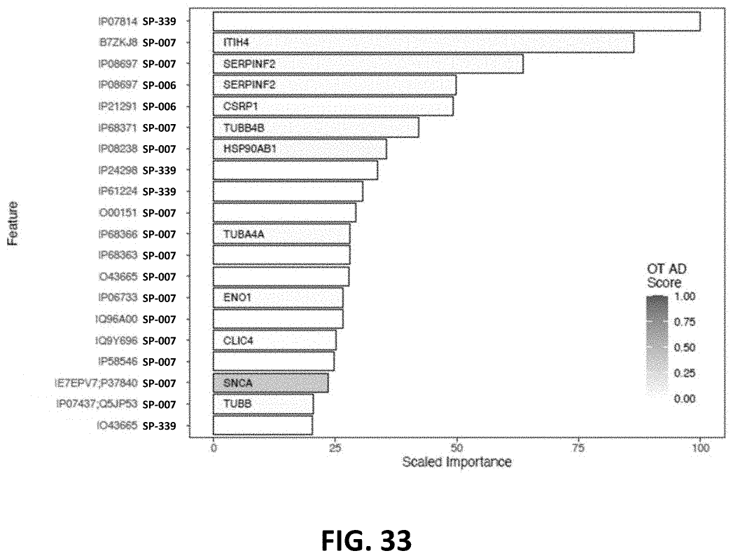

The 20 top features from the 5 particle Random Forest AD versus control sample classifier are provided in FIG. 33, with scaled importance provided on the x-axis and OpenTarget AD score indicated by red shading. As can be seen from the plot, only one feature was associated with a high OpenTarget AD score, while the remaining 19 features had OpenTarget AD scores of less than 0.05. The features are summarized in TABLE 10 below. Of the 20 features, 14 correspond to particle type SP-007.

TABLE 10
Top features for MCI versus control Random Forest classifier

| | Protein | | | |
|---|---|---|---|---|
| Classifier | Group | | OpenTarget | |
| Importance | Uniprot ID | Protein Group | AD Score | Particle |
| 100 | P07814 | Bifunctional glutamate/ | 0 | SP-339 |
| | | proline--tRNA ligase | | |
| 86.458 | B7ZKJ8 | ITIH4 protein | 0.0312 | SP-007 |
| 63.618 | P08697 | Alpha-2-antiplasmin | 0.025 | SP-007 |
| 49.794 | P08697 | Alpha-2-antiplasmin | 0.025 | SP-006 |
| 49.133 | P21291 | Cysteine and glycine- | 0.01221 | SP-006 |
| | | rich protein 1 | | |
| 42.048 | P68371 | Tubulin beta-4B chain | 0.0268 | SP-007 |
| 35.509 | P08238 | Heat shock protein | 0.044 | SP-007 |
| | | HSP 90-beta | | |
| 33.709 | P24298 | Alanine | 0 | SP-339 |
| | | aminotransferase 1 | | |
| 30.569 | P61224 | Ras-related protein | 0 | SP-339 |
| | | Rap-1b | | |
| 29.227 | O00151 | PDZ and LIM | 0 | SP-007 |
| | | domain protein 1 | | |
| 27.974 | P68366 | Tubulin alpha-4A chain | 0.0242 | SP-007 |
| 27.922 | P68363 | Tubulin alpha-1B chain | 0 | SP-007 |
| 27.753 | O43665 | Regulator of G-protein | 0 | SP-007 |
| | | signaling 10 | | |
| 26.56 | P06733 | Alpha-enolase | 0.0142 | SP-007 |
| 26.504 | Q96A00 | Protein phosphatase 1 | 0 | SP-007 |
| | | regulatory subunit 14A | | |
| 25.174 | Q9Y696 | Chloride intracellular | 0.0087 | SP-007 |
| | | channel protein 4 | | |
| 24.833 | P58546 | Myotrophin | 0 | SP-007 |
| 23.530 | E7EPV7; | Alpha-synuclein | 0.3933 | SP-007 |
| | P37840 | | | |
| 20.476 | P07437; | Tubulin beta chain | 0.02581 | SP-007 |
| | Q5JP53 | | | |
| 20.347 | O43665 | Regulator of G-protein | 0 | SP-339 |
| | | signaling 10 | | |

Mild Cognitive Impairment versus Alzheimer's Disease

From the 10-particle panel experiments in Example 1, there was considerable overlap between protein groups which exhibited significant differences for MCI and AD (of the 825 protein groups which exhibited significant differences for MCI and AD samples, 222 were specific for MCI, 151 for AD, and 452 common to AD and MCI). Given the considerable overlap between the MCI and AD protein groups, the ability to significantly discriminate MCI v AD by Random Forest classification was anticipated to be somewhat challenging.

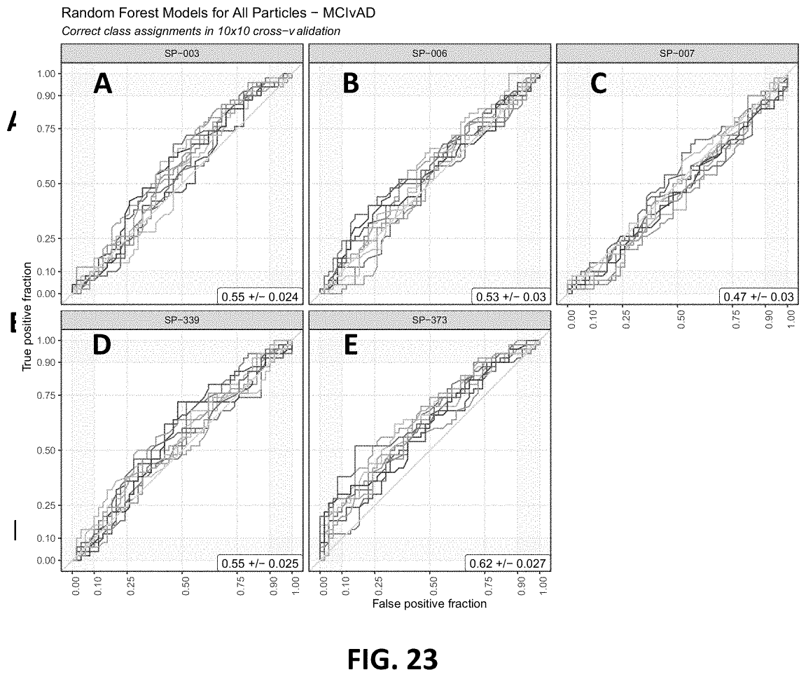

As shown in FIG. 23, the results for the five individual particles in this 50 sample versus 50 sample comparison range from AUC of about 0.47 to AUC of about 0.61. Given the potential for overfitting in high-dimensional data analysis, it is not likely that these performances are statistically significant, although it is worth noting some nanoparticles exhibited ≥0.5 AUC values, which may represent some ability for the classifiers to distinguish between the two pathologies.

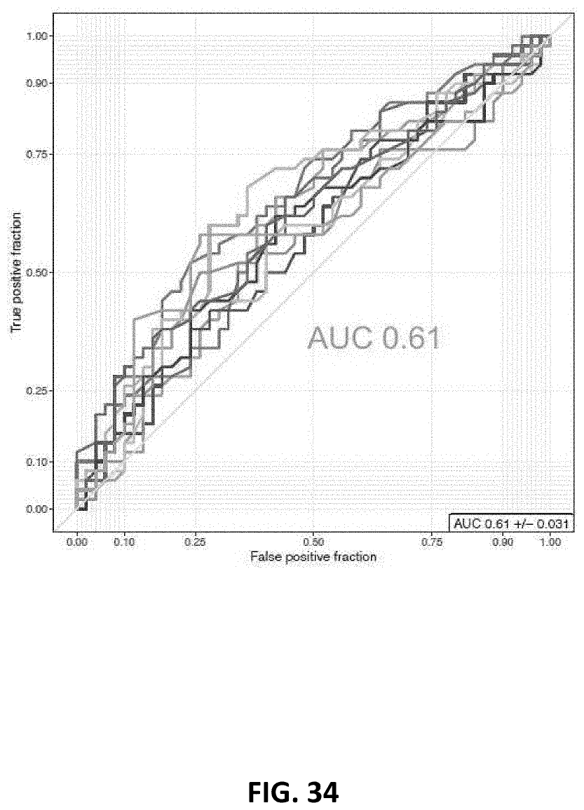

FIG. 34 provides a ROC plot for an MCI versus AD classification model utilizing data from all 5 particles. As exhibited by the AUC of 0.61, utilizing data from ten particles generates a higher performance for the classifier than using data from any single particle alone.

### Example 3

### Comparison of Mild Cognitive Impairment and Alzheimer's Disease Classifiers

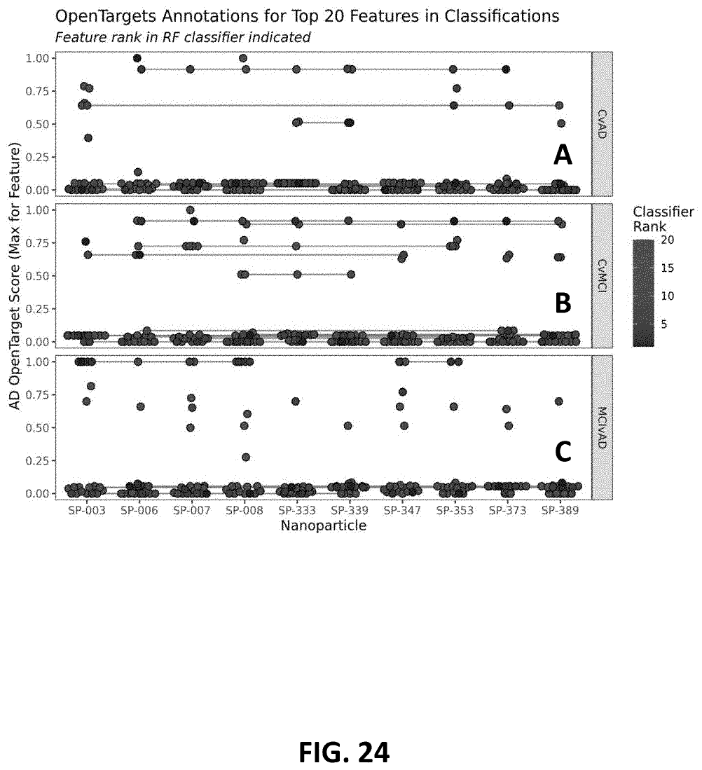

Given an overlap between the univariate analysis of the control versus AD and control versus MCI Random Forest classifiers, the overlap of the top peptide features in classifiers for each of these comparisons can be compared. Models trained with data collected from 10 particle panels were compared, as shown in FIG. 24, with panel A (top) providing peptide features from the control versus AD classifier, panel B (middle) providing peptide features from the control versus mild cognitive impairment classifier, and panel C (bottom) providing peptide features from the MCI versus AD classifier. In each panel, the first column corresponds to particle SP-003, the second column corresponds to particle SP-006, the third column corresponds to particle SP-007, the fourth column corresponds to particle SP-008, the fifth column corresponds to particle SP-333, the sixth column corresponds to particle SP-339, the seventh column corresponds to particle SP-347, the eight column corresponds to particle SP-353, the ninth column corresponds to particle SP-373, and the tenth particle corresponds to particle SP-389.

FIG. 24 displays both the Alzheimer's OpenTargets score (y-axis) for the top 20 peptide features of each particle's classifier as well as the rank within the top 20 for that peptide feature (point fill color, with red indicating lower rank and blue indicating higher rank). FIG. 24 panel A provides peptide features for the control versus AD classifier. FIG. 24 panel B provides peptide features for the control versus MCI classifier. FIG. 24 panel C provides peptide features for the MCI versus AD classifier. In each panel, the columns are ordered, from left to right, by peptide features for SP-003 particles, SP-006 particles, SP-007 particles, SP-008 particles, SP-333 particles, SP-339 particles, SP-347 particles, SP-353 particles, SP-373 particles, and SP-389 particles. Peptides common to multiple particles are highlighted by the horizontal lines linking individual peptide features.

Analogous to the univariate analysis described in Example 1, the majority of the top 20 features for each classifier have very low or no annotated AD OpenTarget score suggesting either that these represent novel, previously unappreciated candidate markers for AD and MCI (the favorable interpretation) or that they represent markers related to potential subject sample stratification as described above. The considerable number of high and low top 20 features shared across particle types in each model comparison suggests a higher degree of confidence in the results (i.e., lack of overfitting), since each classifier is independently built with its own particle peptide data. That being said, the number of top high OpenTarget score features not shared across particle-types indicates that interrogation with a panel of particles rather than any one particle may generate greater degrees of profiling depth, reproducibility, and biological insight.

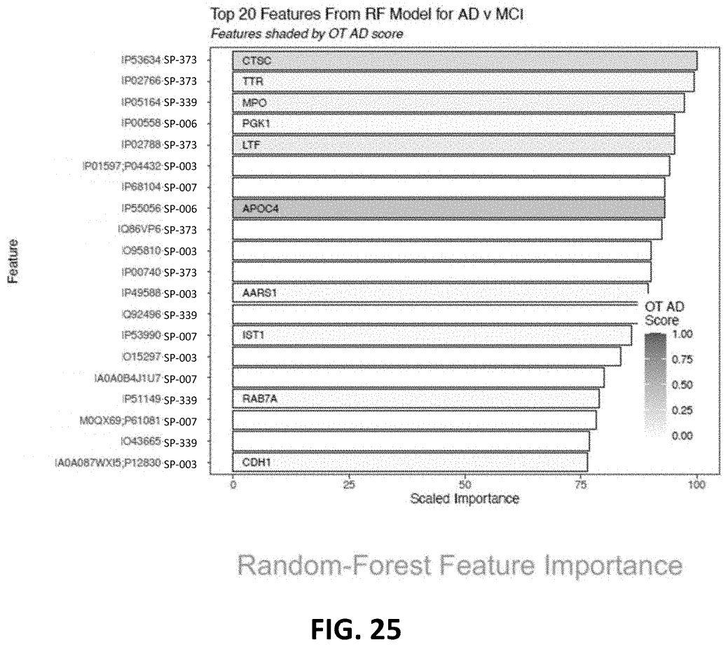

FIG. 25 details the 20 top features of the MCI versus AD model peptide features outlined in FIG. 24, spanning Random Forest importance values of 1 to about 0.76. The shading in the figure indicates OpenTarget AD score. Of the 20 top features, only 2 have high OpenTarget AD scores, and 3 have moderate (up to about 0.6) OpenTarget AD scores, while 15 of the features have scores of zero or close to 0. Furthermore, the 20 top Random Forest features were distributed across only 5 of the 10 particle types, suggesting that some particles provide greater diagnostic utility for differentiating AD and MCI. Specifically, SP-373 and SP-003 each contributed 5 of the 20 top features, while SP-339 and SP-007 each contributed 4. The 20 features summarized in FIG. 25 are detailed in TABLE 11 below.

TABLE 11
Highest importance features for MCI versus AD classifier

| Particle | Peptide ID | Protein Name |
|---|---|---|
| SP-373 | P53634 | Dipeptidyl peptidase 1 |
| SP-373 | P02766 | Transthyretin |
| SP-339 | P05164 | Myeloperoxidase |
| SP-006 | P00558 | Phosphoglycerate kinase 1 |
| SP-373 | P02788 | Lactotransferrin |
| SP-003 | P04432 | Immunoglobulin kappa variable 1D-39 |
| SP-007 | P68104 | Elongation factor 1-alpha 1 |
| SP-006 | P55056 | Apolipoprotein C-IV |
| SP-373 | Q86VP6 | Cullin-associated NEDD8-dissociated |
| | | protein 1 |
| SP-003 | O95810 | Caveolae-associated protein 2 |
| SP-373 | P00740 | Coagulation factor IX |
| SP-003 | P49588 | Alanine--tRNA ligase, cytoplasmic |
| SP-339 | Q92496 | Complement factor H-related protein 4 |
| SP-007 | P53990 | IST1 homolog |
| SP-003 | O15297 | Protein phosphatase 1D |
| SP-007 | A0A0B4J1U7 | Immunoglobulin heavy variable 6-1 |
| SP-339 | P51149 | Ras-related protein Rab-7a |
| SP-007 | M0QX69; P61081 | NEDD8-conjugating enzyme Ubc12 |
| SP-039 | O43665 | Regulator of G-protein signaling 10 |
| SP-339 | P12830 | Cadherin-1 |

Using data derived at the high-resolution (as outlined in Example 1), peptide-level univariate and cross-validated classification analyses on the sample diagnostic groups were performed yielding high-performance models with AD- and MCI-nanoparticle classifiers in excess of 0.90 AUC. The net result was the identification of both pre-existing and novel differences between the groups, with the classifiers combining both for predictive performance. While the results from these specific analyses represent novel opportunities for clinical test development with respect to AD and MCI, the results and analyses also highlight the potential for the methods disclosed herein to be deployed in even larger studies in a practicable and affordable format, resolving one of the key barriers (e.g., small study sizes constrained by complex workflows) to improving protein candidate biomarker discovery.

Using the 200 samples in the respective pairwise sample group comparisons, cross-validated classifier constructions by Random Forest machine-learning, high-performing classification occurred with all nanoparticles. For the AD and MCI classifications versus Controls, all cross-validated ROC AUCs were greater than or equal to 0.90. For the MCI versus AD, classification performance was less refined, with individual nanoparticle ROC AUCs ranging from 0.63 to 0.50. Inspection of the top 20 features in each Random Forest-based classification highlighted the identification of novel combinations of pre-existing and unknown candidate biomarker protein groups, with several instances of the identification of the same protein on different nanoparticles. Taken together, the results of this collaborative study highlight at least two considerations for AD and MCI analysis. First, the particle panel platform is a superior workflow for the collection and identification of proteomics profiling data in a rapid and broad fashion, enabling large-scale studies with enhanced ability to detect novel insights. Second, the specific results from the univariate and cross-validation analyses identify novel candidate markers, both with and without prior appreciation of utility in AD testing, and thus suggest potential for the use of the particle panel platform in biomarker discovery for both diagnostic and therapeutic research and development.

In total, more than 600 peptide features contributed to the classification models. The top 20 peptide features identified on each particle for each biological state comparison (control versus AD, control versus MCI, and AD versus MCI), along with the plasma protein groups from which they are derived, are summarized in TABLE 12.

TABLE 12
Peptides used in trained classifiers

| Random | | | | SEQ | | |
|---|---|---|---|---|---|---|
| Forest | | | | ID | | |
| Importance | Particle | Protein | Peptide | NO: | Rank | Comparison |
| 100 | SP-003 | Myosin-9 | DLQGRDEQSEEK | 1 | 1 | Control |
| | | | | | | versus |
| | | | | | | AD |
| 98.05418897 | SP-003 | Chloride | GVTFNVTTVDTK | 2 | 2 | Control |
| | | intracellular | | | | versus |
| | | channel | | | | AD |
| | | protein 1 | | | | |
| 86.3416921 | SP-003 | Costars | MNVDHEVNLLVEEIHR | 3 | 3 | Control |
| | | family | | | | versus |
| | | protein | | | | AD |
| | | ABRACL | | | | |
| 83.61670642 | SP-003 | Alpha- | TVEGAGSIAAATGFVK | 4 | 4 | Control |
| | | synuclein | | | | versus |
| | | | | | | AD |
| 71.70852814 | SP-003 | Rho-associated | IFQILYANEGESK | 5 | 5 | Control |
| | | protein | | | | versus |
| | | kinase 2 | | | | AD |
| 67.93578237 | SP-003 | Heparin | TLEAQLTPR | 6 | 6 | Control |
| | | cofactor 2 | | | | versus |
| | | | | | | AD |
| 65.92161946 | SP-003 | Transthyretin | ALGISPFHEHAEVVFT | 7 | 7 | Control |
| | | | ANDSGPR | | | versus |
| | | | | | | AD |
| 62.36488241 | SP-003 | Rho-associated | VYYDISTAK | 8 | 8 | Control |
| | | protein | | | | versus |
| | | kinase 2 | | | | AD |
| 61.23995466 | SP-003 | Transthyretin | AADDTWEPFASGK | 9 | 9 | Control |
| | | | | | | versus |
| | | | | | | AD |
| 59.71445123 | SP-003 | RHO-ASSOCIATED | LEGWLSLPVR | 10 | 10 | Control |
| | | PROTEIN | | | | versus |
| | | KINASE 2 | | | | AD |
| 59.13569512 | SP-003 | ZYXIN-2 | AYHPHCFTCVVCARPL | 11 | 11 | Control |
| | | | EGTSFIVDQANRPHCV | | | versus |
| | | | PDYHK | | | AD |
| 58.17483616 | SP-003 | T-plasminogen | GGLFADIASHPWQAAI | 12 | 12 | Control |
| | | activator | FAK | | | versus |
| | | | | | | AD |
| 55.92163941 | SP-003 | PLATELET | ALETMGLWVDCR | 13 | 13 | Control |
| | | GLYCOPROTEIN | | | | versus |
| | | IX | | | | AD |
| 55.83097579 | SP-003 | FIBRINOGEN | DSDWPFCSDEDWNYK | 14 | 14 | Control |
| | | ALPHA | | | | versus |
| | | CHAIN | | | | AD |
| 54.77007967 | SP-003 | RHO-ASSOCIATED | ILFYDSEQDK | 15 | 15 | Control |
| | | PROTEIN | | | | versus |
| | | KINASE 2 | | | | AD |
| 47.18547476 | SP-003 | ADP- | ILMVGLDAAGK | 16 | 16 | Control |
| | | RIBOSYLATION | | | | versus |
| | | FACTOR 1 | | | | AD |
| 42.58983052 | SP-003 | MYOSIN | GNFNYVEFTR | 17 | 17 | Control |
| | | REGULATORY | | | | versus |
| | | LIGHT | | | | AD |
| | | POLYPEPTIDE 9 | | | | |
| 42.31616188 | SP-003 | MYOSIN | LSNDMMGSYAEMK | 18 | 18 | Control |
| | | APOLIPOPROTEIN | | | | versus |
| | | B-100LIGHT | | | | AD |
| | | POLYPEPTIDE 9 | | | | |
| 40.33930014 | SP-003 | EOSINOPHIL | TTFANVVNVCGNQSIR | 19 | 19 | Control |
| | | CATIONIC | | | | versus |
| | | PROTEIN | | | | AD |
| 39.44070946 | SP-003 | C4B-BINDING | GYILVGQAK | 20 | 20 | Control |
| | | PROTEIN ALPHA | | | | versus |
| | | CHAIN | | | | AD |
| 100 | SP-006 | CGMP-INHIBITED | VIEEEQR | 21 | 1 | Control |
| | | 3′,5′-CYCLIC | | | | versus |
| | | PHOSPHO- | | | | AD |
| | | DIESTERASE A | | | | |
| 96.07670314 | SP-006 | CHLORIDE | NSNPALNDNLEK | 22 | 2 | Control |
| | | INTRACELLULAR | | | | versus |
| | | CHANNEL | | | | AD |
| | | PROTEIN 1 | | | | |
| 92.01612832 | SP-006 | APOLIPOPROTEIN | ELLETVVNR | 23 | 3 | Control |
| | | C-II | | | | versus |
| | | | | | | AD |
| 88.83098675 | SP-006 | CHLORIDE | GVTFNVTTVDTK | 2 | 4 | Control |
| | | INTRACELLULAR | | | | versus |
| | | CHANNEL | | | | AD |
| | | PROTEIN 1 | | | | |
| 76.13849151 | SP-006 | P10599 | TAFQEALDAAGDK | 24 | 5 | Control |
| | | | | | | versus |
| | | | | | | AD |
| 75.20677688 | SP-006 | VON WILLEBRAND | EEVFIQQR | 25 | 6 | Control |
| | | FACTOR | | | | versus |
| | | | | | | AD |
| 65.13265835 | SP-006 | ALPHA-2- | LGNQEPGGQTALK | 26 | 7 | Control |
| | | ANTIPLASMIN | | | | versus |
| | | | | | | AD |
| 63.9041753 | SP-006 | PROTEIN | ELPSFLGK | 27 | 8 | Control |
| | | S100-A4 | | | | versus |
| | | | | | | AD |
| 59.12427676 | SP-006 | CYSTEINE AND | GLESTTLADK | 28 | 9 | Control |
| | | GLYCINE-RICH | | | | versus |
| | | PROTEIN 1 | | | | AD |
| 53.76879666 | SP-006 | Ribonuclease | VNPALAELNLR | 29 | 10 | Control |
| | | inhibitor | | | | versus |
| | | | | | | AD |
| 53.30948669 | SP-006 | INTEGRIN | CECGSCVCIQPGSYG | 30 | 11 | Control |
| | | BETA-3 | DTCEK | | | versus |
| | | | | | | AD |
| 51.46888241 | SP-006 | CHLORIDE | LAALNPESNTAGLDI | 31 | 12 | Control |
| | | INTRACELLULAR | FAK | | | versus |
| | | CHANNEL | | | | AD |
| | | PROTEIN I | | | | |
| 49.77811679 | SP-006 | COSTARS FAMILY | CANLFEALVGTLK | 32 | 13 | Control |
| | | PROTEIN ABRACL | | | | versus |
| | | | | | | AD |
| 49.29576029 | SP-006 | VON WILLEBRAND | EYAPGETVK | 33 | 14 | Control |
| | | FACTOR | | | | versus |
| | | | | | | AD |
| 48.0989301 | SP-006 | VON WILLEBRAND | TATLCPQSCEER | 34 | 15 | Control |
| | | FACTOR | | | | versus |
| | | | | | | AD |
| 47.53063992 | SP-006 | VON WILLEBRAND | TPDFCAMSCPPSLVYN | 35 | 16 | Control |
| | | FACTOR | HCEHGCPR | | | versus |
| | | | | | | AD |
| 46.7624093 | SP-006 | GLUCOSE-6- | GYLDDPTVPR | 36 | 17 | Control |
| | | PHOSPHATE 1- | | | | versus |
| | | DEHYDROGENASE | | | | AD |
| 44.63757698 | SP-006 | COSTARS FAMILY | MNVDHEVNLLVEEIHR | 3 | 18 | Control |
| | | PROTEIN ABRACL | | | | versus |
| | | | | | | AD |
| 41.1614827 | SP-006 | VON WILLEBRAND | CLPSACEVVTGSPR | 37 | 19 | Control |
| | | FACTOR | | | | versus |
| | | | | | | AD |
| 41.14283078 | SP-006 | PYRUVATE | IYVDDGLISLQVK | 38 | 20 | Control |
| | | KINASE PKM | | | | versus |
| | | | | | | AD |
| 100 | SP-007 | INTER-ALPHA- | QLGLPGPPDVPDHAAY | 39 | 1 | Control |
| | | TRYPSIN | HPFR | | | versus |
| | | INHIBITOR | | | | AD |
| | | HEAVY CHAIN | | | | |
| | | H4 (ITIH4) | | | | |
| | | PROTEIN | | | | |
| 78.82947285 | SP-007 | TUBULIN | INVYYNEATGGK | 40 | 2 | Control |
| | | BETA-4B CHAIN | | | | versus |
| | | | | | | AD |
| 76.42996407 | SP-007 | INTER-ALPHA- | AGFSWIEVTFK | 41 | 3 | Control |
| | | TRYPSIN | | | | versus |
| | | INHIBITOR | | | | AD |
| | | HEAVY CHAIN | | | | |
| | | H4 (ITIH4) | | | | |
| 76.36313161 | SP-007 | TUBULIN | IMNTFSVVPSPK | 42 | 4 | Control |
| | | BETA-4B CHAIN | | | | versus |
| | | | | | | AD |
| 61.04895229 | SP-007 | APOLIPOPROTEIN | ELLETVVNR | 23 | 5 | Control |
| | | C-II | | | | versus |
| | | | | | | AD |
| 60.90050855 | SP-007 | TUBULIN ALPHA- | FDGALNVDLTEFQTNL | 43 | 6 | Control |
| | | 1B CHAIN | VPYPR | | | versus |
| | | | | | | AD |
| 60.36320849 | SP-007 | Complement | TLDEFTIIQNLQPQYQ | 44 | 7 | Control |
| | | subcomponent | FR | | | versus |
| | | C1r | | | | AD |
| 58.08189049 | SP-007 | INTER-ALPHA- | RLDYQEGPPGVEISCW | 45 | 8 | Control |
| | | TRYPSIN | SVEL | | | versus |
| | | INHIBITOR | | | | AD |
| | | HEAVY CHAIN | | | | |
| | | H4 (ITIH4) | | | | |
| | | PROTEIN | | | | |
| 57.63473493 | SP-007 | INTER-ALPHA- | QGPVNLLSDPEQGVEV | 46 | 9 | Control |
| | | TRYPSIN | TGQYER | | | versus |
| | | INHIBITOR | | | | AD |
| | | HEAVY CHAIN | | | | |
| | | H4 (ITIH4) | | | | |
| | | PROTEIN | | | | |
| 51.42829211 | SP-007 | Tubulin | FWEVISDEHGIDPTGT | 47 | 10 | Control |
| | | beta chain | YHGDSDLQLDR | | | versus |
| | | | | | | AD |
| 48.25100113 | SP-007 | TUBULIN | ALTVPELTQQMFDAK | 48 | 11 | Control |
| | | BETA-4B CHAIN | | | | versus |
| | | | | | | AD |
| 48.20909974 | SP-007 | HEAT SHOCK | YESLTDPSK | 49 | 12 | Control |
| | | PROTEIN HSP | | | | versus |
| | | 90-BETA | | | | AD |
| 47.77206141 | SP-007 | Tubulin | ISVYYNEATGGK | 50 | 13 | Control |
| | | beta chain | | | | versus |
| | | | | | | AD |
| 47.54936488 | SP-007 | TUBULIN | FDLMYAK | 51 | 14 | Control |
| | | ALPHA-1B | | | | versus |
| | | CHAIN | | | | AD |
| 43.51667767 | SP-007 | TUBULIN | LHFFMPGFAPLTSR | 52 | 15 | Control |
| | | BETA-4B CHAIN | | | | versus |
| | | | | | | AD |
| 43.07878166 | SP-007 | TUBULIN | TAVCDIPPR | 53 | 16 | Control |
| | | BETA-4B CHAIN | | | | versus |
| | | | | | | AD |
| 43.02362164 | SP-007 | ALPHA-2- | DFLQSLK | 54 | 17 | Control |
| | | ANTIPLASMIN | | | | versus |
| | | | | | | AD |
| 42.13287389 | SP-007 | ALPHA-2- | SPPGVCSR | 55 | 18 | Control |
| | | ANTIPLASMIN | | | | versus |
| | | | | | | AD |
| 41.86317758 | SP-007 | COAGULATION | HPPVVMNGAVADGILA | 56 | 19 | Control |
| | | FACTOR XIII | SYATGSSVEYR | | | versus |
| | | B CHAIN | | | | AD |
| 41.00786937 | SP-007 | TUBULIN | NSSYFVEWIPNNVK | 57 | 20 | Control |
| | | BETA-4B CHAIN | | | | versus |
| | | | | | | AD |
| 100 | SP-008 | Gc-globulin | LCDNLSTK | 58 | 1 | Control |
| | | (Vitamin | | | | versus |
| | | D-binding | | | | AD |
| | | protein) | | | | |
| 76.62293425 | SP-008 | Gc-globulin | LCMAALK | 59 | 2 | Control |
| | | (Vitamin | | | | versus |
| | | D-binding | | | | AD |
| | | protein) | | | | |
| 55.78953742 | SP-008 | Gc-globulin | SYLSMVGSCCTSASPT | 60 | 3 | Control |
| | | (Vitamin | VCFLK | | | versus |
| | | D-binding | | | | AD |
| | | protein) | | | | |
| 45.53694347 | SP-008 | Gc-globulin | SCESNSPFPVHPGTAE | 61 | 4 | Control |
| | | (Vitamin | CCTK | | | versus |
| | | D-binding | | | | AD |
| | | protein) | | | | |
| 44.35753046 | SP-008 | Docking | GPALLVLGPDAIQLR | 62 | 5 | Control |
| | | protein 3 | | | | versus |
| | | | | | | AD |
| 43.80876105 | SP-008 | HISTIDINE-RICH | KGEVLPLPEANFPSFP | 63 | 6 | Control |
| | | GLYCOPROTEIN | LPHHK | | | versus |
| | | | | | | AD |
| 41.53897203 | SP-008 | Gc-globulin | YTFELSR | 64 | 7 | Control |
| | | (Vitamin | | | | versus |
| | | D-binding | | | | AD |
| | | protein) | | | | |
| 40.93481328 | SP-008 | Gc-globulin | HQPQEFPTYVEPTNDE | 65 | 8 | Control |
| | | (Vitamin | ICEAFRK | | | versus |
| | | D-binding | | | | AD |
| | | protein) | | | | |
| 40.84618967 | SP-008 | APOLIPOPROTEIN | GEVQAMLGQSTEELR | 66 | 9 | Control |
| | | E | | | | versus |
| | | | | | | AD |
| 36.48539472 | SP-008 | APOLIPOPROTEIN | ELLETVVNR | 23 | 10 | Control |
| | | C-II | | | | versus |
| | | | | | | AD |
| 35.14377795 | SP-008 | HISTIDINE-RICH | YKEENDDFASFR | 67 | 11 | Control |
| | | GLYCOPROTEIN | | | | versus |
| | | | | | | AD |
| 34.57380645 | SP-008 | Gc-globulin | EDFTSLSLVLYSR | 68 | 12 | Control |
| | | (Vitamin | | | | versus |
| | | D-binding | | | | AD |
| | | protein) | | | | |
| 34.49707364 | SP-008 | Gc-globulin | SLGECCDVEDSTTCFN | 69 | 13 | Control |
| | | (Vitamin | AK | | | versus |
| | | D-binding | | | | AD |
| | | protein) | | | | |
| 33.22640764 | SP-008 | HISTIDINE-RICH | YWNDCEPPDSR | 70 | 14 | Control |
| | | GLYCOPROTEIN | | | | versus |
| | | | | | | AD |
| 32.98505179 | SP-008 | Hemopexin | VDGALCMEK | 71 | 15 | Control |
| | | | | | | versus |
| | | | | | | AD |
| 32.33291722 | SP-008 | HISTIDINE-RICH | GEVLPLPEANFPSFPL | 72 | 16 | Control |
| | | GLYCOPROTEIN | PHHK | | | versus |
| | | | | | | AD |
| 32.28052055 | SP-008 | Gc-globulin | ELSSFIDK | 73 | 17 | Control |
| | | (Vitamin | | | | versus |
| | | D-binding | | | | AD |
| | | protein) | | | | |
| 31.51408225 | SP-008 | HISTIDINE-RICH | DSPVLIDFFEDTERYR | 74 | 18 | Control |
| | | GLYCOPROTEIN | | | | versus |
| | | | | | | AD |
| 30.93854969 | SP-008 | HISTIDINE-RICH | VIDFNCTTSSVSSALA | 75 | 19 | Control |
| | | GLYCOPROTEIN | NTK | | | versus |
| | | | | | | AD |
| 30.87974466 | SP-008 | HISTIDINE-RICH | IADAHLDR | 76 | 20 | Control |
| | | GLYCOPROTEIN | | | | versus |
| | | | | | | AD |
| 100 | SP-033 | Gc-globulin | SLGECCDVEDSTTCFN | 69 | 1 | Control |
| | | (Vitamin | AK | | | versus |
| | | D-binding | | | | AD |
| | | protein) | | | | |
| 74.13843858 | SP-033 | Gc-globulin | FEDCCQEK | 77 | 2 | Control |
| | | (Vitamin | | | | versus |
| | | D-binding | | | | AD |
| | | protein) | | | | |
| 73.66881137 | SP-033 | Gc-globulin | EDFTSLSLVLYSR | 68 | 3 | Control |
| | | (Vitamin | | | | versus |
| | | D-binding | | | | AD |
| | | protein) | | | | |
| 48.44388916 | SP-033 | Gc-globulin | ELSSFIDK | 73 | 4 | Control |
| | | (Vitamin | | | | versus |
| | | D-binding | | | | AD |
| | | protein) | | | | |
| 46.6435766 | SP-033 | Gc-globulin | LCDNLSTK | 58 | 5 | Control |
| | | (Vitamin | | | | versus |
| | | D-binding | | | | AD |
| | | protein) | | | | |
| 43.68196761 | SP-033 | Gc-globulin | EFSHLGK | 78 | 6 | Control |
| | | (Vitamin | | | | versus |
| | | D-binding | | | | AD |
| | | protein) | | | | |
| 40.57732781 | SP-033 | PROTEIN | LSVEIWDWDLTSR | 79 | 7 | Control |
| | | KINASE C | | | | versus |
| | | BETA TYPE | | | | AD |
| 38.55268327 | SP-033 | Gc-globulin | SCESNSPFPVHPGTAE | 61 | 8 | Control |
| | | (Vitamin | CCTK | | | versus |
| | | D-binding | | | | AD |
| | | protein) | | | | |
| 32.19549124 | SP-033 | Gc-globulin | SYLSMVGSCCTSASPT | 60 | 9 | Control |
| | | (Vitamin | VCFLK | | | versus |
| | | D-binding | | | | AD |
| | | protein) | | | | |
| 32.17440272 | SP-033 | Gc-globulin | CCESASEDCMAK | 80 | 10 | Control |
| | | (Vitamin | | | | versus |
| | | D-binding | | | | AD |
| | | protein) | | | | |
| 32.13643304 | SP-033 | APOLIPOPROTEIN | ELLETVVNR | 23 | 11 | Control |
| | | C-II | | | | versus |
| | | | | | | AD |
| 32.02963387 | SP-033 | Gc-globulin | VLEPTLK | 81 | 12 | Control |
| | | (Vitamin | | | | versus |
| | | D-binding | | | | AD |
| | | protein) | | | | |
| 31.14412441 | SP-033 | Gc-globulin | GQELCADYSENTFTEY | 82 | 13 | Control |
| | | (Vitamin | KK | | | versus |
| | | D-binding | | | | AD |
| | | protein) | | | | |
| 30.41928903 | SP-033 | Gc-globulin | ELPEHTVK | 83 | 14 | Control |
| | | (Vitamin | | | | versus |
| | | D-binding | | | | AD |
| | | protein) | | | | |
| 29.24518567 | SP-033 | Gc-globulin | KFPSGTFEQVSQLVK | 84 | 15 | Control |
| | | (Vitamin | | | | versus |
| | | D-binding | | | | AD |
| | | protein) | | | | |
| 28.3758185 | SP-033 | Gc-globulin | YTFELSR | 64 | 16 | Control |
| | | (Vitamin | | | | versus |
| | | D-binding | | | | AD |
| | | protein) | | | | |
| 27.71521659 | SP-033 | Gc-globulin | HLSLLTTLSNR | 85 | 17 | Control |
| | | (Vitamin | | | | versus |
| | | D-binding | | | | AD |
| | | protein) | | | | |
| 27.70389344 | SP-033 | Gc-globulin | LCMAALK | 59 | 18 | Control |
| | | (Vitamin | | | | versus |
| | | D-binding | | | | AD |
| | | protein) | | | | |
| 26.78598395 | SP-033 | Monocyte | VLDLSCNR | 86 | 19 | Control |
| | | differentiation | | | | versus |
| | | antigen CD14 | | | | AD |
| 26.76722276 | SP-033 | Gc-globulin | GQELCADYSENTFTEY | 87 | 20 | Control |
| | | (Vitamin | K | | | versus |
| | | D-binding | | | | AD |
| | | protein) | | | | |
| 100 | SP-339 | PROTEIN | LSVEIWDWDLTSR | 79 | 1 | Control |
| | | KINASE C | | | | versus |
| | | BETA TYPE | | | | AD |
| 79.19997182 | SP-339 | PROTEIN | CSLNPEWNETFR | 88 | 2 | Control |
| | | KINASE C | | | | versus |
| | | BETA TYPE | | | | AD |
| 50.23146221 | SP-339 | BIFUNCTIONAL | THVADFAPEVAWVTR | 89 | 3 | Control |
| | | GLUTAMATE/ | | | | versus |
| | | PRO LINE--TRNA | | | | AD |
| | | LIGASE | | | | |
| 43.74169647 | SP-339 | T-plasminogen | GGLFADIASHPWQAAI | 12 | 4 | Control |
| | | activator | FAK | | | versus |
| | | | | | | AD |
| 39.24043919 | SP-339 | Apolipoprotein | DGWQWFWSPSTFR | 90 | 5 | Control |
| | | C-IV | | | | versus |
| | | | | | | AD |
| 36.50836618 | SP-339 | Ubiquitin- | LGCDWVATIFSR | 91 | 6 | Control |
| | | associated and | | | | versus |
| | | SH3 domain- | | | | AD |
| | | containing | | | | |
| | | protein B | | | | |
| 35.77444925 | SP-339 | KININOGEN-1 | FKLDDDLEHQGGHVLD | 92 | 7 | Control |
| | | | HGHK | | | versus |
| | | | | | | AD |
| 33.80692266 | SP-339 | EH domain- | LFEAEEQDLFK | 93 | 8 | Control |
| | | containing | | | | versus |
| | | protein 1 | | | | AD |
| 29.97003734 | SP-339 | CHLORIDE | GVTFNVTTVDTK | 2 | 9 | Control |
| | | INTRACELLULAR | | | | versus |
| | | CHANNEL | | | | AD |
| | | PROTEIN 1 | | | | |
| 29.35421782 | SP-339 | Ubiquitin- | HGSALDVLLSMGFPR | 94 | 10 | Control |
| | | associated and | | | | versus |
| | | SH3 domain- | | | | AD |
| | | containing | | | | |
| | | protein B | | | | |
| 29.26487612 | SP-339 | SAA2-SAA4 | AYWDIMISNHQNSNR | 95 | 11 | Control |
| | | READTHROUGH | | | | versus |
| | | | | | | AD |
| 29.18424491 | SP-339 | APOLIPOPROTEIN | ELLETVVNR | 23 | 12 | Control |
| | | C-II | | | | versus |
| | | | | | | AD |
| 28.23221304 | SP-339 | SAA2-SAA4 | EALQGVGDMGR | 96 | 13 | Control |
| | | READTHROUGH | | | | versus |
| | | | | | | AD |
| 27.30231142 | SP-339 | Docking | VWALLYAGGPSGVAR | 97 | 14 | Control |
| | | protein 3 | | | | versus |
| | | | | | | AD |
| 26.7729128 | SP-339 | EH domain- | VHAYIISSLK | 98 | 15 | Control |
| | | containing | | | | versus |
| | | protein 1 | | | | AD |
| 26.68183193 | SP-339 | PEROXIREDOXIN-6 | LPFPIIDDR | 99 | 16 | Control |
| | | | | | | versus |
| | | | | | | AD |
| 26.56224494 | SP-339 | EH domain- | EMPNVFGK | 100 | 17 | Control |
| | | containing | | | | versus |
| | | protein 1 | | | | AD |
| 26.42306321 | SP-339 | Ubiquitin- | GNNILIVAHASSLEAC | 101 | 18 | Control |
| | | associated and | TCQLQGLSPQNSK | | | versus |
| | | SH3 domain- | | | | AD |
| | | containing | | | | |
| | | protein B | | | | |
| 25.48721205 | SP-339 | Extracellular | INVIVLR | 102 | 19 | Control |
| | | matrix | | | | versus |
| | | protein 2 | | | | AD |
| 24.22212679 | SP-339 | CHLORIDE | LAALNPESNTAGLDIF | 31 | 20 | Control |
| | | INTRACELLULAR | AK | | | versus |
| | | CHANNEL | | | | AD |
| | | PROTEIN 1 | | | | |
| 100 | SP-047 | Complement | SHALQLNNR | 103 | 1 | Control |
| | | C4-B | | | | versus |
| | | | | | | AD |
| 96.48819279 | SP-047 | Beta-2- | TFYEPGEEITYSCK | 104 | 2 | Control |
| | | glycoprotein 1 | | | | versus |
| | | | | | | AD |
| 72.41045847 | SP-047 | EH domain- | ELVNNLGEIYQK | 105 | 3 | Control |
| | | containing | | | | versus |
| | | protein 1 | | | | AD |
| 71.77506075 | SP-047 | T-plasminogen | VTNYLDWIRDNMRP | 106 | 4 | Control |
| | | activator | | | | versus |
| | | | | | | AD |
| 61.21045872 | SP-047 | Plasma serine | QLELYLPK | 107 | 5 | Control |
| | | protease | | | | versus |
| | | inhibitor | | | | AD |
| 56.54355041 | SP-047 | PLASMINOGEN | NLDENYCR | 108 | 6 | Control |
| | | | | | | versus |
| | | | | | | AD |
| 47.67627718 | SP-047 | Alpha-2-HS- | FSVVYAK | 109 | 7 | Control |
| | | glycoprotein | | | | versus |
| | | | | | | AD |
| 47.04542048 | SP-047 | HISTIDINE-RICH | YWNDCEPPDSRRPSEI | 110 | 8 | Control |
| | | GLYCOPROTEIN | VIGQCK | | | versus |
| | | | | | | AD |
| 46.78449481 | SP-047 | TRANSGELIN-2 | NVIGLQMGTNR | 111 | 9 | Control |
| | | | | | | versus |
| | | | | | | AD |
| 45.30385807 | SP-047 | Protein | NLIPMDPNGLSDPYVK | 112 | 10 | Control |
| | | kinase C | | | | versus |
| | | alpha type | | | | AD |
| 43.76510888 | SP-047 | Plasma serine | QINDYVAK | 113 | 11 | Control |
| | | protease | | | | versus |
| | | inhibitor | | | | AD |
| 42.44928433 | SP-047 | INTER-ALPHA- | NVVFVIDK | 114 | 12 | Control |
| | | TRYPSIN | | | | versus |
| | | INHIBITOR | | | | AD |
| | | HEAVY CHAIN | | | | |
| | | H4 (ITIH4) | | | | |
| | | PROTEIN | | | | |
| 42.38032268 | SP-047 | HISTIDINE-RICH | YWNDCEPPDSR | 70 | 13 | Control |
| | | GLYCOPROTEIN | | | | versus |
| | | | | | | AD |
| 41.79796255 | SP-047 | RHO-ASSOCIATED | LKDEEISAAAIK | 115 | 14 | Control |
| | | PROTEIN | | | | versus |
| | | KINASE 2 | | | | AD |
| 40.43558875 | SP-047 | EH domain- | DGLLDDEEFALANHLI | 116 | 15 | Control |
| | | containing | K | | | versus |
| | | protein 1 | | | | AD |
| 40.27046477 | SP-047 | T-plasminogen | GGLFADIASHPWQAAI | 12 | 16 | Control |
| | | activator | FAK | | | versus |
| | | | | | | AD |
| 39.29856832 | SP-047 | Protein | STLNPQWNESFTFK | 117 | 17 | Control |
| | | kinase C | | | | versus |
| | | alpha type | | | | AD |
| 39.25638988 | SP-047 | PLASMINOGEN | RWELCDIPR | 118 | 18 | Control |
| | | | | | | versus |
| | | | | | | AD |
| 38.73573455 | SP-047 | PDZ AND LIM | GHFFVEDQIYCEK | 119 | 19 | Control |
| | | DOMAIN | | | | versus |
| | | PROTEIN 1 | | | | AD |
| 38.07808515 | SP-047 | PLASMINOGEN | YEFLNGR | 120 | 20 | Control |
| | | | | | | versus |
| | | | | | | AD |
| 100 | SP-053 | INTER-ALPHA- | LALDNGGLAR | 121 | 1 | Control |
| | | TRYPSIN | | | | versus |
| | | INHIBITOR | | | | AD |
| | | HEAVY CHAIN | | | | |
| | | H4 (ITIH4) | | | | |
| | | PROTEIN | | | | |
| 98.61848265 | SP-053 | TRANSTHYRETIN | TSESGELHGLTTEEEF | 122 | 2 | Control |
| | | | VEGIYK | | | versus |
| | | | | | | AD |
| 85.29424222 | SP-053 | TUBULIN | GHYTEGAELVDSVLDV | 123 | 3 | Control |
| | | BETA-4B CHAIN | VR | | | versus |
| | | | | | | AD |
| 84.30729181 | SP-053 | APOLIPOPROTEIN | ELLETVVNR | 23 | 4 | Control |
| | | C-II | | | | versus |
| | | | | | | AD |
| 72.10882073 | SP-053 | ZYXIN-2 | PLSIEADDNGCFPLDG | 124 | 5 | Control |
| | | | HVLCR | | | versus |
| | | | | | | AD |
| 69.35603984 | SP-053 | TUBULIN | LTTPTYGDLNHLVSAT | 125 | 6 | Control |
| | | BETA-4B CHAIN | MSGVTTCLR | | | versus |
| | | | | | | AD |
| 66.73644143 | SP-053 | TUBULIN | FWEVISDEHGIDPTGT | 126 | 7 | Control |
| | | BETA-4B CHAIN | YHGDSDLQLER | | | versus |
| | | | | | | AD |
| 65.02854784 | SP-053 | INTER-ALPHA- | LQDRGPDVLTATVSGK | 127 | 8 | Control |
| | | TRYPSIN | | | | versus |
| | | INHIBITOR | | | | AD |
| | | HEAVY CHAIN | | | | |
| | | H4 (ITIH4) | | | | |
| | | PROTEIN | | | | |
| 60.32644975 | SP-053 | TUBULIN | FDLMYAK | 51 | 9 | Control |
| | | ALPHA-1B | | | | versus |
| | | CHAIN | | | | AD |
| 59.30168955 | SP-053 | PEROXIREDOXIN-6 | LPFPIIDDR | 99 | 10 | Control |
| | | | | | | versus |
| | | | | | | AD |
| 57.70723599 | SP-053 | HISTIDINE-RICH | DGYLFQLLR | 128 | 11 | Control |
| | | GLYCOPROTEIN | | | | versus |
| | | | | | | AD |
| 53.84294718 | SP-053 | ALPHA-2- | LGNQEPGGQTALK | 26 | 12 | Control |
| | | ANTIPLASMIN | | | | versus |
| | | | | | | AD |
| 51.03990298 | SP-053 | TUBULIN | INVYYNEATGGK | 40 | 13 | Control |
| | | BETA-4B CHAIN | | | | versus |
| | | | | | | AD |
| 45.047738 | SP-053 | Complement | DYFIATCK | 129 | 14 | Control |
| | | subcomponent | | | | versus |
| | | C1r | | | | AD |
| 44.66554904 | SP-053 | Myotrophin | NGDLDEVK | 130 | 15 | Control |
| | | | | | | versus |
| | | | | | | AD |
| 43.2559614 | SP-053 | TUBULIN | LSVDYGK | 131 | 16 | Control |
| | | ALPHA-1B | | | | versus |
| | | CHAIN | | | | AD |
| 43.12316743 | SP-053 | TUBULIN | TIGGGDDSFNTFFSET | 132 | 17 | Control |
| | | ALPHA-1B | GAGK | | | versus |
| | | CHAIN | | | | AD |
| 43.01172638 | SP-053 | Regulator of | EVITNSITQPTLHSFD | 133 | 18 | Control |
| | | G-protein | AAQSR | | | versus |
| | | signaling 18 | | | | AD |
| 41.74538114 | SP-053 | ALPHA-2- | SPPGVCSR | 55 | 19 | Control |
| | | ANTIPLASMIN | | | | versus |
| | | | | | | AD |
| 40.16628137 | SP-053 | TUBULIN | NSSYFVEWIPNNVK | 57 | 20 | Control |
| | | BETA-4B CHAIN | | | | versus |
| | | | | | | AD |
| 100 | SP-373 | APOLIPOPROTEIN | ELLETVVNR | 23 | 1 | Control |
| | | C-II | | | | versus |
| | | | | | | AD |
| 93.36066604 | SP-373 | Complement | IPGIFELGISSQSDR | 134 | 2 | Control |
| | | component C8 | | | | versus |
| | | beta chain | | | | AD |
| 81.36577113 | SP-373 | Cofilin, | HELQANCYEEVK | 135 | 3 | Control |
| | | non-muscle | | | | versus |
| | | isoform | | | | AD |
| | | (Cofilin-1) | | | | |
| 79.50439608 | SP-373 | TRANSTHYRETIN | ALGISPFHEHAEVVFT | 7 | 4 | Control |
| | | | ANDSGPR | | | versus |
| | | | | | | AD |
| 70.32355462 | SP-373 | VON WILLEBRAND | YAGSQVASTSEVLK | 136 | 5 | Control |
| | | FACTOR | | | | versus |
| | | | | | | AD |
| 65.94648938 | SP-373 | HEPARIN | SVNDLYIQK | 137 | 6 | Control |
| | | COFACTOR 2 | | | | versus |
| | | | | | | AD |
| 64.52646043 | SP-373 | HEXOKINASE-1 | ITPELLTR | 138 | 7 | Control |
| | | | | | | versus |
| | | | | | | AD |
| 61.42174948 | SP-373 | Cofilin, | NIILEEGK | 139 | 8 | Control |
| | | non-muscle | | | | versus |
| | | isoform | | | | AD |
| | | (Cofilin-1) | | | | |
| 58.81996884 | SP-373 | HEXOKINASE-1 | FNTSDVSAIEK | 140 | 9 | Control |
| | | | | | | versus |
| | | | | | | AD |
| 57.23539535 | SP-373 | C4a | SHALQLNNR | 103 | 10 | Control |
| | | anaphylatoxin | | | | versus |
| | | | | | | AD |
| 56.65231955 | SP-373 | RECEPTOR-TYPE | VDVYGYVVK | 141 | 11 | Control |
| | | TYROSINE- | | | | versus |
| | | PROTEIN | | | | AD |
| | | PHOSPHATASE C | | | | |
| 56.60853227 | SP-373 | Stomatin | NSTIVFPLPIDMLQGI | 142 | 12 | Control |
| | | | IGAK | | | versus |
| | | | | | | AD |
| 54.27356309 | SP-373 | COAGULATION | SQHLDNFSNQIGK | 143 | 13 | Control |
| | | FACTOR V | | | | versus |
| | | | | | | AD |
| 52.76790237 | SP-373 | HEPARIN | NGNMAGISDQR | 144 | 14 | Control |
| | | COFACTOR 2 | | | | versus |
| | | | | | | AD |
| 52.56579902 | SP-373 | HEXOKINASE-1 | LVDEYSLNAGK | 145 | 15 | Control |
| | | | | | | versus |
| | | | | | | AD |
| 51.36207484 | SP-373 | GLYCERALDEHYDE- | VPTANVSVVDLTCR | 146 | 16 | Control |
| | | 3-PHOSPHATE | | | | versus |
| | | DEHYDROGENASE | | | | AD |
| 50.85006906 | SP-373 | HEPARIN | TLEAQLTPR | 6 | 17 | Control |
| | | COFACTOR 2 | | | | versus |
| | | | | | | AD |
| 48.50582902 | SP-373 | Histone H1.4 | SGVSLAALK | 147 | 18 | Control |
| | | | | | | versus |
| | | | | | | AD |
| 47.97757905 | SP-373 | HEXOKINASE-1 | GAALITAVGVR | 148 | 19 | Control |
| | | | | | | versus |
| | | | | | | AD |
| 46.26538012 | SP-373 | COAGULATION | EKPQSTISGLLGPTLY | 149 | 20 | Control |
| | | FACTOR V | AEVGDIIK | | | versus |
| | | | | | | AD |
| 100 | SP-089 | T-plasminogen | GGLFADIASHPWQAAI | 12 | 1 | Control |
| | | activator | FAK | | | versus |
| | | | | | | AD |
| 66.61483143 | SP-089 | HISTIDINE-RICH | QIGSVYR | 150 | 2 | Control |
| | | GLYCOPROTEIN | | | | versus |
| | | | | | | AD |
| 65.32411729 | SP-089 | HISTIDINE-RICH | PHEHGPPPPPDER | 151 | 3 | Control |
| | | GLYCOPROTEIN | | | | versus |
| | | | | | | AD |
| 61.43405447 | SP-089 | HISTIDINE-RICH | DHSHGPPLPQGPPPLL | 152 | 4 | Control |
| | | GLYCOPROTEIN | PMSCSSCQHATFGTNG | | | versus |
| | | | AQR | | | AD |
| 58.33621522 | SP-089 | T-plasminogen | VYTAQNPSAQALGLGK | 153 | 5 | Control |
| | | activator | | | | versus |
| | | | | | | AD |
| 58.24163085 | SP-089 | HISTIDINE-RICH | IADAHLDRVENTTVYY | 154 | 6 | Control |
| | | GLYCOPROTEIN | LVLDVQESDCSVLSR | | | versus |
| | | | | | | AD |
| 58.10194432 | SP-089 | HISTIDINE-RICH | GEVLPLPEANFPSFPL | 72 | 7 | Control |
| | | GLYCOPROTEIN | PHHK | | | versus |
| | | | | | | AD |
| 57.1657399 | SP-089 | Kinesin-1 | SATLASIDAELQK | 155 | 8 | Control |
| | | heavy chain | | | | versus |
| | | | | | | AD |
| 56.34412777 | SP-089 | HISTIDINE-RICH | RDGYLFQLLR | 156 | 9 | Control |
| | | GLYCOPROTEIN | | | | versus |
| | | | | | | AD |
| 55.84853245 | SP-089 | HISTIDINE-RICH | YWNDCEPPDSR | 70 | 10 | Control |
| | | GLYCOPROTEIN | | | | versus |
| | | | | | | AD |
| 53.90405265 | SP-089 | TRANSTHYRETIN | TSESGELHGLTTEEEF | 122 | 11 | Control |
| | | | VEGIYK | | | versus |
| | | | | | | AD |
| 52.98844057 | SP-089 | HISTIDINE-RICH | ALDLINK | 157 | 12 | Control |
| | | GLYCOPROTEIN | | | | versus |
| | | | | | | AD |
| 52.04751267 | SP-089 | Soluble | ELGCGGPQQPDPAAGR | 158 | 13 | Control |
| | | scavenger | | | | versus |
| | | receptor | | | | AD |
| | | cysteine- | | | | |
| | | rich domain- | | | | |
| | | containing | | | | |
| | | protein SSC5D | | | | |
| 51.92586555 | SP-089 | HISTIDINE-RICH | HPNVFGFCR | 159 | 14 | Control |
| | | GLYCOPROTEIN | | | | versus |
| | | | | | | AD |
| 49.60740453 | SP-089 | C—X—C motif | CQCLQTLQGIHLK | 160 | 15 | Control |
| | | chemokine 2 | | | | versus |
| | | | | | | AD |
| 44.77828276 | SP-089 | FH1/FH2 domain- | LLTMMPTEEER | 161 | 16 | Control |
| | | containing | | | | versus |
| | | protein 1 | | | | AD |
| 44.57194148 | SP-089 | VON WILLEBRAND | IGWPNAPILIQDFETL | 162 | 17 | Control |
| | | FACTOR | PR | | | versus |
| | | | | | | AD |
| 44.49971598 | SP-089 | Cytoplasmic | NAFVTGIAR | 163 | 18 | Control |
| | | FMR1- | | | | versus |
| | | interacting | | | | AD |
| | | protein 1 | | | | |
| 42.22047786 | SP-089 | KINESIN-LIKE | GSLDYRPLTTADPIDE | 164 | 19 | Control |
| | | PROTEIN KIF2A | HR | | | versus |
| | | | | | | AD |
| 40.91061663 | SP-089 | Cytoplasmic | YSNSEVVTGSGR | 165 | 20 | Control |
| | | FMR1- | | | | versus |
| | | interacting | | | | AD |
| | | protein 1 | | | | |
| 100 | SP-003 | Adipsin | RPDSLQHVLLPVLDR | 166 | 1 | Control |
| | | | | | | versus |
| | | | | | | MCI |
| 52.6080765 | SP-003 | COMPLEMENT C3 | LSINTHPSQK | 167 | 2 | Control |
| | | | | | | versus |
| | | | | | | MCI |
| 50.3381192 | SP-003 | COMPLEMENT C3 | SGSDEVQVGQQR | 168 | 3 | Control |
| | | | | | | versus |
| | | | | | | MCI |
| 49.90066767 | SP-003 | PDZ AND LIM | GHFFVEDQIYCEK | 119 | 4 | Control |
| | | DOMAIN | | | | versus |
| | | PROTEIN 1 | | | | MCI |
| 49.43705755 | SP-003 | COMPLEMENT C3 | VHQYFNVELIQPGAVK | 169 | 5 | Control |
| | | | | | | versus |
| | | | | | | MCI |
| 47.60910739 | SP-003 | Vinculin | TNLLQVCER | 170 | 6 | Control |
| | | | | | | versus |
| | | | | | | MCI |
| 47.14697149 | SP-003 | HEPARANASE | SVQLNGLTLK | 171 | 7 | Control |
| | | | | | | versus |
| | | | | | | MCI |
| 45.62558714 | SP-003 | MYOSIN | LSNDMMGSYAEMK | 18 | 8 | Control |
| | | APOLIPOPROTEIN | | | | versus |
| | | B-100LIGHT | | | | MCI |
| | | POLYPEPTIDE 9 | | | | |
| 44.68212395 | SP-003 | COMPLEMENT C3 | FYYIYNEK | 172 | 9 | Control |
| | | | | | | versus |
| | | | | | | MCI |
| 42.98362077 | SP-003 | COMPLEMENT C3 | IWDVVEK | 173 | 10 | Control |
| | | | | | | versus |
| | | | | | | MCI |
| 42.50416538 | SP-003 | COMPLEMENT C3 | TIYTPGSTVLYR | 174 | 11 | Control |
| | | | | | | versus |
| | | | | | | MCI |
| 40.22094484 | SP-003 | TYROSINE- | ELNGTYAIAGGR | 175 | 12 | Control |
| | | PROTEIN | | | | versus |
| | | KINASE SYK | | | | MCI |
| 37.28691456 | SP-003 | Rho guanine | STAALEEDAQILK | 176 | 13 | Control |
| | | nucleotide | | | | versus |
| | | exchange | | | | MCI |
| | | factor 7 | | | | |
| 35.83347612 | SP-003 | COMPLEMENT C3 | VVLVAVDK | 177 | 14 | Control |
| | | | | | | versus |
| | | | | | | MCI |
| 35.43518697 | SP-003 | GTP-binding | LGQHVPTLHPTSEELT | 178 | 15 | Control |
| | | protein SAR1a | IAGMTFTTFDLGGHEQ | | | versus |
| | | | AR | | | MCI |
| 33.97789214 | SP-003 | COMPLEMENT C3 | NTLIIYLDK | 179 | 16 | Control |
| | | | | | | versus |
| | | | | | | MCI |
| 33.26877052 | SP-003 | COMPLEMENT C3 | SNLDEDIIAEENIVSR | 180 | 17 | Control |
| | | | | | | versus |
| | | | | | | MCI |
| 33.09288065 | SP-003 | KINESIN-LIKE | LIDIGNSCR | 181 | 18 | Control |
| | | PROTEIN KIF2A | | | | versus |
| | | | | | | MCI |
| 32.84946733 | SP-003 | COMPLEMENT C3 | TVMVNIENPEGIPVK | 182 | 19 | Control |
| | | | | | | versus |
| | | | | | | MCI |
| 32.72876986 | SP-003 | COMPLEMENT C3 | HQQTVTIPPK | 183 | 20 | Control |
| | | | | | | versus |
| | | | | | | MCI |
| 100 | SP-006 | MYOSIN | LSNDMMGSYAEMK | 18 | 1 | Control |
| | | APOLIPOPROTEIN | | | | versus |
| | | B-100LIGHT | | | | MCI |
| | | POLYPEPTIDE 9 | | | | |
| 82.49224991 | SP-006 | APOLIPOPROTEIN | ELLETVVNR | 23 | 2 | Control |
| | | C-II | | | | versus |
| | | | | | | MCI |
| 41.03739142 | SP-006 | CHLORIDE | YLSNAYAR | 184 | 3 | Control |
| | | INTRACELLULAR | | | | versus |
| | | CHANNEL | | | | MCI |
| | | PROTEIN 1 | | | | |
| 40.59994011 | SP-006 | ALPHA-2- | SPPGVCSR | 55 | 4 | Control |
| | | ANTIPLASMIN | | | | versus |
| | | | | | | MCI |
| 37.57827949 | SP-006 | MYOSIN | SVMAPFTMTIDAHTNG | 185 | 5 | Control |
| | | APOLIPOPROTEIN | NGK | | | versus |
| | | B-100LIGHT | | | | MCI |
| | | POLYPEPTIDE 9 | | | | |
| 35.61839965 | SP-006 | CHLORIDE | GVTFNVTTVDTK | 2 | 6 | Control |
| | | INTRACELLULAR | | | | versus |
| | | CHANNEL | | | | MCI |
| | | PROTEIN 1 | | | | |
| 34.0198624 | SP-006 | ALPHA-2- | LGNQEPGGQTALK | 26 | 7 | Control |
| | | ANTIPLASMIN | | | | versus |
| | | | | | | MCI |
| 32.15312466 | SP-006 | CHROMOGRANIN- | GLSAEPGVVQAK | 186 | 8 | Control |
| | | A | | | | versus |
| | | | | | | MCI |
| 27.09581176 | SP-006 | Complement | LVFQQFDLEPSEGCFY | 187 | 9 | Control |
| | | subcomponent | DYVK | | | versus |
| | | C1r | | | | MCI |
| 25.28062955 | SP-006 | Complement | IACVLPVLMDGIQSHP | 188 | 10 | Control |
| | | component C7 | QK | | | versus |
| | | | | | | MCI |
| 24.61873241 | SP-006 | COMPLEMENT | TLNICEVGTIR | 189 | 11 | Control |
| | | COMPONENT C6 | | | | versus |
| | | | | | | MCI |
| 23.49680272 | SP-006 | Fermitin | QWNVNWDIR | 190 | 12 | Control |
| | | family | | | | versus |
| | | homolog 3 | | | | MCI |
| 23.12596394 | SP-006 | HISTIDINE-RICH | YKEENDDFASFR | 67 | 13 | Control |
| | | GLYCOPROTEIN | | | | versus |
| | | | | | | MCI |
| 22.56313179 | SP-006 | TRANSGELIN-2 | NVIGLQMGTNR | 111 | 14 | Control |
| | | | | | | versus |
| | | | | | | MCI |
| 22.49421562 | SP-006 | COMPLEMENT C3 | FISLGEACK | 191 | 15 | Control |
| | | | | | | versus |
| | | | | | | MCI |
| 22.20760268 | SP-006 | Apolipoprotein | DGWQWFWSPSTFR | 90 | 16 | Control |
| | | C-IV | | | | versus |
| | | | | | | MCI |
| 21.9195247 | SP-006 | PYRUVATE | CCSGAIIVLTK | 192 | 17 | Control |
| | | KINASE PKM | | | | versus |
| | | | | | | MCI |
| 21.70636872 | SP-006 | GLYCERALDEHYDE- | VIISAPSADAPMFVMG | 193 | 18 | Control |
| | | 3-PHOSPHATE | VNHEK | | | versus |
| | | DEHYDROGENASE | | | | MCI |
| 21.63005469 | SP-006 | COMPLEMENT C3 | NTLIIYLDK | 179 | 19 | Control |
| | | | | | | versus |
| | | | | | | MCI |
| 21.53775965 | SP-006 | Integrin | DEITFVSGAPR | 194 | 20 | Control |
| | | alpha-6 | | | | versus |
| | | | | | | MCI |
| 100 | SP-007 | APOLIPOPROTEIN | ELLETVVNR | 23 | 1 | Control |
| | | C-II | | | | versus |
| | | | | | | MCI |
| 52.00778682 | SP-007 | Tubulin | EDLAALEK | 195 | 2 | Control |
| | | alpha chain | | | | versus |
| | | | | | | MCI |
| 44.10859962 | SP-007 | TUBULIN | LSVDYGK | 131 | 3 | Control |
| | | ALPHA-1B CHAIN | | | | versus |
| | | | | | | MCI |
| 42.44701988 | SP-007 | ALPHA-2- | LGNQEPGGQTALK | 26 | 4 | Control |
| | | ANTIPLASMIN | | | | versus |
| | | | | | | MCI |
| 40.40486401 | SP-007 | TUBULIN | NSSYFVEWIPNNVK | 57 | 5 | Control |
| | | BETA-4B CHAIN | | | | versus |
| | | | | | | MCI |
| 38.57192196 | SP-007 | INTER-ALPHA- | QGPVNLLSDPEQGVEV | 46 | 6 | Control |
| | | TRYPSIN | TGQYER | | | versus |
| | | INHIBITOR | | | | MCI |
| | | HEAVY CHAIN | | | | |
| | | H4 (ITIH4) | | | | |
| | | PROTEIN | | | | |
| 36.41538832 | SP-007 | TUBULIN | INVYYNEATGGK | 40 | 7 | Control |
| | | BETA-4B CHAIN | | | | versus |
| | | | | | | MCI |
| 35.99087967 | SP-007 | CHROMOGRANIN- | SEALAVDGAGKPGAEE | 196 | 8 | Control |
| | | A | AQDPEGK | | | versus |
| | | | | | | MCI |
| 34.8275035 | SP-007 | TUBULIN | FDGALNVDLTEFQTNL | 43 | 9 | Control |
| | | ALPHA-1B | VPYPR | | | versus |
| | | CHAIN | | | | MCI |
| 31.68281443 | SP-007 | CHROMOGRANIN- | CIVEVISDTLSK | 197 | 10 | Control |
| | | A | | | | versus |
| | | | | | | MCI |
| 31.20403173 | SP-007 | INTER-ALPHA- | AGFSWIEVTFK | 41 | 11 | Control |
| | | TRYPSIN | | | | versus |
| | | INHIBITOR | | | | MCI |
| | | HEAVY CHAIN | | | | |
| | | H4 (ITIH4) | | | | |
| | | PROTEIN | | | | |
| 29.69268349 | SP-007 | TUBULIN | GHYTEGAELVDSVLDV | 198 | 12 | Control |
| | | BETA-4B CHAIN | VRK | | | versus |
| | | | | | | MCI |
| 29.55616247 | SP-007 | TUBULIN | EIIDPVLDR | 199 | 13 | Control |
| | | ALPHA-4A | | | | versus |
| | | CHAIN | | | | MCI |
| 29.26354533 | SP-007 | CHROMOGRANIN- | GLSAEPGWQAK | 186 | 14 | Control |
| | | A | | | | versus |
| | | | | | | MCI |
| 29.20368891 | SP-007 | Complement | HSCQAECSSELYTEAS | 200 | 15 | Control |
| | | subcomponent | GYISSLEYPR | | | versus |
| | | C1r | | | | MCI |
| 29.19821979 | SP-007 | CHROMOGRANIN- | EEEEEMAVVPQGLFR | 201 | 16 | Control |
| | | A | | | | versus |
| | | | | | | MCI |
| 28.38299177 | SP-007 | Tubulin | AILVDLEPGTMDSVR | 202 | 17 | Control |
| | | beta chain | | | | versus |
| | | | | | | MCI |
| 27.01204971 | SP-007 | TUBULIN | AVFVDLEPTVIDEIR | 203 | 18 | Control |
| | | ALPHA-4A | | | | versus |
| | | CHAIN | | | | MCI |
| 26.91756186 | SP-007 | Apolipoprotein | MREWFSETFQK | 204 | 19 | Control |
| | | C-I | | | | versus |
| | | | | | | MCI |
| 26.10125409 | SP-007 | Complement | LPVANPQACENWLR | 205 | 20 | Control |
| | | subcomponent | | | | versus |
| | | C1r | | | | MCI |
| 100 | SP-008 | Gc-globulin | LCDNLSTK | 58 | 1 | Control |
| | | (Vitamin | | | | versus |
| | | D-binding | | | | MCI |
| | | protein) | | | | |
| 99.1888572 | SP-008 | PDZ AND LIM | GCTDNLTLTVAR | 206 | 2 | Control |
| | | DOMAIN | | | | versus |
| | | PROTEIN 1 | | | | MCI |
| 81.3239688 | SP-008 | PDZ AND LIM | SAMPFTASPASSTTAR | 207 | 3 | Control |
| | | DOMAIN | | | | versus |
| | | PROTEIN 1 | | | | MCI |
| 76.49448498 | SP-008 | PDZ AND LIM | MNLASEPQEVLHIGSA | 208 | 4 | Control |
| | | DOMAIN | HNR | | | versus |
| | | PROTEIN 1 | | | | MCI |
| 76.45028057 | SP-008 | RAS GTPASE- | HSQSMIEDAQLPLEQK | 209 | 5 | Control |
| | | ACTIVATING- | | | | versus |
| | | LIKE | | | | MCI |
| | | PROTEIN IQGAP2 | | | | |
| 69.54229964 | SP-008 | APOLIPOPROTEIN | ELLETVVNR | 23 | 6 | Control |
| | | C-II | | | | versus |
| | | | | | | MCI |
| 62.7054236 | SP-008 | INSULIN-LIKE | FFQYDTWK | 210 | 7 | Control |
| | | GROWTH FACTOR | | | | versus |
| | | II | | | | MCI |
| 60.09300964 | SP-008 | PDZ AND LIM | QSTSFLVLQEILESEE | 211 | 8 | Control |
| | | DOMAIN | K | | | versus |
| | | PROTEIN I | | | | MCI |
| 59.54792899 | SP-008 | PDZ AND LIM | DFEQPLAISR | 212 | 9 | Control |
| | | DOMAIN | | | | versus |
| | | PROTEIN 1 | | | | MCI |
| 58.9634541 | SP-008 | Albumin | LDELRDEGK | 213 | 10 | Control |
| | | | | | | versus |
| | | | | | | MCI |
| 58.22715246 | SP-008 | T-plasminogen | VTNYLDWIRDNMRP | 106 | 11 | Control |
| | | activator | | | | versus |
| | | | | | | MCI |
| 57.55234831 | SP-008 | PROTEIN | LSVEIWDWDLTSR | 79 | 12 | Control |
| | | KINASE C | | | | versus |
| | | BETA TYPE | | | | MCI |
| 56.56114133 | SP-008 | RHO GTPASE- | LQLFGQDFSHAAR | 214 | 13 | Control |
| | | ACTIVATING | | | | versus |
| | | PROTEIN 45 | | | | MCI |
| 56.0241425 | SP-008 | HISTIDINE-RICH | GEVLPLPEANFPSFPL | 72 | 14 | Control |
| | | GLYCOPROTEIN | PHHK | | | versus |
| | | | | | | MCI |
| 54.47165235 | SP-008 | HISTIDINE-RICH | KGEVLPLPEANFPSFP | 63 | 15 | Control |
| | | GLYCOPROTEIN | LPHHK | | | versus |
| | | | | | | MCI |
| 54.34239551 | SP-008 | HISTIDINE-RICH | KYWNDCEPPDSR | 215 | 16 | Control |
| | | GLYCOPROTEIN | | | | versus |
| | | | | | | MCI |
| 54.32384573 | SP-008 | PROTEIN | ASVDGWFK | 216 | 17 | Control |
| | | KINASE C | | | | versus |
| | | BETA TYPE | | | | MCI |
| 52.06525817 | SP-008 | GMP reductase 1 | MTSILEAVPQVK | 217 | 18 | Control |
| | | | | | | versus |
| | | | | | | MCI |
| 51.0202079 | SP-008 | PDZ AND LIM | VITNQYNNPAGLYSSE | 218 | 19 | Control |
| | | DOMAIN | NISNFNNALESK | | | versus |
| | | PROTEIN 1 | | | | MCI |
| 50.59509894 | SP-008 | ZYXIN-2 | PQVQLHVQSQTQPVSL | 219 | 20 | Control |
| | | | ANTQPR | | | versus |
| | | | | | | MCI |
| 100 | SP-033 | SAA2-SAA4 | EALQGVGDMGR | 96 | 1 | Control |
| | | READTHROUGH | | | | versus |
| | | | | | | MCI |
| 45.61419232 | SP-033 | APOLIPOPROTEIN | ELLETVVNR | 23 | 2 | Control |
| | | C-II | | | | versus |
| | | | | | | MCI |
| 44.67935982 | SP-033 | Gc-globulin | LCDNLSTK | 58 | 3 | Control |
| | | (Vitamin | | | | versus |
| | | D-binding | | | | MCI |
| | | protein) | | | | |
| 42.71966834 | SP-033 | Gc-globulin | EDFTSLSLVLYSR | 68 | 4 | Control |
| | | (Vitamin | | | | versus |
| | | D-binding | | | | MCI |
| | | protein) | | | | |
| 41.58710534 | SP-033 | Ubiquitin- | HGSALDVLLSMGFPR | 94 | 5 | Control |
| | | associated and | | | | versus |
| | | SH3 domain- | | | | MCI |
| | | containing | | | | |
| | | protein B | | | | |
| 41.14248013 | SP-033 | Alpha-actinin-1 | TINEVENQILTR | 220 | 6 | Control |
| | | | | | | versus |
| | | | | | | MCI |
| 39.47820595 | SP-033 | FIBRONECTIN | ISCTIANR | 221 | 7 | Control |
| | | | | | | versus |
| | | | | | | MCI |
| 34.92912501 | SP-033 | PROTEIN | LSVEIWDWDLTSR | 79 | 8 | Control |
| | | KINASE C | | | | versus |
| | | BETA TYPE | | | | MCI |
| 34.27228018 | SP-033 | Ubiquitin- | LAQNIDVK | 222 | 9 | Control |
| | | associated and | | | | versus |
| | | SH3 domain- | | | | MCI |
| | | containing | | | | |
| | | protein B | | | | |
| 33.53018652 | SP-033 | T-plasminogen | GGLFADIASHPWQAAI | 12 | 10 | Control |
| | | activator | FAK | | | versus |
| | | | | | | MCI |
| 32.755592 | SP-033 | Peptidyl- | VNPTVFFDIAVDGEPL | 223 | 11 | Control |
| | | prolycis- | GR | | | versus |
| | | transisomerase | | | | MCI |
| | | A | | | | |
| 32.69280719 | SP-033 | Tropomodulin-3 | MLEENTNILK | 224 | 12 | Control |
| | | | | | | versus |
| | | | | | | MCI |
| 32.66714788 | SP-033 | COMPLEMENT C3 | IHWESASLLR | 225 | 13 | Control |
| | | | | | | versus |
| | | | | | | MCI |
| 31.68865891 | SP-033 | INSULIN-LIKE | FFQYDTWK | 210 | 14 | Control |
| | | GROWTH FACTOR | | | | versus |
| | | II | | | | MCI |
| 31.0614241 | SP-033 | CHROMOGRANIN- | HSGFEDELSEVLENQS | 226 | 15 | Control |
| | | A | SQAELK | | | versus |
| | | | | | | MCI |
| 30.33456976 | SP-033 | Apolipoprotein | NILTSNNIDVK | 227 | 16 | Control |
| | | D | | | | versus |
| | | | | | | MCI |
| 28.01105704 | SP-033 | HISTIDINE-RICH | HPNVFGFCR | 159 | 17 | Control |
| | | GLYCOPROTEIN | | | | versus |
| | | | | | | MCI |
| 27.33113337 | SP-033 | Gc-globulin | VCSQYAAYGEK | 228 | 18 | Control |
| | | (Vitamin | | | | versus |
| | | D-binding | | | | MCI |
| | | protein) | | | | |
| 26.72366309 | SP-033 | Gc-globulin | ELSSFIDK | 73 | 19 | Control |
| | | (Vitamin | | | | versus |
| | | D-binding | | | | MCI |
| | | protein) | | | | |
| 24.96817303 | SP-033 | RHO GTPASE- | LQLFGQDFSHAAR | 214 | 20 | Control |
| | | ACTIVATING | | | | versus |
| | | PROTEIN 45 | | | | MCI |
| 100 | SP-339 | SAA2-SAA4 | EALQGVGDMGR | 96 | 1 | Control |
| | | READTHROUGH | | | | versus |
| | | | | | | MCI |
| 93.34863875 | SP-339 | BIFUNCTIONAL | THVADFAPEVAWVTR | 89 | 2 | Control |
| | | GLUTAMATE/ | | | | versus |
| | | PRO LINE--TRNA | | | | MCI |
| | | LIGASE | | | | |
| 72.14827258 | SP-339 | COMPLEMENT C3 | SSLSVPYVIVPLK | 229 | 3 | Control |
| | | | | | | versus |
| | | | | | | MCI |
| 63.09298868 | SP-339 | Apolipoprotein | DGWQWFWSPSTFR | 90 | 4 | Control |
| | | C-IV | | | | versus |
| | | | | | | MCI |
| 62.9513466 | SP-339 | Coagulation | SALVLQYLR | 230 | 5 | Control |
| | | factor IX | | | | versus |
| | | | | | | MCI |
| 59.78131335 | SP-339 | PDZ AND LIM | VWSPLVTEEGK | 231 | 6 | Control |
| | | DOMAIN | | | | versus |
| | | PROTEIN 1 | | | | MCI |
| 48.48382375 | SP-339 | COMPLEMENT C3 | NTLIIYLDK | 179 | 7 | Control |
| | | | | | | versus |
| | | | | | | MCI |
| 47.61634478 | SP-339 | Regulator of | EIYMTFLSSK | 232 | 8 | Control |
| | | G-protein | | | | versus |
| | | signaling 10 | | | | MCI |
| 45.47801154 | SP-339 | Regulator of | LQDQIFNLMK | 233 | 9 | Control |
| | | G-protein | | | | versus |
| | | signaling 10 | | | | MCI |
| 44.25036008 | SP-339 | PROTEIN | LSVEIWDWDLTSR | 79 | 10 | Control |
| | | KINASE C | | | | versus |
| | | BETA TYPE | | | | MCI |
| 43.00574531 | SP-339 | COMPLEMENT C3 | AGDFLEANYMNLQR | 234 | 11 | Control |
| | | | | | | versus |
| | | | | | | MCI |
| 42.23286124 | SP-339 | TYROSINE- | YWPLYGEDPITFAPFK | 235 | 12 | Control |
| | | PROTEIN | | | | versus |
| | | PHOSPHATASE | | | | MCI |
| | | NON-RECEPTOR | | | | |
| | | TYPE 12 | | | | |
| 42.1749385 | SP-339 | SAA2-SAA4 | AYWDIMISNHQNSNR | 95 | 13 | Control |
| | | READTHROUGH | | | | versus |
| | | | | | | MCI |
| 41.12870802 | SP-339 | GTP-binding | TAEEICESSSK | 236 | 14 | Control |
| | | protein 2 | | | | versus |
| | | | | | | MCI |
| 40.64218506 | SP-339 | COMPLEMENT C3 | LVAYYTLIGASGQR | 237 | 15 | Control |
| | | | | | | versus |
| | | | | | | MCI |
| 40.41363247 | SP-339 | Hepatocyte | YIPYTLYSVFNPSDHD | 238 | 16 | Control |
| | | growth factor | LVLIR | | | versus |
| | | activator | | | | MCI |
| 38.76688065 | SP-339 | COMPLEMENT C3 | NTMILEICTR | 239 | 17 | Control |
| | | | | | | versus |
| | | | | | | MCI |
| 38.08001108 | SP-339 | T-plasminogen | VTNYLDWIRDNMRP | 106 | 18 | Control |
| | | activator | | | | versus |
| | | | | | | MCI |
| 37.93801219 | SP-339 | COMPLEMENT C3 | ENEGFTVTAEGK | 240 | 19 | Control |
| | | | | | | versus |
| | | | | | | MCI |
| 36.89405083 | SP-339 | LEUKOCYTE | LHEWTKPENLDFIEVN | 241 | 20 | Control |
| | | ELASTASE | VSLPR | | | versus |
| | | INHIBITOR | | | | MCI |
| 100 | SP-047 | COMPLEMENT C3 | DAPDHQELNLDVSLQL | 242 | 1 | Control |
| | | | PSR | | | versus |
| | | | | | | MCI |
| 49.55281413 | SP-047 | RHO GTPASE- | IVEVEQDNK | 243 | 2 | Control |
| | | ACTIVATING | | | | versus |
| | | PROTEIN 45 | | | | MCI |
| 48.68548382 | SP-047 | T-plasminogen | GGLFADIASHPWQAAI | 12 | 3 | Control |
| | | activator | FAK | | | versus |
| | | | | | | MCI |
| 48.56958459 | SP-047 | PDZ AND LIM | GHFFVEDQIYCEK | 119 | 4 | Control |
| | | DOMAIN | | | | versus |
| | | PROTEIN 1 | | | | MCI |
| 47.67346809 | SP-047 | MYOSIN | LSNDMMGSYAEMK | 18 | 5 | Control |
| | | APOLIPOPROTEIN | | | | versus |
| | | B-100LIGHT | | | | MCI |
| | | POLYPEPTIDE 9 | | | | |
| 44.74522742 | SP-047 | COMPLEMENT C3 | DFDFVPPVVR | 244 | 6 | Control |
| | | | | | | versus |
| | | | | | | MCI |
| 42.30980795 | SP-047 | MYOSIN-9 | KLEGDSTDLSDQIAEL | 245 | 7 | Control |
| | | | QAQIAELK | | | versus |
| | | | | | | MCI |
| 42.09549698 | SP-047 | TRANSGELIN-2 | TLMNLGGLAVAR | 246 | 8 | Control |
| | | | | | | versus |
| | | | | | | MCI |
| 39.47325164 | SP-047 | MYOSIN-9 | NMDPLNDNIATLLHQS | 247 | 9 | Control |
| | | | SDK | | | versus |
| | | | | | | MCI |
| 39.35673063 | SP-047 | T-plasminogen | VTNYLDWIRDNMRP | 106 | 10 | Control |
| | | activator | | | | versus |
| | | | | | | MCI |
| 38.25696187 | SP-047 | TRANSGELIN-2 | NVIGLQMGTNR | 111 | 11 | Control |
| | | | | | | versus |
| | | | | | | MCI |
| 38.25339765 | SP-047 | tRNA | WIADGQR | 248 | 12 | Control |
| | | (guanine(10)- | | | | versus |
| | | N2)-methyl- | | | | MCI |
| | | transferase | | | | |
| | | homolog | | | | |
| 34.06098898 | SP-047 | TRANSGELIN-2 | NMACVQR | 249 | 13 | Control |
| | | | | | | versus |
| | | | | | | MCI |
| 33.59486324 | SP-047 | O43294 | PYCQPCFLK | 250 | 14 | Control |
| | | | | | | versus |
| | | | | | | MCI |
| 33.27684836 | SP-047 | Septin | NLSLSGHVGFDSLPDQ | 251 | 15 | Control |
| | | | LVNK | | | versus |
| | | | | | | MCI |
| 32.97857573 | SP-047 | ADP- | LGQSVTTIPTVGFNVE | 252 | 16 | Control |
| | | RIBOSYLATION | TVTYK | | | versus |
| | | FACTOR 6 | | | | MCI |
| 32.21061778 | SP-047 | MYOSIN-9 | ANLQIDQINTDLNLER | 253 | 17 | Control |
| | | | | | | versus |
| | | | | | | MCI |
| 29.92017276 | SP-047 | INSULIN-LIKE | FFQYDTWK | 210 | 18 | Control |
| | | GROWTH FACTOR | | | | versus |
| | | II | | | | MCI |
| 29.69748301 | SP-047 | COMPLEMENT C3 | GYTQQLAFR | 254 | 19 | Control |
| | | | | | | versus |
| | | | | | | MCI |
| 29.55558918 | SP-047 | COAGULATION | AWGESTPLANKPGK | 255 | 20 | Control |
| | | FACTOR V | | | | versus |
| | | | | | | MCI |
| 100 | SP-053 | APOLIPOPROTEIN | ELLETVVNR | 23 | 1 | Control |
| | | C-II | | | | versus |
| | | | | | | MCI |
| 46.00711614 | SP-053 | TUBULIN | EIIDPVLDR | 199 | 2 | Control |
| | | ALPHA-4A | | | | versus |
| | | CHAIN | | | | MCI |
| 37.78867896 | SP-053 | TUBULIN | INVYYNEATGGK | 40 | 3 | Control |
| | | BETA-4B CHAIN | | | | versus |
| | | | | | | MCI |
| 34.9368689 | SP-053 | TUBULIN | EDAANNYAR | 256 | 4 | Control |
| | | ALPHA-1B | | | | versus |
| | | CHAIN | | | | MCI |
| 34.90887498 | SP-053 | ALPHA-2- | SPPGVCSR | 55 | 5 | Control |
| | | ANTIPLASMIN | | | | versus |
| | | | | | | MCI |
| 32.46226385 | SP-053 | ALPHA-2- | LGNQEPGGQTALK | 26 | 6 | Control |
| | | ANTIPLASMIN | | | | versus |
| | | | | | | MCI |
| 30.52614402 | SP-053 | CHROMOGRANIN- | EAVEEPSSK | 257 | 7 | Control |
| | | A | | | | versus |
| | | | | | | MCI |
| 29.98517026 | SP-053 | TUBULIN | EVDEQMLNVQNK | 258 | 8 | Control |
| | | BETA-4B CHAIN | | | | versus |
| | | | | | | MCI |
| 28.7914205 | SP-053 | Complement | PVNPVEQR | 259 | 9 | Control |
| | | subcomponent | | | | versus |
| | | C1r | | | | MCI |
| 28.19277735 | SP-053 | CHROMOGRANIN- | AEGNNQAPGEEEEEEE | 260 | 10 | Control |
| | | A | EATNTHPPASLPSQK | | | versus |
| | | | | | | MCI |
| 28.08138931 | SP-053 | ZYXIN-2 | PLSIEADDNGCFPLDG | 124 | 11 | Control |
| | | | HVLCR | | | versus |
| | | | | | | MCI |
| 27.4320136 | SP-053 | TUBULIN | RAFVHWYVGEGMEEGE | 261 | 12 | Control |
| | | ALPHA-1B | FSEAR | | | versus |
| | | CHAIN | | | | MCI |
| 26.57053589 | SP-053 | TUBULIN | EVDQQLLSVQTR | 262 | 13 | Control |
| | | BETA-1 CHAIN | | | | versus |
| | | | | | | MCI |
| 26.4751614 | SP-053 | Complement | DYFIATCK | 129 | 14 | Control |
| | | subcomponent | | | | versus |
| | | C1r | | | | MCI |
| 26.20196072 | SP-053 | ALPHA-1- | GVCEETSGAYEK | 263 | 15 | Control |
| | | MICROGLOBULIN/ | | | | versus |
| | | BIKUNIN | | | | MCI |
| | | PRECURSOR | | | | Control |
| 25.93549517 | SP-053 | TUBULIN | DVNAAIAAIK | 264 | 16 | versus |
| | | ALPHA-4A | | | | MCI |
| | | CHAIN | | | | Control |
| 24.93121346 | SP-053 | TUBULIN | FDLMYAK | 51 | 17 | versus |
| | | ALPHA-1B | | | | MCI |
| | | CHAIN | | | | Control |
| 24.78470488 | SP-053 | Complement | NIGEFCGK | 265 | 18 | versus |
| | | subcomponent | | | | MCI |
| | | C1r | | | | Control |
| 23.09079081 | SP-053 | CHROMOGRANIN- | EEEEEMAVVPQGLFR | 201 | 19 | versus |
| | | A | | | | MCI |
| | | | | | | Control |
| 22.93044943 | SP-053 | TUBULIN | FWEVISDEHGIDPTGT | 126 | 20 | versus |
| | | BETA-4B CHAIN | YHGDSDLQLER | | | MCI |
| | | | | | | Control |
| 100 | SP-373 | APOLIPOPROTEIN | ELLETVVNR | 23 | 1 | versus |
| | | C-II | | | | MCI |
| | | | | | | Control |
| 31.24348961 | SP-373 | GLYCERALDEHYDE- | VIISAPSADAPMFVMG | 193 | 2 | versus |
| | | 3-PHOSPHATE | VNHEK | | | MCI |
| | | DEHYDROGENASE | | | | Control |
| 28.79520818 | SP-373 | PEPTIDYL- | SEETLDEGPPK | 266 | 3 | versus |
| | | PROLYLCIS- | | | | MCI |
| | | TRANSISOMERASE | | | | Control |
| | | FKBP3 | | | | |
| 28.74504836 | SP-373 | RAS GTPASE- | TEISLVLTSK | 267 | 4 | Control |
| | | ACTIVATING- | | | | versus |
| | | LIKE PROTEIN | | | | MCI |
| | | IQGAP2 | | | | |
| 27.35087686 | SP-373 | ELASTIN | VLDSEGQLR | 268 | 5 | Control |
| | | MICROFIBRIL | | | | versus |
| | | INTERFACE- | | | | MCI |
| | | LOCATED | | | | |
| | | PROTEIN 1 | | | | |
| | | (EMILIN-1) | | | | |
| 25.68545393 | SP-373 | CALCIUM | VSYLQLSFWK | 269 | 6 | Control |
| | | HOMEOSTASIS | | | | versus |
| | | MODULATOR | | | | MCI |
| | | PROTEIN 5 | | | | |
| 25.58647037 | SP-373 | PROHIBITIN | QVSDDLTER | 270 | 7 | Control |
| | | | | | | versus |
| | | | | | | MCI |
| 25.29497611 | SP-373 | MYOSIN | MDMTFSK | 271 | 8 | Control |
| | | APOLIPOPROTEIN | | | | versus |
| | | B-100LIGHT | | | | MCI |
| | | POLYPEPTIDE 9 | | | | |
| 24.8340514 | SP-373 | ANNEXIN A7 | SEIDLVQIK | 272 | 9 | Control |
| | | | | | | versus |
| | | | | | | MCI |
| 24.80495213 | SP-373 | RHO GTPASE- | NLCQELEAK | 273 | 10 | Control |
| | | ACTIVATING | | | | versus |
| | | PROTEIN 18 | | | | MCI |
| 24.16611838 | SP-373 | HIGH MOBILITY | SEHPGLSIGDTAK | 274 | 11 | Control |
| | | GROUP PROTEIN | | | | versus |
| | | B2 | | | | MCI |
| 23.73543475 | SP-373 | RECEPTOR-TYPE | YVDILPYDYNR | 275 | 12 | Control |
| | | TYROSINE- | | | | versus |
| | | PROTEIN | | | | MCI |
| | | PHOSPHATASE C | | | | |
| 22.69462844 | SP-373 | HEXOKINASE-1 | TTVGVDGSLYK | 276 | 13 | Control |
| | | | | | | versus |
| | | | | | | MCI |
| 22.08420764 | SP-373 | GLYCERALDEHYDE- | VPTANVSVVDLTCRLE | 277 | 14 | Control |
| | | 3-PHOSPHATE | K | | | versus |
| | | DEHYDROGENASE | | | | MCI |
| 21.91196133 | SP-373 | Coagulation | SQHLDNFSNQIGK | 143 | 15 | Control |
| | | factor V | | | | versus |
| | | | | | | MCI |
| 21.64778699 | SP-373 | RAS GTPASE- | DLNLMDIK | 278 | 16 | Control |
| | | ACTIVATING-LIKE | | | | versus |
| | | PROTEIN IQGAP2 | | | | MCI |
| 21.30689802 | SP-373 | TALIN-1 | TMQFEPSTMVYDACR | 279 | 17 | Control |
| | | | | | | versus |
| | | | | | | MCI |
| 20.85877432 | SP-373 | FIBRONECTIN | FGFCPMAAHEEICTTN | 280 | 18 | Control |
| | | | EGVMYR | | | versus |
| | | | | | | MCI |
| 20.78658291 | SP-373 | RAS GTPASE- | AAFYEEQINYYDTYIK | 281 | 19 | Control |
| | | ACTIVATING-LIKE | | | | versus |
| | | PROTEIN IQGAP2 | | | | MCI |
| 20.54304151 | SP-373 | GLYCERALDEHYDE- | WGDAGAEYVVESTGVF | 282 | 20 | Control |
| | | 3-PHOSPHATE | TTMEK | | | versus |
| | | DEHYDROGENASE | | | | MCI |
| 100 | SP-089 | T-plasminogen | GGLFADIASHPNVQAA | 12 | 1 | Control |
| | | activator | IFAK | | | versus |
| | | | | | | MCI |
| 53.64689031 | SP-089 | KINESIN-LIKE | GSLDYRPLTTADPIDE | 164 | 2 | Control |
| | | PROTEIN KIF2A | HR | | | versus |
| | | | | | | MCI |
| 48.67064799 | SP-089 | TRANSTHYRETIN | TSESGELHGLTTEEEF | 122 | 3 | Control |
| | | | VEGIYK | | | versus |
| | | | | | | MCI |
| 45.6749048 | SP-089 | PLASMINOGEN | TPENFPCK | 283 | 4 | Control |
| | | | | | | versus |
| | | | | | | MCI |
| 42.8810212 | SP-089 | INSULIN-LIKE | FFQYDTWK | 210 | 5 | Control |
| | | GROWTH FACTOR | | | | versus |
| | | II | | | | MCI |
| 42.72323632 | SP-089 | APOLIPOPROTEIN | ELLETVVNR | 23 | 6 | Control |
| | | C-II | | | | versus |
| | | | | | | MCI |
| 38.67363729 | SP-089 | Complement | LPVANPQACENWLR | 205 | 7 | Control |
| | | subcomponent | | | | versus |
| | | C1r | | | | MCI |
| 38.31219904 | SP-089 | PDZ AND LIM | VAASIGNAQK | 284 | 8 | Control |
| | | DOMAIN | | | | versus |
| | | PROTEIN 1 | | | | MCI |
| 38.23161839 | SP-089 | HISTIDINE-RICH | GEVLPLPEANFPSFPL | 72 | 9 | Control |
| | | GLYCOPROTEIN | PHHK | | | versus |
| | | | | | | MCI |
| 36.52786957 | SP-089 | HISTIDINE-RICH | YWNDCEPPDSR | 70 | 10 | Control |
| | | GLYCOPROTEIN | | | | versus |
| | | | | | | MCI |
| 36.36910782 | SP-089 | COMPLEMENT C3 | ADIGCTPGSGK | 285 | 11 | Control |
| | | | | | | versus |
| | | | | | | MCI |
| 36.35545137 | SP-089 | RHO GTPASE- | LQLFGQDFSHAAR | 214 | 12 | Control |
| | | ACTIVATING | | | | versus |
| | | PROTEIN 45 | | | | MCI |
| 35.98716156 | SP-089 | HISTIDINE-RICH | VIDFNCTTSSVSSALA | 75 | 13 | Control |
| | | GLYCOPROTEIN | NTK | | | versus |
| | | | | | | MCI |
| 34.65400672 | SP-089 | COMPLEMENT C3 | TVMVNIENPEGIPVK | 182 | 14 | Control |
| | | | | | | versus |
| | | | | | | MCI |
| 34.1500677 | SP-089 | TRANSTHYRETIN | ALGISPFHEHAEVVFT | 7 | 15 | Control |
| | | | ANDSGPR | | | versus |
| | | | | | | MCI |
| 34.05993098 | SP-089 | COMPLEMENT C3 | VYAYYNLEESCTR | 286 | 16 | Control |
| | | | | | | versus |
| | | | | | | MCI |
| 33.78381011 | SP-089 | HISTIDINE-RICH | RDGYLFQLLR | 156 | 17 | Control |
| | | GLYCOPROTEIN | | | | versus |
| | | | | | | MCI |
| 33.69678158 | SP-089 | COMPLEMENT C3 | ENEGFTVTAEGK | 240 | 18 | Control |
| | | | | | | versus |
| | | | | | | MCI |
| 33.39201728 | SP-089 | T-plasminogen | GTHSLTESGASCLPWN | 287 | 19 | Control |
| | | activator | SMILIGK | | | versus |
| | | | | | | MCI |
| 32.40288166 | SP-089 | HISTIDINE-RICH | DHSHGPPLPQGPPPLL | 152 | 20 | Control |
| | | GLYCOPROTEIN | PMSCSSCQHATFGTNG | | | versus |
| | | | AQR | | | MCI |
| 100 | SP-003 | APOLIPOPROTEIN | GEVQAMLGQSTEELR | 66 | 1 | MCI |
| | | E | | | | versus |
| | | | | | | AD |
| 82.0187898 | SP-003 | APOLIPOPROTEIN | GEVQAMLGQSTEELRV | 288 | 2 | MCI |
| | | E | R | | | versus |
| | | | | | | AD |
| 66.8886223 | SP-003 | HISTIDINE-RICH | KYWNDCEPPDSR | 215 | 3 | MCI |
| | | GLYCOPROTEIN | | | | versus |
| | | | | | | AD |
| 64.64582858 | SP-003 | HEPARIN | TLEAQLTPR | 6 | 4 | MCI |
| | | COFACTOR 2 | | | | versus |
| | | | | | | AD |
| 59.45066708 | SP-003 | APOLIPOPROTEIN | LVQYRGEVQAMLGQST | 289 | 5 | MCI |
| | | E | EELR | | | versus |
| | | | | | | AD |
| 54.93863485 | SP-003 | APOLIPOPROTEIN | WVQTLSEQVQEELLSS | 290 | 6 | MCI |
| | | E | QVTQELR | | | versus |
| | | | | | | AD |
| 53.39734339 | SP-003 | FRUCTOSE- | YASICQQNGIVPIVEP | 291 | 7 | MCI |
| | | BISPHOSPHATE | EILPDGDHDLK | | | versus |
| | | ALDOLASE A | | | | AD |
| 52.89089507 | SP-003 | COMPLEMENT C3 | TGLQEVEVK | 292 | 8 | MCI |
| | | | | | | versus |
| | | | | | | AD |
| 51.72380853 | SP-003 | COMPLEMENT C3 | VELLHNPAFCSLATTK | 293 | 9 | MCI |
| | | | | | | versus |
| | | | | | | AD |
| 49.16183793 | SP-003 | Dynamin GTPase | SSVLENFVGR | 294 | 10 | MCI |
| | | | | | | versus |
| | | | | | | AD |
| 49.16035546 | SP-003 | HEPARIN | FAFNLYR | 295 | 11 | MCI |
| | | COFACTOR 2 | | | | versus |
| | | | | | | AD |
| 49.02328166 | SP-003 | MYELOPEROXIDASE | NQINALTSFVDASMVY | 296 | 12 | MCI |
| | | | GSEEPLAR | | | versus |
| | | | | | | AD |
| 48.85635954 | SP-003 | CREATINE KINASE | ELFDPIISDR | 297 | 13 | MCI |
| | | M-TYPE | | | | versus |
| | | | | | | AD |
| 48.79475279 | SP-003 | CORONIN-1A | QVALWDTK | 298 | 14 | MCI |
| | | | | | | versus |
| | | | | | | AD |
| 48.78070473 | SP-003 | IMMUNOGLOBULIN | DSTYSLSSTLTLSK | 299 | 15 | MCI |
| | | KAPPA CONSTANT | | | | versus |
| | | | | | | AD |
| 48.12320759 | SP-003 | APOLIPOPROTEIN | QQTEWQSGQR | 300 | 16 | MCI |
| | | E | | | | versus |
| | | | | | | AD |
| 47.89480637 | SP-003 | VITRONECTIN | LIRDVWGIEGPIDAAF | 301 | 17 | MCI |
| | | | TR | | | versus |
| | | | | | | AD |
| 46.89969004 | SP-003 | COMPLEMENT C3 | SGSDEVQVGQQR | 168 | 18 | MCI |
| | | | | | | versus |
| | | | | | | AD |
| 46.25688128 | SP-003 | HISTIDINE-RICH | LPPLRKGEVLPLPEAN | 302 | 19 | MCI |
| | | GLYCOPROTEIN | FPSFPLPHHK | | | versus |
| | | | | | | AD |
| 45.77845972 | SP-003 | Lipoprotein | GLGDVDQLVK | 303 | 20 | MCI |
| | | lipase | | | | versus |
| | | | | | | AD |
| 100 | SP-006 | APOLIPOPROTEIN | LTPYADEFK | 304 | 1 | MCI |
| | | A-IV | | | | versus |
| | | | | | | AD |
| 96.87707759 | SP-006 | ALPHA-2- | VSNQTLSLFFTVLQDV | 305 | 2 | MCI |
| | | MACROGLOBUL1N | PVRDLKPAIVK | | | versus |
| | | | | | | AD |
| 94.04369919 | SP-006 | HEPARIN | FAFNLYR | 295 | 3 | MCI |
| | | COFACTOR 2 | | | | versus |
| | | | | | | AD |
| 86.15650112 | SP-006 | FIBRONECTIN | TFYSCTTEGR | 306 | 4 | MCI |
| | | | | | | versus |
| | | | | | | AD |
| 81.49136186 | SP-006 | APOLIPOPROTEIN | VLRENADSLQASLRPH | 307 | 5 | MCI |
| | | A-IV | ADELK | | | versus |
| | | | | | | AD |
| 74.14025432 | SP-006 | COMPLEMENT | YGFCEAADQFHVLDEV | 308 | 6 | MCI |
| | | COMPONENT C8 | R | | | versus |
| | | GAMMA CHAIN | | | | AD |
| 72.44799623 | SP-006 | COMPLEMENT | NSGLTEEEAK | 309 | 7 | MCI |
| | | COMPONENT C6 | | | | versus |
| | | | | | | AD |
| 71.3219151 | SP-006 | APOLIPOPROTEIN | AELQEGAR | 310 | 8 | MCI |
| | | A-I | | | | versus |
| | | | | | | AD |
| 71.1667507 | SP-006 | APOLIPOPROTEIN | DRLDEVK | 311 | 9 | MCI |
| | | E | | | | versus |
| | | | | | | AD |
| 71.08440946 | SP-006 | Neuropilin | FVSDYETHGAGFSIR | 312 | 10 | MCI |
| | | | | | | versus |
| | | | | | | AD |
| 68.938054 | SP-006 | Thymidine | MLAAQGVDPGLAR | 313 | 11 | MCI |
| | | phosphorylase | | | | versus |
| | | | | | | AD |
| 68.86443502 | SP-006 | MYOSIN | SVGFHLPSR | 314 | 12 | MCI |
| | | APOLIPOPROTEIN | | | | versus |
| | | B-100LIGHT | | | | AD |
| | | POLYPEPTIDE 9 | | | | |
| 67.73667821 | SP-006 | Alpha-enolase | LMIEMDGTENK | 315 | 13 | MCI |
| | | | | | | versus |
| | | | | | | AD |
| 67.10683008 | SP-006 | L-LACTATE | LKDDEVAQLK | 316 | 14 | MCI |
| | | DEHYDROGENASE | | | | versus |
| | | B CHAIN | | | | AD |
| 65.12473024 | SP-006 | IMMUNOGLOBULIN | AEDTAVYYCAR | 317 | 15 | MCI |
| | | HEAVY VARIABLE | | | | versus |
| | | 3-33 | | | | AD |
| 64.76594886 | SP-006 | BETA-ALA-HIS | YPSLSIHGIEGAFDEP | 318 | 16 | MCI |
| | | DIPEPTIDASE | GTK | | | versus |
| | | | | | | AD |
| 64.47641449 | SP-006 | HEPARIN | FTVDRPFLFLIYEHR | 319 | 17 | MCI |
| | | COFACTOR 2 | | | | versus |
| | | | | | | AD |
| 64.3443722 | SP-006 | FIBRONECTIN | EINLAPDSSSVVVSGL | 320 | 18 | MCI |
| | | | MVATK | | | versus |
| | | | | | | AD |
| 64.27187839 | SP-006 | TENASCIN-X | EEPPRPEFLEQPLLGE | 321 | 19 | MCI |
| | | | LTVTGVTPDSLR | | | versus |
| | | | | | | AD |
| 64.06630655 | SP-006 | ADIPONECTIN | IFYNQQNHYDGSTGK | 322 | 20 | MCI |
| | | | | | | versus |
| | | | | | | AD |
| 100 | SP-007 | INSULIN-LIKE | PLHTLMHGQGVCMELA | 323 | 1 | MCI |
| | | GROWTH FACTOR- | EIEAIQESLQPSDKDE | | | versus |
| | | BINDING | GDHPNNSFSPCSAHDR | | | AD |
| | | PROTEIN 4 | R | | | |
| 70.08596243 | SP-007 | APPETITE- | FNAPFDVGIK | 324 | 2 | MCI |
| | | REGULATING | | | | versus |
| | | HORMONE | | | | AD |
| 63.7630777 | SP-007 | HEPARIN | GGETAQSADPQWEQLN | 325 | 3 | MCI |
| | | COFACTOR 2 | NK | | | versus |
| | | | | | | AD |
| 63.28074817 | SP-007 | Immunoglobulin | PGQSPQLLIYLGSNR | 326 | 4 | MCI |
| | | kappa variable | | | | versus |
| | | 2-28 | | | | AD |
| 62.42456732 | SP-007 | APOLIPOPROTEIN | GEVQAMLGQSTEELRV | 288 | 5 | MCI |
| | | E | R | | | versus |
| | | | | | | AD |
| 60.81344386 | SP-007 | COAGULATION | AQMDLSGR | 327 | 6 | MCI |
| | | FACTOR XIII | | | | versus |
| | | A CHAIN | | | | AD |
| 60.0399555 | SP-007 | INVERTED | CSNEEVAAMIR | 328 | 7 | MCI |
| | | FORMIN-2 | | | | versus |
| | | | | | | AD |
| 59.83230328 | SP-007 | COMPLEMENT | CIHPCIITEENMNK | 329 | 8 | MCI |
| | | FACTOR H- | | | | versus |
| | | RELATED | | | | AD |
| | | PROTEIN 4 | | | | |
| 59.70518863 | SP-007 | PEROXIREDOXIN- | ATAVMPDGQFK | 330 | 9 | MCI |
| | | 1 | | | | versus |
| | | | | | | AD |
| 59.65335633 | SP-007 | Protein | EVLLEVQK | 331 | 10 | MCI |
| | | kinase C | | | | versus |
| | | and casein | | | | AD |
| | | kinase | | | | |
| | | substrate in | | | | |
| | | neurons | | | | |
| | | protein 2 | | | | |
| 59.51111759 | SP-007 | PLASMINOGEN | LFLEPTRK | 332 | 11 | MCI |
| | | | | | | versus |
| | | | | | | AD |
| 57.89837591 | SP-007 | RHO GTPASE- | DLYQLNPNAEWVIK | 333 | 12 | MCI |
| | | ACTIVATING | | | | versus |
| | | PROTEIN 18 | | | | AD |
| 57.79048777 | SP-007 | CHROMOGRANIN- | EDSLEAGLPLQVR | 334 | 13 | MCI |
| | | A | | | | versus |
| | | | | | | AD |
| 56.83996559 | SP-007 | Vitronectin | CTEGFNVDK | 335 | 14 | MCI |
| | | | | | | versus |
| | | | | | | AD |
| 56.65459231 | SP-007 | All-trans- | IIGIDINSEK | 336 | 15 | MCI |
| | | retinol | | | | versus |
| | | dehydrogenase | | | | AD |
| 55.90926583 | SP-007 | PEROXIREDOXIN- | IRFHDFLGDSWGILFS | 337 | 16 | MCI |
| | | 6 | HPR | | | versus |
| | | | | | | AD |
| 55.44353831 | SP-007 | CALNEXIN | GTLSGWILSK | 338 | 17 | MCI |
| | | | | | | versus |
| | | | | | | AD |
| 54.85014123 | SP-007 | THROMBOSPONDIN- | GAGSLELYLDCIQVDS | 339 | 18 | MCI |
| | | 4 | VHNLPR | | | versus |
| | | | | | | AD |
| 54.70488696 | SP-007 | APOLIPOPROTEIN | SLAELGGHLDQQVEEF | 340 | 19 | MCI |
| | | A-IV | RR | | | versus |
| | | | | | | AD |
| 54.64459824 | SP-007 | DIHYDRO- | PVAIGGK | 341 | 20 | MCI |
| | | LIPOYLLYSINE- | | | | versus |
| | | RESIDUE | | | | AD |
| | | SUCCINYL- | | | | |
| | | TRANSFERASE | | | | |
| | | COMPONENT OF | | | | |
| | | 2-OXOGLUTARATE | | | | |
| | | DEHYDROGENASE | | | | |
| | | COMPLEX | | | | |
| 100 | SP-008 | SECRETED | DYYVSTAVCR | 342 | 1 | MCI |
| | | PHOSPHOPROTEIN | | | | versus |
| | | 24 | | | | AD |
| 98.65507932 | SP-008 | APOLIPOPROTEIN | DRLDEVK | 311 | 2 | MCI |
| | | E | | | | versus |
| | | | | | | AD |
| 93.55240523 | SP-008 | APOLIPOPROTEIN | ELQAAQAR | 343 | 3 | MCI |
| | | E | | | | versus |
| | | | | | | AD |
| 85.23776975 | SP-008 | ADAM DEC1 | HLLGPDYTETLYSPR | 344 | 4 | MCI |
| | | | | | | versus |
| | | | | | | AD |
| 84.14630823 | SP-008 | 59 kDa serine/ | EVPFADLSNMEIGMK | 345 | 5 | MCI |
| | | threonine- | | | | versus |
| | | protein kinase | | | | AD |
| 84.03167104 | SP-008 | Immunoglobulin | GLEWIGYIYYSGSTNY | 346 | 6 | MCI |
| | | heavy variable | NPSLK | | | versus |
| | | 4-61 | | | | AD |
| 80.29721263 | SP-008 | CORONIN-1C | VTWDSSFCAVNPR | 347 | 7 | MCI |
| | | | | | | versus |
| | | | | | | AD |
| 77.78166551 | SP-008 | APOLIPOPROTEIN | LEEQAQQIR | 348 | 8 | MCI |
| | | E | | | | versus |
| | | | | | | AD |
| 77.12366467 | SP-008 | APOLIPOPROTEIN | LLPHANEVSQK | 349 | 9 | MCI |
| | | A-IV | | | | versus |
| | | | | | | AD |
| 75.8488657 | SP-008 | CLUSTERIN | RELDESLQVAER | 350 | 10 | MCI |
| | | | | | | versus |
| | | | | | | AD |
| 74.76145716 | SP-008 | APOLIPOPROTEIN | LAVYQAGAR | 351 | 11 | MCI |
| | | E | | | | versus |
| | | | | | | AD |
| 73.98569797 | SP-008 | CYSTATIN-C | RALDFAVGEYNK | 352 | 12 | MCI |
| | | | | | | versus |
| | | | | | | AD |
| 72.74660023 | SP-008 | PROTEIN | EAAQFAR | 353 | 13 | MCI |
| | | PHOSPHATASE ID | | | | versus |
| | | | | | | AD |
| 71.77697804 | SP-008 | HAPTOGLOBIN | NPANPVQR | 354 | 14 | MCI |
| | | | | | | versus |
| | | | | | | AD |
| 71.68113743 | SP-008 | FIBRONECTIN | FLATTPNSLLVSWQPP | 355 | 15 | MCI |
| | | | R | | | versus |
| | | | | | | AD |
| 71.48559542 | SP-008 | HEPARIN | NFGYTLR | 356 | 16 | MCI |
| | | COFACTOR 2 | | | | versus |
| | | | | | | AD |
| 71.05250257 | SP-008 | LACTO- | SEEEVAAR | 357 | 17 | MCI |
| | | TRANSFERRIN | | | | versus |
| | | | | | | AD |
| 70.85100243 | SP-008 | G protein- | ISDLGLAVHVPEGQTI | 358 | 18 | MCI |
| | | coupled | K | | | versus |
| | | receptor | | | | AD |
| | | kinase | | | | |
| 70.776017 | SP-008 | APOLIPOPROTEIN | LEPYADQLR | 359 | 19 | MCI |
| | | A-IV | | | | versus |
| | | | | | | AD |
| 70.31298681 | SP-008 | ALPHA-1- | FLENEDRR | 360 | 20 | MCI |
| | | ANTITRYPSIN | | | | versus |
| | | | | | | AD |
| 100 | SP-033 | PROTHROMBIN | ETWTANVGK | 361 | 1 | MCI |
| | | | | | | versus |
| | | | | | | AD |
| 98.93456816 | SP-033 | Immunoglobulin | WQQGNIFSCSVMHEAL | 362 | 2 | MCI |
| | | heavy constant | HNR | | | versus |
| | | gamma 3 | | | | AD |
| 90.94141161 | SP-033 | Coagulation | YLDSTFTK | 363 | 3 | MCI |
| | | factor V | | | | versus |
| | | | | | | AD |
| 87.98574023 | SP-033 | MYELOPEROXIDASE | AVSNEIVR | 364 | 4 | MCI |
| | | | | | | versus |
| | | | | | | AD |
| 84.13045541 | SP-033 | WASAVASL- | GAPPLPPIPR | 365 | 5 | MCI |
| | | INTERACTING | | | | versus |
| | | PROTEIN | | | | AD |
| | | FAMILY | | | | |
| | | MEMBER 1 | | | | |
| 82.84495261 | SP-033 | Alpha-atrial | NLLDHLEEK | 366 | 6 | MCI |
| | | natriuretic | | | | versus |
| | | peptide | | | | AD |
| 78.61738354 | SP-033 | FATTY ACID- | PNMIISVNGDVITIK | 367 | 7 | MCI |
| | | BINDING | | | | versus |
| | | PROTEIN, | | | | AD |
| | | ADIPOCYTE | | | | |
| 75.68680096 | SP-033 | Coagulation | WIISSLTPK | 368 | 8 | MCI |
| | | factor V | | | | versus |
| | | | | | | AD |
| 75.30412127 | SP-033 | VITRONECTIN | GNPEQTPVLK | 369 | 9 | MCI |
| | | | | | | versus |
| | | | | | | AD |
| 73.87776969 | SP-033 | Coagulation | ENQFDPPIVAR | 370 | 10 | MCI |
| | | factor V | | | | versus |
| | | | | | | AD |
| 73.20687953 | SP-033 | INTER-ALPHA- | PGLDHTEASFSPR | 371 | 11 | MCI |
| | | TRYPSIN | | | | versus |
| | | INHIBITOR | | | | AD |
| | | HEAVY CHAIN | | | | |
| | | H4 (ITIH4) | | | | |
| | | PROTEIN | | | | |
| 72.56633908 | SP-033 | COMPLEMENT C3 | LESEETMVLEAHDAQG | 372 | 12 | MCI |
| | | | DVPVTVTVHDFPGKK | | | versus |
| | | | | | | AD |
| 72.29108495 | SP-033 | MANNOSYL- | FDGGVEAIATR | 373 | 13 | MCI |
| | | OLIGO- | | | | versus |
| | | SACCHARIDE | | | | AD |
| | | 1,2-ALPHA- | | | | |
| | | MANNOSIDASE | | | | |
| | | IA | | | | |
| 72.10758221 | SP-033 | TRANSFORMING | ILGDPEALRDLLNNHI | 374 | 14 | MCI |
| | | GROWTH FACTOR- | LK | | | versus |
| | | BETA-INDUCED | | | | AD |
| | | PROTEIN IG-H3 | | | | |
| 71.969007 | SP-033 | KINESIN-LIKE | LIDIGNSCR | 181 | 15 | MCI |
| | | PROTEIN KIF2A | | | | versus |
| | | | | | | AD |
| 71.78581733 | SP-033 | COMPLEMENT C3 | ENEGFTVTAEGK | 240 | 16 | MCI |
| | | | | | | versus |
| | | | | | | AD |
| 71.5286681 | SP-033 | COAGULATION | LAAEFASK | 375 | 17 | MCI |
| | | FACTOR V | | | | versus |
| | | | | | | AD |
| 71.49636476 | SP-033 | COAGULATION | EYTYEWSISEDSGPTH | 376 | 18 | MCI |
| | | FACTOR V | DDPPCLTHIYYSHENL | | | versus |
| | | | IEDFNSGLIGPLLICK | | | AD |
| 71.1772234 | SP-033 | COAGULATION | HEDTLTLFPMR | 377 | 19 | MCI |
| | | FACTOR V | | | | versus |
| | | | | | | AD |
| 71.05488574 | SP-033 | IMMUNOGLOBULIN | NTLYLQMSSLR | 378 | 20 | MCI |
| | | HEAVY VARIABLE | | | | versus |
| | | 3-64D | | | | AD |
| 100 | SP-339 | APOLIPOPROTEIN | LGEVNTYAGDLQK | 379 | 1 | MCI |
| | | A-IV | | | | versus |
| | | | | | | AD |
| 87.55144981 | SP-339 | COMPLEMENT C3 | VTLEERLDK | 380 | 2 | MCI |
| | | | | | | versus |
| | | | | | | AD |
| 84.34427086 | SP-339 | COMPLEMENT C3 | CCEDGMRENPMR | 381 | | MCI |
| | | | | | | versus |
| | | | | | | AD |
| 83.72084468 | SP-339 | APOLIPOPROTEIN | SLAPYAQDTQEK | 382 | 4 | MCI |
| | | A-IV | | | | versus |
| | | | | | | AD |
| 77.59715734 | SP-339 | APOLIPOPROTEIN | IDQNVEELK | 383 | 5 | MCI |
| | | A-IV | | | | versus |
| | | | | | | AD |
| 76.22733698 | SP-339 | HEPARIN | TLEAQLTPR | 6 | 6 | MCI |
| | | COFACTOR 2 | | | | versus |
| | | | | | | AD |
| 69.73548208 | SP-339 | COMPLEMENT C3 | ENEGFTVTAEGK | 240 | 7 | MCI |
| | | | | | | versus |
| | | | | | | AD |
| 69.36805278 | SP-339 | INTER-ALPHA- | LPEGSVSLIILLTDGD | 384 | 8 | MCI |
| | | TRYPSIN | PTVGETNPR | | | versus |
| | | INHIBITOR | | | | AD |
| | | HEAVY CHAIN | | | | |
| | | H4 (ITIH4) | | | | |
| | | PROTEIN | | | | |
| 67.6682195 | SP-339 | FIBRONECTIN | GEWTCIAYSQLR | 385 | 9 | MCI |
| | | | | | | versus |
| | | | | | | AD |
| 65.72573295 | SP-339 | COMPLEMENT C3 | VYAYYNLEESCTR | 286 | 10 | MCI |
| | | | | | | versus |
| | | | | | | AD |
| 60.99078071 | SP-339 | ALPHA-2- | LHTEAQIQEEGTVVEL | 386 | 11 | MCI |
| | | MACROGLOBULIN | TGR | | | versus |
| | | | | | | AD |
| 60.49278383 | SP-339 | COMPLEMENT C5 | ADNFLLENTLPAQSTF | 387 | 12 | MCI |
| | | | TLAISAYALSLGDK | | | versus |
| | | | | | | AD |
| 60.05733118 | SP-339 | TRANSALDOLASE | LLGELLQDNAK | 388 | 13 | MCI |
| | | | | | | versus |
| | | | | | | AD |
| 58.73876563 | SP-339 | COMPLEMENT C3 | GLEVTITAR | 389 | 14 | MCI |
| | | | | | | versus |
| | | | | | | AD |
| 57.98487578 | SP-339 | CARTILAGE | GTFTLHVPQDTER | 390 | 15 | MCI |
| | | INTERMEDIATE | | | | versus |
| | | LAYER PROTEIN | | | | AD |
| | | 1 | | | | |
| 57.91043825 | SP-339 | COMPLEMENT C3 | TVMVNIENPEGIPVK | 182 | 16 | MCI |
| | | | | | | versus |
| | | | | | | AD |
| 57.56493854 | SP-339 | Major prion | VVEQMCITQYER | 391 | 17 | MCI |
| | | protein | | | | versus |
| | | | | | | AD |
| 56.52952931 | SP-339 | Tetranectin | SRLDTLAQEVALLK | 392 | 18 | MCI |
| | | | | | | versus |
| | | | | | | AD |
| 55.24545418 | SP-339 | COMPLEMENT C3 | VHQYFNVELIQPGAVK | 169 | 19 | MCI |
| | | | | | | versus |
| | | | | | | AD |
| 54.82105638 | SP-339 | LACTO- | DGAGDVAFIR | 393 | 20 | MCI |
| | | TRANSFERRIN | | | | versus |
| | | | | | | AD |
| 100 | SP-047 | DNAJ HOMOLOG | LIESAEELIR | 394 | 1 | MCI |
| | | SUBFAMILY C | | | | versus |
| | | MEMBER 3 | | | | AD |
| 73.74623937 | SP-047 | Hepatocyte | NPDGSEAPWCFTLRPG | 395 | 2 | MCI |
| | | growth factor- | MR | | | versus |
| | | like protein | | | | AD |
| 68.99711966 | SP-047 | ZYXIN-2 | EVEELEQLTQQLMQDM | 396 | 3 | MCI |
| | | | EHPQR | | | versus |
| | | | | | | AD |
| 66.82969551 | SP-047 | APOLIPOPROTEIN | SWFEPLVEDMQRQWAG | 397 | 4 | MCI |
| | | E | LVEK | | | versus |
| | | | | | | AD |
| 64.72339084 | SP-047 | FIBRONECTIN | PISINYR | 398 | 5 | MCI |
| | | | | | | versus |
| | | | | | | AD |
| 64.35711993 | SP-047 | FIBRINOGEN | ALTDMPQMR | 399 | 6 | MCI |
| | | ALPHA CHAIN | | | | versus |
| | | | | | | AD |
| 62.31198364 | SP-047 | LATENT- | EIPSLDQEK | 400 | 7 | MCI |
| | | TRANSFORMING | | | | versus |
| | | GROWTH FACTOR | | | | AD |
| | | BETA-BINDING | | | | |
| | | PROTEIN 1 | | | | |
| 60.17561975 | SP-047 | APOLIPOPROTEIN | LHELQEK | 401 | 8 | MCI |
| | | A-I | | | | versus |
| | | | | | | AD |
| 58.43738662 | SP-047 | LACTO- | FFSASCVPGADK | 402 | 9 | MCI |
| | | TRANSFERRIN | | | | versus |
| | | | | | | AD |
| 57.77409199 | SP-047 | Neutrophil | WYVVGLAGNAILR | 403 | 10 | MCI |
| | | gelatinase- | | | | versus |
| | | associated | | | | AD |
| | | lipocalin | | | | |
| 57.6915553 | SP-047 | GLUTAMATE | HGGTIPIVPTAEFQDR | 404 | 11 | MCI |
| | | DEHYDROGENASE 1 | | | | versus |
| | | | | | | AD |
| 55.47479792 | SP-047 | COAGULATION | SWWGDYWEPFR | 405 | 12 | MCI |
| | | FACTOR V | | | | versus |
| | | | | | | AD |
| 55.36236137 | SP-047 | MYOSIN | DFSAEYEEDGKYEGLQ | 406 | 13 | MCI |
| | | APOLIPOPROTEIN | EWEGK | | | versus |
| | | B-100LIGHT | | | | AD |
| | | POLYPEPTIDE 9 | | | | |
| 55.31416531 | SP-047 | HEPARIN | NGNMAGISDQR | 144 | 14 | MCI |
| | | COFACTOR 2 | | | | versus |
| | | | | | | AD |
| 54.99881636 | SP-047 | KALLISTATIN | LGFTDLFSK | 407 | 15 | MCI |
| | | | | | | versus |
| | | | | | | AD |
| 54.98327299 | SP-047 | CLUSTERIN | TLLSNLEEAK | 408 | 16 | MCI |
| | | | | | | versus |
| | | | | | | AD |
| 54.7162131 | SP-047 | FIBRONECTIN | IGDTWSK | 409 | 17 | MCI |
| | | | | | | versus |
| | | | | | | AD |
| 54.1733967 | SP-047 | STROMAL | LPDSPALAK | 410 | 18 | MCI |
| | | INTERACTION | | | | versus |
| | | MOLECULE 1 | | | | AD |
| 53.66408803 | SP-047 | APOLIPOPROTEIN | MEEMGSR | 411 | 19 | MCI |
| | | E | | | | versus |
| | | | | | | AD |
| 53.51853847 | SP-047 | TYROSINE- | NYLGGFALSVAHGR | 412 | 20 | MCI |
| | | PROTEIN | | | | versus |
| | | KINASE SYK | | | | AD |
| 100 | SP-053 | PRO-GLUCAGON | HADGSFSDEMNTILDN | 413 | 1 | MCI |
| | | | LAAR | | | versus |
| | | | | | | AD |
| 39.13984367 | SP-053 | APOLIPOPROTEIN | MEEMGSR | 411 | 2 | MCI |
| | | E | | | | versus |
| | | | | | | AD |
| 38.93831585 | SP-053 | APOLIPOPROTEIN | LVQYRGEVQAMLGQST | 414 | 3 | MCI |
| | | E | EELRVR | | | versus |
| | | | | | | AD |
| 38.49445717 | SP-053 | FIBRONECTIN | PGVVYEGQLISIQQYG | 415 | 4 | MCI |
| | | | HQEVTR | | | versus |
| | | | | | | AD |
| 38.41317786 | SP-053 | TALIN-1 | ALDGAFTEENR | 416 | 5 | MCI |
| | | | | | | versus |
| | | | | | | AD |
| 38.40936649 | SP-053 | KININOGEN-1 | AATGECTATVGKR | 417 | 6 | MCI |
| | | | | | | versus |
| | | | | | | AD |
| 35.81372224 | SP-053 | ENDOPLASMIN | TVLDLAVVLFETATLR | 418 | 7 | MCI |
| | | | | | | versus |
| | | | | | | AD |
| 35.65432917 | SP-053 | FIBRONECTIN | VTIMWTPPESAVTGYR | 419 | 8 | MCI |
| | | | VDVIPVNLPGEHGQR | | | versus |
| | | | | | | AD |
| 34.88669041 | SP-053 | IMMUNOGLOBULIN | ASSLESGVPSR | 420 | 9 | MCI |
| | | KAPPA | | | | versus |
| | | VARIABLE 1-5 | | | | AD |
| 33.8696803 | SP-053 | APOLIPOPROTEIN | RVEPYGENFNK | 421 | 10 | MCI |
| | | A-IV | | | | versus |
| | | | | | | AD |
| 33.83925706 | SP-053 | TALIN-1 | ALCGFTEAAAQAAYLV | 422 | 11 | MCI |
| | | | GVSDPNSQAGQQGLVE | | | versus |
| | | | PTQFAR | | | AD |
| 33.11977563 | SP-053 | FIBRONECTIN | FGFCPMAAHEEICTTN | 280 | 12 | MCI |
| | | | EGVMYR | | | versus |
| | | | | | | AD |
| 32.47684448 | SP-053 | SECRETOGRANIN-1 | LLRDPADASEAHESSS | 423 | 13 | MCI |
| | | | R | | | versus |
| | | | | | | AD |
| 32.11139386 | SP-053 | NIDOGEN-1 | FYDRSDIDAVYVTTNG | 424 | 14 | MCI |
| | | | IIATSEPPAK | | | versus |
| | | | | | | AD |
| 31.66439705 | SP-053 | COMPLEMENT C3 | IWDVVEK | 173 | 15 | MCI |
| | | | | | | versus |
| | | | | | | AD |
| 31.34439117 | SP-053 | ELONGATION | QTVAVGVIK | 425 | 16 | MCI |
| | | FACTOR 1- | | | | versus |
| | | ALPHA 1 | | | | AD |
| 31.25913767 | SP-053 | CERULOPLASMIN | MYYSAVDPTK | 426 | 17 | MCI |
| | | | | | | versus |
| | | | | | | AD |
| 30.80754753 | SP-053 | VON WILLEBRAND | QTMVDSSCR | 427 | 18 | MCI |
| | | FACTOR | | | | versus |
| | | | | | | AD |
| 30.75555305 | SP-053 | Tubulin | ALTVPELTQQVFDAK | 428 | 19 | MCI |
| | | beta chain | | | | versus |
| | | | | | | AD |
| 30.48834788 | SP-053 | MYOSIN | GIISALLVPPETEEAK | 429 | 20 | MCI |
| | | APOLIPOPROTEIN | | | | versus |
| | | B-100LIGHT | | | | AD |
| | | POLYPEPTIDE 9 | | | | |
| 100 | SP-373 | APOLIPOPROTEIN | QLTPYAQR | 430 | 1 | MCI |
| | | A-IV | | | | versus |
| | | | | | | AD |
| 90.50230884 | SP-373 | FIBRONECTIN | FLATTPNSLLVSWQPP | 355 | 2 | MCI |
| | | | R | | | versus |
| | | | | | | AD |
| 79.33832918 | SP-373 | FIBRONECTIN | DLQFVEVTDVK | 431 | 3 | MCI |
| | | | | | | versus |
| | | | | | | AD |
| 69.46416233 | SP-373 | APOLIPOPROTEIN | IDQNVEELK | 383 | 4 | MCI |
| | | A-IV | | | | versus |
| | | | | | | AD |
| 66.51350303 | SP-373 | APOLIPOPROTEIN | ISASAEELR | 432 | 5 | MCI |
| | | A-IV | | | | versus |
| | | | | | | AD |
| 65.70012007 | SP-373 | FIBRONECTIN | VPGTSTSATLTGLTR | 433 | 6 | MCI |
| | | | | | | versus |
| | | | | | | AD |
| 62.87735334 | SP-373 | Elongation | EHALLAYTLGVK | 434 | 7 | MCI |
| | | factor 1- | | | | versus |
| | | alpha 1 | | | | AD |
| 62.69969224 | SP-373 | APOLIPOPROTEIN | VLRENADSLQASLRPH | 307 | 8 | MCI |
| | | A-IV | ADELK | | | versus |
| | | | | | | AD |
| 60.37086685 | SP-373 | FIBRONECTIN | EYLGAICSCTCFGGQR | 435 | 9 | MCI |
| | | | | | | versus |
| | | | | | | AD |
| 58.22512618 | SP-373 | MULTIMERIN-1 | MTDQVNYQAMK | 436 | 10 | MCI |
| | | | | | | versus |
| | | | | | | AD |
| 56.59472254 | SP-373 | EUKARYOTIC | TGFQAVTGK | 437 | 11 | MCI |
| | | TRANSLATION | | | | versus |
| | | INITIATION | | | | AD |
| | | FACTOR 2 | | | | |
| | | SUBUNIT 2 | | | | |
| 56.58917515 | SP-373 | TRANSTHYRETIN | ALGISPFHEHAEVVFT | 7 | 12 | MCI |
| | | | ANDSGPR | | | versus |
| | | | | | | AD |
| 54.71635474 | SP-373 | L-LACTATE | DYSVTANSK | 438 | 13 | MCI |
| | | DEHYDROGENASE | | | | versus |
| | | B CHAIN | | | | AD |
| 52.68073883 | SP-373 | FIBRONECTIN | VDVIPVNLPGEHGQR | 439 | 14 | MCI |
| | | | | | | versus |
| | | | | | | AD |
| 52.27811245 | SP-373 | ADENYLYL | EPAVLELEGK | 440 | 15 | MCI |
| | | CYCLASE- | | | | versus |
| | | ASSOCIATED | | | | AD |
| | | PROTEIN 1 | | | | |
| 51.53679945 | SP-373 | LACTO- | CLAENAGDVAFVK | 441 | 16 | MCI |
| | | TRANSFERRIN | | | | versus |
| | | | | | | AD |
| 50.47571929 | SP-373 | MYOSIN-9 | LDPHLVLDQLR | 442 | 17 | MCI |
| | | | | | | versus |
| | | | | | | AD |
| 50.13751967 | SP-373 | FIBRONECTIN | YSFCTDHTVLVQTR | 443 | 18 | MCI |
| | | | | | | versus |
| | | | | | | AD |
| 49.52838551 | SP-373 | HEPARANASE | FLILLGSPK | 444 | 19 | MCI |
| | | | | | | versus |
| | | | | | | AD |
| 48.809654 | SP-373 | FIBRONECTIN | TEIDKPSQMQVTDVQD | 445 | 20 | MCI |
| | | | NSISVK | | | versus |
| | | | | | | AD |
| 100 | SP-089 | SEROTRANSFERRIN | LCMGSGLNLCEPNNK | 446 | 1 | MCI |
| | | | | | | versus |
| | | | | | | AD |
| 96.33619097 | SP-089 | APOLIPOPROTEIN | SELTQQLNALFQDK | 447 | 2 | MCI |
| | | A-IV | | | | versus |
| | | | | | | AD |
| 96.06571876 | SP-089 | COMPLEMENT C3 | IFTVNHK | 448 | 3 | MCI |
| | | | | | | versus |
| | | | | | | AD |
| 95.33775425 | SP-089 | COMPLEMENT C3 | DFDFVPPVVR | 244 | 4 | MCI |
| | | | | | | versus |
| | | | | | | AD |
| 93.19556313 | SP-089 | MYELOPEROXIDASE | NQADCIPFFR | 449 | 5 | MCI |
| | | | | | | versus |
| | | | | | | AD |
| 87.60859264 | SP-089 | CHORDIN-LIKE | VLVHTSVSPSPDNLR | 450 | 6 | MCI |
| | | PROTEIN 2 | | | | versus |
| | | | | | | AD |
| 87.45474488 | SP-089 | COMPLEMENT C3 | TGLQEVEVK | 292 | 7 | MCI |
| | | | | | | versus |
| | | | | | | AD |
| 84.42787022 | SP-089 | FIBRONECTIN | VFAVSHGR | 451 | 8 | MCI |
| | | | | | | versus |
| | | | | | | AD |
| 83.7712725 | SP-089 | ZYMOGEN | VWSDYVGGR | 452 | 9 | MCI |
| | | GRANULE | | | | versus |
| | | MEMBRANE | | | | AD |
| | | PROTEIN 16 | | | | |
| 81.6400209 | SP-089 | FIBRONECTIN | TFYSCTTEGR | 306 | 10 | MCI |
| | | | | | | versus |
| | | | | | | AD |
| 81.26271798 | SP-089 | COAGULATION | VYSGILNQSEIK | 453 | 11 | MCI |
| | | FACTOR XI | | | | versus |
| | | | | | | AD |
| 80.77474453 | SP-089 | FILAMIN-A | IPEISIQDMTAQVTSP | 454 | 12 | MCI |
| | | | SGK | | | versus |
| | | | | | | AD |
| 79.95970675 | SP-089 | LIM AND SH3 | GFSVVADTPELQR | 455 | 13 | MCI |
| | | DOMAIN | | | | versus |
| | | PROTEIN 1 | | | | AD |
| 79.95205166 | SP-089 | APOLIPOPROTEIN | EAVEHLQK | 456 | 14 | MCI |
| | | A-IV | | | | versus |
| | | | | | | AD |
| 79.78863061 | SP-089 | ALPHA-1- | ECLQTCR | 457 | 15 | MCI |
| | | MICROGLOBULIN/ | | | | versus |
| | | BIKUNIN | | | | AD |
| | | PRECURSOR | | | | |
| 79.32702503 | SP-089 | BIFUNCTIONAL | THVADFAPEVAWVTR | 89 | 16 | MCI |
| | | GLUTAMATE/ | | | | versus |
| | | PROLINE-TRNA | | | | AD |
| | | LIGASE | | | | |
| 77.61892591 | SP-089 | PHOSPHOLIPID | FLEQELETITIPDLR | 458 | 17 | MCI |
| | | TRANSFER | | | | versus |
| | | PROTEIN | | | | AD |
| 77.50420047 | SP-089 | ELONGATION | EHALLAYTLGVK | 434 | 18 | MCI |
| | | FACTOR 1- | | | | versus |
| | | ALPHA 1 | | | | AD |
| 77.23635687 | SP-089 | PLASMINOGEN | FSPATHPSEGLEENYC | 459 | 19 | MCI |
| | | | R | | | versus |
| | | | | | | AD |
| 76.85992801 | SP-089 | Immunoglobulin | GLEWLGR | 460 | 20 | MCI |
| | | heavy variable | | | | versus |
| | | 6-1 | | | | AD |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method, comprising:
obtaining a data set comprising protein or peptide information from biomolecule coronas that correspond to physiochemically distinct particles incubated with a biofluid sample from a subject; and
using a classifier to identify the biofluid sample being indicative of a biological state comprising healthy state, a neurocognitive disorder, or a neurodegenerative disease, in the subject, based on the data set.

2. The method of claim 1, wherein the neurocognitive disorder comprises a mild cognitive impairment (MCI).

3. The method of claim 1, wherein the neurodegenerative disease comprises Alzheimer's disease (AD).

4. The method of claim 3, wherein the protein information comprises expression information for a protein provided in TABLE 8.

5. (canceled)

6. The method of claim 1, wherein obtaining a data set comprises contacting the biofluid sample with the physiochemically distinct particles to form the biomolecule coronas.

7. The method of claim 1, wherein the physiochemically distinct particles comprise lipid particles, metal particles, silica particles, or polymer particles.

8. The method of claim 1, wherein the physiochemically distinct particles comprise polystyrene particles, magnetizable particles, dextran particles, silica particles, dimethylamine particles, carboxylate particles, amino particles, benzoic acid particles, or agglutinin particles.

9. The method of claim 1, wherein obtaining a data set comprises detecting proteins of the biomolecule coronas by mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof.

10. The method of claim 1, wherein obtaining a data set comprises detecting the proteins of the biomolecule coronas by mass spectrometry.

11. The method of claim 1, wherein obtaining a data set comprises measuring a readout indicative of the presence, absence or amount of proteins of the biomolecule coronas.

12. The method of claim 1, wherein the method further comprises administering a neurocognitive disorder treatment or a neurodegenerative disease treatment to the subject based on the biological state.

13.-15. (canceled)

16. A method of evaluating a status of a biological state, comprising: measuring biomarkers in a biofluid sample from a subject suspected of having a neurocognitive disorder or a neurodegenerative disease to obtain biomarker measurements, wherein the biomarkers comprise one or more biomarkers selected from a table or figure included herein.

17.-18. (canceled)

19. The method of claim 16, wherein the biomarkers comprise two or more biomarkers selected from Table 11 for discriminating between the neurocognitive disorder and the neurodegenerative disease.

26. The method of claim 16, further comprising applying a classifier to the biomarker measurements.

27.-31. (canceled)

32. A method, comprising:
(a) assaying a biological sample from a subject to identify biomolecules;
(b) using a trained classifier to identify that the sample or the subject is positive or negative for Alzheimer's disease (AD) or mild cognitive impairment (MCI) based on the biomolecules identified in (a), wherein the trained classifier is trained using data from training samples comprising known healthy samples and known Alzheimer's disease (AD) or mild cognitive impairment (MCI) samples, and wherein the training samples were assayed using a plurality of particles having physicochemically distinct properties to yield the data.

33.-35. (canceled)

36. The method of claim 32, wherein the data comprises proteomic data identifying a presence or an absence of proteins in the training samples.

37.-39. (canceled)

40. The method of claim 32, wherein the plurality of particles having physicochemically distinct properties comprise two or more particles described herein.

41. The method of claim 32, wherein the assaying comprises performing mass spectrometry or ELISA, and wherein the biomolecules comprise protein.

42. The method of claim 32, wherein the assaying comprises targeted mass spectrometry.

43. The method of claim 32, wherein the trained classifier comprises a trained algorithm.
